(19) United States
(12) Patent Application Publication
(10) Pub. No.: US 2017/0092873 A1
(43) Pub. Date: Mar. 30, 2017

(54) COMPOSITION FOR ORGANIC OPTOELECTRONIC DEVICE, ORGANIC OPTOELECTRONIC DEVICE AND DISPLAY DEVICE

(71) Applicant: SAMSUNG SDI CO., LTD., Yongin-si (KR)

(72) Inventors: Giwook KANG, Suwon-si (KR); Younhwan KIM, Suwon-si (KR); Youngkwon KIM, Suwon-si (KR); Dongyeong KIM, Suwon-si (KR); Hun KIM, Suwon-si (KR); Jaejin OH, Suwon-si (KR); Pyeongseok CHO, Suwon-si (KR); Eun Sun YU, Suwon-si (KR)

(21) Appl. No.: 15/272,607

(22) Filed: Sep. 22, 2016

(30) Foreign Application Priority Data
Sep. 25, 2015 (KR) 10-2015-0136653

(51) Int. Cl. H01L 51/00; C09K 11/02; C07D 405/12; C07D 209/86; C07C 211/54; C09K 11/06; C07D 251/24

(52) U.S. Cl. CPC H01L 51/0067; C09K 11/06; H01L 51/0085; C09K 11/025; C07D 251/24; H01L 51/0054; C07D 209/86; H01L 51/0072; C07C 211/54; H01L 51/0059; H01L 51/0061; C07D 405/12; H01L 51/5016

(57) ABSTRACT

A composition for an organic optoelectronic device includes at least one first compound represented by Chemical Formula 1, at least one second compound of a compound represented by Chemical Formula 2, and a compound consisting of a combination of a moiety represented by Chemical Formula 3 and a moiety represented by Chemical Formula 4, and at least one third compound represented by Chemical Formula 5, an organic optoelectronic device includes the same, and a display device includes the organic optoelectronic device. Chemical Formulae 1 to 5 are the same as described in the detailed description.

### CROSS-REFERENCE TO RELATED APPLICATION

This application claims priority to and the benefit of Korean Patent Application No. 10-2015-0136653 filed in the Korean Intellectual Property Office on Sep. 25, 2015, the entire contents of which are incorporated herein by reference.

### BACKGROUND OF THE INVENTION

(a) Field of the Invention

An organic optoelectronic device and a display device are disclosed.

(b) Description of the Related Art

An organic optoelectronic device is a device that converts electrical energy into photoenergy, and vice versa.

An organic optoelectronic device may be classified as follows in accordance with its driving principles. One is an optoelectronic device where excitons are generated by photoenergy, separated into electrons and holes, and are transferred to different electrodes to generate electrical energy, and the other is a light emitting device where a voltage or a current is supplied to an electrode to generate photoenergy from electrical energy.

Examples of the organic optoelectronic device may be an organic photoelectric device, an organic light emitting diode, an organic solar cell, and an organic photo conductor drum.

Of these, an organic light emitting diode (OLED) has recently drawn attention due to an increase in demand for flat panel displays. The organic light emitting diode converts electrical energy into light by applying current to an organic light emitting material and has a structure in which an organic layer is interposed between an anode and a cathode.

Efficiency of an organic light emitting diode is considered to be one of the critical factors for realizing a long life-span full color display. Accordingly, much research on developing an organic light emitting diode having high efficiency by using a phosphorescent material has been made. The present disclosure is to provide an organic light emitting diode by using a phosphorescent material having high efficiency in order to solve this problem.

### SUMMARY OF THE INVENTION

An embodiment provides a composition for an organic optoelectronic device having high efficiency and long life-span.

Another embodiment provides an organic optoelectronic device including the composition for an organic optoelectronic device.

Yet another embodiment provides a display device including the organic optoelectronic device.

According to one embodiment, a composition for an organic optoelectronic device includes at least one first compound represented by Chemical Formula 1, at least one second compound of a compound represented by Chemical Formula 2, and a compound consisting of a combination of a moiety represented by Chemical Formula 3 and a moiety represented by Chemical Formula 4, and at least one third compound represented by Chemical Formula 5.

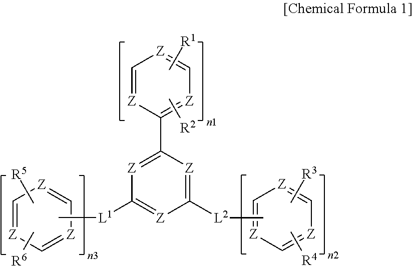

In Chemical Formula 1,

Z is independently N, C, or CR^{a},

at least one of Z is N,

R¹ to R⁶ and R^{a} are independently hydrogen, deuterium, a substituted or unsubstituted C1 to C10 alkyl group, a substituted or unsubstituted C6 to C30 aryl group, a substituted or unsubstituted C2 to C30 heteroaryl group, or a combination thereof,

R¹ to R⁶ and R^{a} are independently present or adjacent groups are linked to each other to provide a ring,

L¹ and L² are independently a single bond, a substituted or unsubstituted C6 to C30 arylene group, a substituted or unsubstituted C2 to C30 heteroarylene group, or a combination thereof,

n1 is 1,

n2 and n3 are independently an integer of 0 or 1, and

1≦n2+n3≦2;

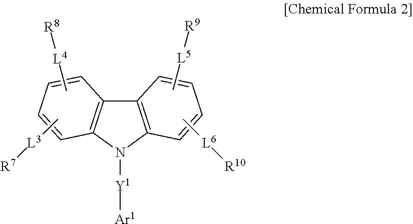

wherein, in Chemical Formula 2,

L³ to L⁶ and Y¹ are independently a single bond, a substituted or unsubstituted C6 to C30 arylene group, a substituted or unsubstituted C2 to C30 heteroarylene group, or a combination thereof,

Ar¹ is a substituted or unsubstituted C6 to C30 aryl group, a substituted or unsubstituted C2 to C30 heterocyclic group, or a combination thereof,

R⁷ to R¹⁰ are independently hydrogen, deuterium, a substituted or unsubstituted C1 to C20 alkyl group, a substituted or unsubstituted C6 to C50 aryl group, a substituted or unsubstituted C2 to C50 heterocyclic group, or a combination thereof, and

at least one of R⁷ to R¹⁰ and Ar¹ includes a substituted or unsubstituted triphenylene group or a substituted or unsubstituted carbazole group,

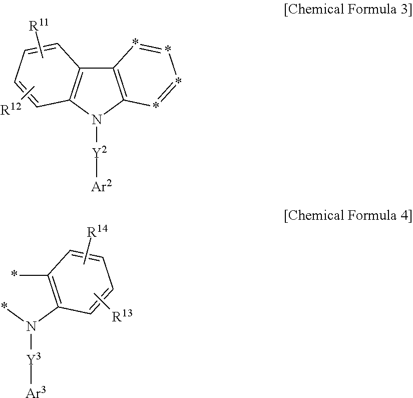

wherein, in Chemical Formulae 3 and 4,

Y² and Y³ are independently a single bond, a substituted or unsubstituted C6 to C30 arylene group, a substituted or unsubstituted C2 to C30 heteroarylene group, or a combination thereof,

Ar² and Ar³ are independently a substituted or unsubstituted C6 to C30 aryl group, a substituted or unsubstituted C2 to C30 heterocyclic group, or a combination thereof,

R¹¹ to R¹⁴ are independently hydrogen, deuterium, a substituted or unsubstituted C1 to C20 alkyl group, a substituted or unsubstituted C6 to C50 aryl group, a substituted or unsubstituted C2 to C50 heterocyclic group, or a combination thereof,

two adjacent *'s of Chemical Formula 3 are linked to two *'s of Chemical Formula 4 to provide a fused ring, and in Chemical Formula 3, *'s that do not provide a fused ring are independently CR^{c}, and

R^{c} is hydrogen, deuterium, a substituted or unsubstituted C1 to C10 alkyl group, a substituted or unsubstituted C6 to C12 aryl group, a substituted or unsubstituted C2 to C12 heterocyclic group, or a combination thereof;

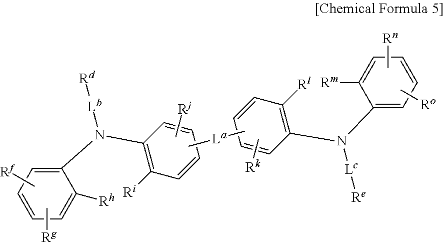

wherein, in Chemical Formula 5,

R^{d} and R^{e} are independently, a substituted or unsubstituted C6 to C30 aryl group, a substituted or unsubstituted C2 to C30 heterocyclic group, or a combination thereof

R^{f} to R^{o} are independently hydrogen, deuterium, a substituted or unsubstituted C1 to C30 alkyl group, a substituted or unsubstituted C3 to C30 cycloalkyl group, a substituted or unsubstituted C6 to C30 aryl group, a substituted or unsubstituted C2 to C30 heterocyclic group, a substituted or unsubstituted C6 to C30 arylamine group, a substituted or unsubstituted C1 to C30 alkoxy group, a substituted or unsubstituted C3 to C40 silyl group, a substituted or unsubstituted C3 to C40 silyloxy group, a substituted or unsubstituted C1 to C30 alkylthiol group, a substituted or unsubstituted C6 to C30 arylthiol group, a halogen, a halogen-containing group, a cyano group, a hydroxyl group, an amino group, a nitro group, or a combination thereof,

R^{h}, Rⁱ, R^{l}, and R^{m} are independently present or are linked to each other to provide a ring, provided that when R^{h} and Rⁱ are linked to each other to provide a ring, R^{l} and R^{m} are not linked to each other, and when R^{l} and R^{m} are linked to each other to provide a ring, R^{h} and Rⁱ are not linked to each other, and

L^{a} to L^{c} are independently a single bond, a substituted or unsubstituted C6 to C30 arylene group, a substituted or unsubstituted C2 to C30 heteroarylene group, or a combination thereof,

wherein “substituted” of Chemical Formulae 1 to 5 refers to replacement of at least one hydrogen by deuterium, a halogen, a hydroxyl group, a C1 to C40 silyl group, a C1 to C30 alkyl group, a C3 to C30 cycloalkyl group, a C2 to C30 heterocycloalkyl group, a C6 to C30 aryl group, a C2 to C30 heterocyclic group, a C1 to C20 alkoxy group, a C1 to C10 trifluoroalkyl group, or a cyano group.

According to another embodiment, an organic optoelectronic device including the composition for an organic optoelectronic device is provided.

According to another embodiment, a display device including the organic optoelectronic device is provided.

An organic optoelectronic device having high efficiency and long life-span may be realized.

### BRIEF DESCRIPTION OF THE DRAWINGS

FIGS. 1 and 2 are schematic cross-sectional views showing organic optoelectronic devices according to example embodiments.

### DETAILED DESCRIPTION

Hereinafter, embodiments of the present disclosure are described in detail. However, these embodiments are exemplary, the present invention is not limited thereto and the present invention is defined by the scope of claims.

In the present specification, when a definition is not otherwise provided, the term “substituted” refers to replacement of at least one hydrogen of a substituent or a compound by deuterium, a halogen, a hydroxyl group, a C1 to C40 silyl group, a C1 to C30 alkyl group, a C3 to C30 cycloalkyl group, a C2 to C30 heterocycloalkyl group, a C6 to C30 aryl group, a C2 to C30 heterocyclic group, a C1 to C20 alkoxy group, a C1 to C10 trifluoroalkyl group, or a cyano group.

In an embodiment of the invention, the term “substituted” may refer to replacement of at least one hydrogen of a substituent or a compound by deuterium, a C1 to C10 alkyl group or a C6 to C30 aryl group.

In addition, two adjacent substituents of the substituted C1 to C40 silyl group, the C1 to C30 alkyl group, the C3 to C30 cycloalkyl group, the C2 to C30 heterocycloalkyl group, the C6 to C30 aryl group, the C2 to C30 heterocyclic group, or the C1 to C20 alkoxy group may be fused to form a ring. For example, the substituted C6 to C30 aryl group may be fused with another adjacent substituted C6 to C30 aryl group to form a substituted or unsubstituted fluorene ring.

In the present specification, when specific definition is not otherwise provided, “hetero” refers to one including 1 to 3 hetero atoms selected from N, O, S, P, and Si, and remaining carbons in one compound or substituent.

In the present specification, when a definition is not otherwise provided, “alkyl group” refers to an aliphatic hydrocarbon group. The alkyl group may be “a saturated alkyl group” without any double bond or triple bond.

The alkyl group may be a C1 to C30 alkyl group. More specifically, the alkyl group may be a C1 to C20 alkyl group or a C I to C10 alkyl group. For example, a C1 to C4 alkyl group may have 1 to 4 carbon atoms in alkyl chain which may be selected from methyl, ethyl, propyl, iso-propyl, n-butyl, iso-butyl, sec-butyl, and t-butyl.

Specific examples of the alkyl group may be a methyl group, an ethyl group, a propyl group, an isopropyl group, a butyl group, an isobutyl group, a t-butyl group, a pentyl group, a hexyl group, a cyclopropyl group, a cyclobutyl group, a cyclopentyl group, a cyclohexyl group, and the like.

In the present specification, “aryl group” refers to a group including at least one hydrocarbon aromatic moiety, and

all the elements of the hydrocarbon aromatic moiety have p-orbitals which form conjugation, for example a phenyl group, a naphthyl group, and the like,

two or more hydrocarbon aromatic moieties may be linked by a sigma bond and may be, for example a biphenyl group, a terphenyl group, a quarterphenyl group, and the like, and

two or more hydrocarbon aromatic moieties are fused directly or indirectly to provide a non-aromatic fused ring. For example, it may be a fluorenyl group.

The aryl group may include a monocyclic, polycyclic or fused ring polycyclic (i.e., rings sharing adjacent pairs of carbon atoms) functional group.

In the present specification, “heterocyclic group” is a generic concept of a heteroaryl group, and may include at least one hetero atom selected from N, O, S, P, and Si instead of carbon (C) in a cyclic compound such as aryl group, a cycloalkyl group, a fused ring thereof, or a combination thereof. When the heterocyclic group is a fused ring, the entire ring or each ring of the heterocyclic group may include one or more heteroatoms.

For example, a “heteroaryl group” may refer to aryl group including at least one hetero atom selected from N, O, S, P, and Si instead of carbon (C). Two or more heteroaryl groups are linked by a sigma bond directly, or when the C2 to C60 heteroaryl group includes two or more rings, the two or more rings may be fused. When the heteroaryl group is a fused ring, each ring may include 1 to 3 hetero atoms.

Specific examples of the heteroaryl group may be a pyridinyl group, a pyrimidinyl group, a pyrazinyl group, a pyridazinyl group, a triazinyl group, a quinolinyl group, an isoquinolinyl group, and the like.

More specifically, the substituted or unsubstituted C6 to C30 aryl group and/or the substituted or unsubstituted C2 to C30 heterocyclic group may be a substituted or unsubstituted phenyl group, a substituted or unsubstituted naphthyl group, a substituted or unsubstituted anthracenyl group, a substituted or unsubstituted phenanthryl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted chrysenyl group, a substituted or unsubstituted triphenylenyl group, a substituted or unsubstituted perylenyl group, a substituted or unsubstituted fluorenyl group, a substituted or unsubstituted spirofluorenyl group, a substituted or unsubstituted indenyl group, a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridinyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzthiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted carbazolyl group, a substituted or unsubstituted dibenzofuranyl group, or a substituted or unsubstituted dibenzothiophenyl group, a substituted or unsubstituted benzothiophenepyrimidinyl group, a substituted or unsubstituted benzothiophenepyridyl group, a substituted or unsubstituted benzofuranpyrimidinyl group, a substituted or unsubstituted benzofuranpyridyl group, a substituted or unsubstituted benzofuranpyrrolyl group, a substituted or unsubstituted benzothiophenepyrrolyl group, a substituted or unsubstituted benzothiophenethiazolyl group, a substituted or unsubstituted benzofuranthiazolyl group, a substituted or unsubstituted thiazoloquinolinyl group, a substituted or unsubstituted oxazoloquinolinyl group, or a combination thereof, but are not limited thereto.

In the present specification, a single bond refers to a direct bond not by carbon or a hetero atom except carbon, and specifically the meaning that L is a single bond means that a substituent linked to L directly bonds with a central core. That is, in the present specification, the single bond does not refer to methylene that is bonded via carbon.

In the specification, hole characteristics refer to an ability to donate an electron to form a hole when an electric field is applied and that a hole formed in the anode may be easily injected into the emission layer, and a hole formed in an emission layer may be easily transported into an anode and transported in the emission layer due to conductive characteristics according to a highest occupied molecular orbital (HOMO) level.

In addition, electron characteristics refer to an ability to accept an electron when an electric field is applied and that an electron formed in a cathode may be easily injected into the emission layer, and an electron formed in an emission layer may be easily transported into a cathode and transported in the emission layer due to conductive characteristics according to a lowest unoccupied molecular orbital (LUMO) level.

Hereinafter, a composition for an organic optoelectronic device according to an embodiment is described.

According to one embodiment, a composition for an organic optoelectronic device includes at least one first compound represented by Chemical Formula 1, at least one second compound of a compound represented by Chemical Formula 2, and a compound consisting of a combination of a moiety represented by Chemical Formula 3 and a moiety represented by Chemical Formula 4, and at least one third compound of a compound represented by Chemical Formula 5.

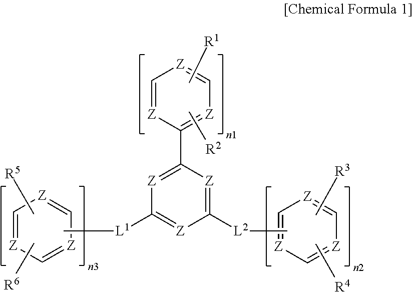

In Chemical Formula 1, Z is independently N, C, or CR^{a}, at least one of Z is N, R¹ to R⁶ and R^{a} are independently hydrogen, deuterium, a substituted or unsubstituted C1 to C10 alkyl group, a substituted or unsubstituted C6 to C30 aryl group, a substituted or unsubstituted C2 to C30 heteroaryl group, or a combination thereof, R¹ to R⁶ and R^{a} are independently present or adjacent groups are linked to each other to provide a ring, L¹ and L² are independently a single bond, a substituted or unsubstituted C6 to C30 arylene group, a substituted or unsubstituted C2 to C30 heteroarylene group, or a combination thereof, n1 is 1, n2 and n3 are independently an integer of 0 or 1, and 1≦n2+n3≦2;

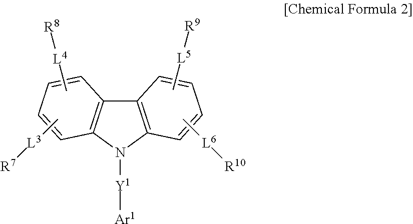

wherein, in Chemical Formula 2, L³ to L⁶ and Y¹ are independently a single bond, a substituted or unsubstituted C6 to C30 arylene group, a substituted or unsubstituted C2 to C30 heteroarylene group, or a combination thereof, Ar¹ is a substituted or unsubstituted C6 to C30 aryl group, a substituted or unsubstituted C2 to C30 heterocyclic group, or a combination thereof, R⁷ to R¹⁰ are independently hydrogen, deuterium, a substituted or unsubstituted C1 to C20 alkyl group, a substituted or unsubstituted C6 to C50 aryl group, a substituted or unsubstituted C2 to C50 heterocyclic group, or a combination thereof, and at least one of R⁷ to R¹⁰ and Ar¹ includes a substituted or unsubstituted triphenylene group or a substituted or unsubstituted carbazole group,

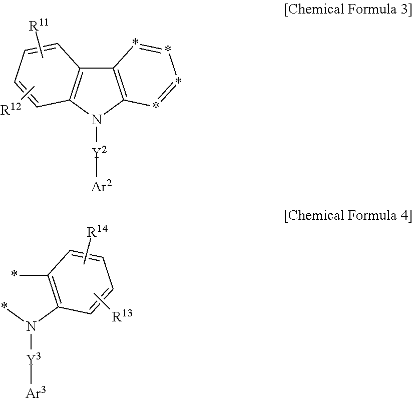

wherein, in Chemical Formulae 3 and 4, Y² and Y³ are independently a single bond, a substituted or unsubstituted C6 to C30 arylene group, a substituted or unsubstituted C2 to C30 heteroarylene group, or a combination thereof Ar² and Ar³ are independently a substituted or unsubstituted C6 to C30 aryl group, a substituted or unsubstituted C2 to C30 heterocyclic group, or a combination thereof, R¹¹ to R¹⁴ are independently hydrogen, deuterium, a substituted or unsubstituted C1 to C20 alkyl group, a substituted or unsubstituted C6 to C50 aryl group, a substituted or unsubstituted C2 to C50 heterocyclic group, or a combination thereof two adjacent *'s of Chemical Formula 3 are linked to two *'s of Chemical Formula 4 to provide a fused ring, and in Chemical Formula 3, *'s that do not provide a fused ring are independently CR^{c}, and R^{c} is hydrogen, deuterium, a substituted or unsubstituted C1 to C10 alkyl group, a substituted or unsubstituted C6 to C12 aryl group, a substituted or unsubstituted C2 to C12 heterocyclic group, or a combination thereof;

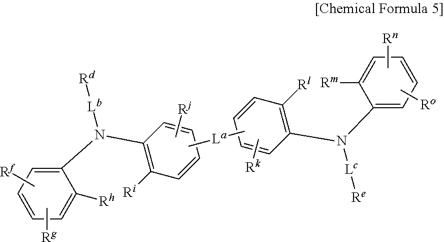

wherein, in Chemical Formula 5,

R^{d} and R^{e} are independently, a substituted or unsubstituted C6 to C30 aryl group, a substituted or unsubstituted C2 to C30 heterocyclic group, or a combination thereof

R^{f} to R^{o} are independently hydrogen, deuterium, a substituted or unsubstituted C1 to C30 alkyl group, a substituted or unsubstituted C3 to C30 cycloalkyl group, a substituted or unsubstituted C6 to C30 aryl group, a substituted or unsubstituted C2 to C30 heterocyclic group, a substituted or unsubstituted C6 to C30 arylamine group, a substituted or unsubstituted C1 to C30 alkoxy group, a substituted or unsubstituted C3 to C40 silyl group, a substituted or unsubstituted C3 to C40 silyloxy group, a substituted or unsubstituted C1 to C30 alkylthiol group, a substituted or unsubstituted C6 to C30 arylthiol group, a halogen, a halogen-containing group, a cyano group, a hydroxyl group, an amino group, a nitro group, or a combination thereof

R^{h}, Rⁱ, R^{l}, and R^{m} are independently present or are linked to each other to provide a ring, provided that when R^{h} and Rⁱ are linked to each other to provide a ring. R^{l} and R^{m} are not linked to each other, and when R^{l} and R^{m} are linked to each other to provide a ring, R^{h} and Rⁱ are not linked to each other, and

L^{a} to L^{c} are independently a single bond, a substituted or unsubstituted C6 to C30 arylene group, a substituted or unsubstituted C2 to C30 heteroarylene group, or a combination thereof,

wherein “substituted” of Chemical Formulae 1 to 5 refers to replacement of at least one hydrogen by deuterium, a halogen, a hydroxyl group, a C1 to C40 silyl group, a C1 to C30 alkyl group, a C3 to C30 cycloalkyl group, a C2 to C30 heterocycloalkyl group, a C6 to C30 aryl group, a C2 to C30 heterocyclic group, a C1 to C20 alkoxy group, a C1 to C10 trifluoroalkyl group, or a cyano group.

The first compound includes a ring containing at least one nitrogen and thus may have a structure of easily accepting elections when an electric field is applied thereto and thus bipolar characteristics in which electron characteristics are relatively strong as the injection amount of the electrons is increased. The second compound includes a carbazole moiety and thus may have bipolar characteristics in which hole characteristics are relatively strong.

When the first and second compounds are used together for an emission layer, charge mobility and stability may be increased, and thus luminous efficiency and life-span characteristics may be improved.

A conventional emission layer including the first compound and the second compound shows remarkably decreased hole transport capability due to a trap phenomenon caused by a HOMO energy level difference of a host from that of a dopant and increases a driving voltage of an organic optoelectronic device because of an injection wall by a HOMO energy level of a host relative to a hole transport layer.

Therefore, the third compound having a high HOMO energy level than the second compound and improved hole injection and hole transport capability is added and thus the trap phenomenon between a dopant and a host is decreased or minimized and an injection wall of a hole between hole transport layer and the emission layer is lowered and a driving voltage is remarkably reduced, resulting in improvement of luminous efficiency performance of a device.

L¹ and L² of Chemical Formula 1 according to an embodiment of the present invention may independently be a single bond, a substituted or unsubstituted C6 to C30 arylene group, or a substituted or unsubstituted C2 to C30 heteroarylene group as described above, and

specifically a substituted or unsubstituted C6 to C30 arylene group. For example, they may be a single bond, a substituted or unsubstituted phenylene group, a substituted or unsubstituted biphenylene group, a substituted or unsubstituted terphenylene group, a substituted or unsubstituted quaterphenylene group, a substituted or unsubstituted naphthylene group, a substituted or unsubstituted anthracenylene group, a substituted or unsubstituted triphenylenyl group, or a substituted or unsubstituted phenanthrenylene group.

Specific structures of the linking group are the same as Group 2 in the present specification.

R¹ and R² of Chemical Formula 1 according to an embodiment may independently be hydrogen, deuterium, a substituted or unsubstituted C1 to C10 alkyl group, a substituted or unsubstituted C6 to C30 aryl group, a substituted or unsubstituted C2 to C30 heteroaryl group, or a combination thereof as described above, and

specifically hydrogen, deuterium, or a substituted or unsubstituted C6 to C30 aryl group. For example, R¹ and R² may independently be selected from hydrogen, a substituted or unsubstituted phenyl group, or a substituted or unsubstituted biphenyl group, or a combination thereof, but is not limited thereto.

R³ to R⁶ of Chemical Formula 1 according to an embodiment may be hydrogen, deuterium, a substituted or unsubstituted C1 to C10 alkyl group, a substituted or unsubstituted C6 to C30 aryl group, or a substituted or unsubstituted C2 to C30 heteroaryl group, or a combination thereof as described above, and R³ to R⁶ are independently present or adjacent groups are linked to each other to provide a ring,

specifically they may be hydrogen, deuterium, a substituted or unsubstituted C6 to C30 aryl group, or a substituted or unsubstituted C2 to C30 heteroaryl group,

examples of the substituted or unsubstituted C6 to C30 aryl group may be a substituted or unsubstituted phenyl group, a substituted or unsubstituted biphenyl group, a substituted or unsubstituted quaterphenyl group, a substituted or unsubstituted naphthyl group, a substituted or unsubstituted phenanthrenyl group, or a combination thereof, and

examples of the substituted or unsubstituted C2 to C30 heteroaryl group may be a substituted or unsubstituted pyridinyl group, a substituted or unsubstituted pyrimidinyl group, a substituted or unsubstituted quinolinyl group, a substituted or unsubstituted isoquinolinyl group, a substituted or unsubstituted quinazolinyl group, a substituted or unsubstituted phenanthrolinyl group, or a combination thereof.

Adjacent groups of R³ to R⁶ may be linked to each other to form substituted or unsubstituted naphthyl group, a substituted or unsubstituted quinolinyl group, a substituted or unsubstituted isoquinolinyl group, a substituted or unsubstituted quinazolinyl group, a substituted or unsubstituted phenanthrolinyl group, a substituted or unsubstituted triphenylenyl group, and the like.

Specific examples of the R³ to R⁶ may be selected from hydrogen, or substituents of Group 1, but are not limited thereto.

For example, the first compound may be represented by one of Chemical Formula 1-I to Chemical Formula 1-III.

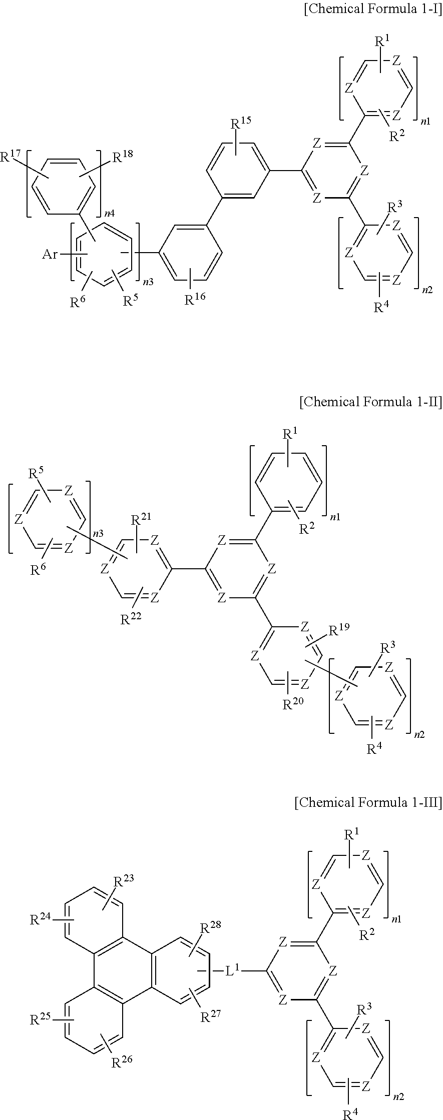

In Chemical Formulae 1-I to 1-III, Z, R¹ to R⁶, L¹, and n1 to n3 are the same as defined above,

R¹⁵ to R²⁸ are independently hydrogen, deuterium, a substituted or unsubstituted C1 to C10 alkyl group, a substituted or unsubstituted C6 to C12 aryl group, a substituted or unsubstituted C2 to C30 heteroaryl group, or a combination thereof,

Ar is a substituted or unsubstituted C6 to C30 aryl group, a substituted or unsubstituted C2 to C30 heterocyclic group, or a combination thereof

R¹ to R⁶, R^{a}, and R¹⁷ and R¹⁸ are independently present or adjacent groups are linked to each other to provide a ring,

n4 is an integer ranging from 0 to 2, and

“substituted” is the same as defined above.

According to one embodiment, Chemical Formula 1-I may be represented by one of Chemical Formulae 1-IA to 1-IC.

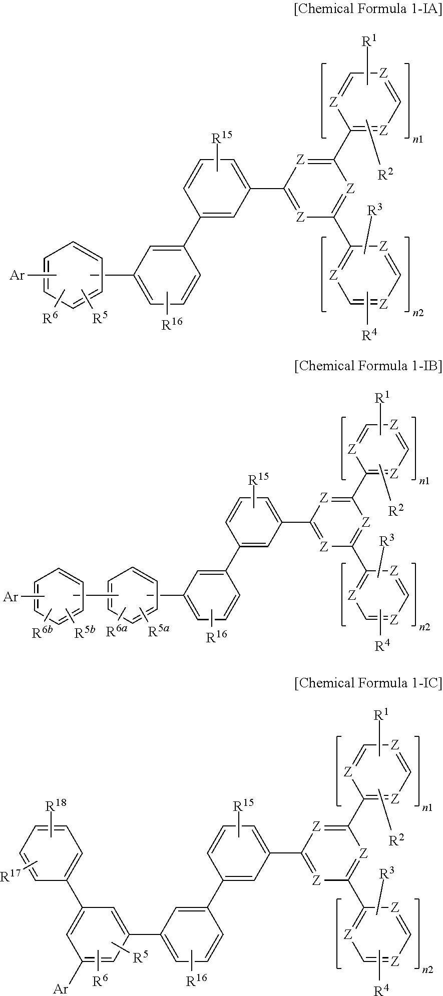

In Chemical Formulae 1-IA to 1-IC, Z, R¹ to R⁶ , R¹⁵ to R¹⁸, n1 and n2 are the same as above, R^{5a} and R^{5b}, R^{6a} and R^{6b}, and Ar are the same as the R⁵ and R⁶, and

“substituted” is the same as defined above.

Specifically, herein Ar is a substituted or unsubstituted C6 to C30 aryl group, a substituted or unsubstituted C2 to C30 heterocyclic group, or a combination thereof.

Specifically, Chemical Formula 1-IA may be represented by Chemical Formulae 1-I-1a or 1-I-2a according to a substitution position of Ar, but is not limited thereto.

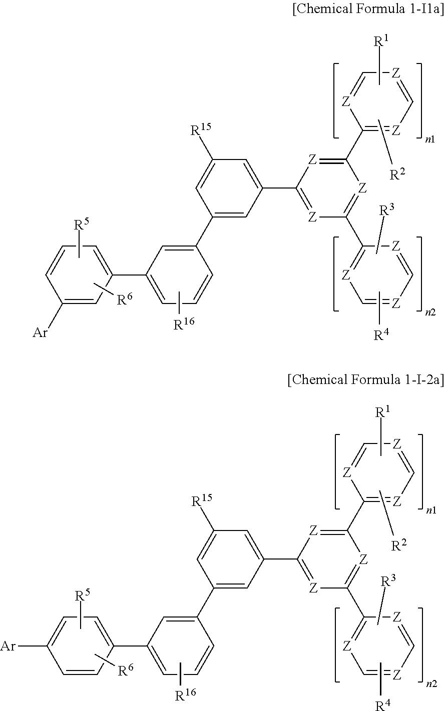

Specifically, Chemical Formula 1-IB may be represented by Chemical Formula 1-I-1b to 1-I-7b according to a linking group of an aryl group moiety and a substitution position of Ar, but is not limited thereto.

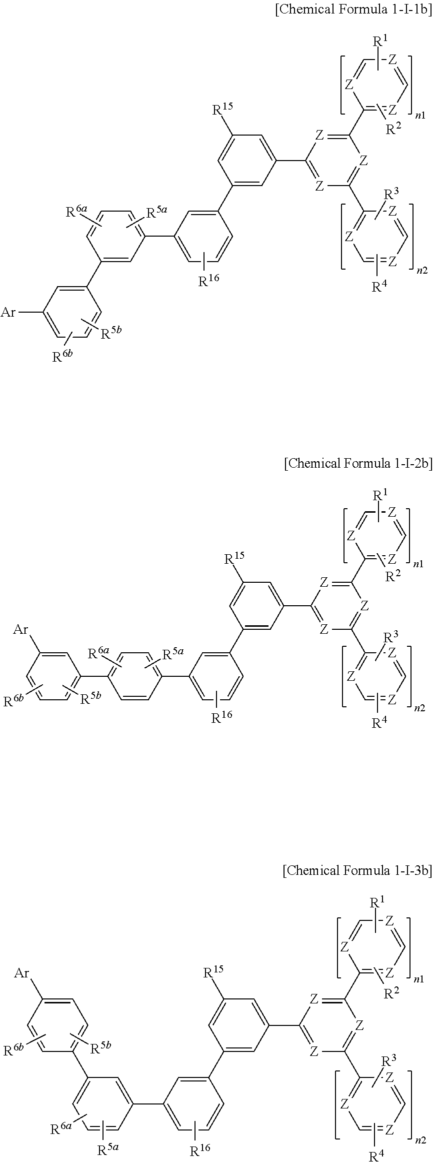

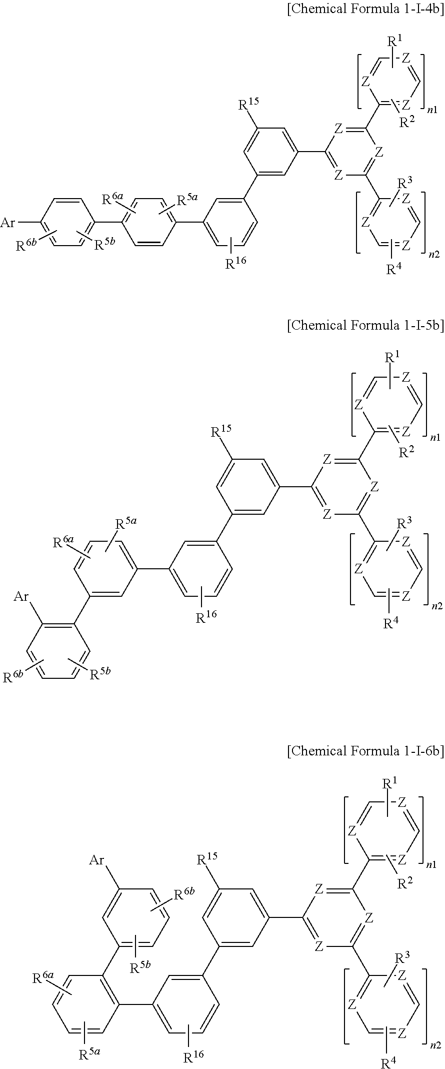

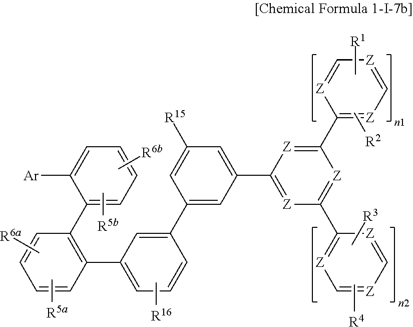

Specifically, Chemical Formula 1-IC may be represented by Chemical Formula 1-I-1c wherein a linking position of R¹⁵ is fixed, but is not limited thereto.

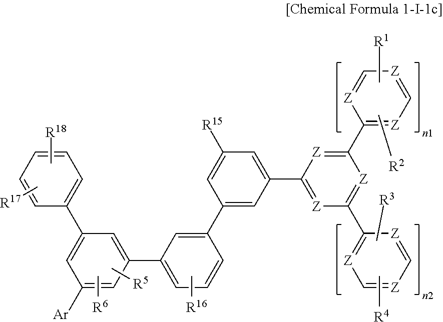

In Chemical Formulae 1-I-1a to 1-I-2a, 1-I-1b to 1-I-7b and 1-I-1 c, Z, R¹ to R⁶, R^{5a}, R^{5b}, R^{6a}, R^{6b}, R¹⁵ to R¹⁸, n1, n2, and Ar are the same as described above.

On the other hand, in Chemical Formula 1-I, n1 may be, for example an integer of 1, n2 is an integer of 1, and Chemical Formula 1-I may be represented by Chemical Formula 1-I-c or 1-I-d, but is not limited thereto.

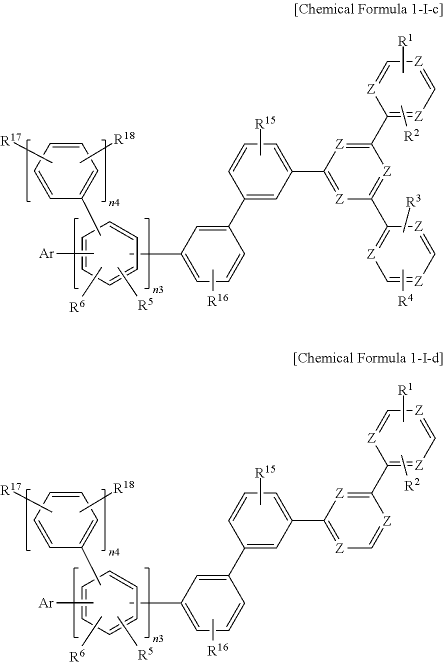

The Ar may be, for example, a substituted or unsubstituted phenyl group, a substituted or unsubstituted biphenyl group, a substituted or unsubstituted terphenyl group, a substituted or unsubstituted naphthyl group, a substituted or unsubstituted pyridinyl group, a substituted or unsubstituted pyrimidinyl group, a substituted or unsubstituted triazinyl group, a substituted or unsubstituted quinolinyl group, a substituted or unsubstituted isoquinolinyl group, a substituted or unsubstituted phenanthrolinyl group, or a substituted or unsubstituted quinazolinyl group.

More specifically, the Ar may be selected from substituted or unsubstituted groups of Group 1, but is not limited thereto.

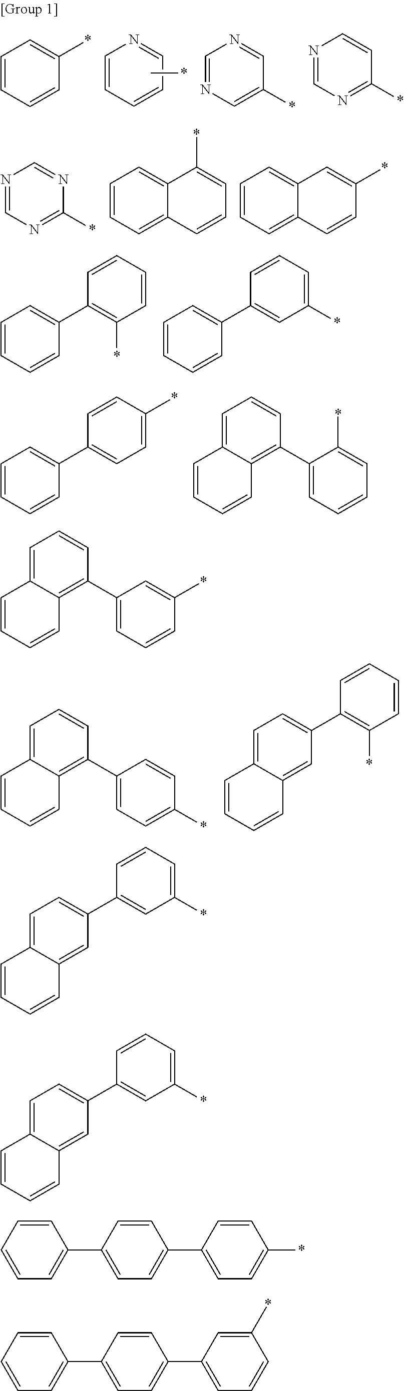

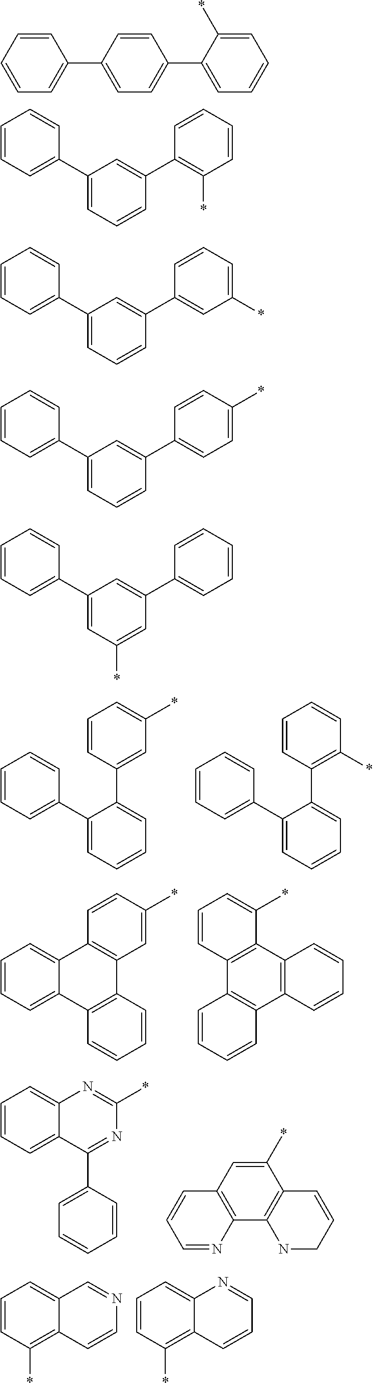

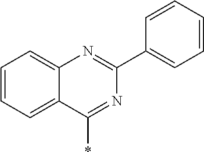 [SMILES: *c1nc(-c2ccccc2)nc2ccccc12]

In Group 1, * is a linking point.

Chemical Formula 1-I may be, for example, represented by one of Chemical Formulae 1-I-e to 1-I-n according to the position and the number of nitrogen, but is not limited thereto.

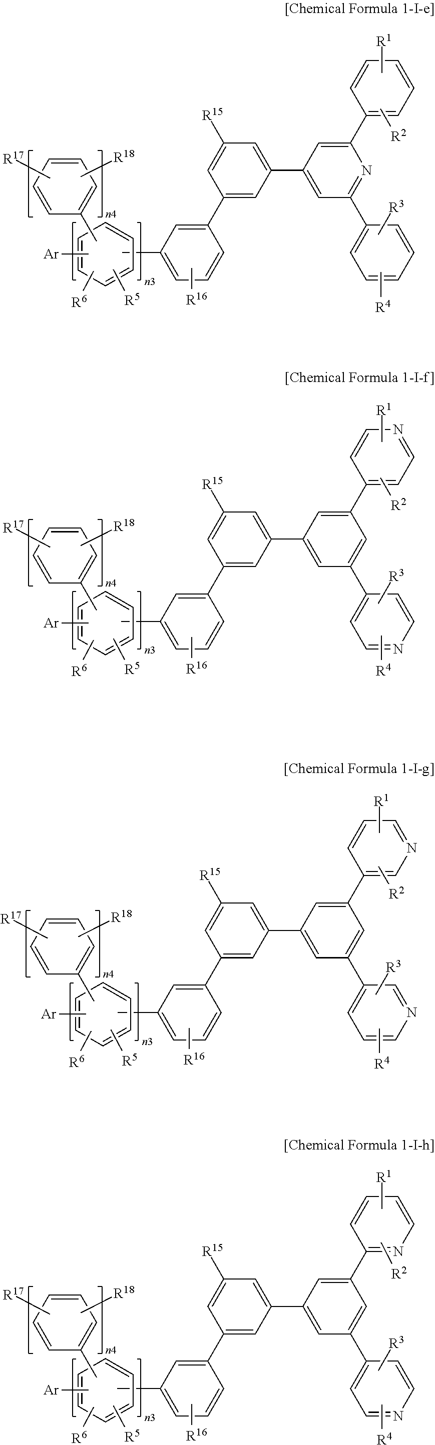

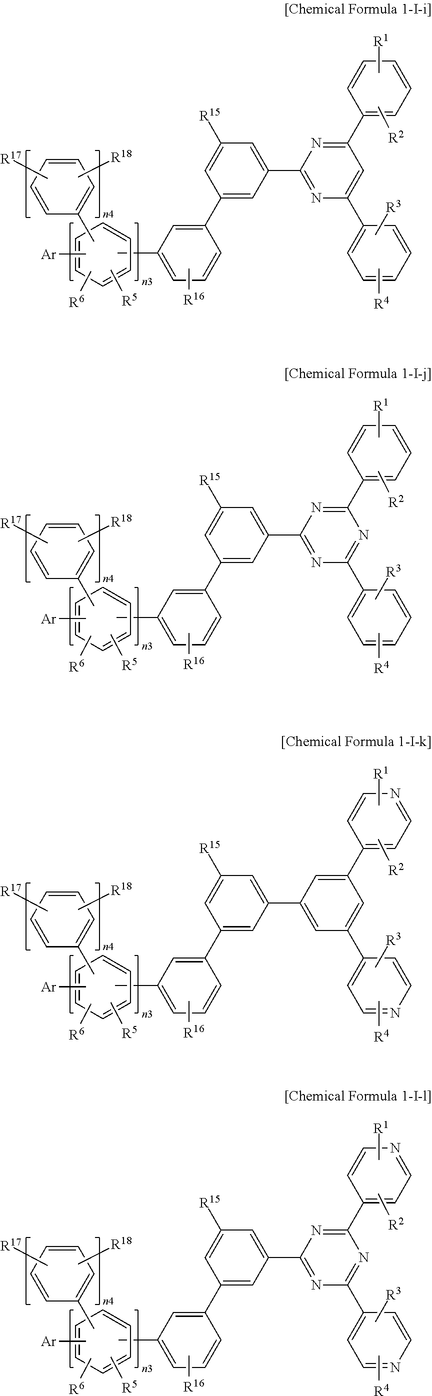

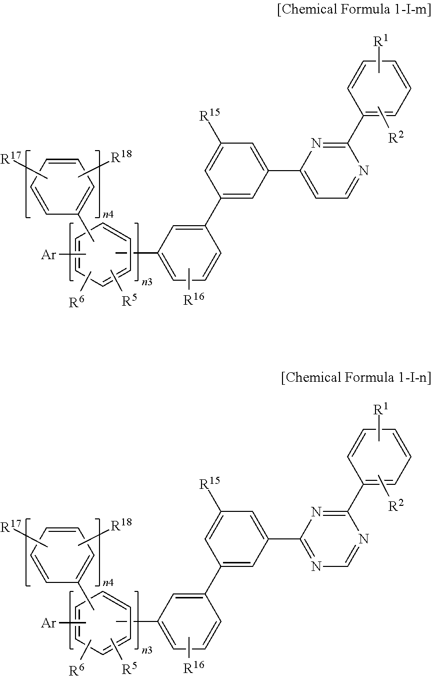

In Chemical Formulae 1-I-e to 1-I-n, definitions of R¹ to R⁶, R¹⁵ to R¹⁸, Ar, and n1 to n4 are the same as described above.

According to one embodiment, Chemical Formula 1-I may be represented by Chemical Formula 1-IIA or 1-IIB.

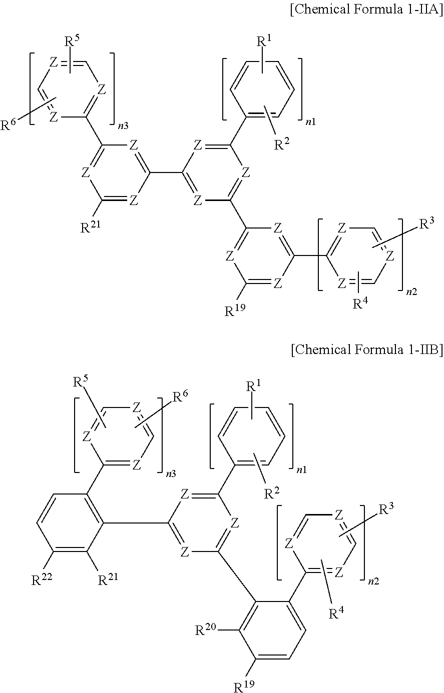

In Chemical Formulae 1-IIA and 1-IIB, Z, R¹ to R⁶, R¹⁹ to R²², and n1 to n3 are the same as described above, and

specifically, R¹ and R² of Chemical Formula 1-II may be hydrogen, deuterium, or a substituted or unsubstituted C6 to C12 aryl group. For example, they may be all hydrogen, but are not limited thereto.

Specifically, R³ to R⁶ of Chemical Formula 1-II may independently be a substituted or unsubstituted phenyl group, a substituted or unsubstituted biphenyl group, a substituted or unsubstituted terphenyl group, a substituted or unsubstituted naphthyl group, a substituted or unsubstituted pyridyl group, a substituted or unsubstituted pyrimidinyl group, a substituted or unsubstituted triazinyl group, a substituted or unsubstituted quinolinyl group, a substituted or unsubstituted isoquinolinyl group, a substituted or unsubstituted phenanthrolinyl group, or a substituted or unsubstituted quinazolinyl group. For example, they may be selected from the substituted or unsubstituted groups of the Group 1, but are not limited thereto.

Specifically, R¹⁹ to R²² of Chemical Formula 1-II may independently be hydrogen, a substituted or unsubstituted phenyl group, a substituted or unsubstituted biphenyl group, a substituted or unsubstituted naphthyl group, or a substituted or unsubstituted pyridinyl group. For example, they may be selected from the substituted or unsubstituted groups of the Group 1.

Herein, “substituted” is the same as defined above.

Chemical Formula 1-II may be, for example represented by one of Chemical Formulae 1-II-a to 1-II-h according to the position and the number of nitrogen, but is not limited thereto.

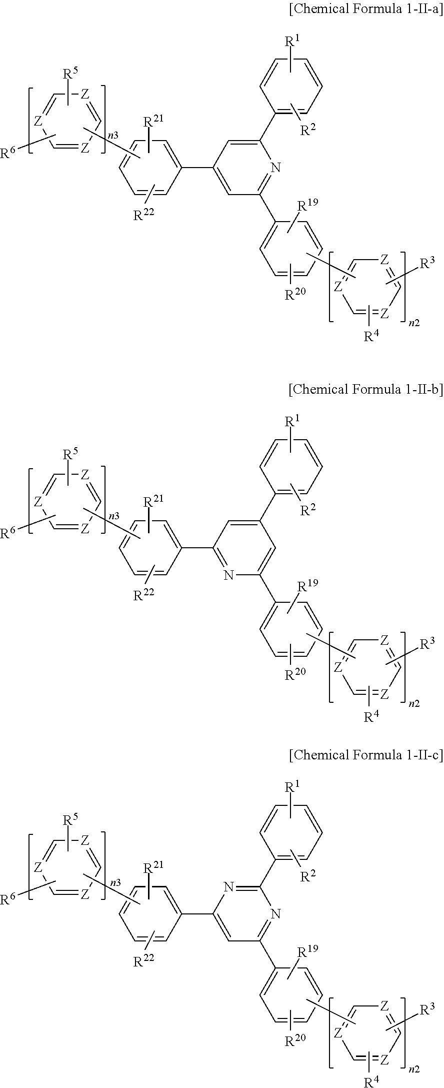

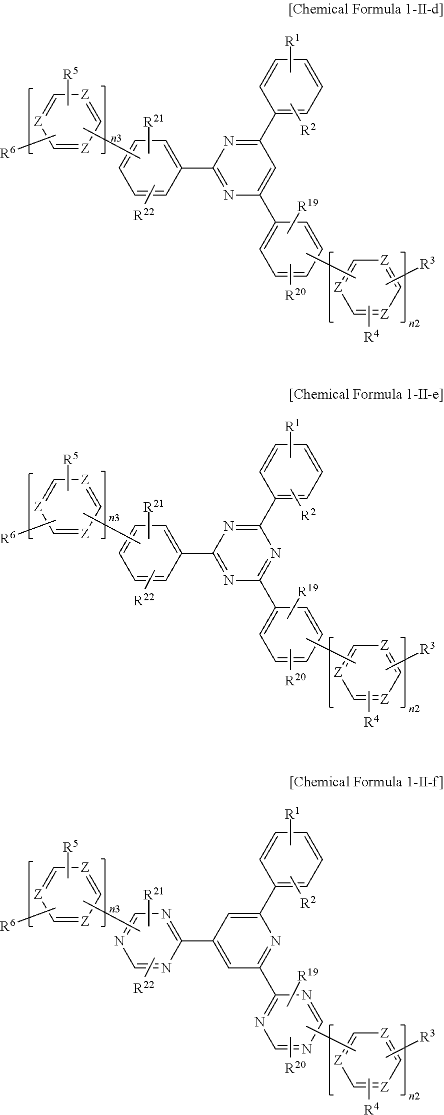

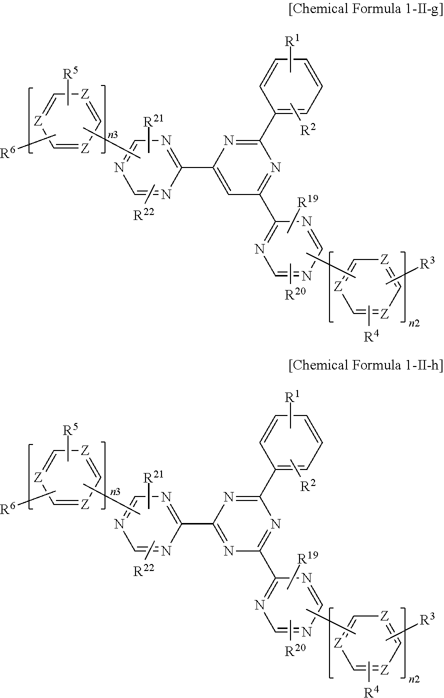

In Chemical Formulae 1-II-a to 1-II-h, Z, R¹ to R⁶, R¹⁹ to R²², and n2 and n3 are the same as described above.

According to one embodiment, Chemical Formula 1-III may be represented by Chemical Formula 1-IIIA or 1-IIIB according to a linking position of a triphenylene group.

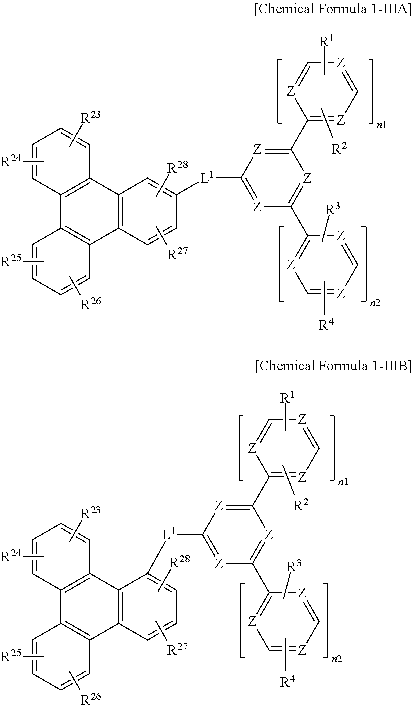

In Chemical Formulae 1-IIIA and 1-IIIB, Z, R¹ to R⁴, R²³ to R²⁸, L¹, n1, and n2 are the same as described above.

Specifically, R¹ to R⁴ and R²³ to R²⁸ of Chemical Formula 1-III may independently be hydrogen, deuterium, a substituted or unsubstituted C1 to C10 alkyl group, a substituted or unsubstituted C6 to C12 aryl group, or a combination thereof, L¹ is a substituted or unsubstituted phenylene group, a substituted or unsubstituted biphenylene group, or a substituted or unsubstituted terphenylene group.

6-membered rings substituting the triphenylene group indicate all the 6-membered rings directly or indirectly linked to the triphenylene group and include 6-membered rings consisting of a carbon atom, a nitrogen atom, or a combination thereof.

In Chemical Formula 1-III, the number of the 6-membered rings substituting the triphenylene group may be less than or equal to 6.

The first compound represented by Chemical Formula 1-III includes a triphenylene group and at least one nitrogen-containing heteroaryl group.

The first compound includes a ring containing at least one nitrogen and thus may have a structure of easily accepting electrons when an electric field is applied thereto and lower a driving voltage of an organic optoelectronic device.

In addition, the first compound represented by Chemical Formula 1-III includes both a triphenylene structure of easily accepting holes and a nitrogen-containing ring moiety easily accepting electrons and thus may form a bipolar structure, appropriately balance flows of the holes and the electrons, and improve efficiency of an organic optoelectronic device.

For example, a structure of Chemical Formula 1-III without a linking group (L¹) may be, for example, represented by Chemical Formula 1-III-a or 1-III-b.

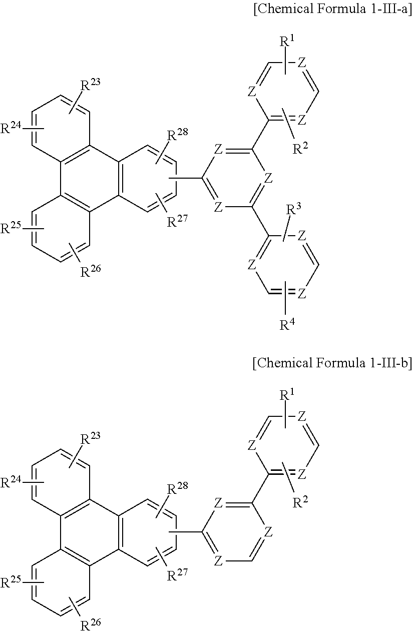

In Chemical Formulae 1-III-a and 1-III-b, Z, R¹ to R⁴, and R²³ to R²⁸ are the same as described above.

For example, in Chemical Formula 1-III without a linking group (L¹), L¹ may be a substituted or unsubstituted phenylene group substituted or unsubstituted biphenylene group or a substituted or unsubstituted terphenylene group, or a substituted or unsubstituted quaterphenylene group. The L¹ may be, for example, one selected from substituted or unsubstituted groups of Group 2.

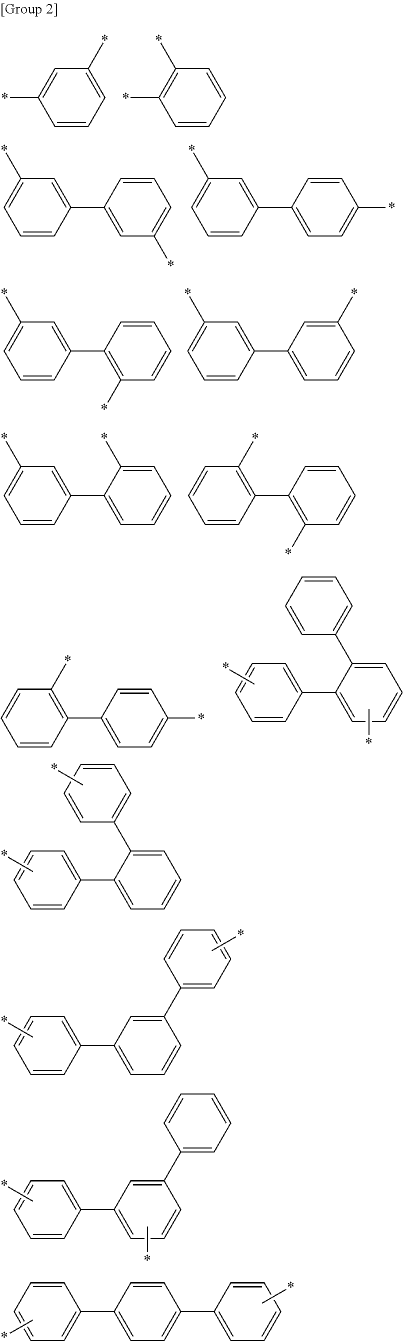

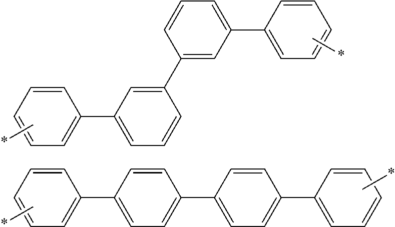

In Group 2, * is a linking point.

The first compound represented by Chemical Formula 1-III may have at least two kink structures, for example, two to four kink structures.

The first compound represented by Chemical Formula 1-III has the above kink structure and thus may appropriately localize a triphenylene structure easily accepting holes and a nitrogen-containing ring moiety easily accepting electrons in the above bipolar structure and controls a flow of a conjugated system and show excellent bipolar characteristics. In addition, the first compound represented by Chemical Formula 1-III may be effectively prevented from stacking of organic compounds due to the structure and decrease process stability and simultaneously, a deposition temperature. This stacking prevention effect may be further increased when the first compound represented by Chemical Formula 1-III includes the linking group (L¹).

The first compound represented by Chemical Formula 1-III having the linking group (L¹) may be, for example represented by Chemical Formulae 1-III-c to 1-III-t.

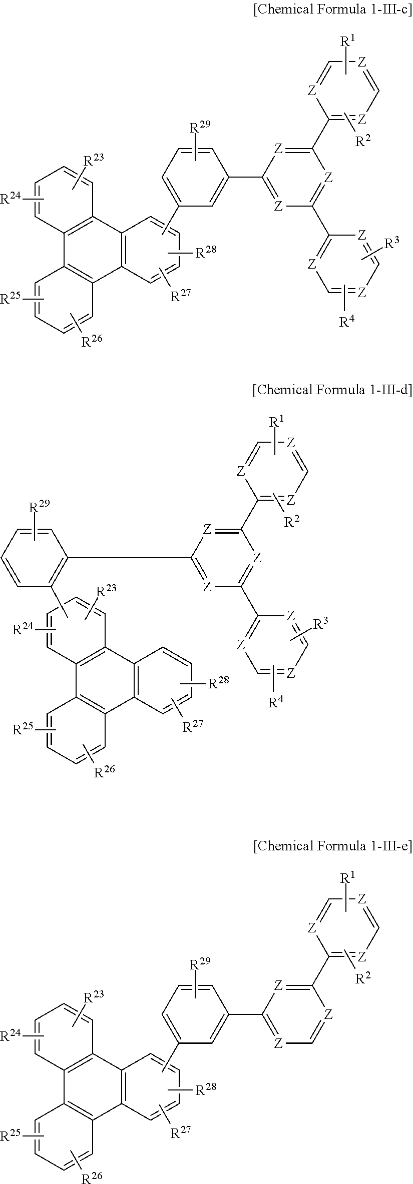

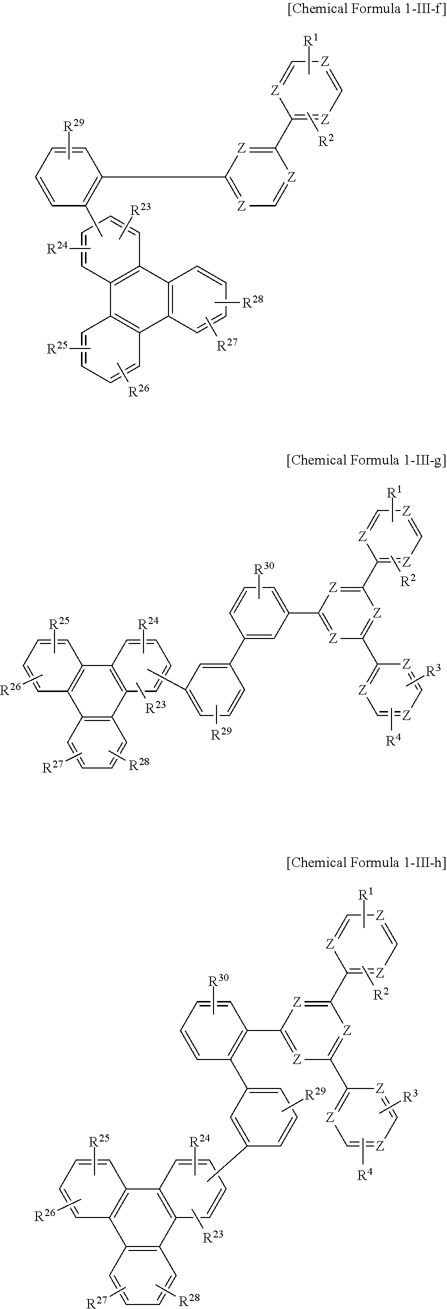

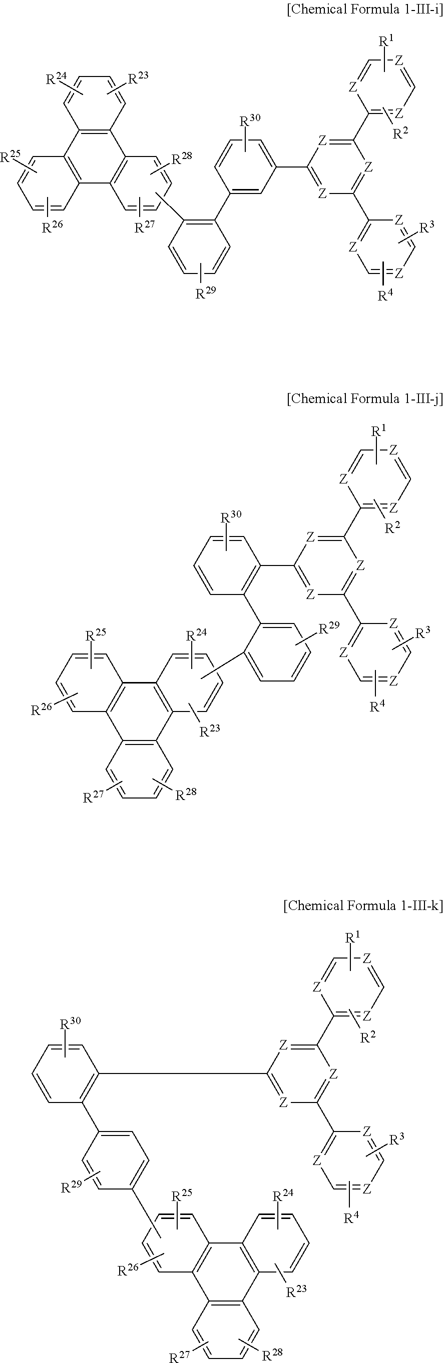

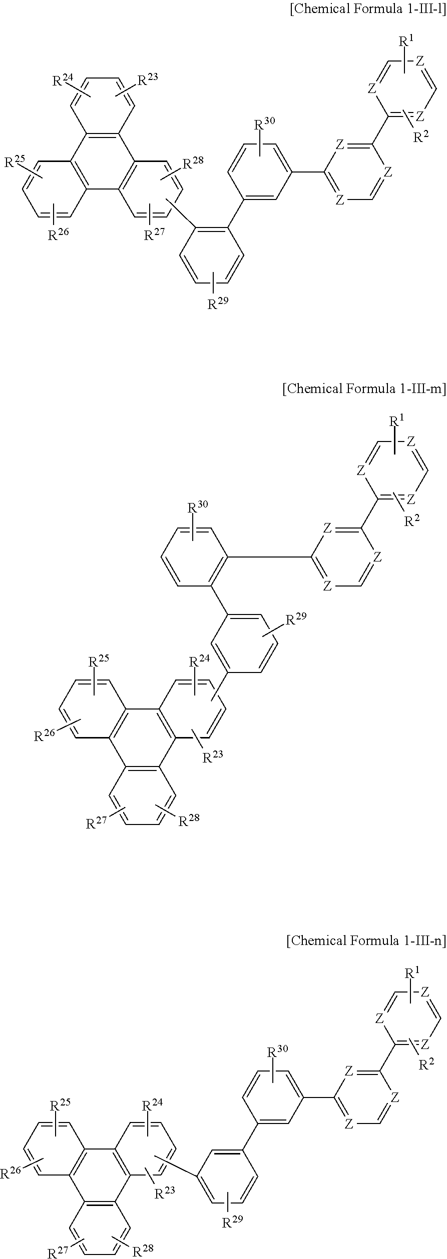

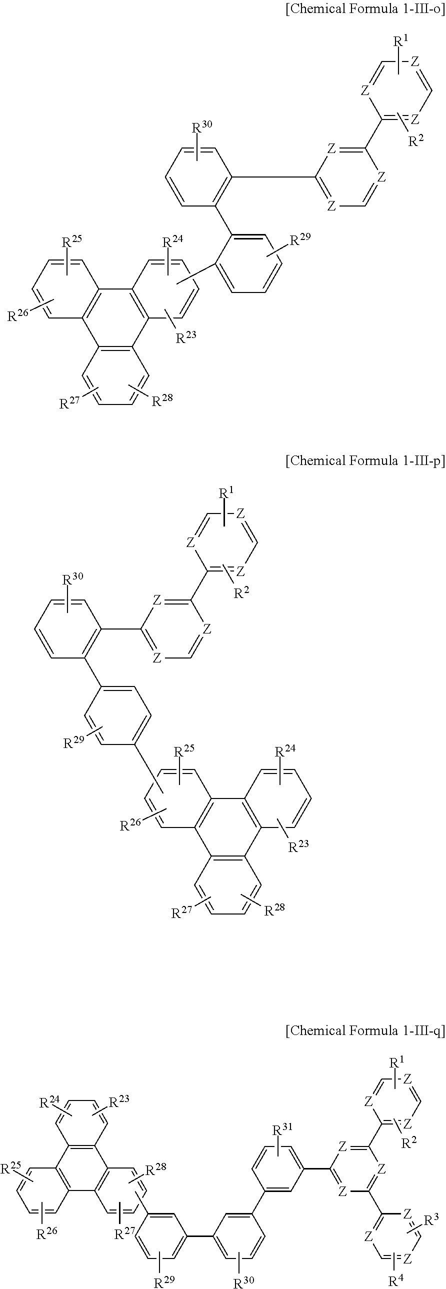

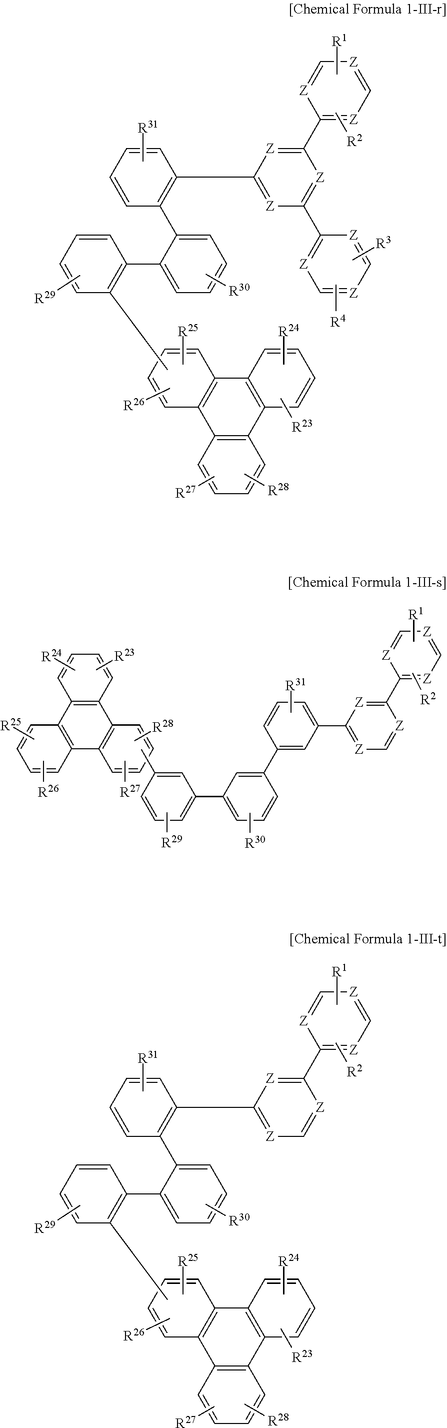

In Chemical Formulae 1-III-c to 1-III-t, Z, R¹ to R⁴, and R²³ to R²⁸ are the same as above, and R²⁹ to R³¹ are the same as R²³ to R²⁸.

The first compound represented by Chemical Formula 1 may be, for example, compounds of Group A but is not limited thereto.

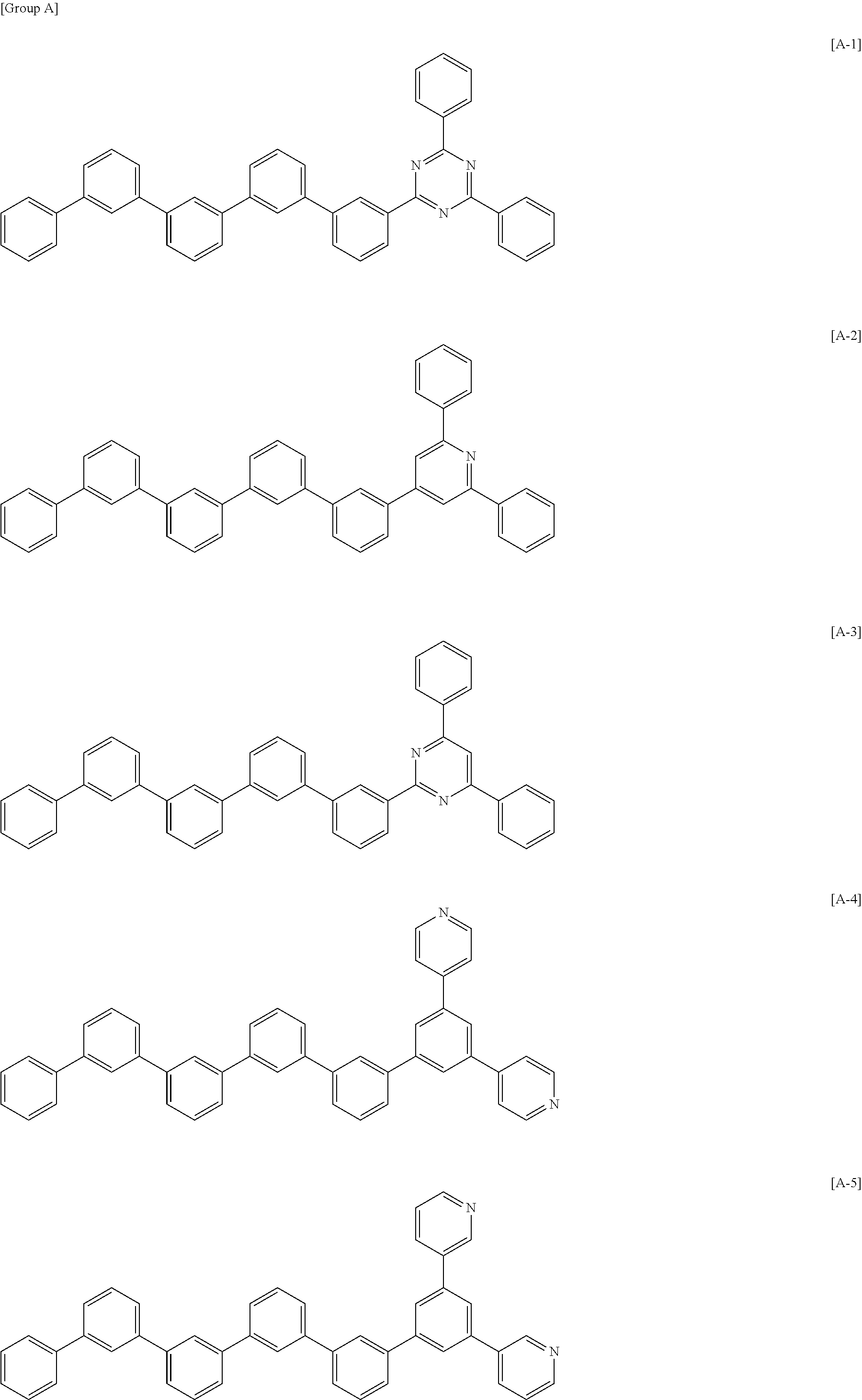 [SMILES: c1ccc(-c2cccc(-c3cccc(-c4cccc(-c5cccc(-c6nc(-c7ccccc7)nc(-c7ccccc7)n6)c5)c4)c3)c2)cc1 ; c1ccc(-c2cccc(-c3cccc(-c4cccc(-c5cccc(-c6cc(-c7ccccc7)nc(-c7ccccc7)c6)c5)c4)c3)c2)cc1 ; c1ccc(-c2cccc(-c3cccc(-c4cccc(-c5cccc(-c6nc(-c7ccccc7)cc(-c7ccccc7)n6)c5)c4)c3)c2)cc1 ; c1ccc(-c2cccc(-c3cccc(-c4cccc(-c5cccc(-c6cc(-c7ccncc7)cc(-c7ccncc7)c6)c5)c4)c3)c2)cc1 ; c1ccc(-c2cccc(-c3cccc(-c4cccc(-c5cccc(-c6cc(-c7cccnc7)cc(-c7cccnc7)c6)c5)c4)c3)c2)cc1]

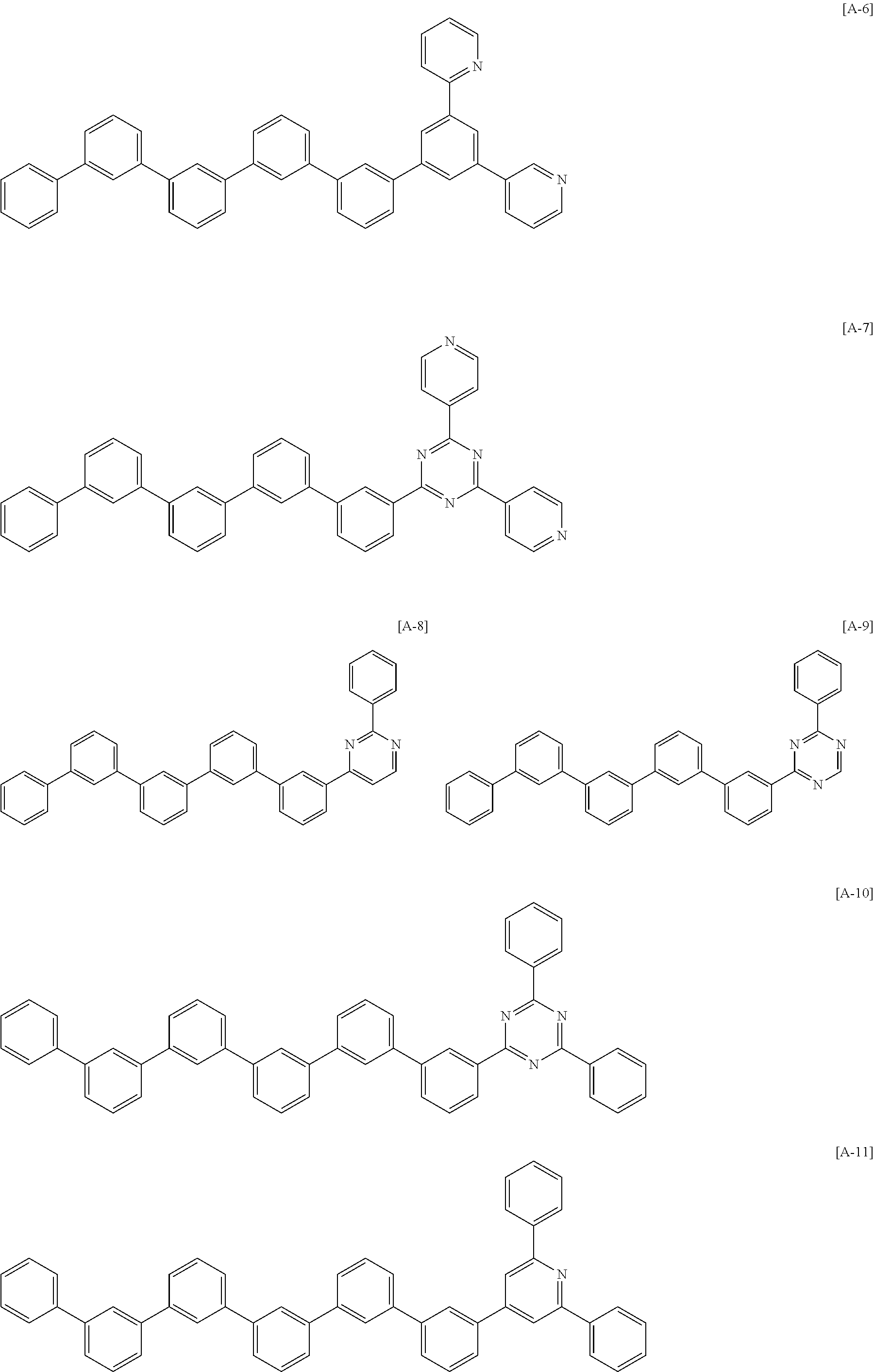

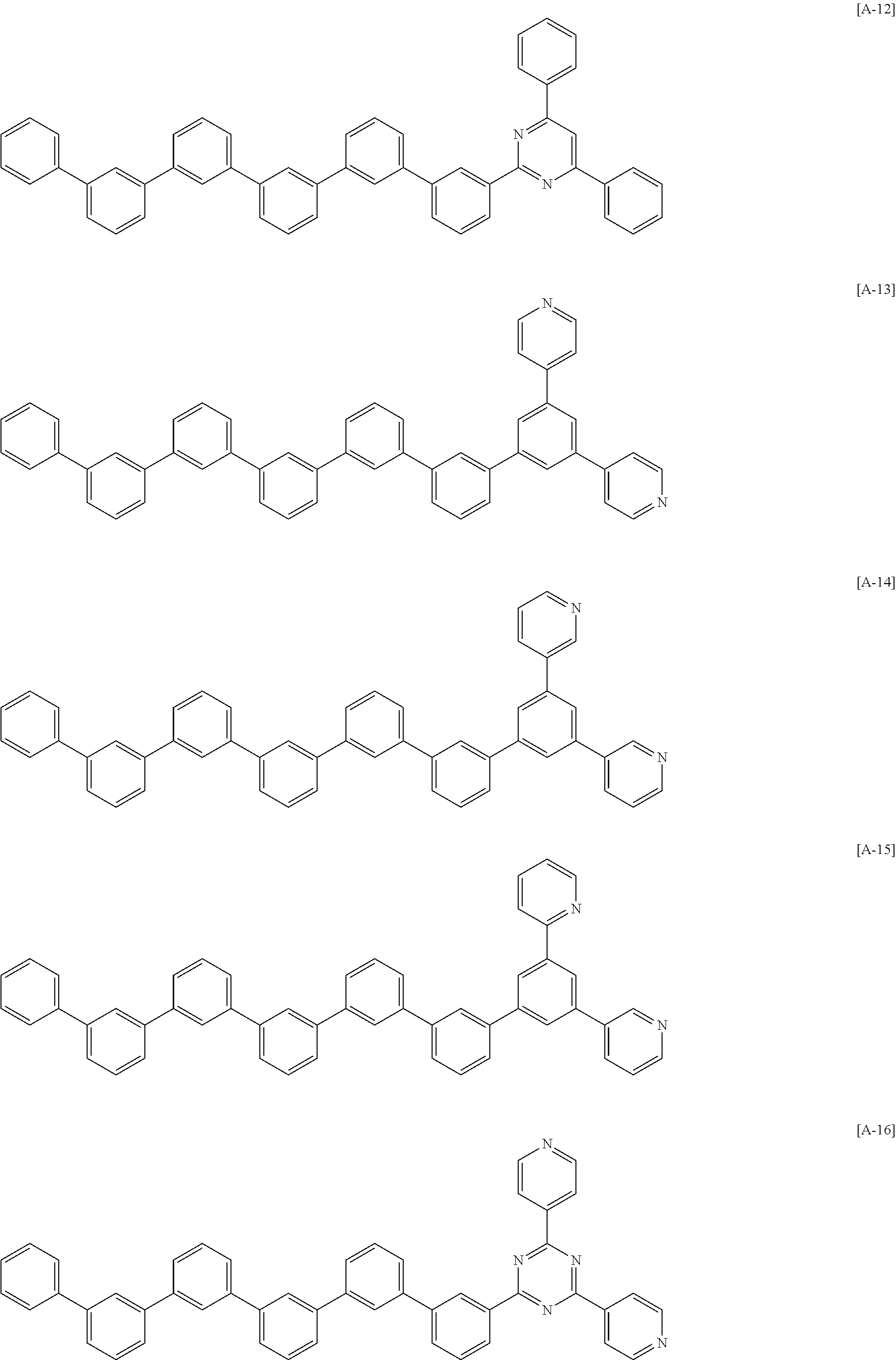 [SMILES: c1ccc(-c2cccc(-c3cccc(-c4cccc(-c5cccc(-c6cccc(-c7nc(-c8ccccc8)cc(-c8ccccc8)n7)c6)c5)c4)c3)c2)cc1 ; c1ccc(-c2cccc(-c3cccc(-c4cccc(-c5cccc(-c6cccc(-c7cc(-c8ccncc8)cc(-c8ccncc8)c7)c6)c5)c4)c3)c2)cc1 ; c1ccc(-c2cccc(-c3cccc(-c4cccc(-c5cccc(-c6cccc(-c7cc(-c8cccnc8)cc(-c8cccnc8)c7)c6)c5)c4)c3)c2)cc1 ; c1ccc(-c2cccc(-c3cccc(-c4cccc(-c5cccc(-c6cccc(-c7cc(-c8cccnc8)cc(-c8ccccn8)c7)c6)c5)c4)c3)c2)cc1 ; c1ccc(-c2cccc(-c3cccc(-c4cccc(-c5cccc(-c6cccc(-c7nc(-c8ccncc8)nc(-c8ccncc8)n7)c6)c5)c4)c3)c2)cc1]

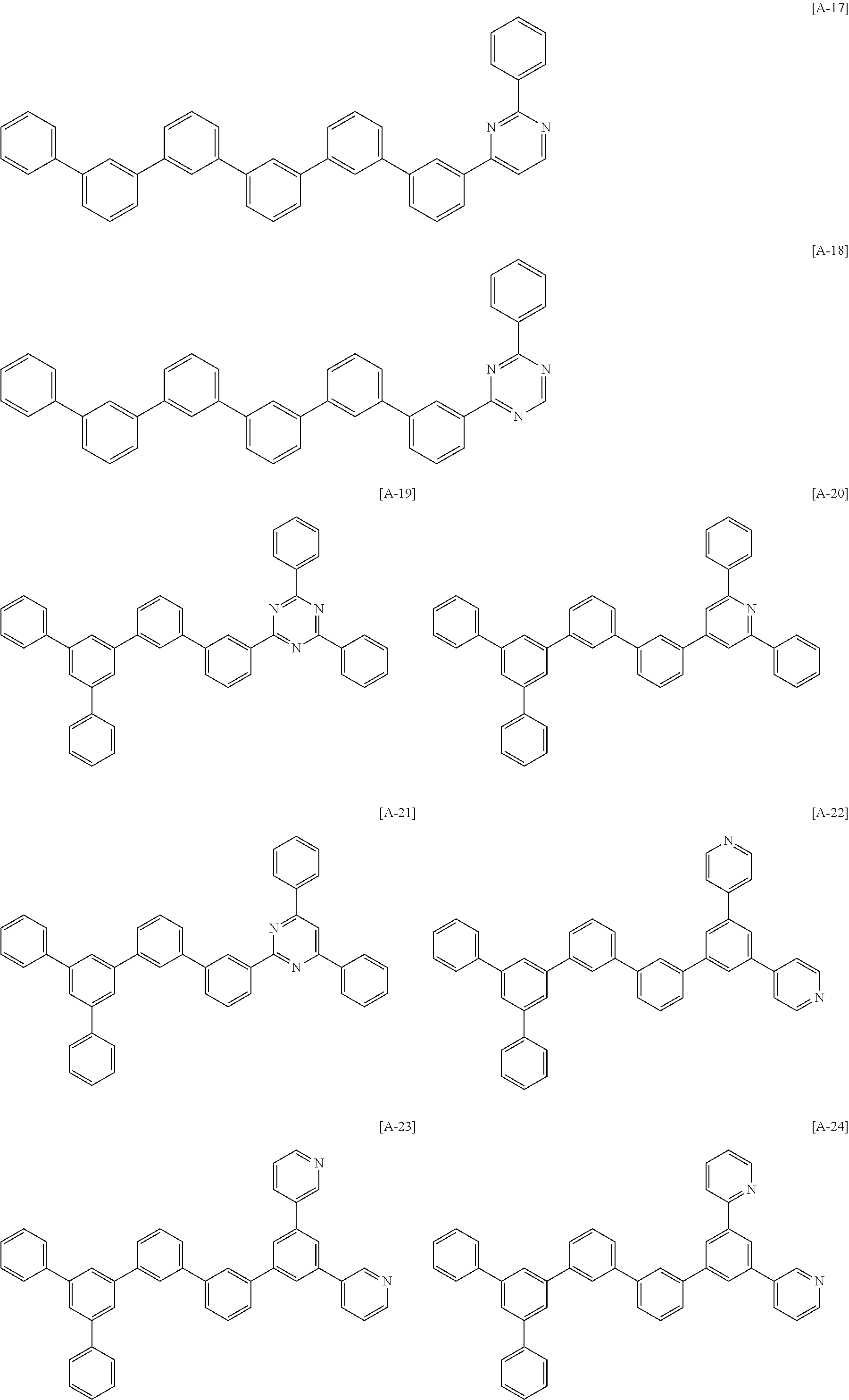

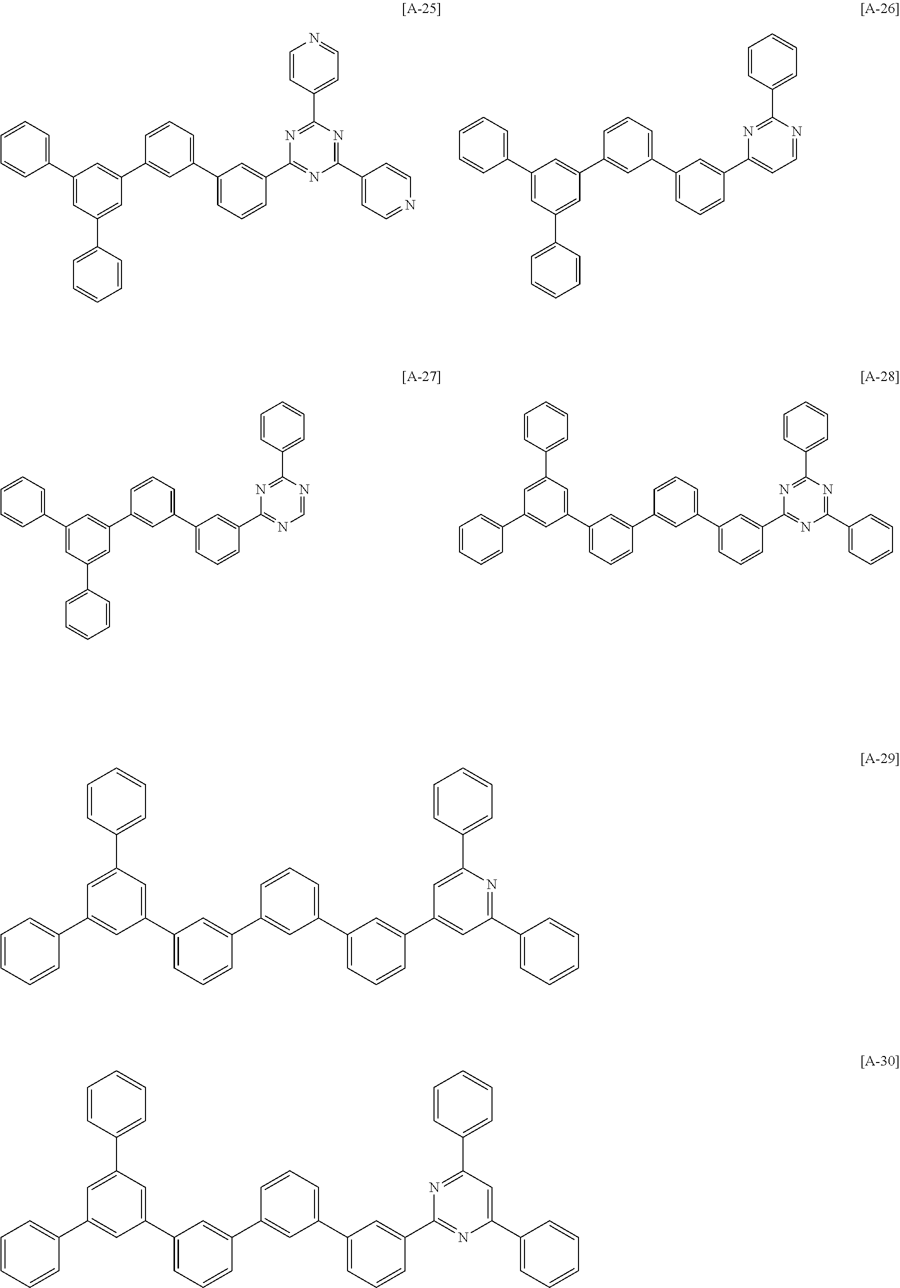

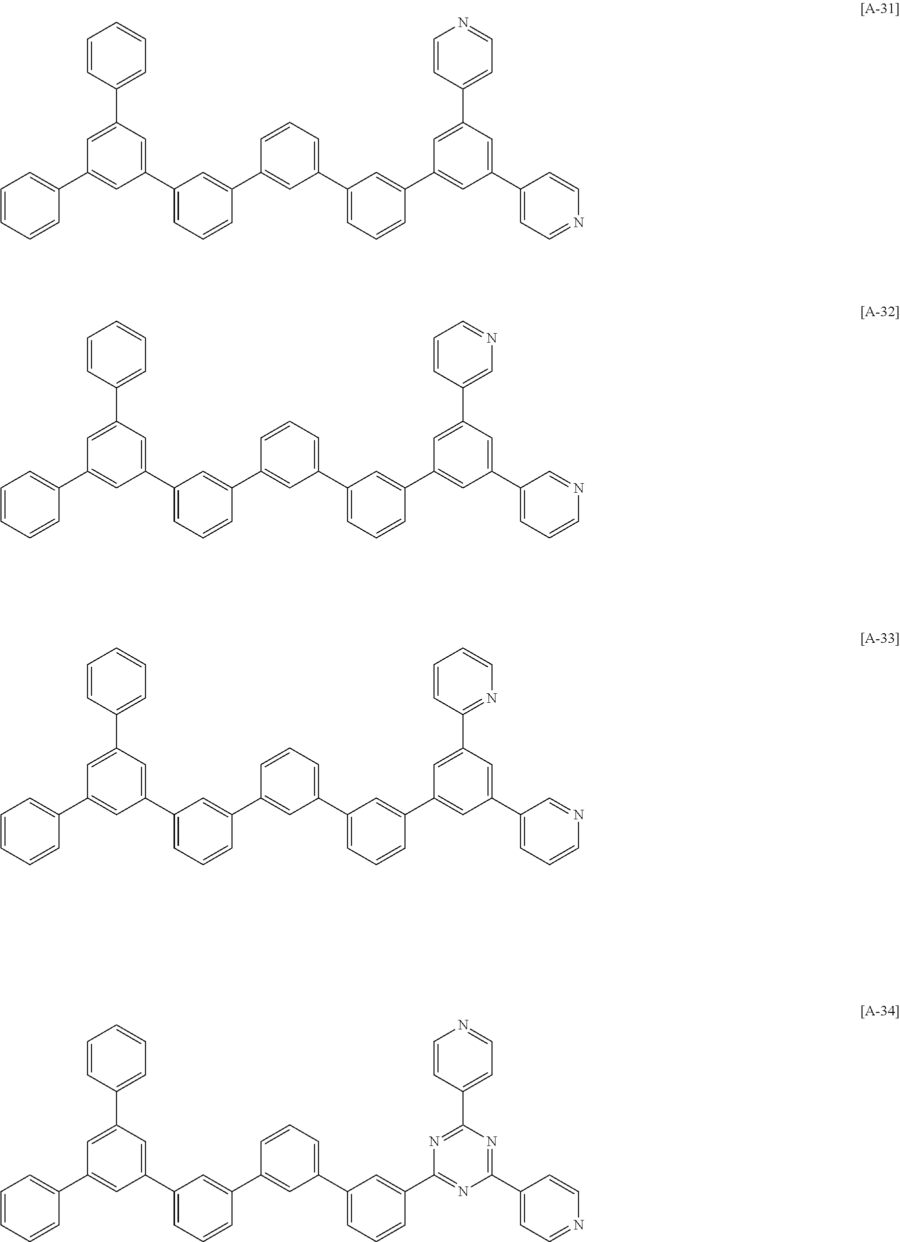 [SMILES: c1ccc(-c2cc(-c3ccccc3)cc(-c3cccc(-c4cccc(-c5cccc(-c6cc(-c7ccncc7)cc(-c7ccncc7)c6)c5)c4)c3)c2)cc1 ; c1ccc(-c2cc(-c3ccccc3)cc(-c3cccc(-c4cccc(-c5cccc(-c6cc(-c7cccnc7)cc(-c7cccnc7)c6)c5)c4)c3)c2)cc1 ; c1ccc(-c2cc(-c3ccccc3)cc(-c3cccc(-c4cccc(-c5cccc(-c6cc(-c7cccnc7)cc(-c7ccccn7)c6)c5)c4)c3)c2)cc1 ; c1ccc(-c2cc(-c3ccccc3)cc(-c3cccc(-c4cccc(-c5cccc(-c6nc(-c7ccncc7)nc(-c7ccncc7)n6)c5)c4)c3)c2)cc1]

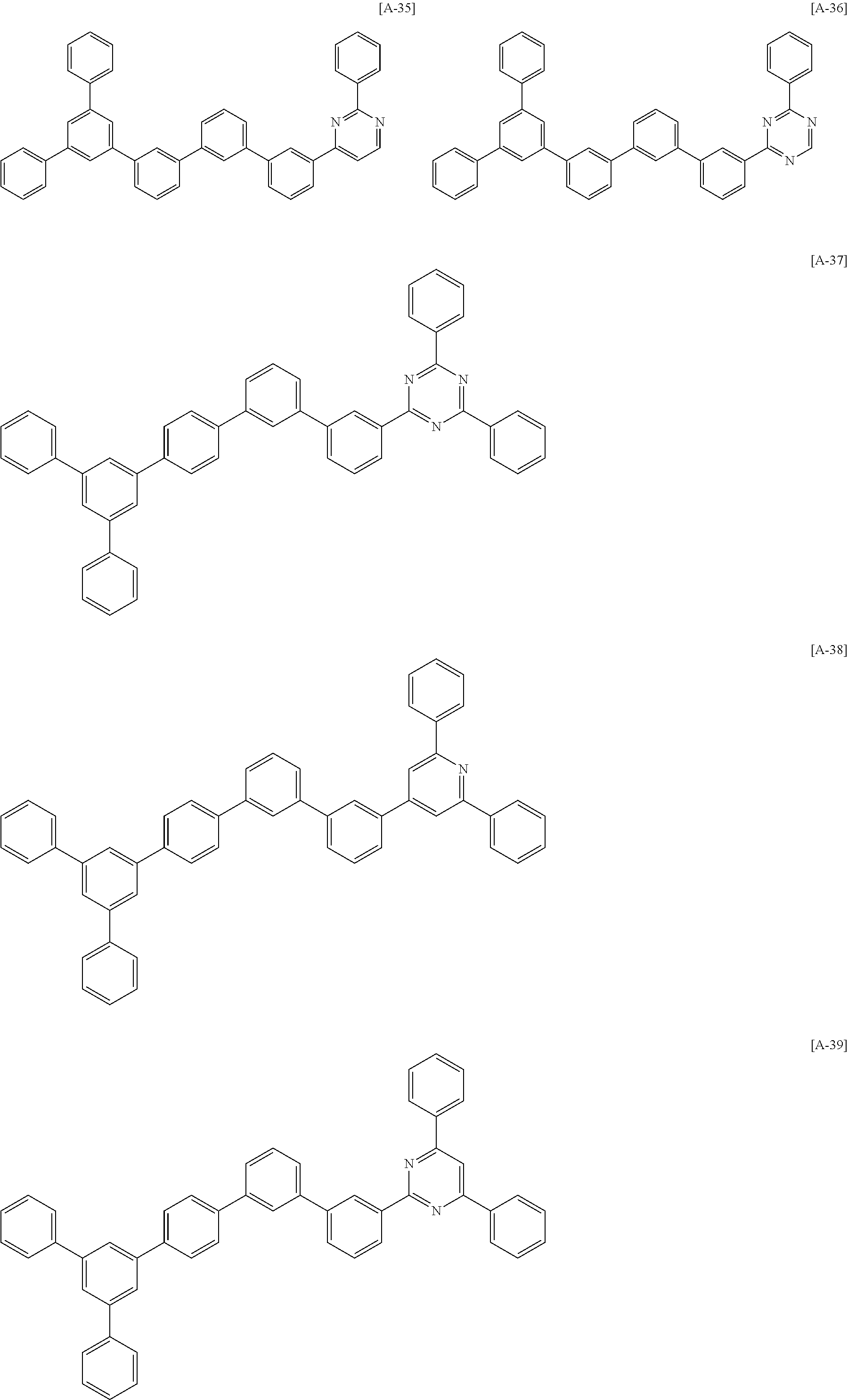 [SMILES: c1ccc(-c2cc(-c3ccccc3)cc(-c3cccc(-c4cccc(-c5cccc(-c6ccnc(-c7ccccc7)n6)c5)c4)c3)c2)cc1 ; c1ccc(-c2cc(-c3ccccc3)cc(-c3cccc(-c4cccc(-c5cccc(-c6ncnc(-c7ccccc7)n6)c5)c4)c3)c2)cc1 ; c1ccc(-c2cc(-c3ccccc3)cc(-c3ccc(-c4cccc(-c5cccc(-c6nc(-c7ccccc7)nc(-c7ccccc7)n6)c5)c4)cc3)c2)cc1 ; c1ccc(-c2cc(-c3ccccc3)cc(-c3ccc(-c4cccc(-c5cccc(-c6cc(-c7ccccc7)nc(-c7ccccc7)c6)c5)c4)cc3)c2)cc1 ; c1ccc(-c2cc(-c3ccccc3)cc(-c3ccc(-c4cccc(-c5cccc(-c6nc(-c7ccccc7)cc(-c7ccccc7)n6)c5)c4)cc3)c2)cc1]

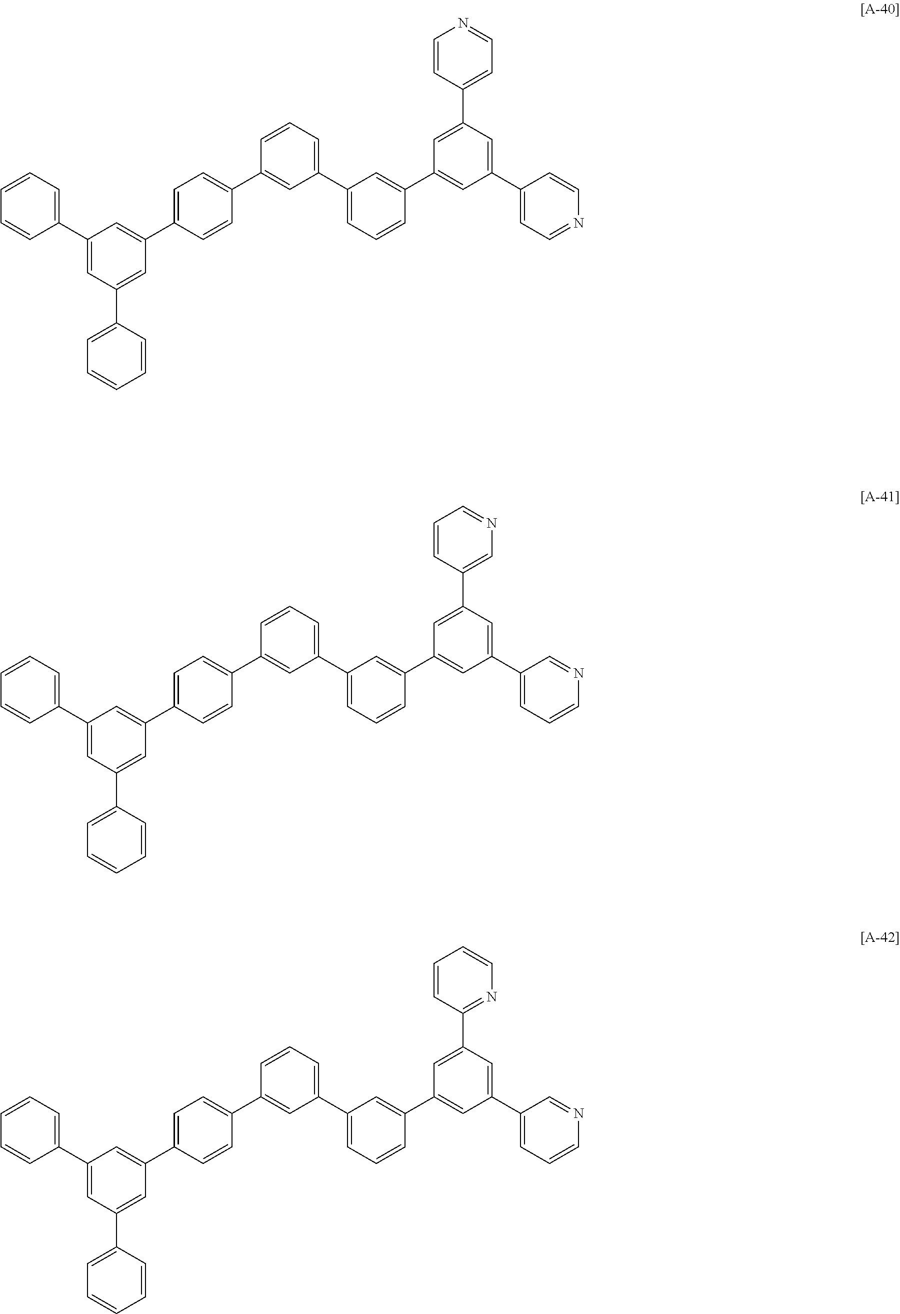 [SMILES: c1ccc(-c2cc(-c3ccccc3)cc(-c3ccc(-c4cccc(-c5cccc(-c6cc(-c7ccncc7)cc(-c7ccncc7)c6)c5)c4)cc3)c2)cc1 ; c1ccc(-c2cc(-c3ccccc3)cc(-c3ccc(-c4cccc(-c5cccc(-c6cc(-c7cccnc7)cc(-c7cccnc7)c6)c5)c4)cc3)c2)cc1 ; c1ccc(-c2cc(-c3ccccc3)cc(-c3ccc(-c4cccc(-c5cccc(-c6cc(-c7cccnc7)cc(-c7ccccn7)c6)c5)c4)cc3)c2)cc1]

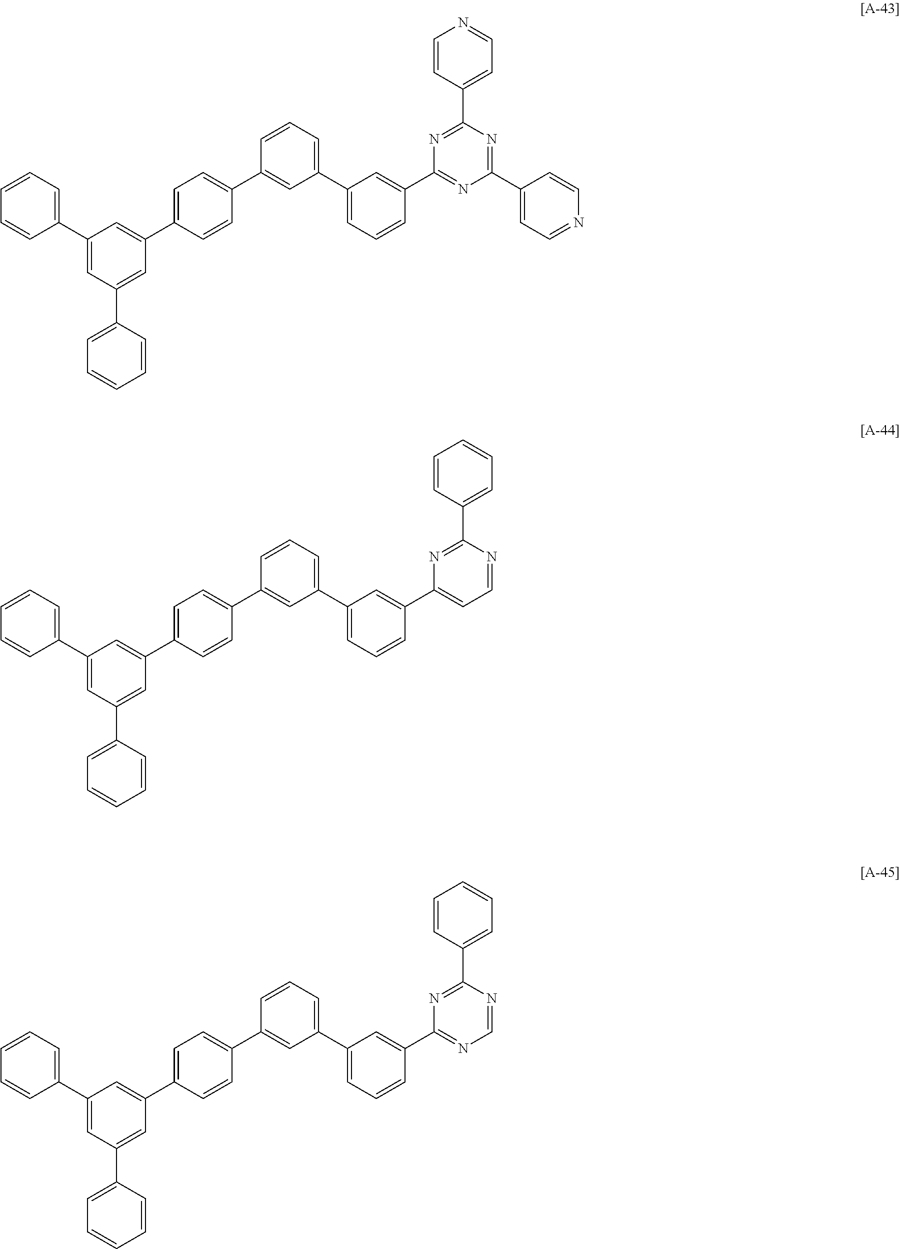 [SMILES: c1ccc(-c2cc(-c3ccccc3)cc(-c3ccc(-c4cccc(-c5cccc(-c6nc(-c7ccncc7)nc(-c7ccncc7)n6)c5)c4)cc3)c2)cc1 ; c1ccc(-c2cc(-c3ccccc3)cc(-c3ccc(-c4cccc(-c5cccc(-c6ccnc(-c7ccccc7)n6)c5)c4)cc3)c2)cc1 ; c1ccc(-c2cc(-c3ccccc3)cc(-c3ccc(-c4cccc(-c5cccc(-c6ncnc(-c7ccccc7)n6)c5)c4)cc3)c2)cc1]

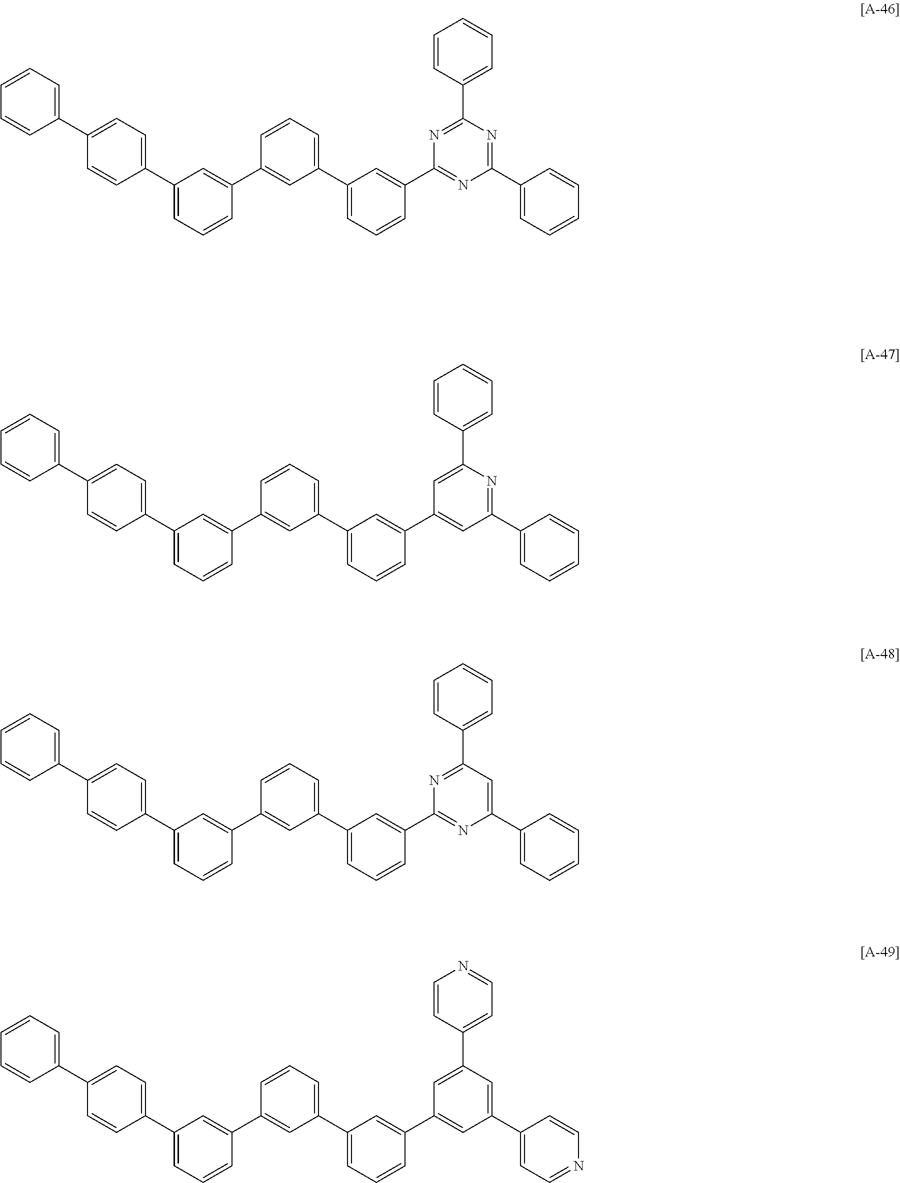 [SMILES: c1ccc(-c2ccc(-c3cccc(-c4cccc(-c5cccc(-c6nc(-c7ccccc7)nc(-c7ccccc7)n6)c5)c4)c3)cc2)cc1 ; c1ccc(-c2ccc(-c3cccc(-c4cccc(-c5cccc(-c6cc(-c7ccccc7)nc(-c7ccccc7)c6)c5)c4)c3)cc2)cc1 ; c1ccc(-c2ccc(-c3cccc(-c4cccc(-c5cccc(-c6nc(-c7ccccc7)cc(-c7ccccc7)n6)c5)c4)c3)cc2)cc1 ; c1ccc(-c2ccc(-c3cccc(-c4cccc(-c5cccc(-c6cc(-c7ccncc7)cc(-c7ccncc7)c6)c5)c4)c3)cc2)cc1]

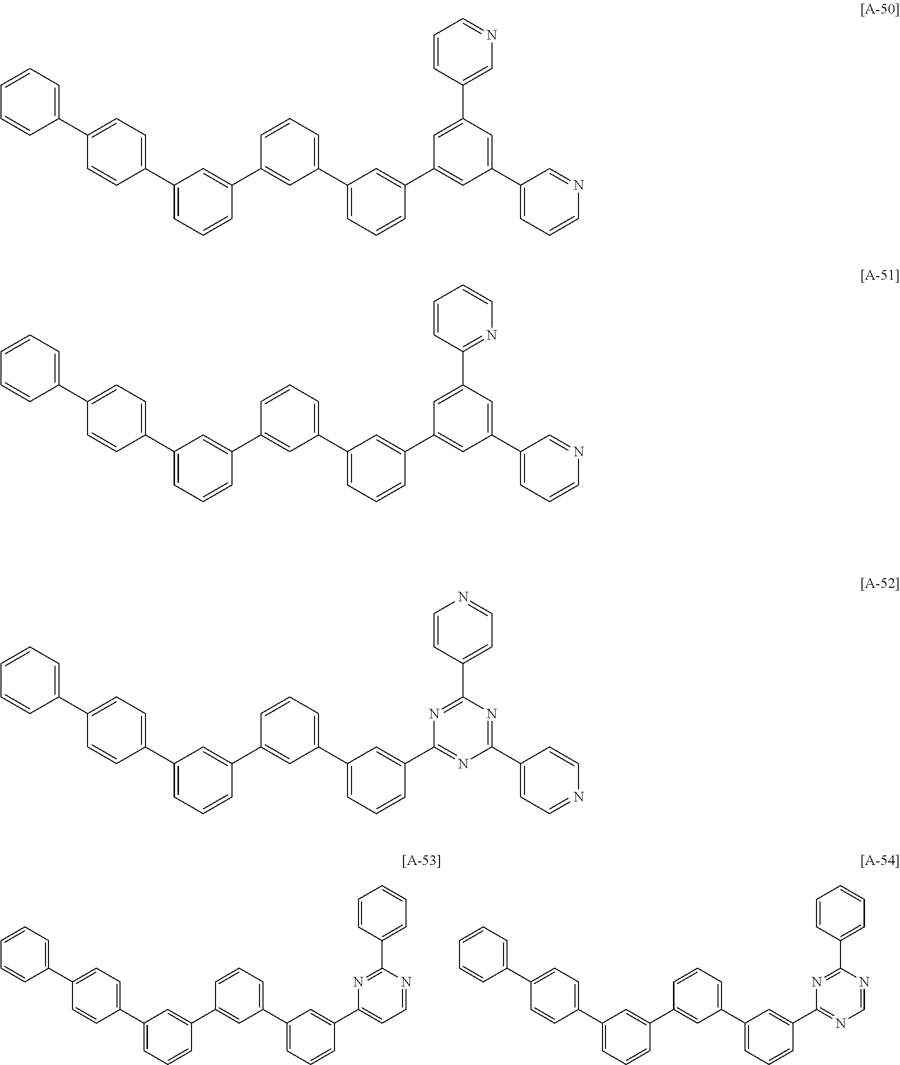 [SMILES: c1ccc(-c2ccc(-c3cccc(-c4cccc(-c5cccc(-c6cc(-c7cccnc7)cc(-c7cccnc7)c6)c5)c4)c3)cc2)cc1 ; c1ccc(-c2ccc(-c3cccc(-c4cccc(-c5cccc(-c6cc(-c7cccnc7)cc(-c7ccccn7)c6)c5)c4)c3)cc2)cc1 ; c1ccc(-c2ccc(-c3cccc(-c4cccc(-c5cccc(-c6nc(-c7ccncc7)nc(-c7ccncc7)n6)c5)c4)c3)cc2)cc1 ; c1ccc(-c2ccc(-c3cccc(-c4cccc(-c5cccc(-c6ccnc(-c7ccccc7)n6)c5)c4)c3)cc2)cc1 ; c1ccc(-c2ccc(-c3cccc(-c4cccc(-c5cccc(-c6ncnc(-c7ccccc7)n6)c5)c4)c3)cc2)cc1]

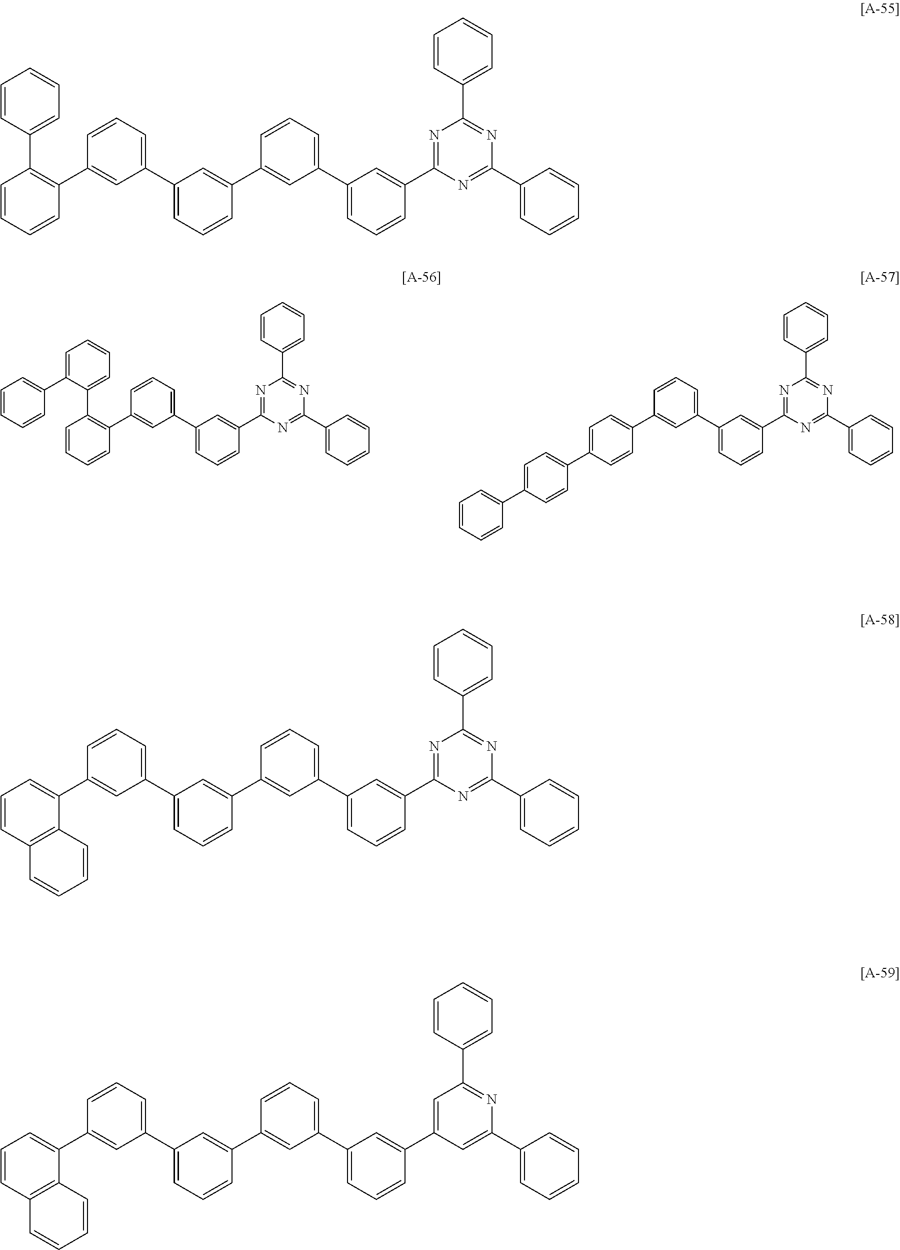 [SMILES: c1ccc(-c2nc(-c3ccccc3)nc(-c3cccc(-c4cccc(-c5cccc(-c6cccc(-c7ccccc7-c7ccccc7)c6)c5)c4)c3)n2)cc1 ; c1ccc(-c2nc(-c3ccccc3)nc(-c3cccc(-c4cccc(-c5ccccc5-c5ccccc5-c5ccccc5)c4)c3)n2)cc1 ; c1ccc(-c2ccc(-c3ccc(-c4cccc(-c5cccc(-c6nc(-c7ccccc7)nc(-c7ccccc7)n6)c5)c4)cc3)cc2)cc1 ; c1ccc(-c2nc(-c3ccccc3)nc(-c3cccc(-c4cccc(-c5cccc(-c6cccc(-c7cccc8ccccc78)c6)c5)c4)c3)n2)cc1 ; c1ccc(-c2cc(-c3cccc(-c4cccc(-c5cccc(-c6cccc(-c7cccc8ccccc78)c6)c5)c4)c3)cc(-c3ccccc3)n2)cc1]

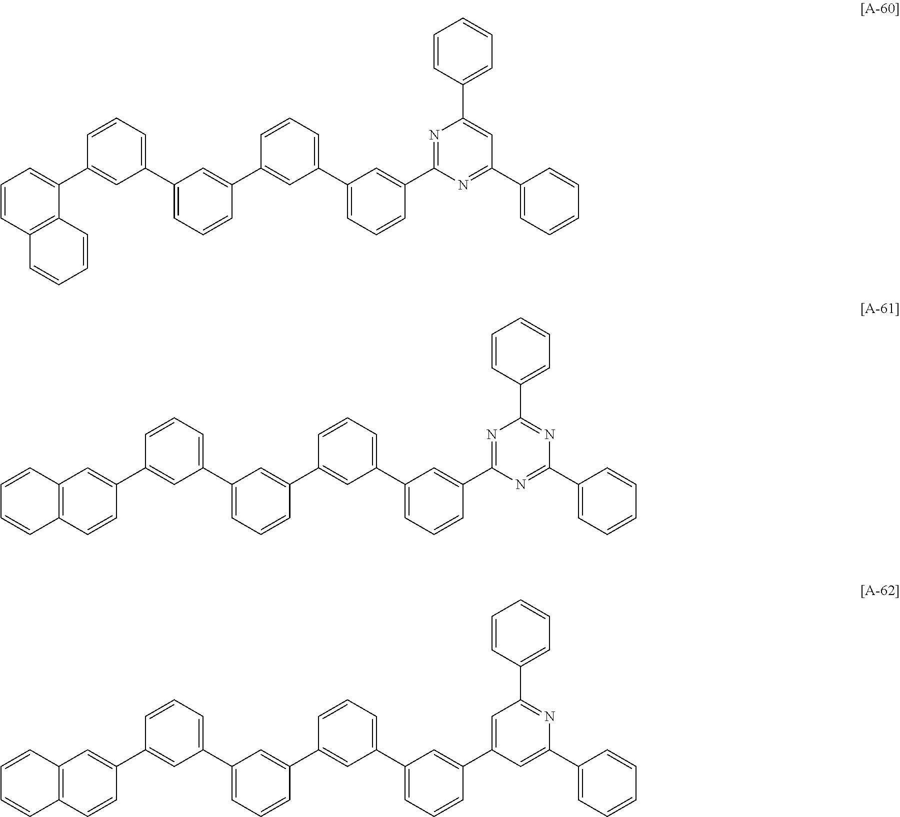 [SMILES: c1ccc(-c2cc(-c3ccccc3)nc(-c3cccc(-c4cccc(-c5cccc(-c6cccc(-c7cccc8ccccc78)c6)c5)c4)c3)n2)cc1 ; c1ccc(-c2nc(-c3ccccc3)nc(-c3cccc(-c4cccc(-c5cccc(-c6cccc(-c7ccc8ccccc8c7)c6)c5)c4)c3)n2)cc1 ; c1ccc(-c2cc(-c3cccc(-c4cccc(-c5cccc(-c6cccc(-c7ccc8ccccc8c7)c6)c5)c4)c3)cc(-c3ccccc3)n2)cc1]

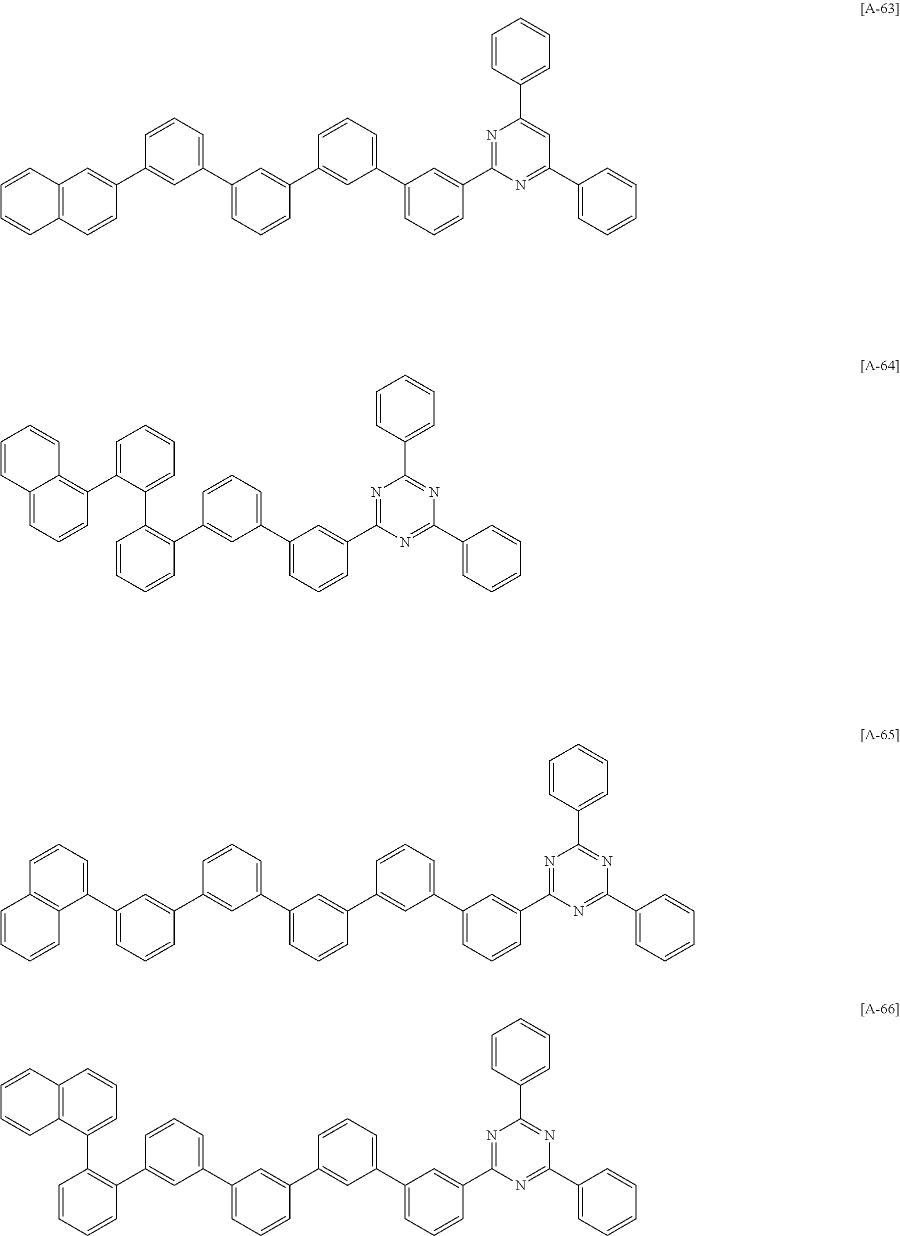 [SMILES: c1ccc(-c2cc(-c3ccccc3)nc(-c3cccc(-c4cccc(-c5cccc(-c6cccc(-c7ccc8ccccc8c7)c6)c5)c4)c3)n2)cc1 ; c1ccc(-c2nc(-c3ccccc3)nc(-c3cccc(-c4cccc(-c5ccccc5-c5ccccc5-c5cccc6ccccc56)c4)c3)n2)cc1 ; c1ccc(-c2nc(-c3ccccc3)nc(-c3cccc(-c4cccc(-c5cccc(-c6cccc(-c7cccc(-c8cccc9ccccc89)c7)c6)c5)c4)c3)n2)cc1 ; c1ccc(-c2nc(-c3ccccc3)nc(-c3cccc(-c4cccc(-c5cccc(-c6cccc(-c7ccccc7-c7cccc8ccccc78)c6)c5)c4)c3)n2)cc1]

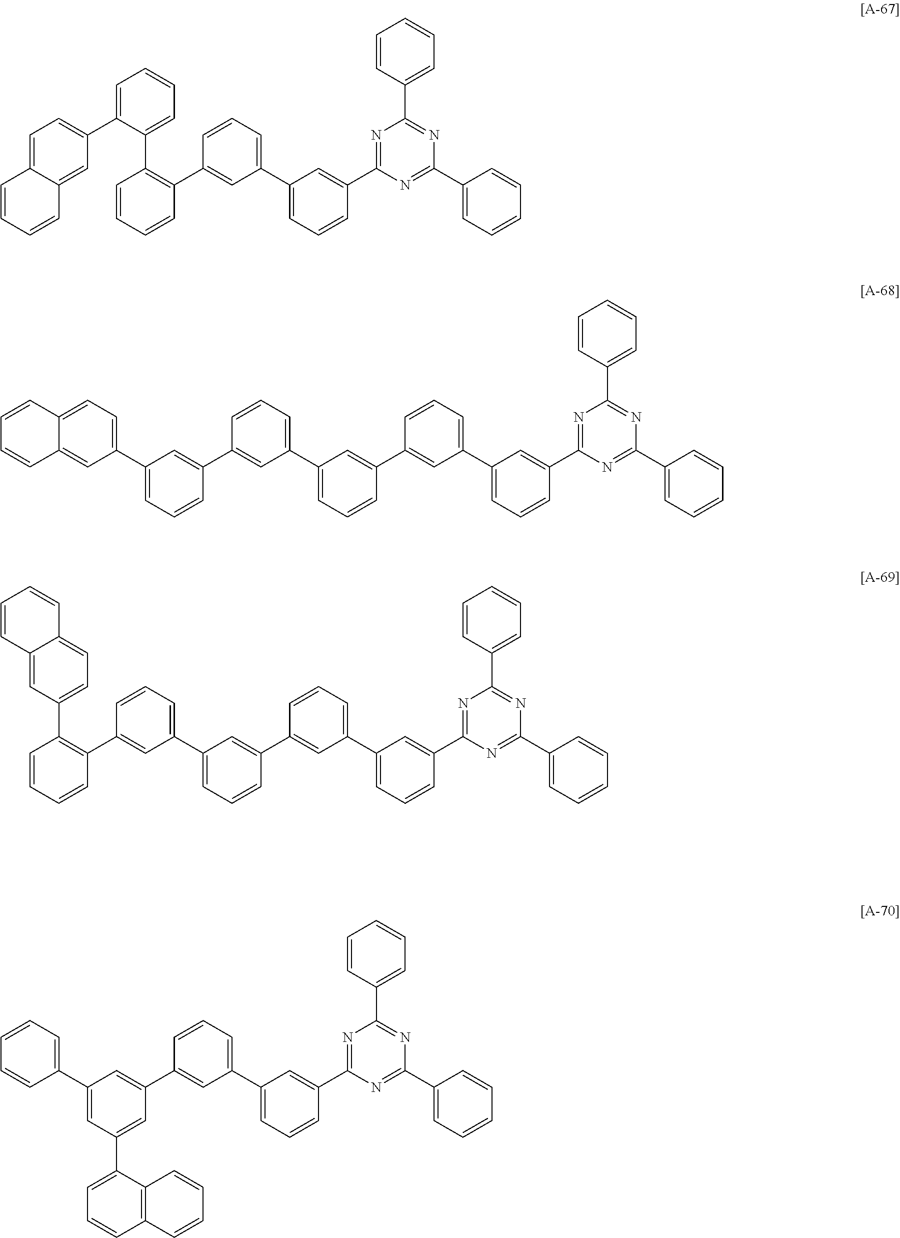 [SMILES: c1ccc(-c2nc(-c3ccccc3)nc(-c3cccc(-c4cccc(-c5ccccc5-c5ccccc5-c5ccc6ccccc6c5)c4)c3)n2)cc1 ; c1ccc(-c2nc(-c3ccccc3)nc(-c3cccc(-c4cccc(-c5cccc(-c6cccc(-c7cccc(-c8ccc9ccccc9c8)c7)c6)c5)c4)c3)n2)cc1 ; c1ccc(-c2nc(-c3ccccc3)nc(-c3cccc(-c4cccc(-c5cccc(-c6cccc(-c7ccccc7-c7ccc8ccccc8c7)c6)c5)c4)c3)n2)cc1 ; c1ccc(-c2cc(-c3cccc(-c4cccc(-c5nc(-c6ccccc6)nc(-c6ccccc6)n5)c4)c3)cc(-c3cccc4ccccc34)c2)cc1]

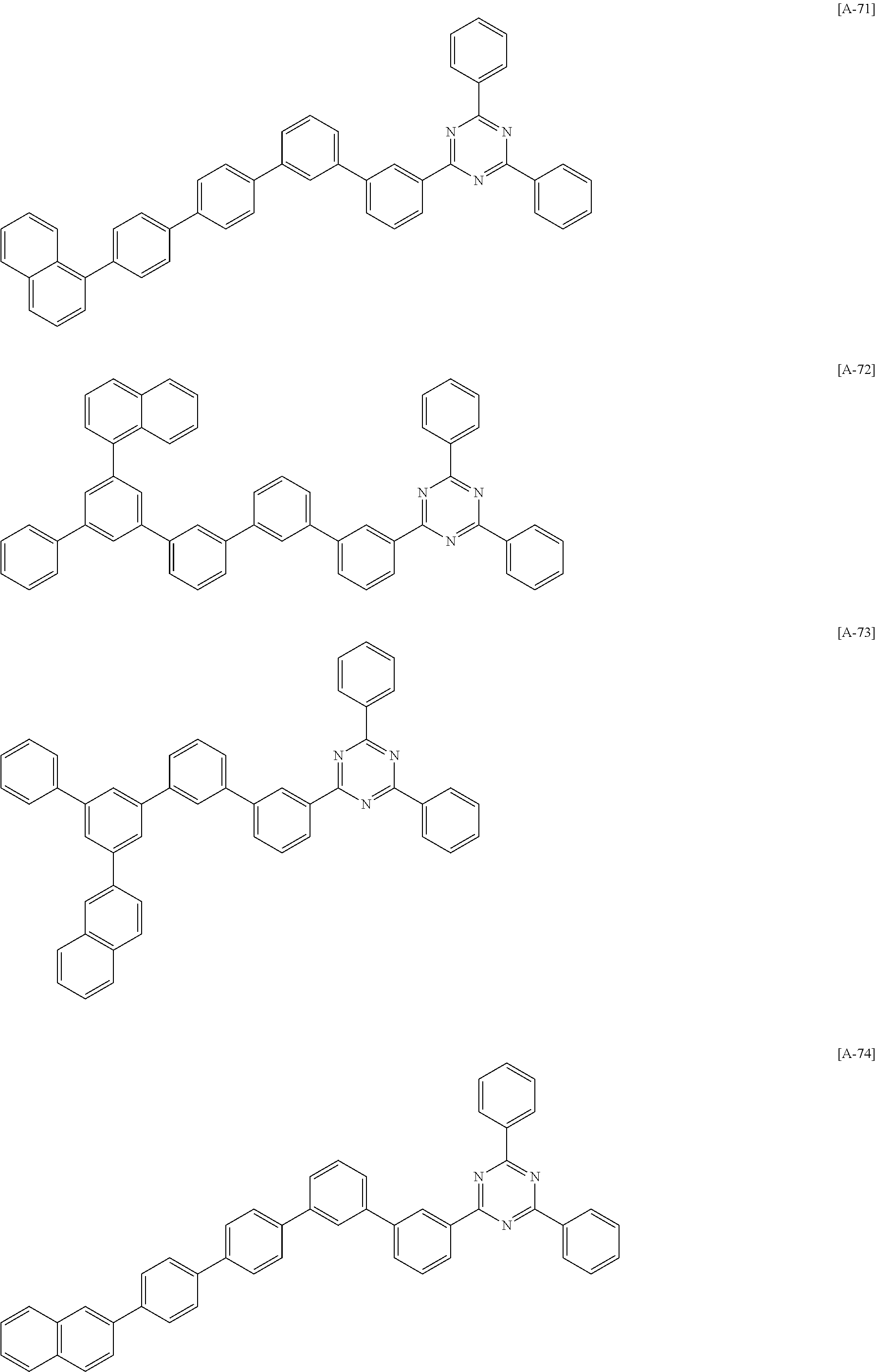 [SMILES: c1ccc(-c2nc(-c3ccccc3)nc(-c3cccc(-c4cccc(-c5ccc(-c6ccc(-c7cccc8ccccc78)cc6)cc5)c4)c3)n2)cc1 ; c1ccc(-c2cc(-c3cccc(-c4cccc(-c5cccc(-c6nc(-c7ccccc7)nc(-c7ccccc7)n6)c5)c4)c3)cc(-c3cccc4ccccc34)c2)cc1 ; c1ccc(-c2cc(-c3cccc(-c4cccc(-c5nc(-c6ccccc6)nc(-c6ccccc6)n5)c4)c3)cc(-c3ccc4ccccc4c3)c2)cc1 ; c1ccc(-c2nc(-c3ccccc3)nc(-c3cccc(-c4cccc(-c5ccc(-c6ccc(-c7ccc8ccccc8c7)cc6)cc5)c4)c3)n2)cc1]

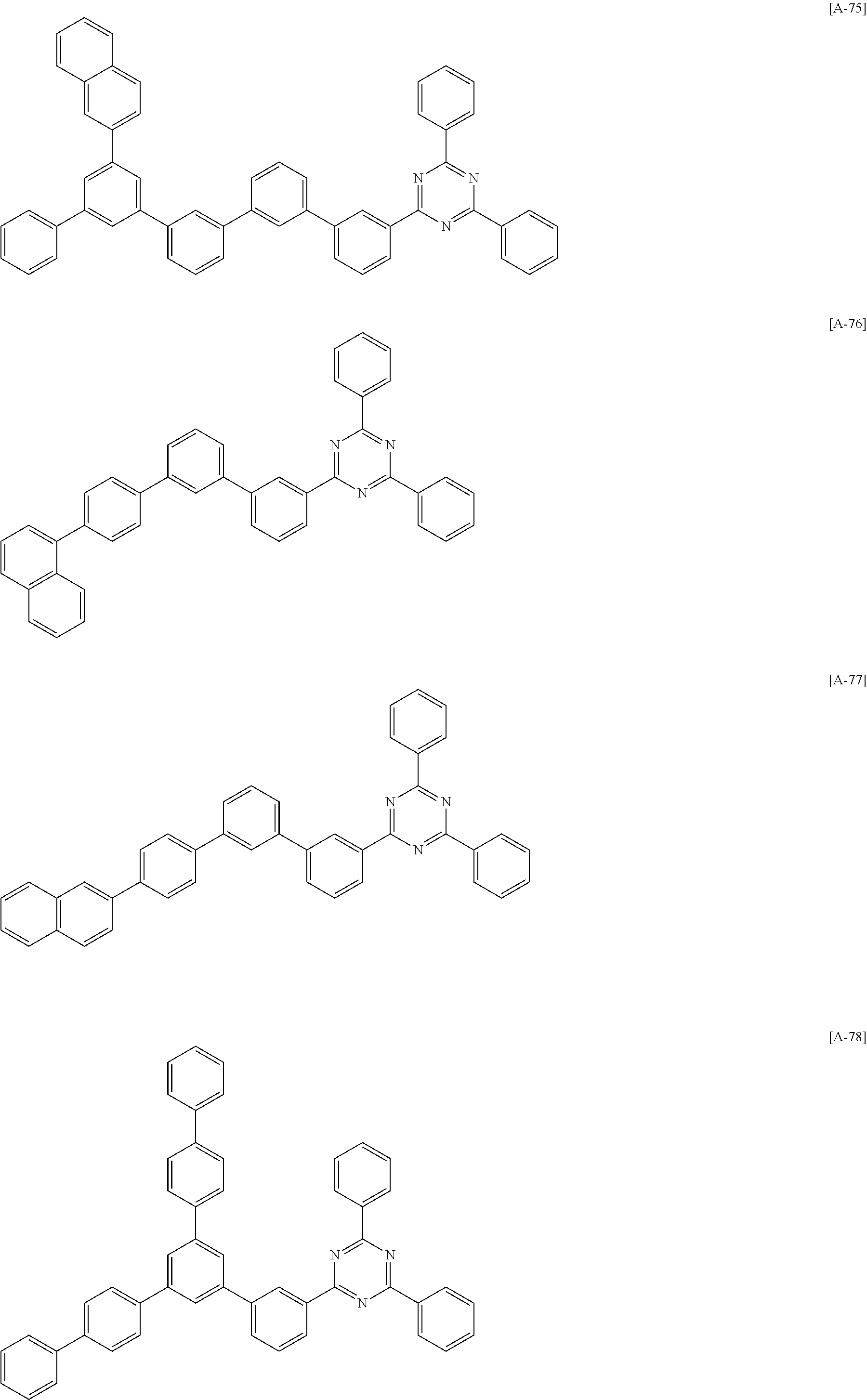 [SMILES: c1ccc(-c2cc(-c3cccc(-c4cccc(-c5cccc(-c6nc(-c7ccccc7)nc(-c7ccccc7)n6)c5)c4)c3)cc(-c3ccc4ccccc4c3)c2)cc1 ; c1ccc(-c2nc(-c3ccccc3)nc(-c3cccc(-c4cccc(-c5ccc(-c6cccc7ccccc67)cc5)c4)c3)n2)cc1 ; c1ccc(-c2nc(-c3ccccc3)nc(-c3cccc(-c4cccc(-c5ccc(-c6ccc7ccccc7c6)cc5)c4)c3)n2)cc1 ; c1ccc(-c2ccc(-c3cc(-c4ccc(-c5ccccc5)cc4)cc(-c4cccc(-c5nc(-c6ccccc6)nc(-c6ccccc6)n5)c4)c3)cc2)cc1]

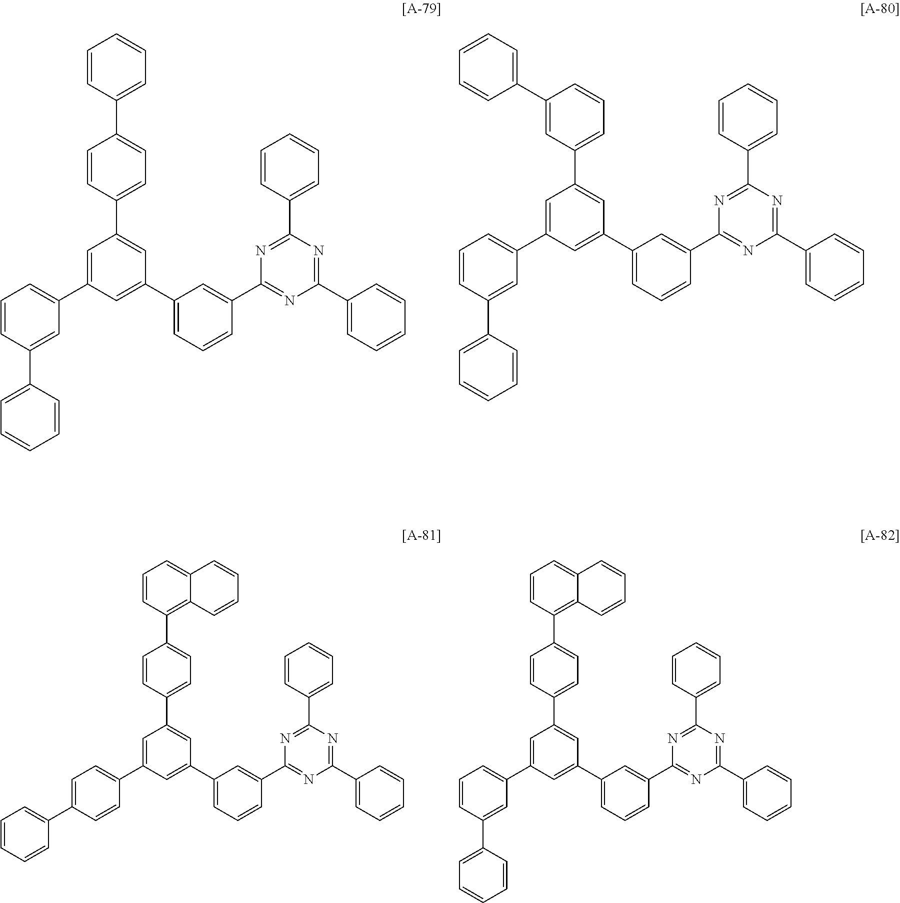 [SMILES: c1ccc(-c2ccc(-c3cc(-c4cccc(-c5ccccc5)c4)cc(-c4cccc(-c5nc(-c6ccccc6)nc(-c6ccccc6)n5)c4)c3)cc2)cc1 ; c1ccc(-c2cccc(-c3cc(-c4cccc(-c5ccccc5)c4)cc(-c4cccc(-c5nc(-c6ccccc6)nc(-c6ccccc6)n5)c4)c3)c2)cc1 ; c1ccc(-c2ccc(-c3cc(-c4ccc(-c5cccc6ccccc56)cc4)cc(-c4cccc(-c5nc(-c6ccccc6)nc(-c6ccccc6)n5)c4)c3)cc2)cc1 ; c1ccc(-c2cccc(-c3cc(-c4ccc(-c5cccc6ccccc56)cc4)cc(-c4cccc(-c5nc(-c6ccccc6)nc(-c6ccccc6)n5)c4)c3)c2)cc1]

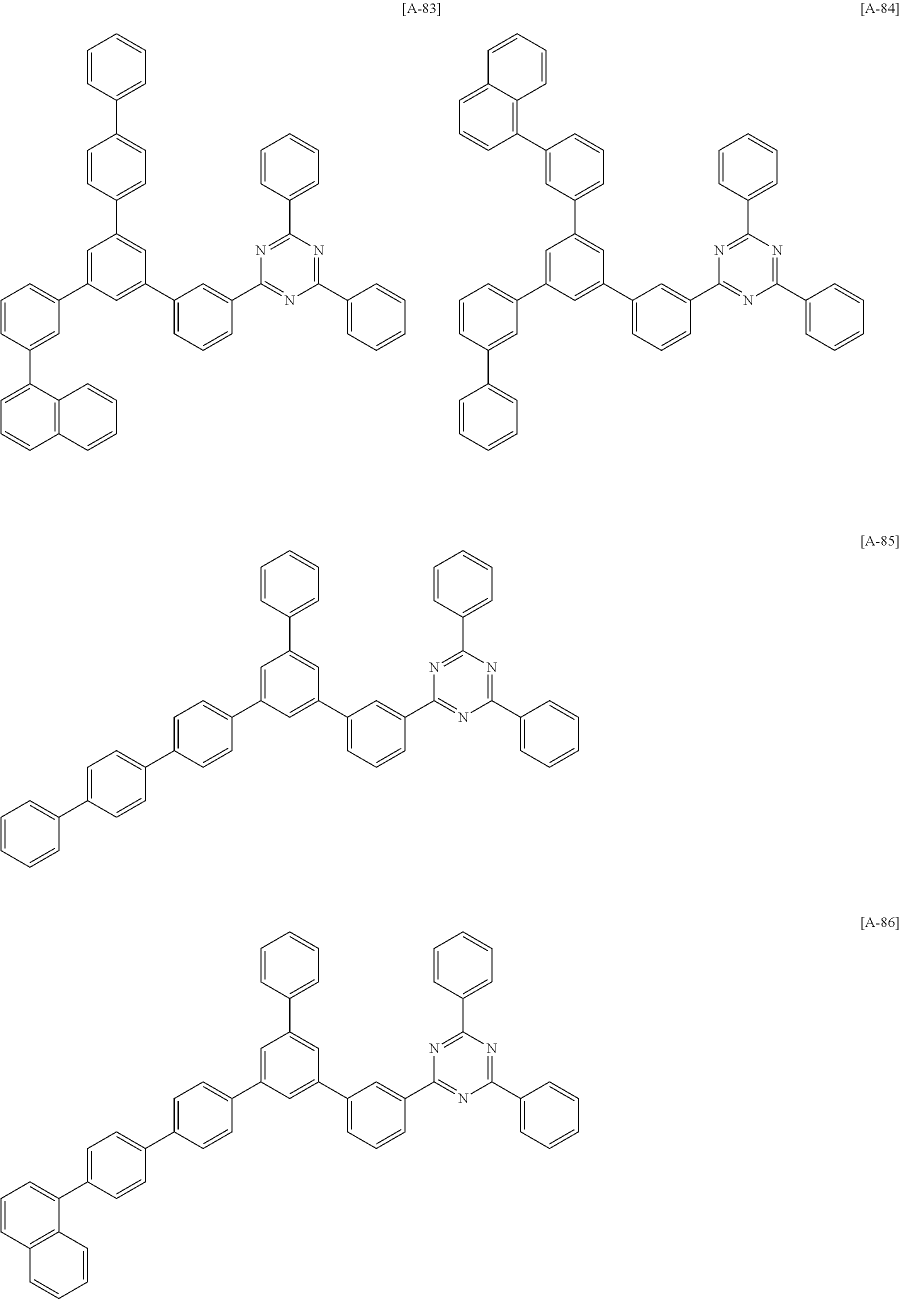 [SMILES: c1ccc(-c2ccc(-c3cc(-c4cccc(-c5nc(-c6ccccc6)nc(-c6ccccc6)n5)c4)cc(-c4cccc(-c5cccc6ccccc56)c4)c3)cc2)cc1 ; c1ccc(-c2cccc(-c3cc(-c4cccc(-c5nc(-c6ccccc6)nc(-c6ccccc6)n5)c4)cc(-c4cccc(-c5cccc6ccccc56)c4)c3)c2)cc1 ; c1ccc(-c2ccc(-c3ccc(-c4cc(-c5ccccc5)cc(-c5cccc(-c6nc(-c7ccccc7)nc(-c7ccccc7)n6)c5)c4)cc3)cc2)cc1 ; c1ccc(-c2cc(-c3ccc(-c4ccc(-c5cccc6ccccc56)cc4)cc3)cc(-c3cccc(-c4nc(-c5ccccc5)nc(-c5ccccc5)n4)c3)c2)cc1]

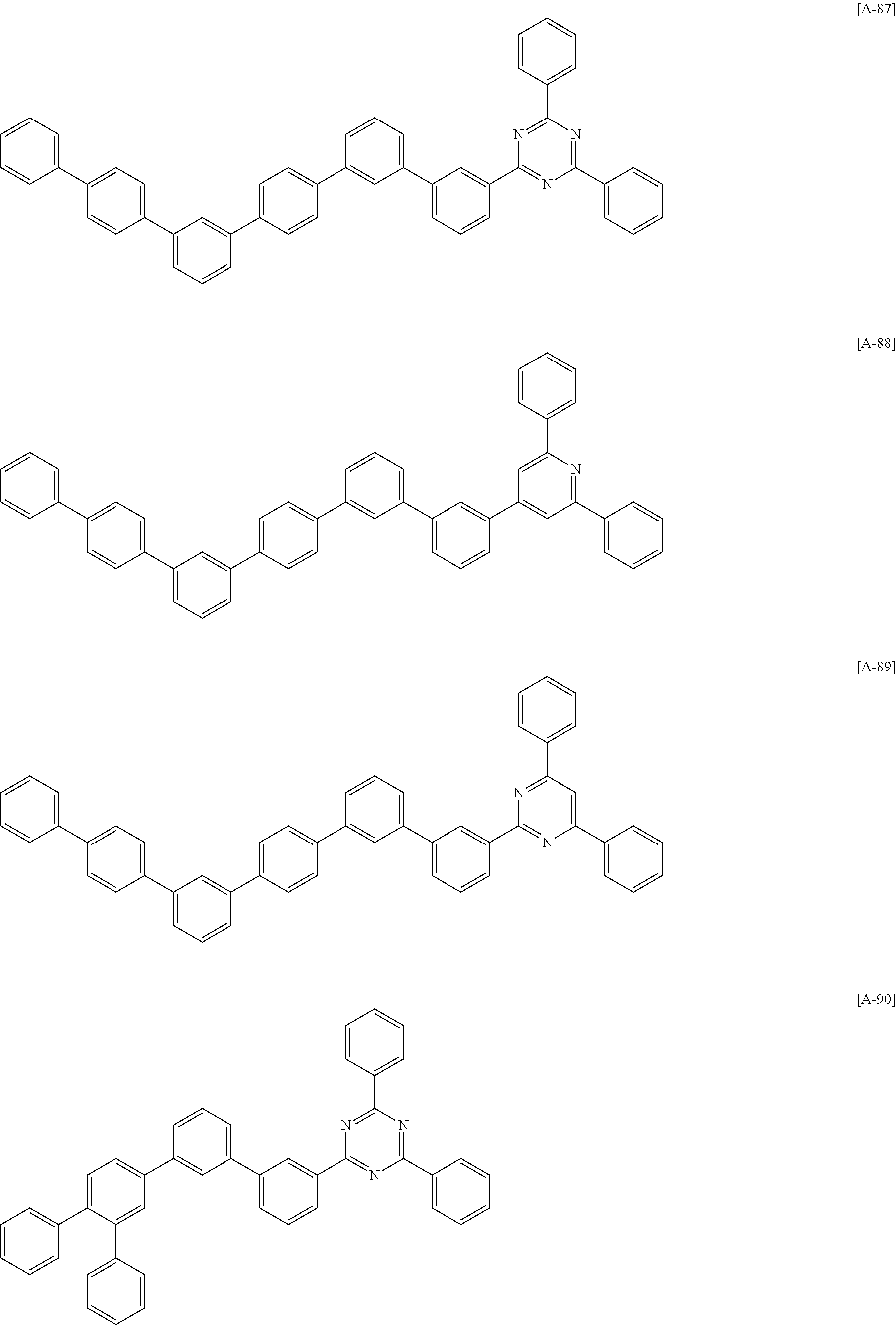 [SMILES: c1ccc(-c2ccc(-c3cccc(-c4ccc(-c5cccc(-c6cccc(-c7nc(-c8ccccc8)nc(-c8ccccc8)n7)c6)c5)cc4)c3)cc2)cc1 ; c1ccc(-c2ccc(-c3cccc(-c4ccc(-c5cccc(-c6cccc(-c7cc(-c8ccccc8)nc(-c8ccccc8)c7)c6)c5)cc4)c3)cc2)cc1 ; c1ccc(-c2ccc(-c3cccc(-c4ccc(-c5cccc(-c6cccc(-c7nc(-c8ccccc8)cc(-c8ccccc8)n7)c6)c5)cc4)c3)cc2)cc1 ; c1ccc(-c2nc(-c3ccccc3)nc(-c3cccc(-c4cccc(-c5ccc(-c6ccccc6)c(-c6ccccc6)c5)c4)c3)n2)cc1]

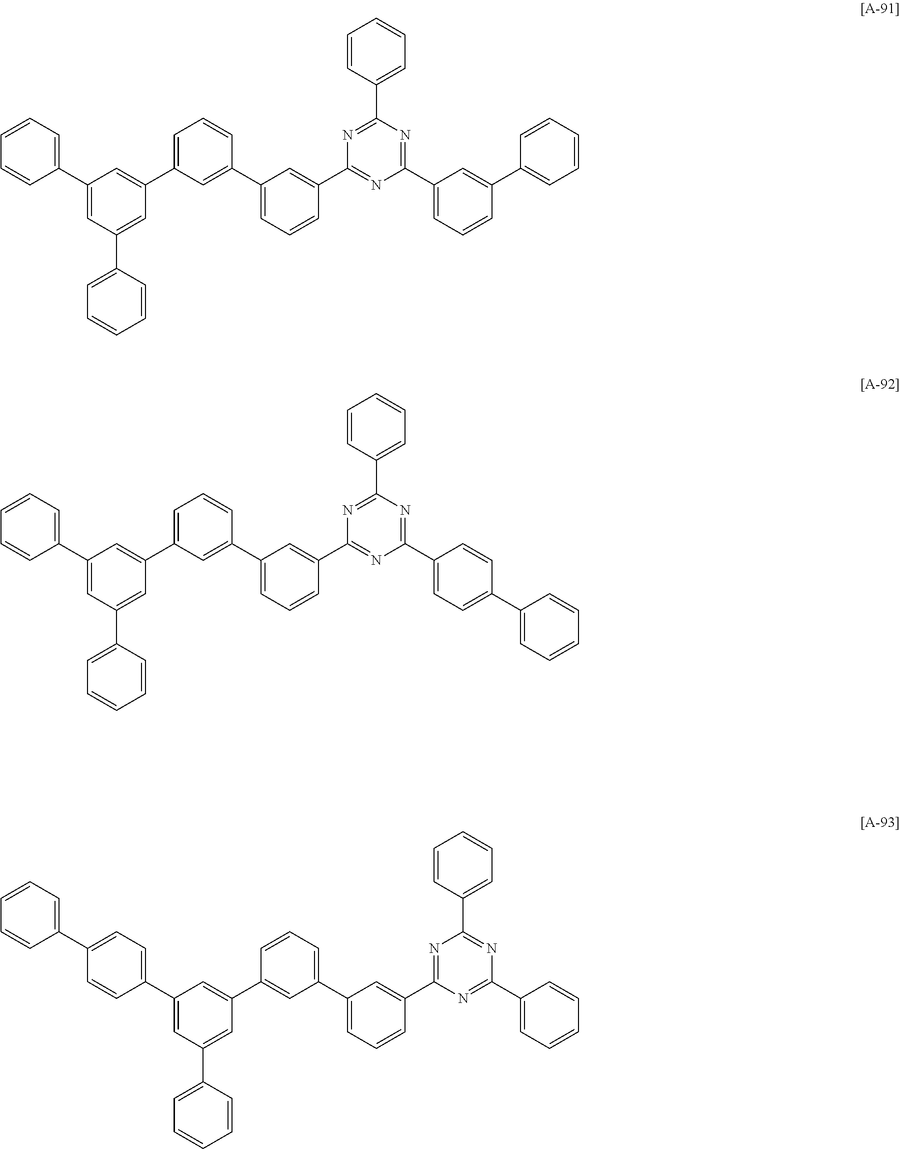 [SMILES: c1ccc(-c2cc(-c3ccccc3)cc(-c3cccc(-c4cccc(-c5nc(-c6ccccc6)nc(-c6cccc(-c7ccccc7)c6)n5)c4)c3)c2)cc1 ; c1ccc(-c2ccc(-c3nc(-c4ccccc4)nc(-c4cccc(-c5cccc(-c6cc(-c7ccccc7)cc(-c7ccccc7)c6)c5)c4)n3)cc2)cc1 ; c1ccc(-c2ccc(-c3cc(-c4ccccc4)cc(-c4cccc(-c5cccc(-c6nc(-c7ccccc7)nc(-c7ccccc7)n6)c5)c4)c3)cc2)cc1]

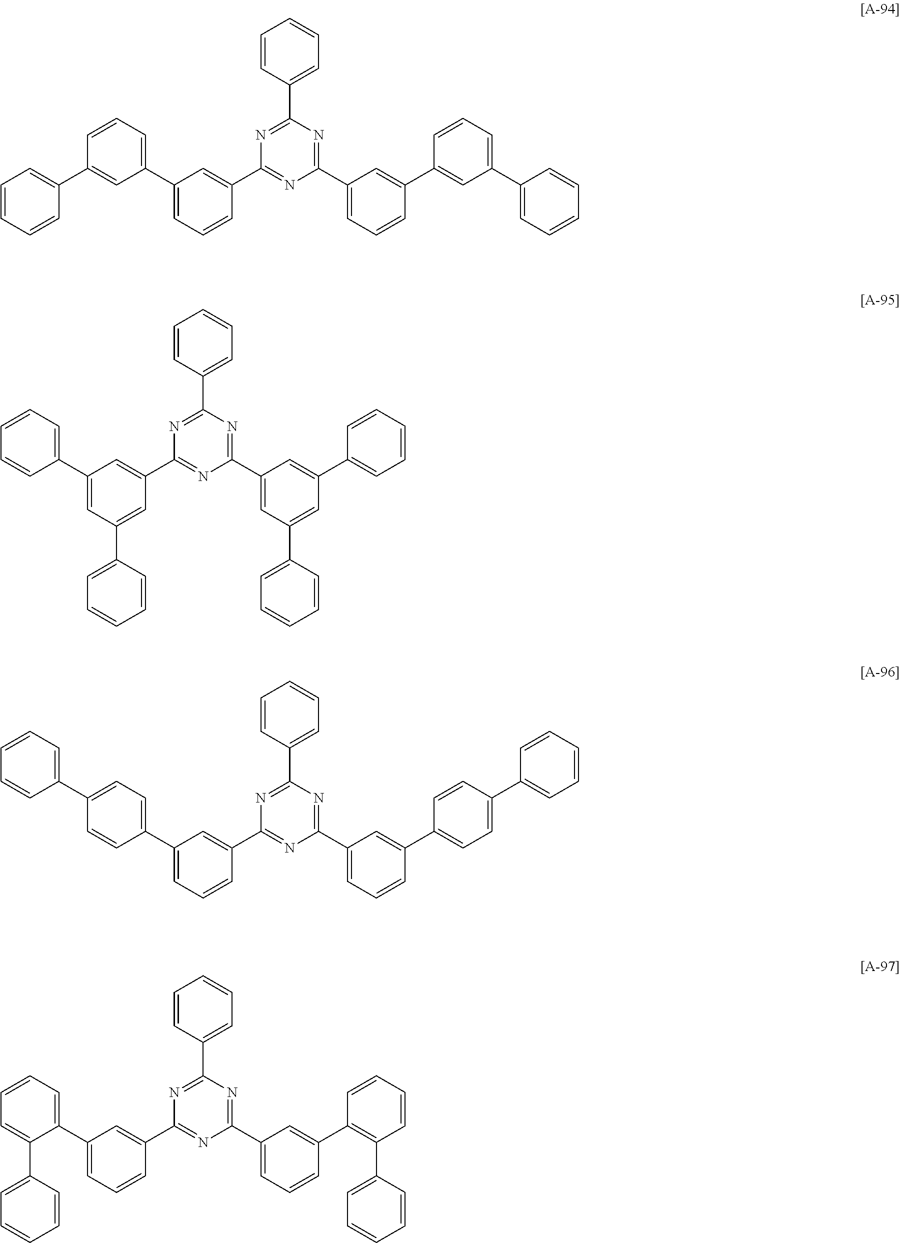 [SMILES: c1ccc(-c2cccc(-c3cccc(-c4nc(-c5ccccc5)nc(-c5cccc(-c6cccc(-c7ccccc7)c6)c5)n4)c3)c2)cc1 ; c1ccc(-c2cc(-c3ccccc3)cc(-c3nc(-c4ccccc4)nc(-c4cc(-c5ccccc5)cc(-c5ccccc5)c4)n3)c2)cc1 ; c1ccc(-c2ccc(-c3cccc(-c4nc(-c5ccccc5)nc(-c5cccc(-c6ccc(-c7ccccc7)cc6)c5)n4)c3)cc2)cc1 ; c1ccc(-c2nc(-c3cccc(-c4ccccc4-c4ccccc4)c3)nc(-c3cccc(-c4ccccc4-c4ccccc4)c3)n2)cc1]

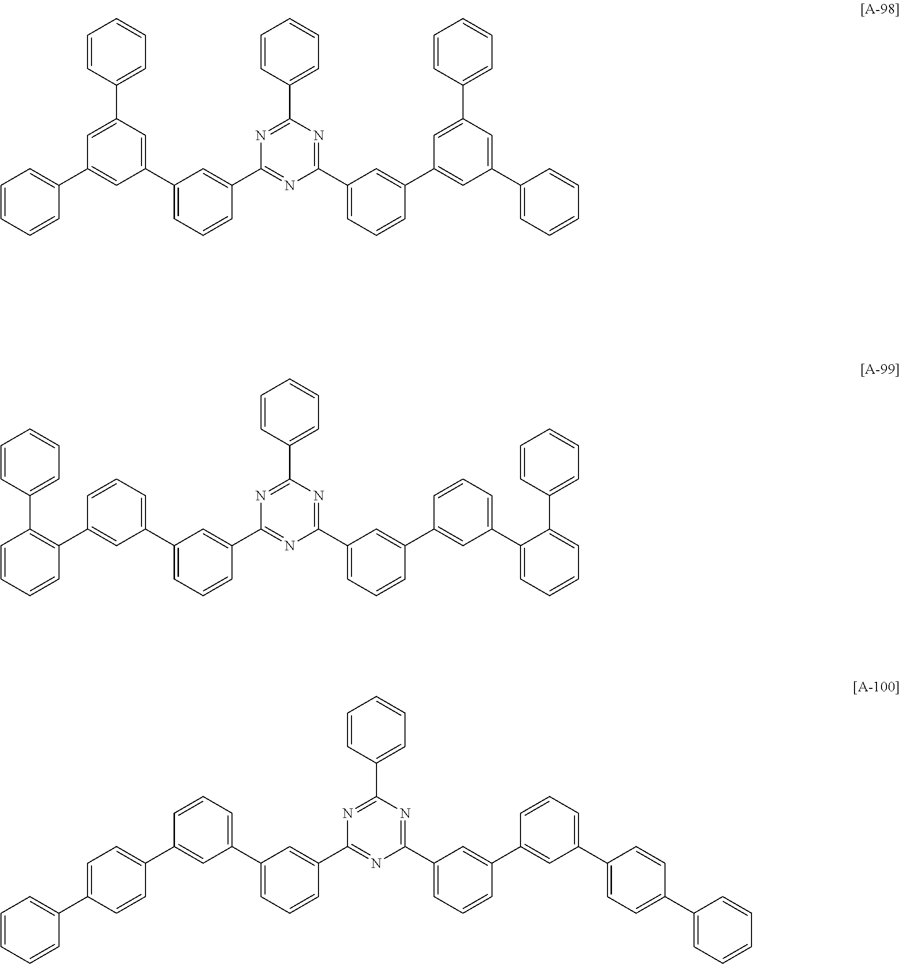 [SMILES: c1ccc(-c2cc(-c3ccccc3)cc(-c3cccc(-c4nc(-c5ccccc5)nc(-c5cccc(-c6cc(-c7ccccc7)cc(-c7ccccc7)c6)c5)n4)c3)c2)cc1 ; c1ccc(-c2nc(-c3cccc(-c4cccc(-c5ccccc5-c5ccccc5)c4)c3)nc(-c3cccc(-c4cccc(-c5ccccc5-c5ccccc5)c4)c3)n2)cc1 ; c1ccc(-c2ccc(-c3cccc(-c4cccc(-c5nc(-c6ccccc6)nc(-c6cccc(-c7cccc(-c8ccc(-c9ccccc9)cc8)c7)c6)n5)c4)c3)cc2)cc1]

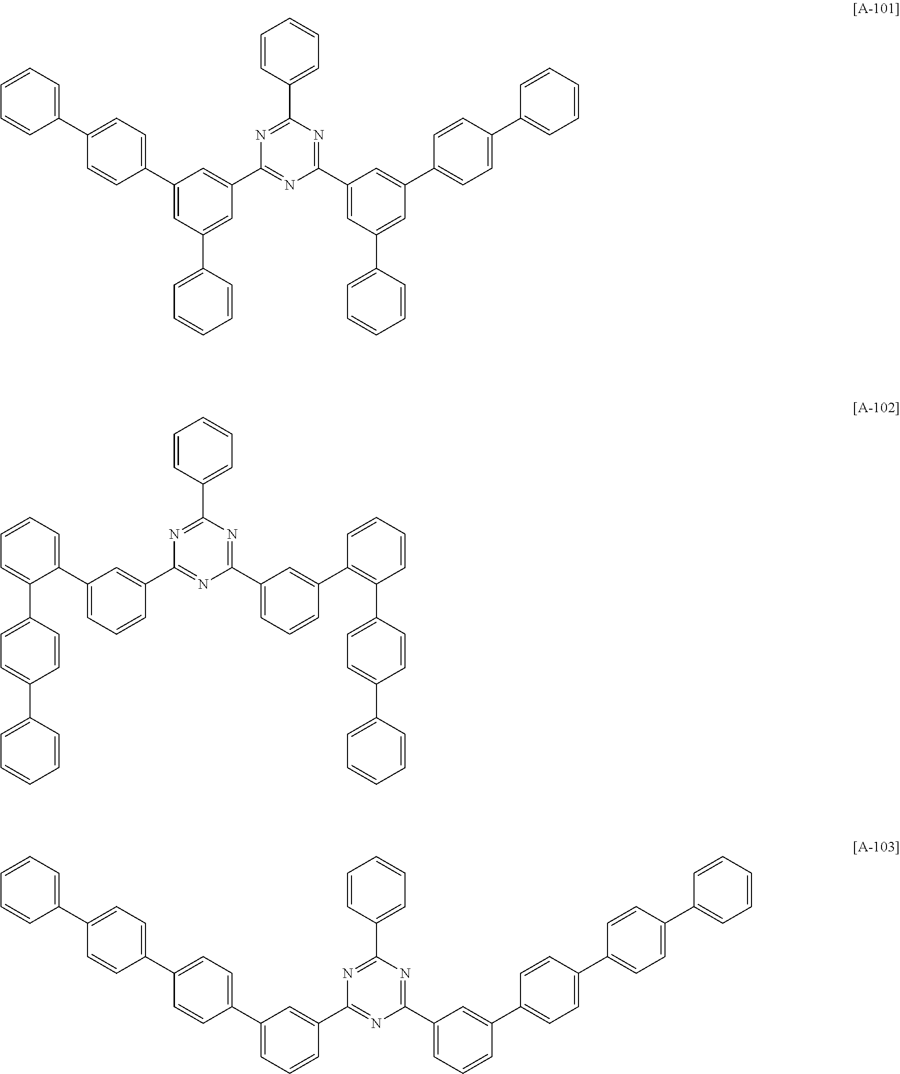 [SMILES: c1ccc(-c2ccc(-c3cc(-c4ccccc4)cc(-c4nc(-c5ccccc5)nc(-c5cc(-c6ccccc6)cc(-c6ccc(-c7ccccc7)cc6)c5)n4)c3)cc2)cc1 ; c1ccc(-c2ccc(-c3ccccc3-c3cccc(-c4nc(-c5ccccc5)nc(-c5cccc(-c6ccccc6-c6ccc(-c7ccccc7)cc6)c5)n4)c3)cc2)cc1 ; c1ccc(-c2ccc(-c3ccc(-c4cccc(-c5nc(-c6ccccc6)nc(-c6cccc(-c7ccc(-c8ccc(-c9ccccc9)cc8)cc7)c6)n5)c4)cc3)cc2)cc1]

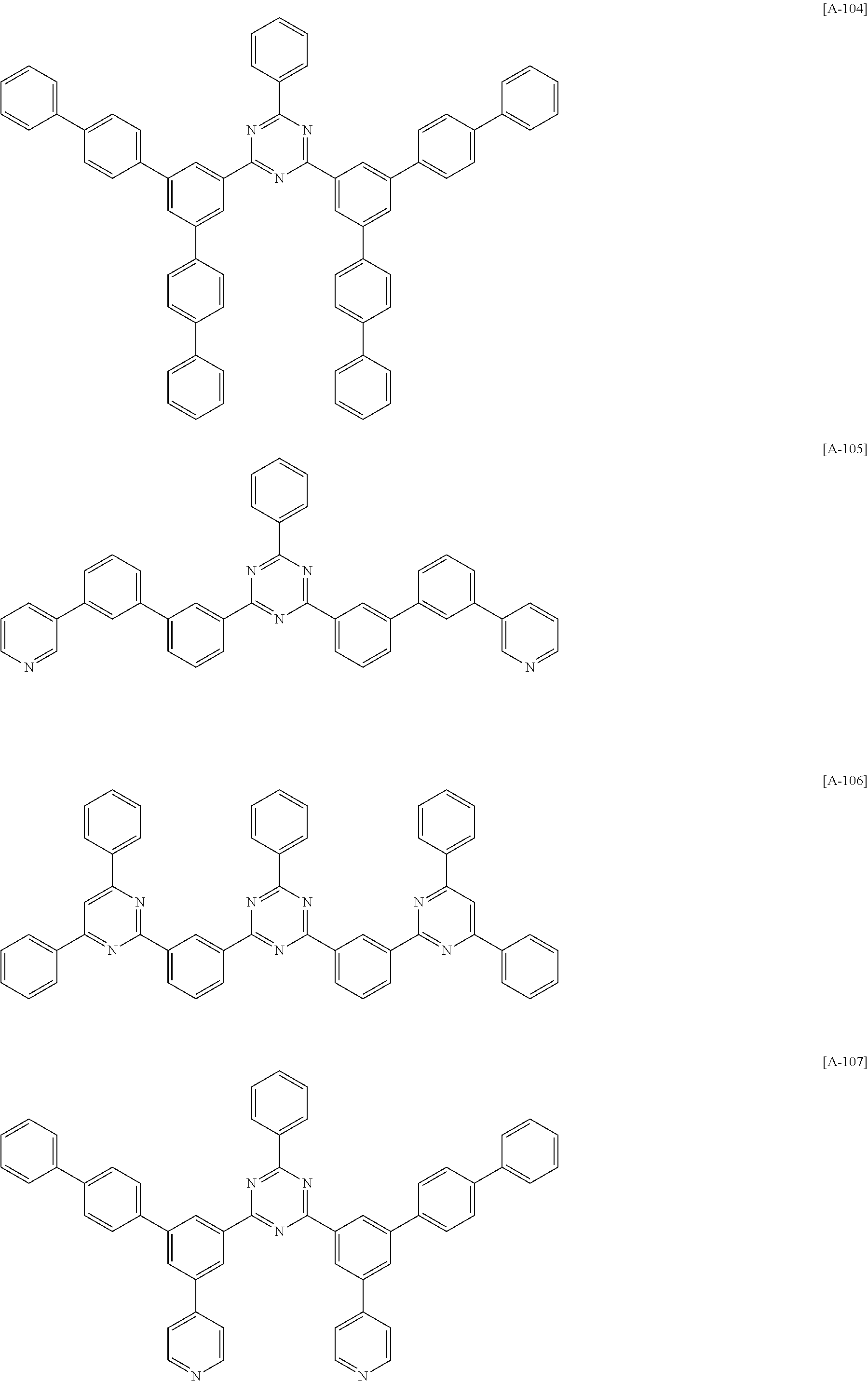 [SMILES: c1ccc(-c2ccc(-c3cc(-c4ccc(-c5ccccc5)cc4)cc(-c4nc(-c5ccccc5)nc(-c5cc(-c6ccc(-c7ccccc7)cc6)cc(-c6ccc(-c7ccccc7)cc6)c5)n4)c3)cc2)cc1 ; c1ccc(-c2nc(-c3cccc(-c4cccc(-c5cccnc5)c4)c3)nc(-c3cccc(-c4cccc(-c5cccnc5)c4)c3)n2)cc1 ; c1ccc(-c2cc(-c3ccccc3)nc(-c3cccc(-c4nc(-c5ccccc5)nc(-c5cccc(-c6nc(-c7ccccc7)cc(-c7ccccc7)n6)c5)n4)c3)n2)cc1 ; c1ccc(-c2ccc(-c3cc(-c4ccncc4)cc(-c4nc(-c5ccccc5)nc(-c5cc(-c6ccncc6)cc(-c6ccc(-c7ccccc7)cc6)c5)n4)c3)cc2)cc1]

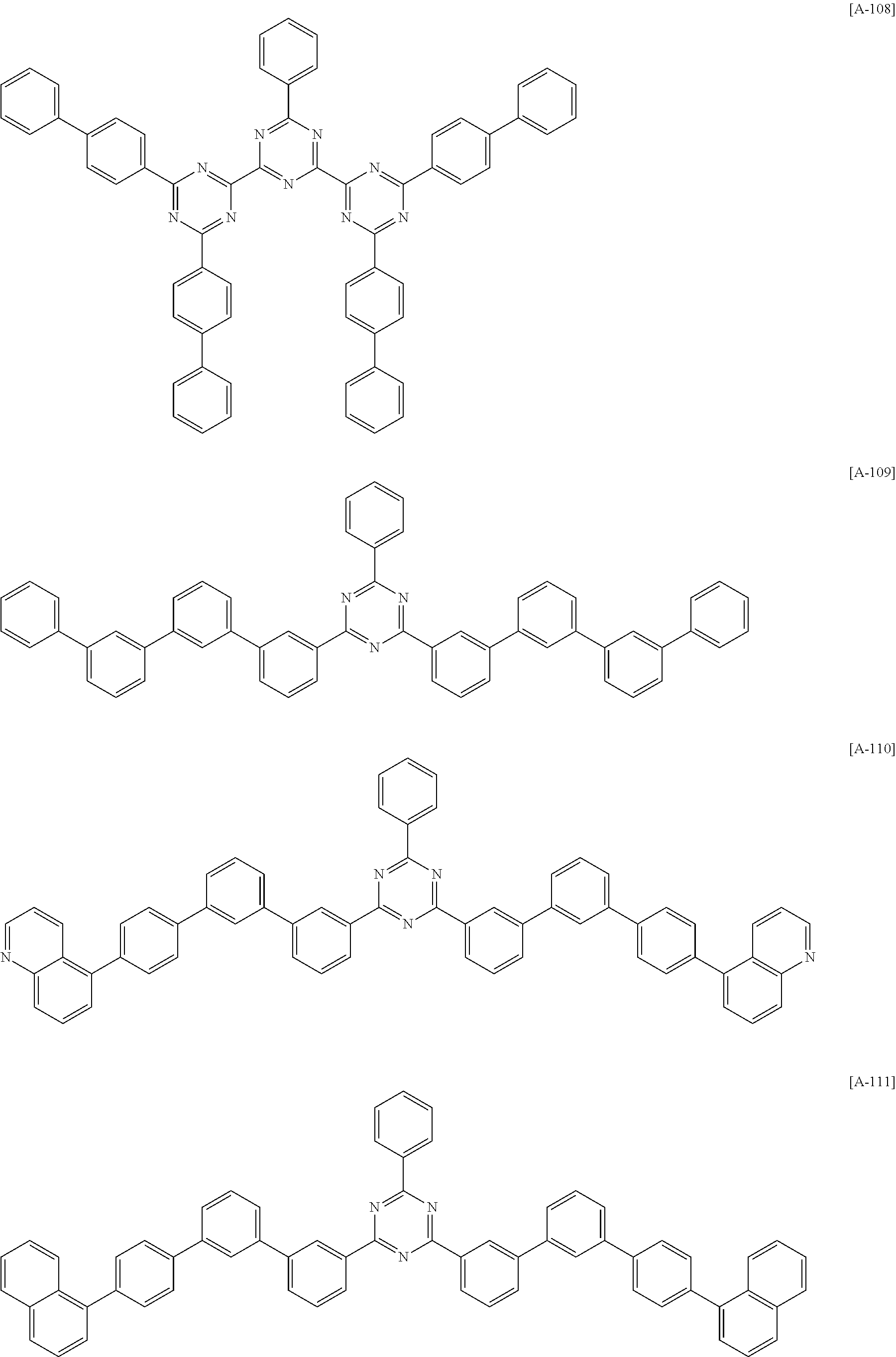 [SMILES: c1ccc(-c2ccc(-c3nc(-c4ccc(-c5ccccc5)cc4)nc(-c4nc(-c5ccccc5)nc(-c5nc(-c6ccc(-c7ccccc7)cc6)nc(-c6ccc(-c7ccccc7)cc6)n5)n4)n3)cc2)cc1 ; c1ccc(-c2cccc(-c3cccc(-c4cccc(-c5nc(-c6ccccc6)nc(-c6cccc(-c7cccc(-c8cccc(-c9ccccc9)c8)c7)c6)n5)c4)c3)c2)cc1 ; c1ccc(-c2nc(-c3cccc(-c4cccc(-c5ccc(-c6cccc7ncccc67)cc5)c4)c3)nc(-c3cccc(-c4cccc(-c5ccc(-c6cccc7ncccc67)cc5)c4)c3)n2)cc1 ; c1ccc(-c2nc(-c3cccc(-c4cccc(-c5ccc(-c6cccc7ccccc67)cc5)c4)c3)nc(-c3cccc(-c4cccc(-c5ccc(-c6cccc7ccccc67)cc5)c4)c3)n2)cc1]

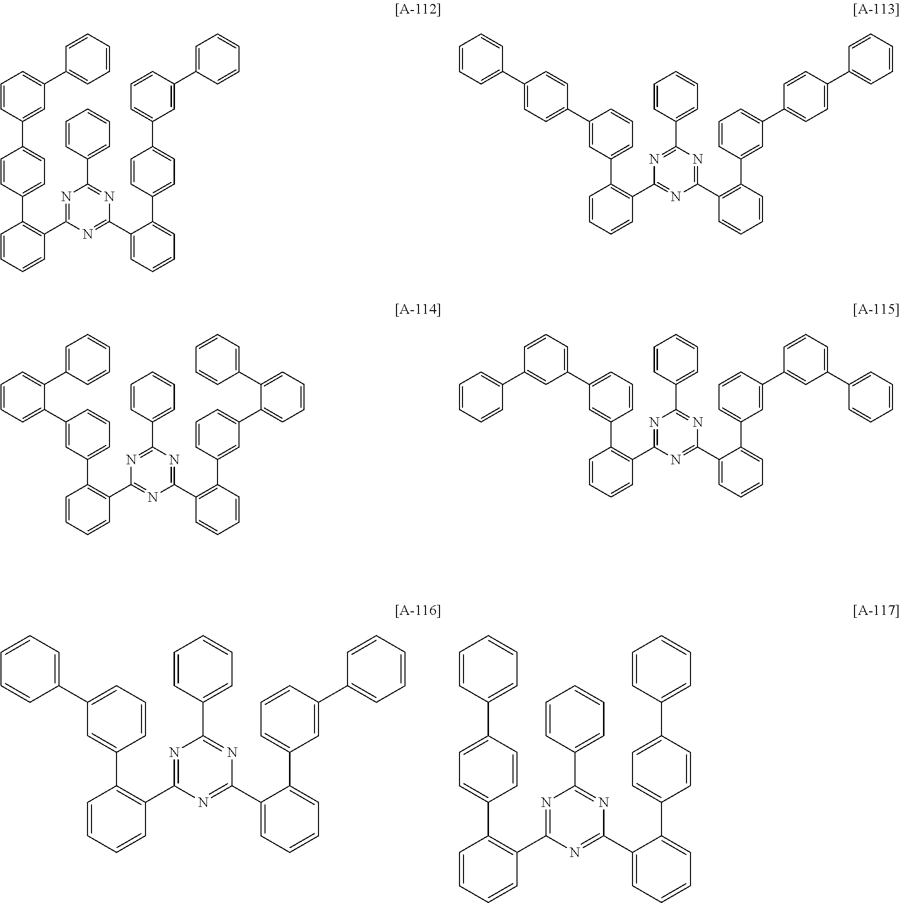

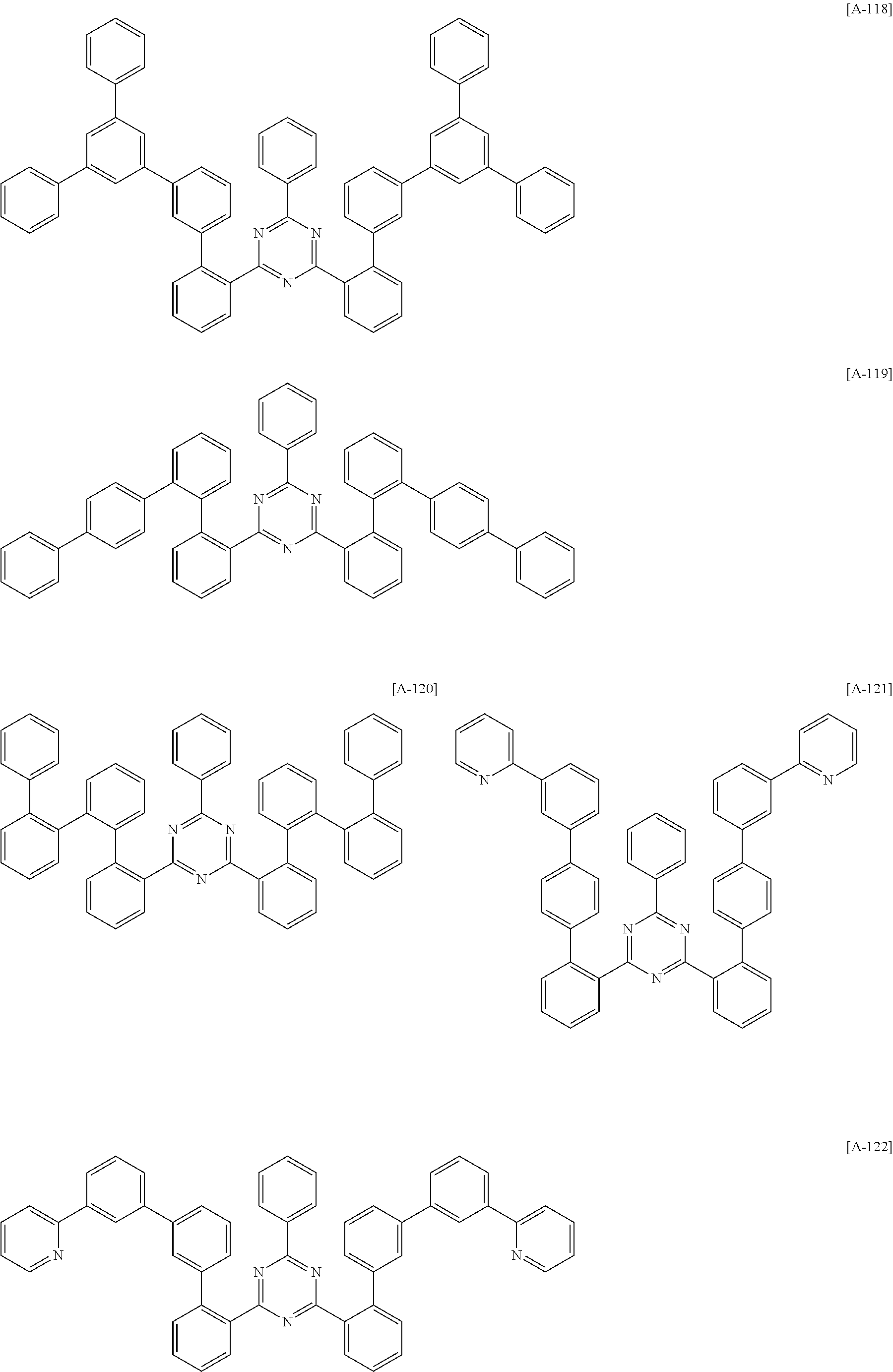

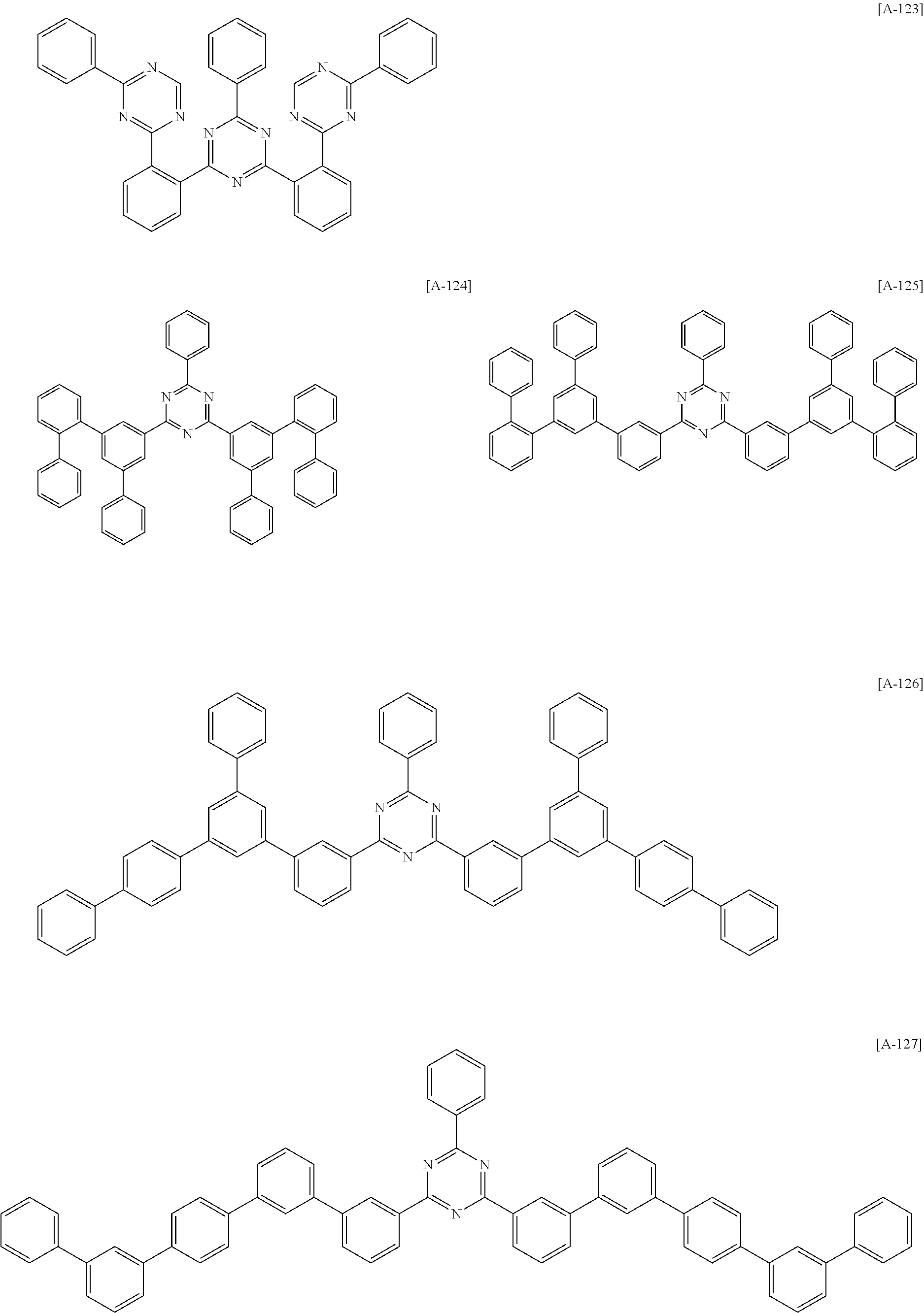

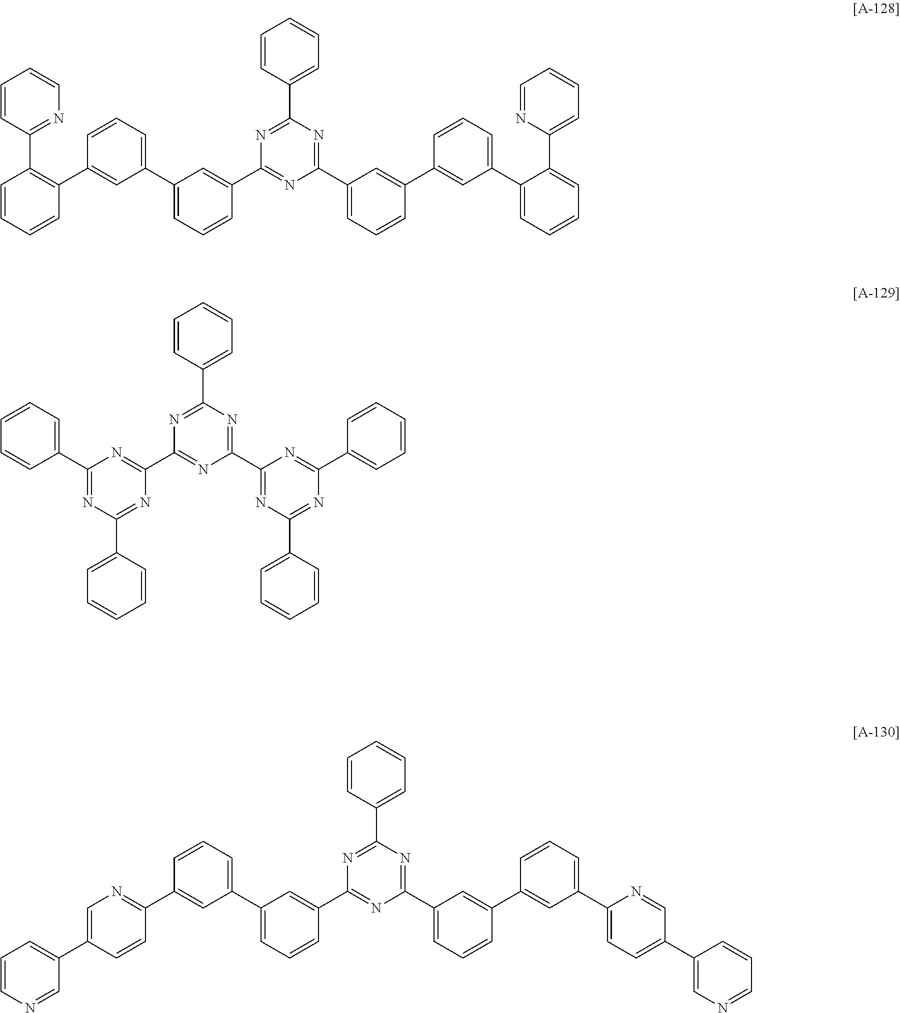 [SMILES: c1ccc(-c2nc(-c3cccc(-c4cccc(-c5ccccc5-c5ccccn5)c4)c3)nc(-c3cccc(-c4cccc(-c5ccccc5-c5ccccn5)c4)c3)n2)cc1 ; c1ccc(-c2nc(-c3ccccc3)nc(-c3nc(-c4ccccc4)nc(-c4nc(-c5ccccc5)nc(-c5ccccc5)n4)n3)n2)cc1 ; c1ccc(-c2nc(-c3cccc(-c4cccc(-c5ccc(-c6cccnc6)cn5)c4)c3)nc(-c3cccc(-c4cccc(-c5ccc(-c6cccnc6)cn5)c4)c3)n2)cc1]

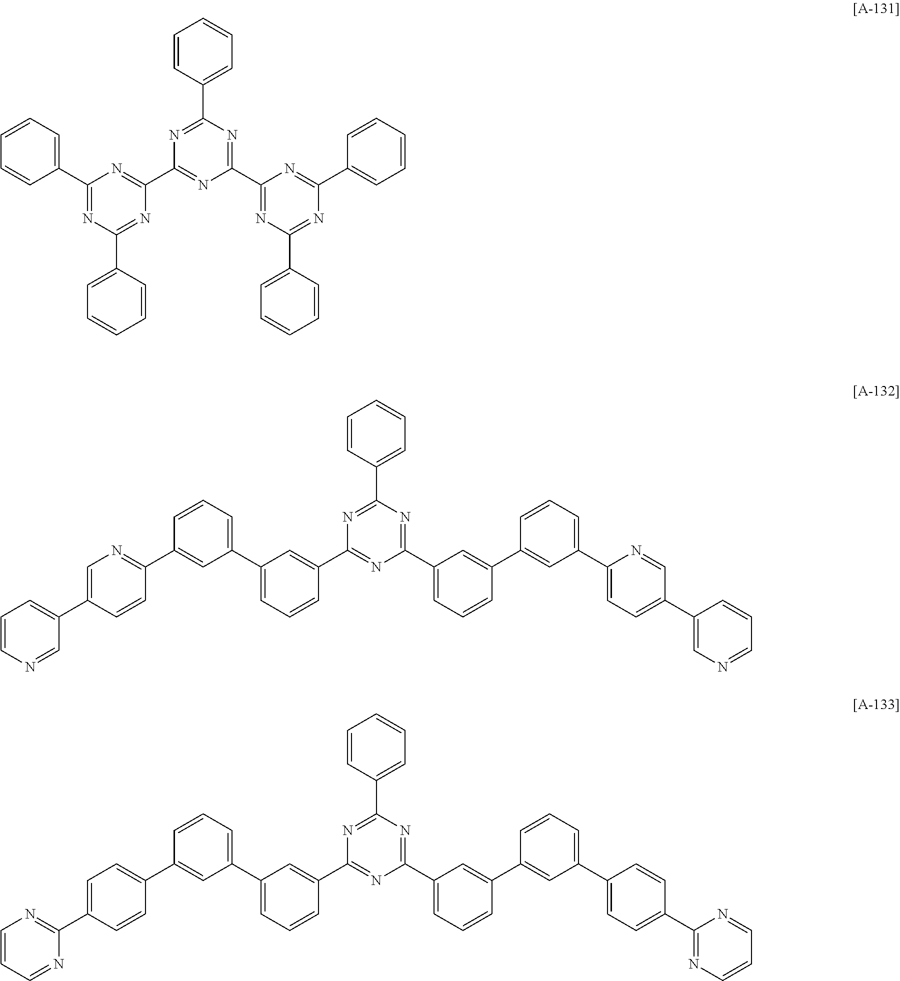 [SMILES: c1ccc(-c2nc(-c3ccccc3)nc(-c3nc(-c4ccccc4)nc(-c4nc(-c5ccccc5)nc(-c5ccccc5)n4)n3)n2)cc1 ; c1ccc(-c2nc(-c3cccc(-c4cccc(-c5ccc(-c6cccnc6)cn5)c4)c3)nc(-c3cccc(-c4cccc(-c5ccc(-c6cccnc6)cn5)c4)c3)n2)cc1 ; c1ccc(-c2nc(-c3cccc(-c4cccc(-c5ccc(-c6ncccn6)cc5)c4)c3)nc(-c3cccc(-c4cccc(-c5ccc(-c6ncccn6)cc5)c4)c3)n2)cc1]

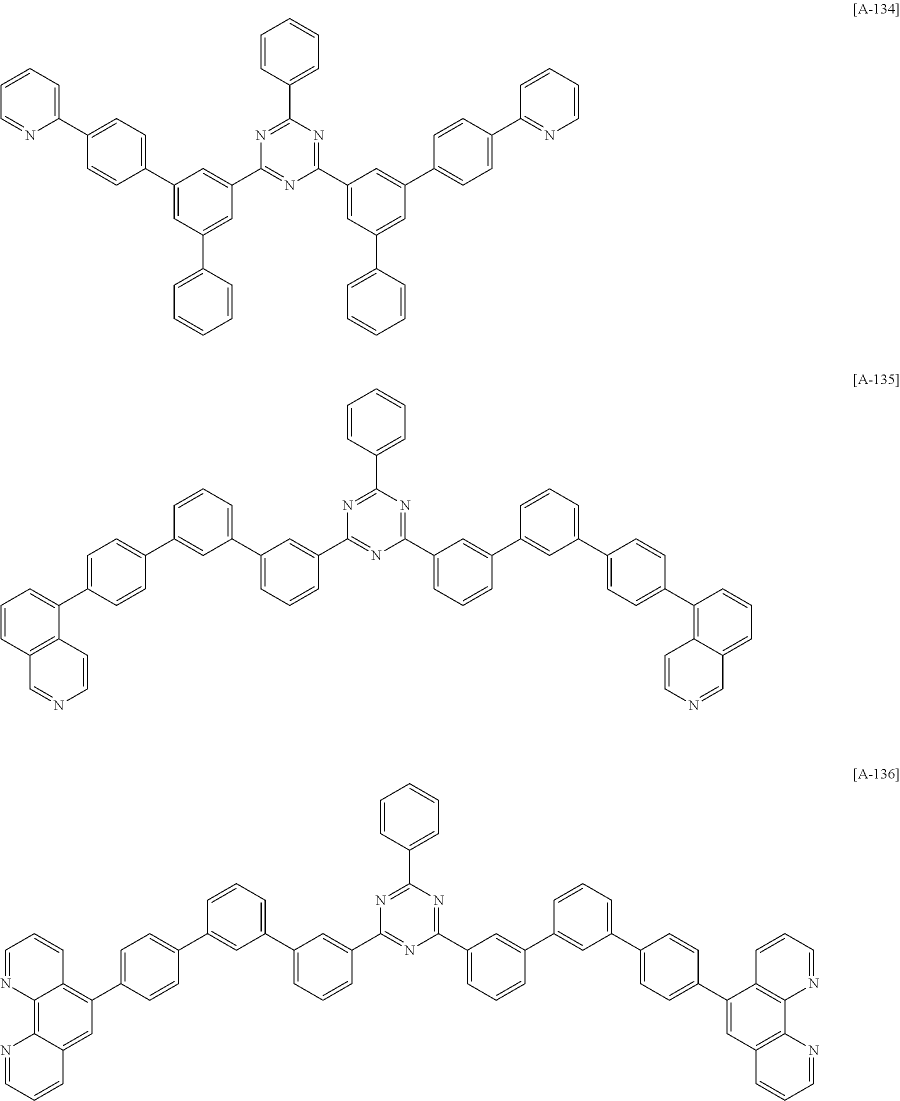 [SMILES: c1ccc(-c2cc(-c3ccc(-c4ccccn4)cc3)cc(-c3nc(-c4ccccc4)nc(-c4cc(-c5ccccc5)cc(-c5ccc(-c6ccccn6)cc5)c4)n3)c2)cc1 ; c1ccc(-c2nc(-c3cccc(-c4cccc(-c5ccc(-c6cccc7cnccc67)cc5)c4)c3)nc(-c3cccc(-c4cccc(-c5ccc(-c6cccc7cnccc67)cc5)c4)c3)n2)cc1 ; c1ccc(-c2nc(-c3cccc(-c4cccc(-c5ccc(-c6cc7cccnc7c7ncccc67)cc5)c4)c3)nc(-c3cccc(-c4cccc(-c5ccc(-c6cc7cccnc7c7ncccc67)cc5)c4)c3)n2)cc1]

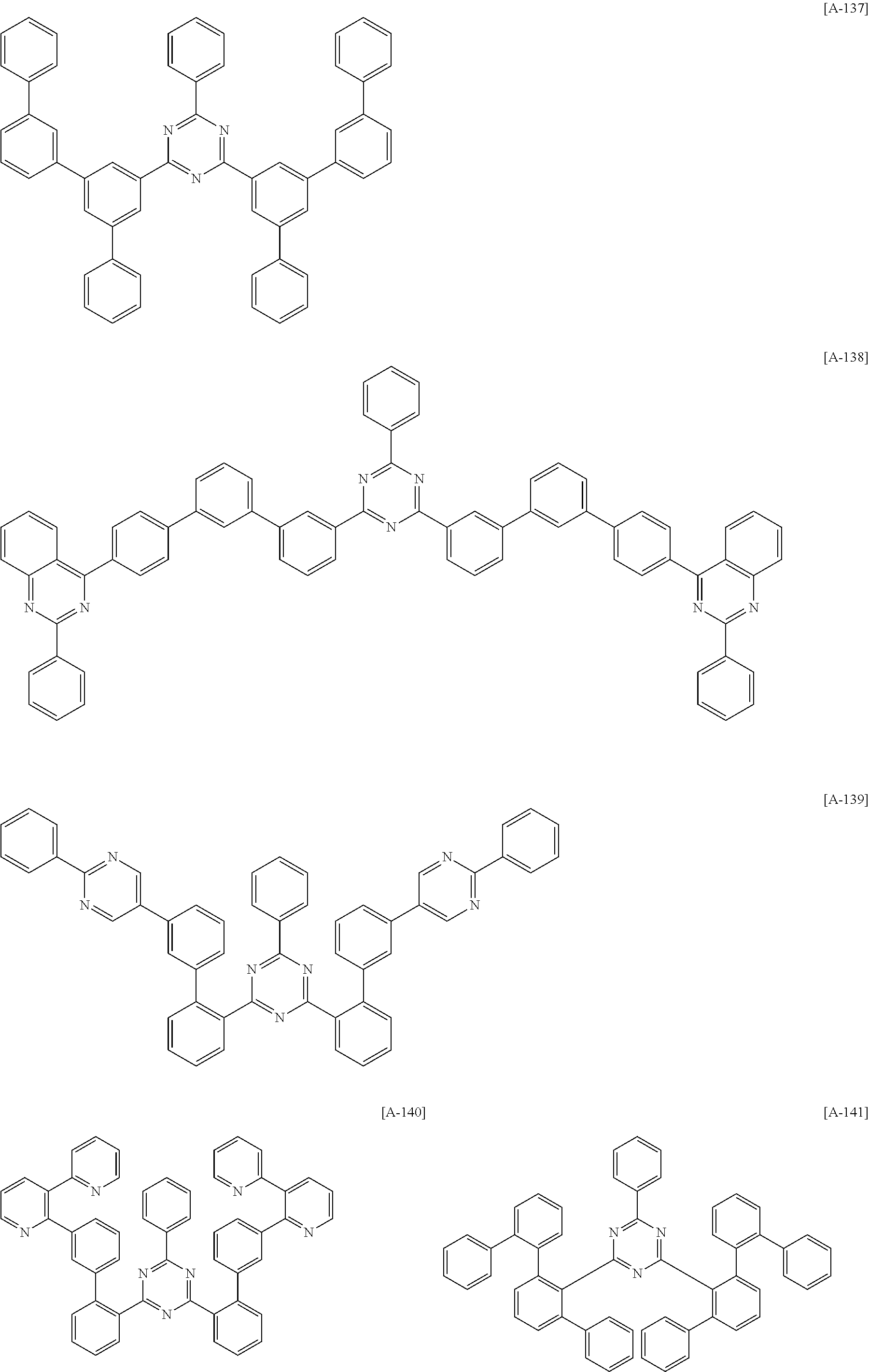

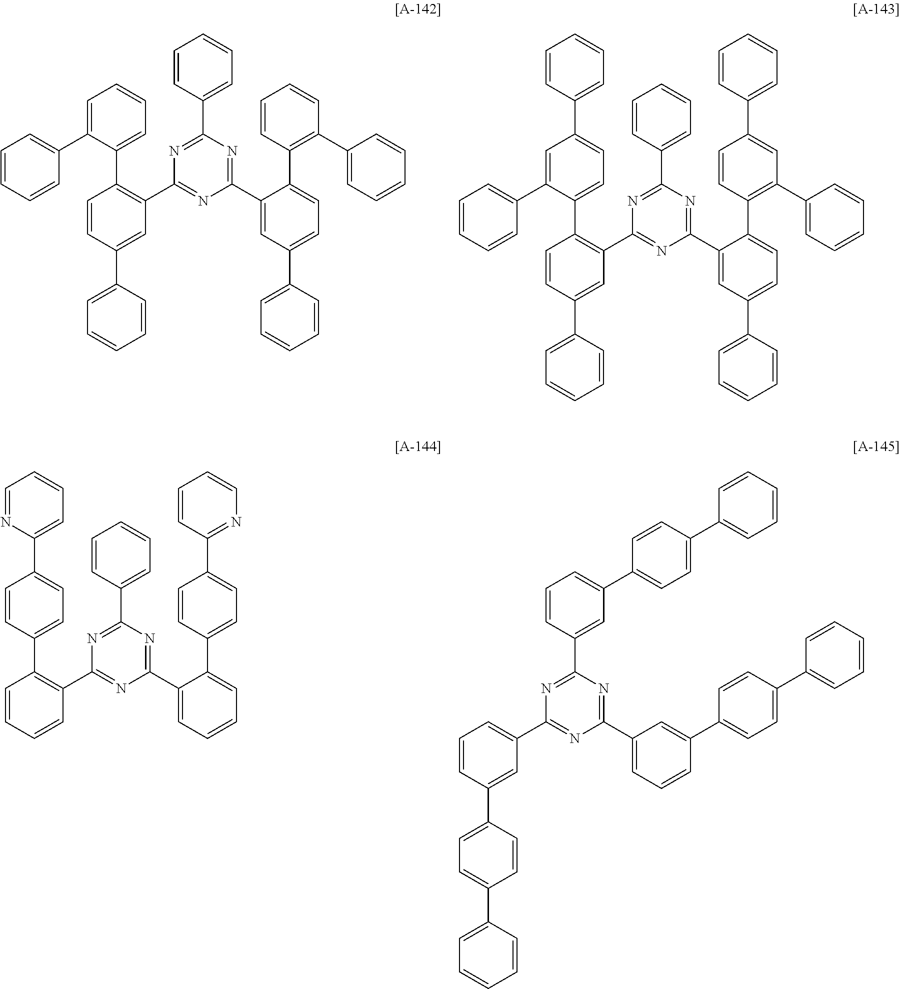 [SMILES: c1ccc(-c2ccc(-c3ccccc3-c3ccccc3)c(-c3nc(-c4ccccc4)nc(-c4cc(-c5ccccc5)ccc4-c4ccccc4-c4ccccc4)n3)c2)cc1 ; c1ccc(-c2ccc(-c3ccc(-c4ccccc4)cc3-c3nc(-c4ccccc4)nc(-c4cc(-c5ccccc5)ccc4-c4ccc(-c5ccccc5)cc4-c4ccccc4)n3)c(-c3ccccc3)c2)cc1 ; c1ccc(-c2nc(-c3ccccc3-c3ccc(-c4ccccn4)cc3)nc(-c3ccccc3-c3ccc(-c4ccccn4)cc3)n2)cc1 ; c1ccc(-c2ccc(-c3cccc(-c4nc(-c5cccc(-c6ccc(-c7ccccc7)cc6)c5)nc(-c5cccc(-c6ccc(-c7ccccc7)cc6)c5)n4)c3)cc2)cc1]

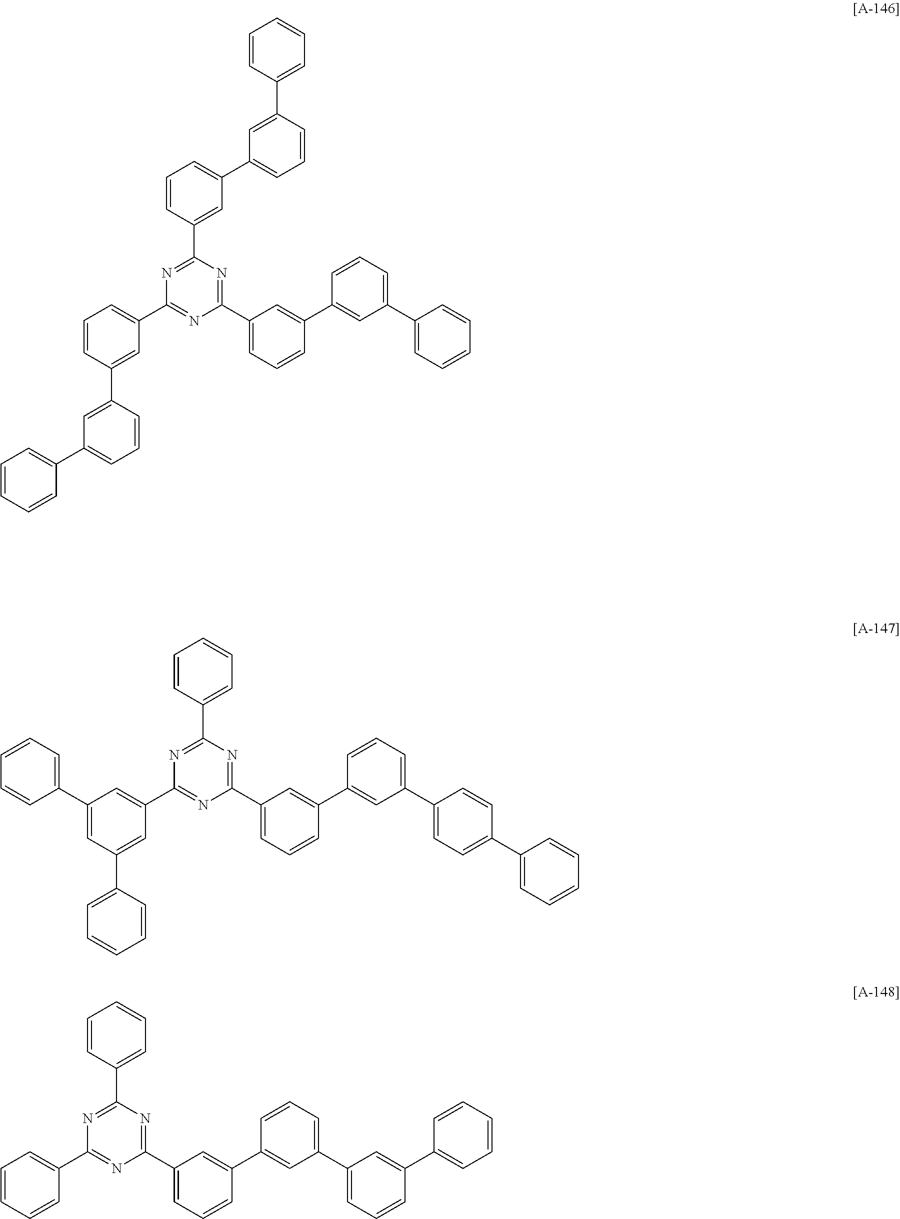 [SMILES: c1ccc(-c2cccc(-c3cccc(-c4nc(-c5cccc(-c6cccc(-c7ccccc7)c6)c5)nc(-c5cccc(-c6cccc(-c7ccccc7)c6)c5)n4)c3)c2)cc1 ; c1ccc(-c2ccc(-c3cccc(-c4cccc(-c5nc(-c6ccccc6)nc(-c6cc(-c7ccccc7)cc(-c7ccccc7)c6)n5)c4)c3)cc2)cc1 ; c1ccc(-c2cccc(-c3cccc(-c4cccc(-c5nc(-c6ccccc6)nc(-c6ccccc6)n5)c4)c3)c2)cc1]

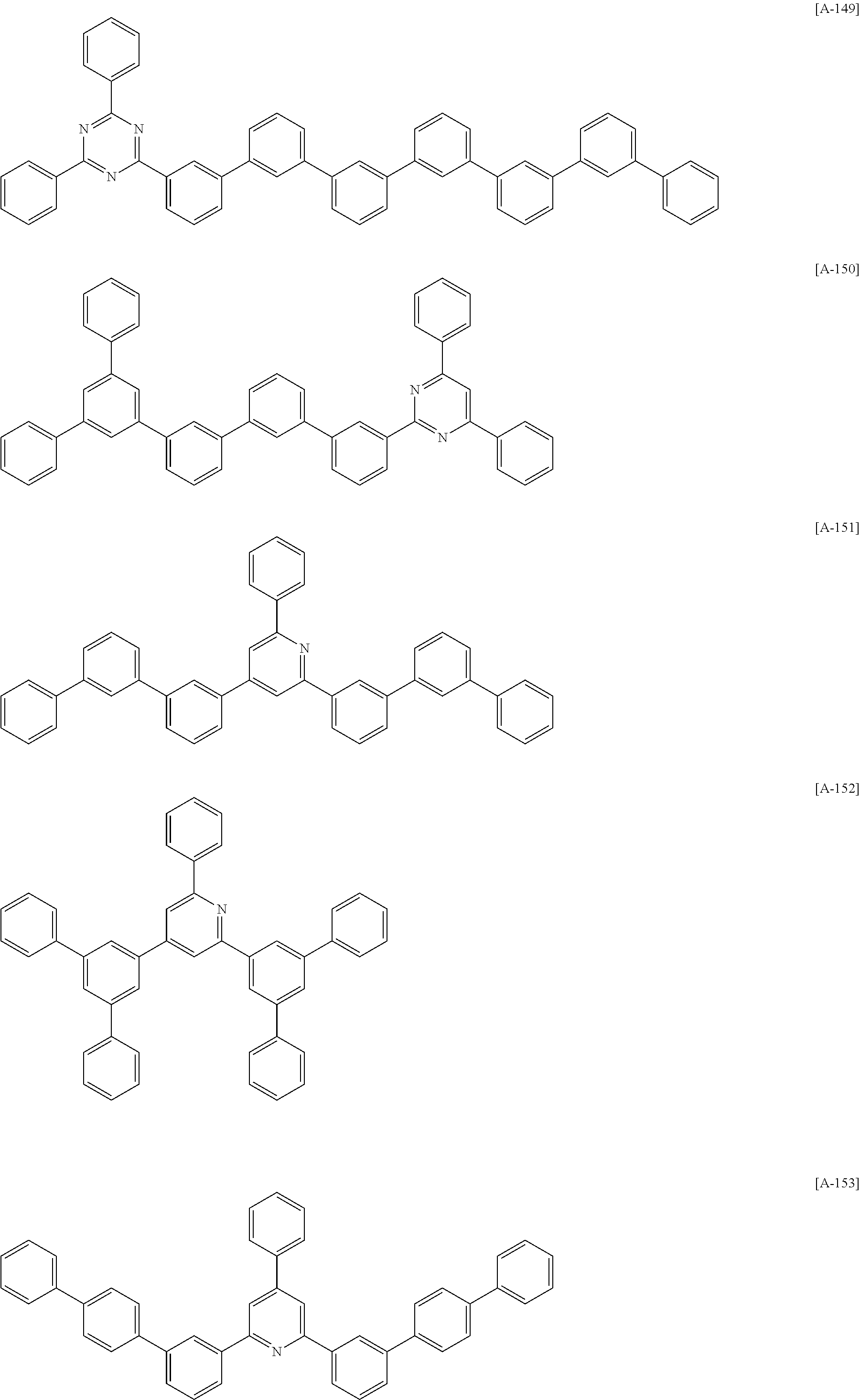 [SMILES: c1ccc(-c2cccc(-c3cccc(-c4cccc(-c5cccc(-c6cccc(-c7cccc(-c8nc(-c9ccccc9)nc(-c9ccccc9)n8)c7)c6)c5)c4)c3)c2)cc1 ; c1ccc(-c2cc(-c3ccccc3)cc(-c3cccc(-c4cccc(-c5cccc(-c6nc(-c7ccccc7)cc(-c7ccccc7)n6)c5)c4)c3)c2)cc1 ; c1ccc(-c2cccc(-c3cccc(-c4cc(-c5ccccc5)nc(-c5cccc(-c6cccc(-c7ccccc7)c6)c5)c4)c3)c2)cc1 ; c1ccc(-c2cc(-c3ccccc3)cc(-c3cc(-c4ccccc4)nc(-c4cc(-c5ccccc5)cc(-c5ccccc5)c4)c3)c2)cc1 ; c1ccc(-c2ccc(-c3cccc(-c4cc(-c5ccccc5)cc(-c5cccc(-c6ccc(-c7ccccc7)cc6)c5)n4)c3)cc2)cc1]

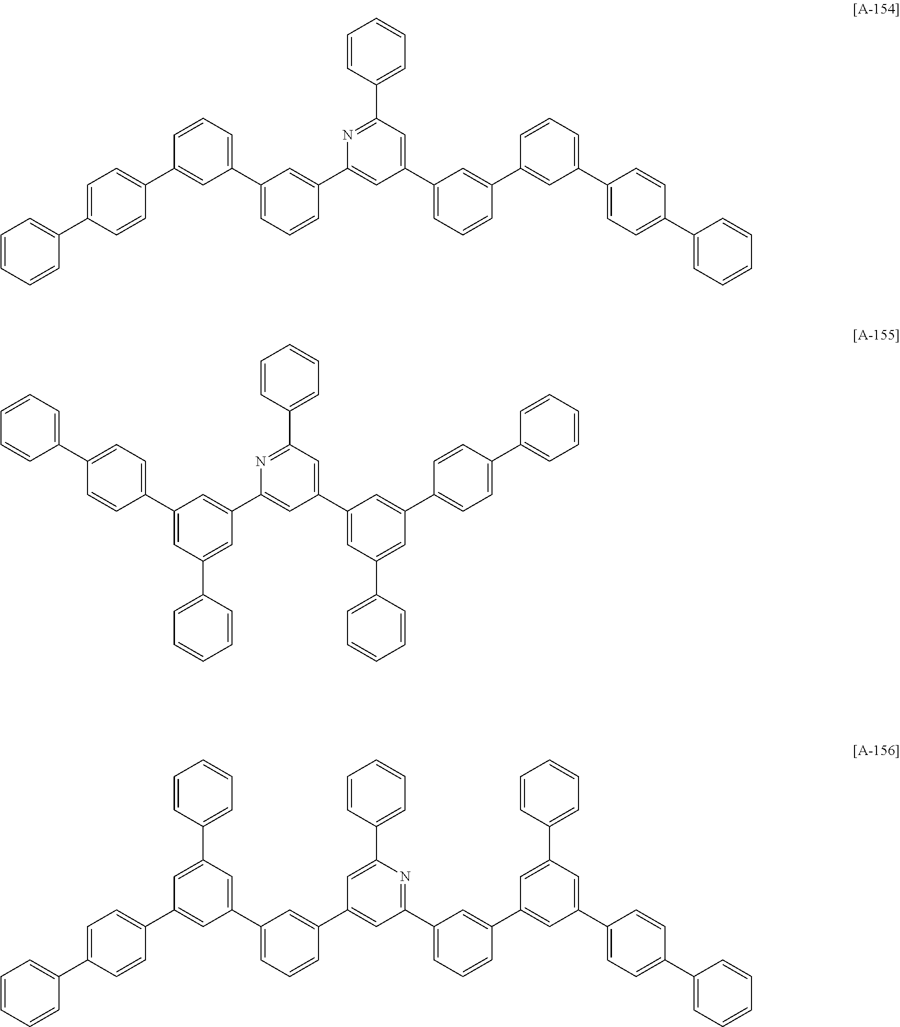 [SMILES: c1ccc(-c2ccc(-c3cccc(-c4cccc(-c5cc(-c6ccccc6)nc(-c6cccc(-c7cccc(-c8ccc(-c9ccccc9)cc8)c7)c6)c5)c4)c3)cc2)cc1 ; c1ccc(-c2ccc(-c3cc(-c4ccccc4)cc(-c4cc(-c5ccccc5)nc(-c5cc(-c6ccccc6)cc(-c6ccc(-c7ccccc7)cc6)c5)c4)c3)cc2)cc1 ; c1ccc(-c2ccc(-c3cc(-c4ccccc4)cc(-c4cccc(-c5cc(-c6ccccc6)nc(-c6cccc(-c7cc(-c8ccccc8)cc(-c8ccc(-c9ccccc9)cc8)c7)c6)c5)c4)c3)cc2)cc1]

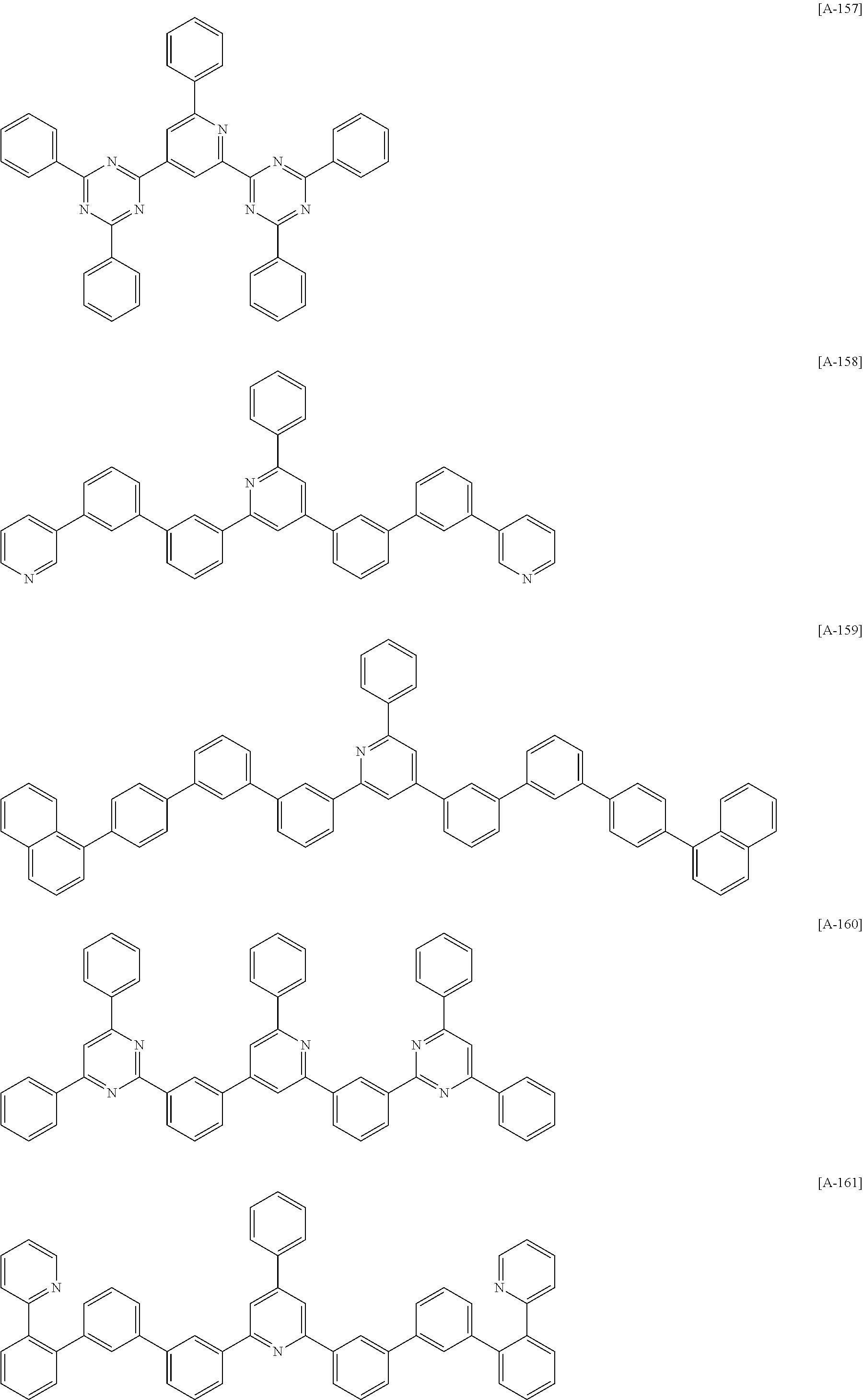

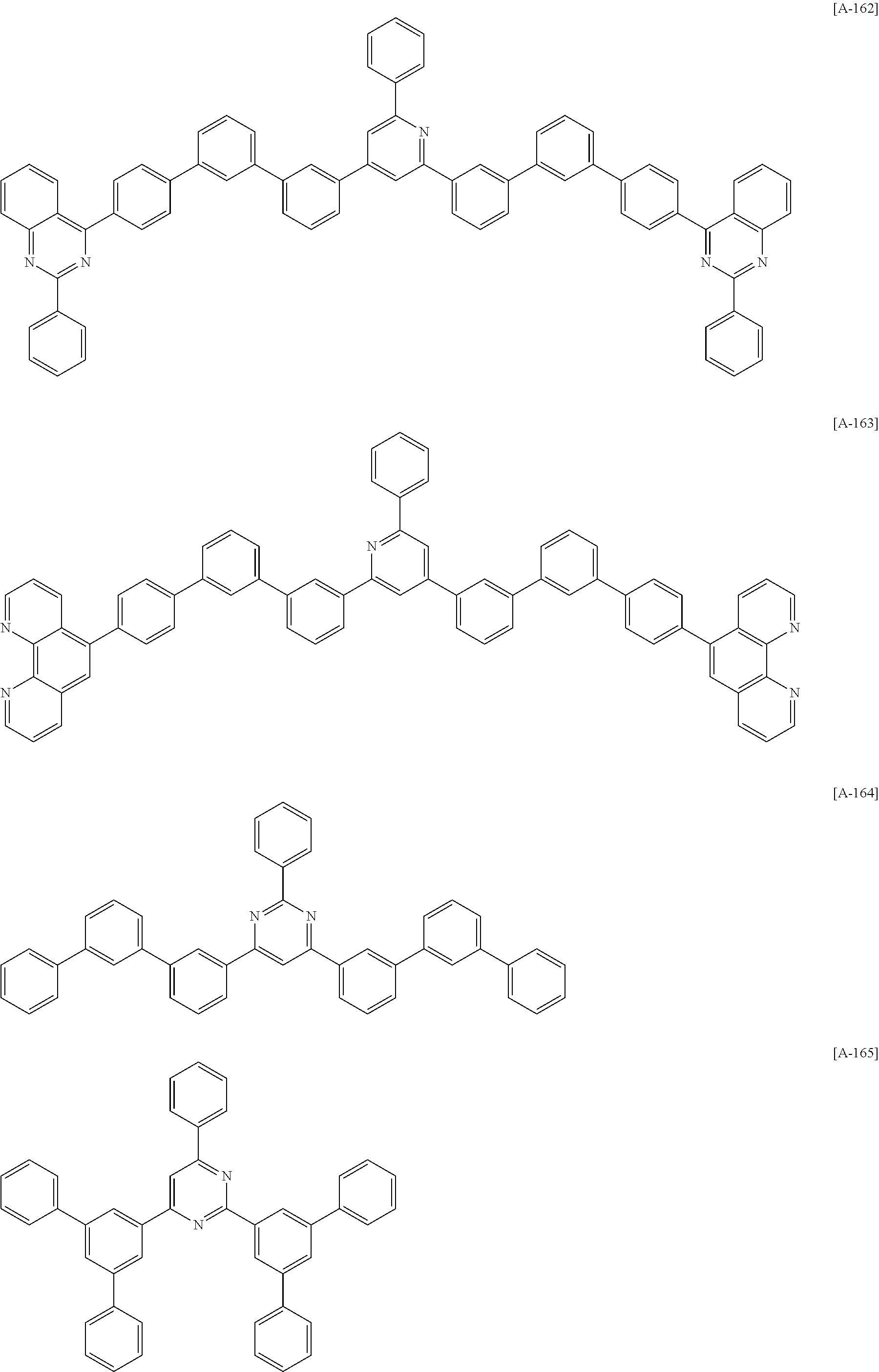 [SMILES: c1ccc(-c2cc(-c3cccc(-c4cccc(-c5ccc(-c6nc(-c7ccccc7)nc7ccccc67)cc5)c4)c3)cc(-c3cccc(-c4cccc(-c5ccc(-c6nc(-c7ccccc7)nc7ccccc67)cc5)c4)c3)n2)cc1 ; c1ccc(-c2cc(-c3cccc(-c4cccc(-c5ccc(-c6cc7cccnc7c7ncccc67)cc5)c4)c3)cc(-c3cccc(-c4cccc(-c5ccc(-c6cc7cccnc7c7ncccc67)cc5)c4)c3)n2)cc1 ; c1ccc(-c2cccc(-c3cccc(-c4cc(-c5cccc(-c6cccc(-c7ccccc7)c6)c5)nc(-c5ccccc5)n4)c3)c2)cc1 ; c1ccc(-c2cc(-c3ccccc3)cc(-c3cc(-c4ccccc4)nc(-c4cc(-c5ccccc5)cc(-c5ccccc5)c4)n3)c2)cc1]

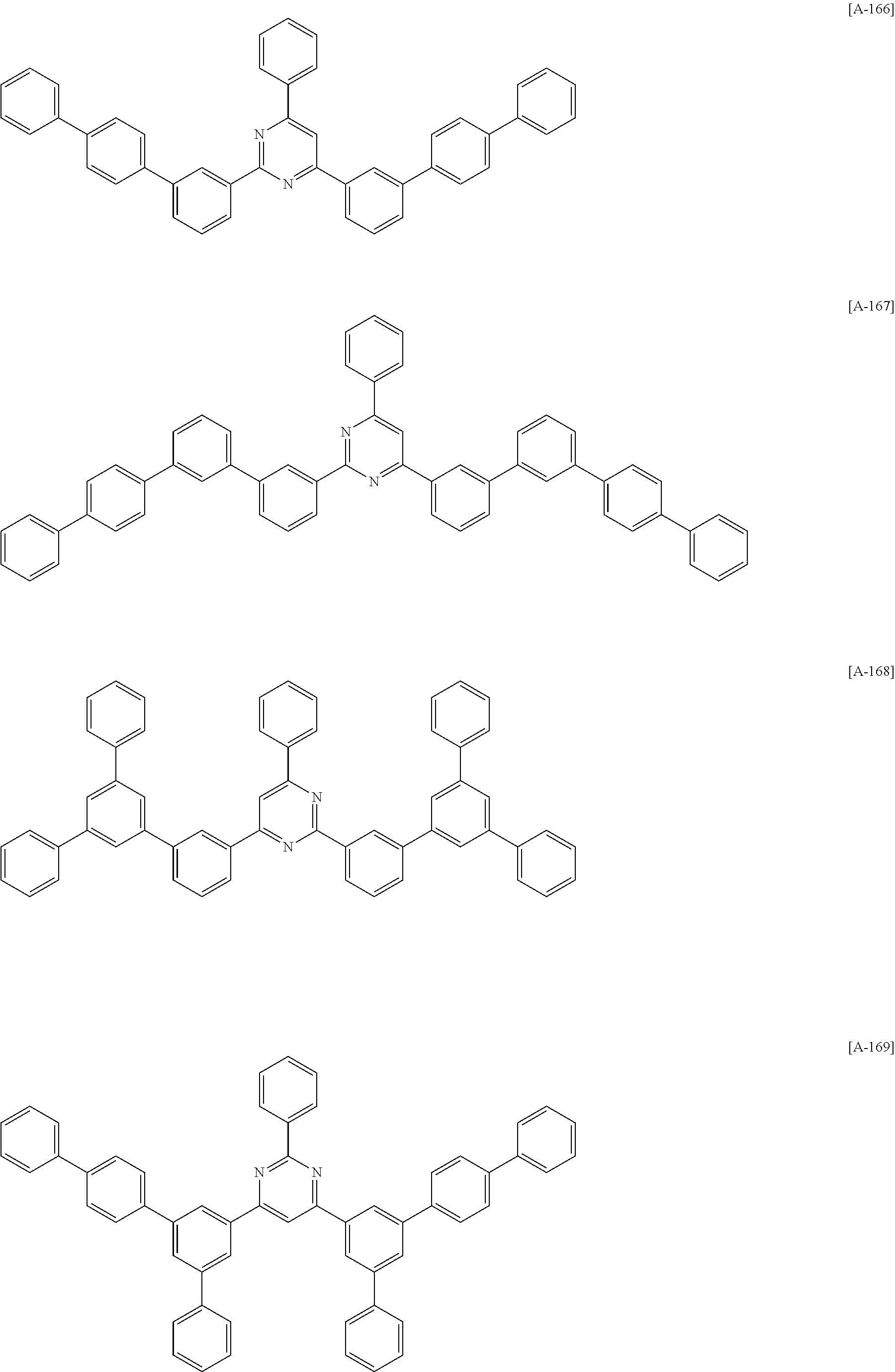 [SMILES: c1ccc(-c2ccc(-c3cccc(-c4cc(-c5ccccc5)nc(-c5cccc(-c6ccc(-c7ccccc7)cc6)c5)n4)c3)cc2)cc1 ; c1ccc(-c2ccc(-c3cccc(-c4cccc(-c5cc(-c6ccccc6)nc(-c6cccc(-c7cccc(-c8ccc(-c9ccccc9)cc8)c7)c6)n5)c4)c3)cc2)cc1 ; c1ccc(-c2cc(-c3ccccc3)cc(-c3cccc(-c4cc(-c5ccccc5)nc(-c5cccc(-c6cc(-c7ccccc7)cc(-c7ccccc7)c6)c5)n4)c3)c2)cc1 ; c1ccc(-c2ccc(-c3cc(-c4ccccc4)cc(-c4cc(-c5cc(-c6ccccc6)cc(-c6ccc(-c7ccccc7)cc6)c5)nc(-c5ccccc5)n4)c3)cc2)cc1]

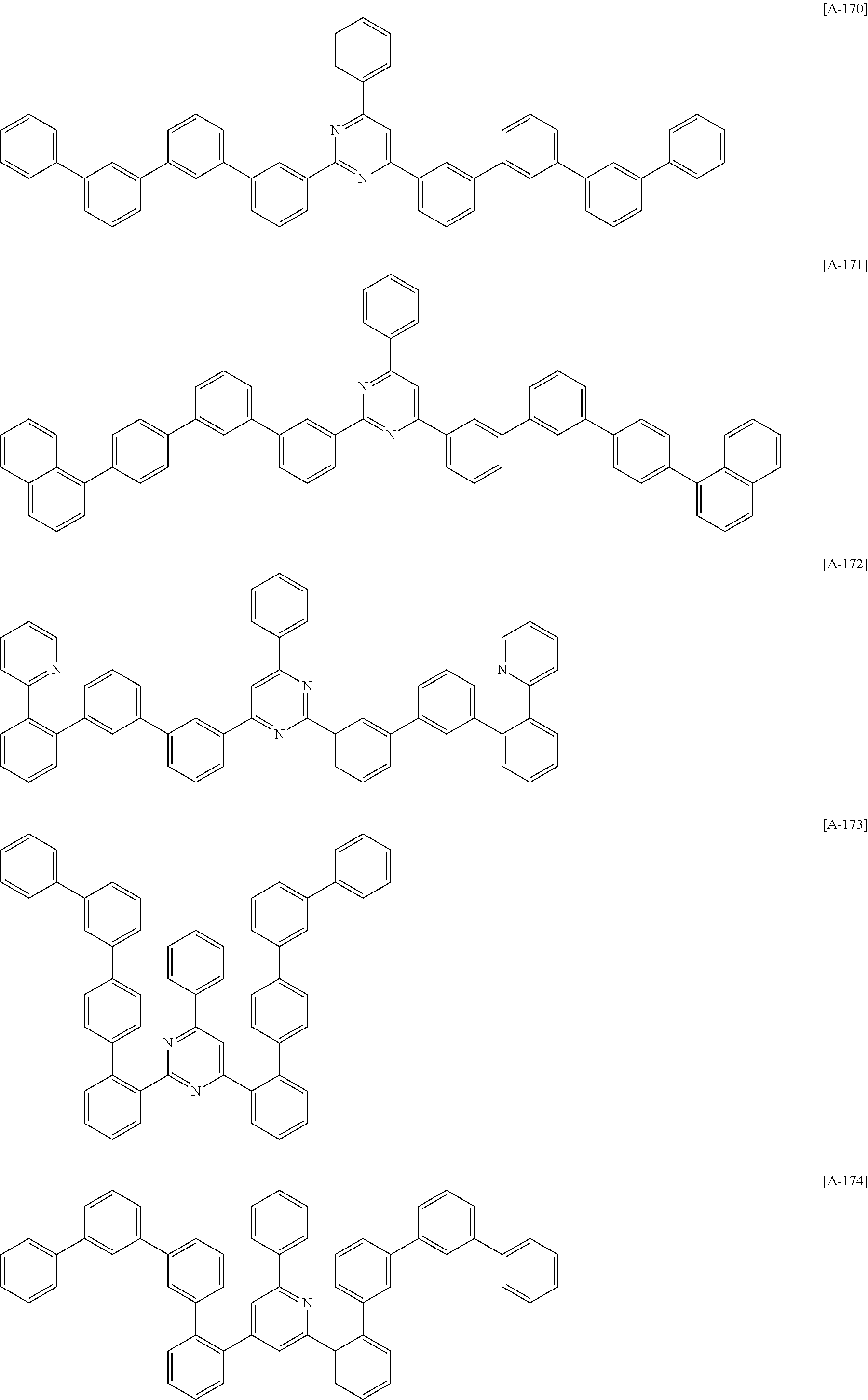

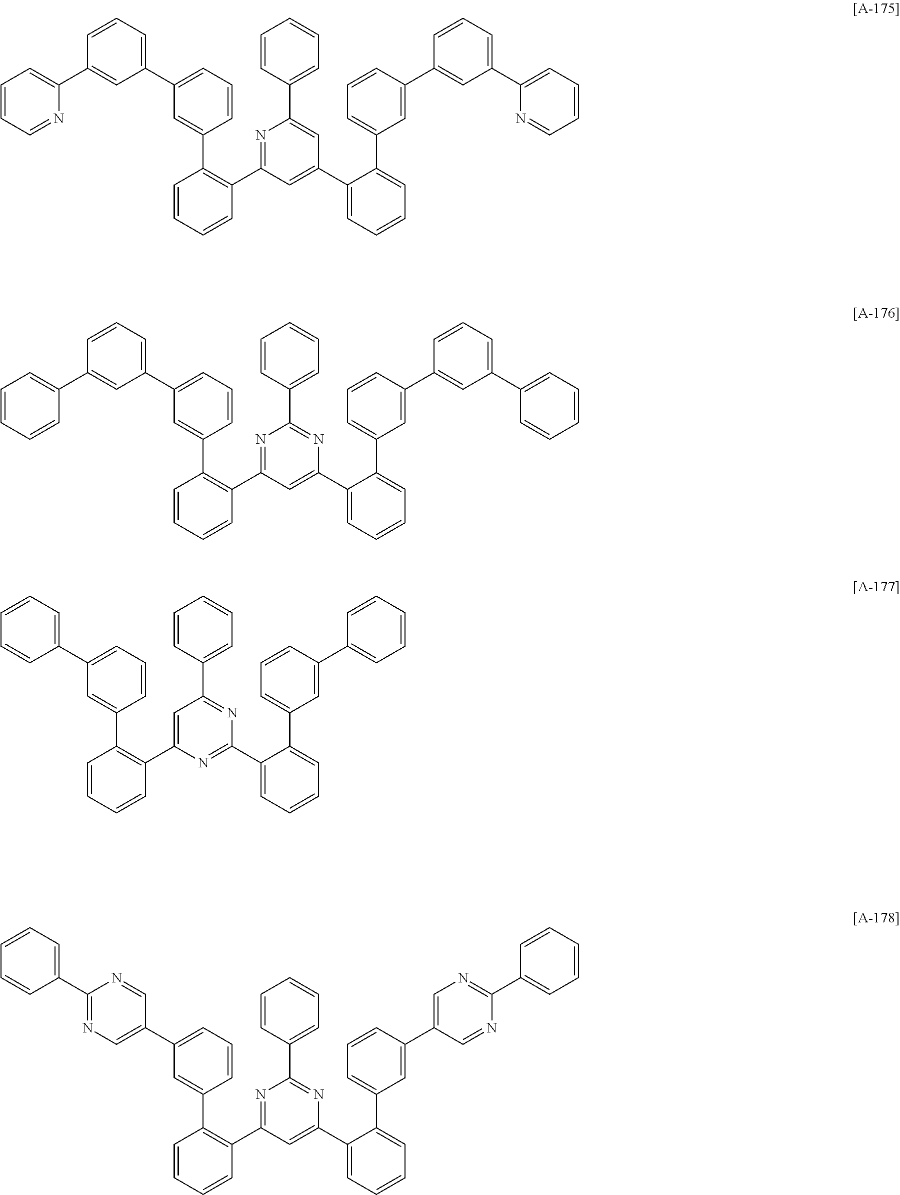 [SMILES: c1ccc(-c2cc(-c3ccccc3-c3cccc(-c4cccc(-c5ccccn5)c4)c3)cc(-c3ccccc3-c3cccc(-c4cccc(-c5ccccn5)c4)c3)n2)cc1 ; c1ccc(-c2cccc(-c3cccc(-c4ccccc4-c4cc(-c5ccccc5-c5cccc(-c6cccc(-c7ccccc7)c6)c5)nc(-c5ccccc5)n4)c3)c2)cc1 ; c1ccc(-c2cccc(-c3ccccc3-c3cc(-c4ccccc4)nc(-c4ccccc4-c4cccc(-c5ccccc5)c4)n3)c2)cc1 ; c1ccc(-c2ncc(-c3cccc(-c4ccccc4-c4cc(-c5ccccc5-c5cccc(-c6cnc(-c7ccccc7)nc6)c5)nc(-c5ccccc5)n4)c3)cn2)cc1]

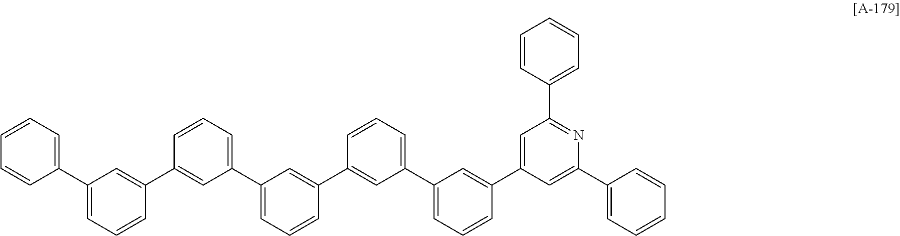 [SMILES: c1ccc(-c2cccc(-c3cccc(-c4cccc(-c5cccc(-c6cccc(-c7cc(-c8ccccc8)nc(-c8ccccc8)c7)c6)c5)c4)c3)c2)cc1]

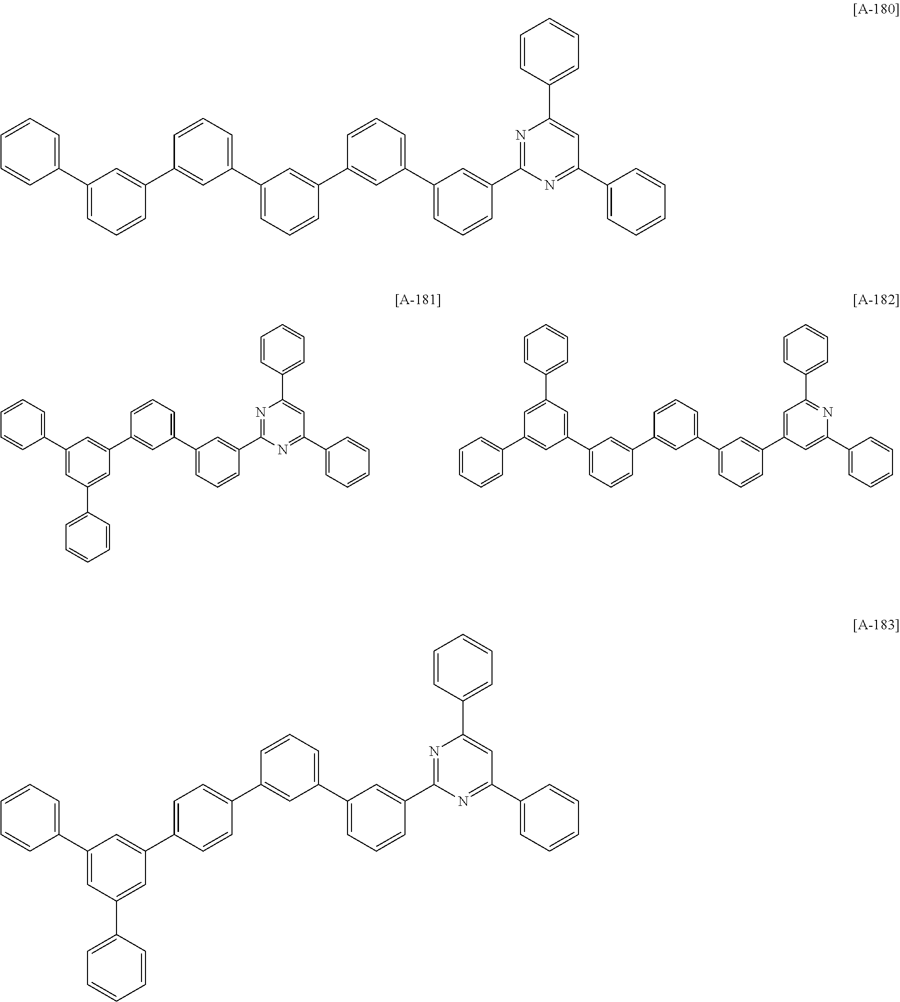 [SMILES: c1ccc(-c2cccc(-c3cccc(-c4cccc(-c5cccc(-c6cccc(-c7nc(-c8ccccc8)cc(-c8ccccc8)n7)c6)c5)c4)c3)c2)cc1 ; c1ccc(-c2cc(-c3ccccc3)cc(-c3cccc(-c4cccc(-c5nc(-c6ccccc6)cc(-c6ccccc6)n5)c4)c3)c2)cc1 ; c1ccc(-c2cc(-c3ccccc3)cc(-c3cccc(-c4cccc(-c5cccc(-c6cc(-c7ccccc7)nc(-c7ccccc7)c6)c5)c4)c3)c2)cc1 ; c1ccc(-c2cc(-c3ccccc3)cc(-c3ccc(-c4cccc(-c5cccc(-c6nc(-c7ccccc7)cc(-c7ccccc7)n6)c5)c4)cc3)c2)cc1]

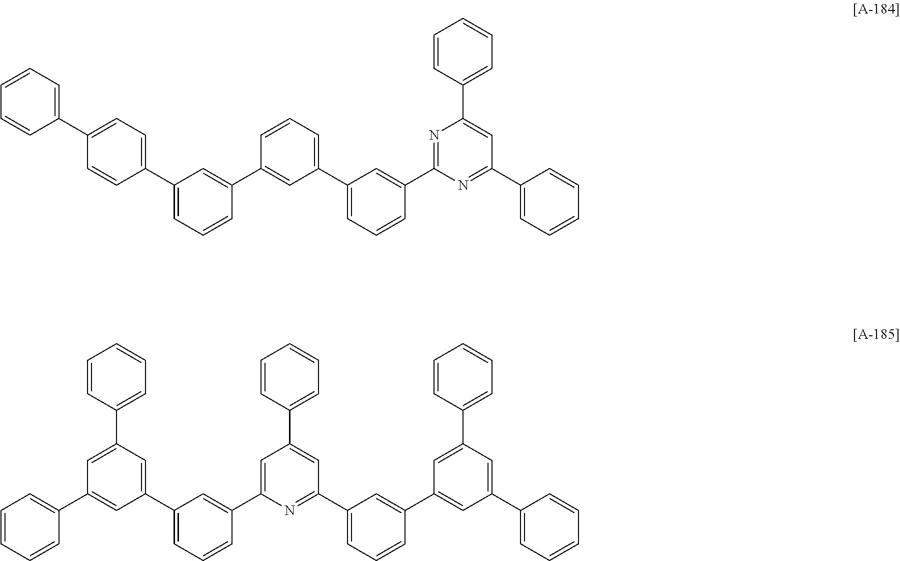 [SMILES: c1ccc(-c2ccc(-c3cccc(-c4cccc(-c5cccc(-c6nc(-c7ccccc7)cc(-c7ccccc7)n6)c5)c4)c3)cc2)cc1 ; c1ccc(-c2cc(-c3ccccc3)cc(-c3cccc(-c4cc(-c5ccccc5)cc(-c5cccc(-c6cc(-c7ccccc7)cc(-c7ccccc7)c6)c5)n4)c3)c2)cc1]

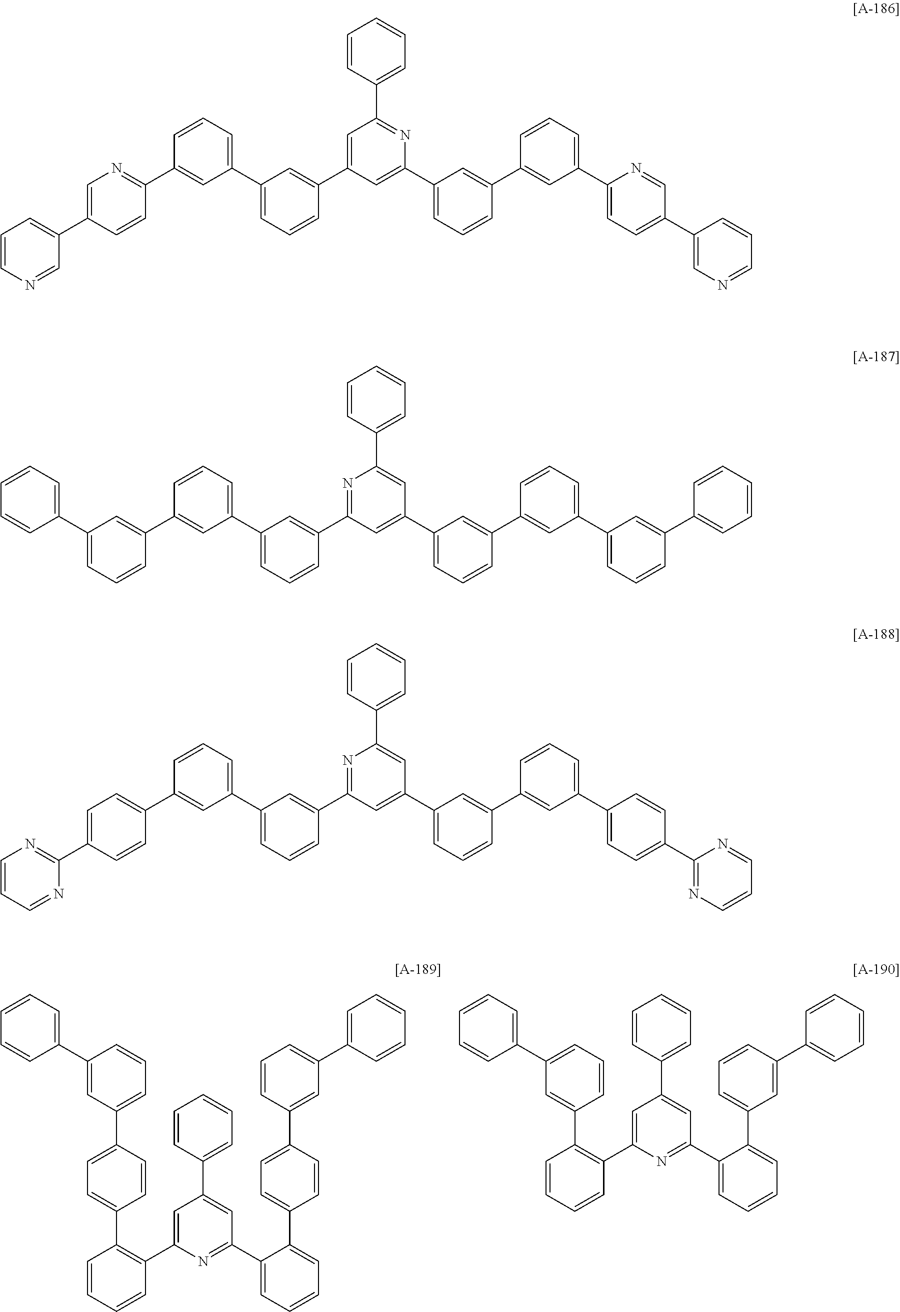

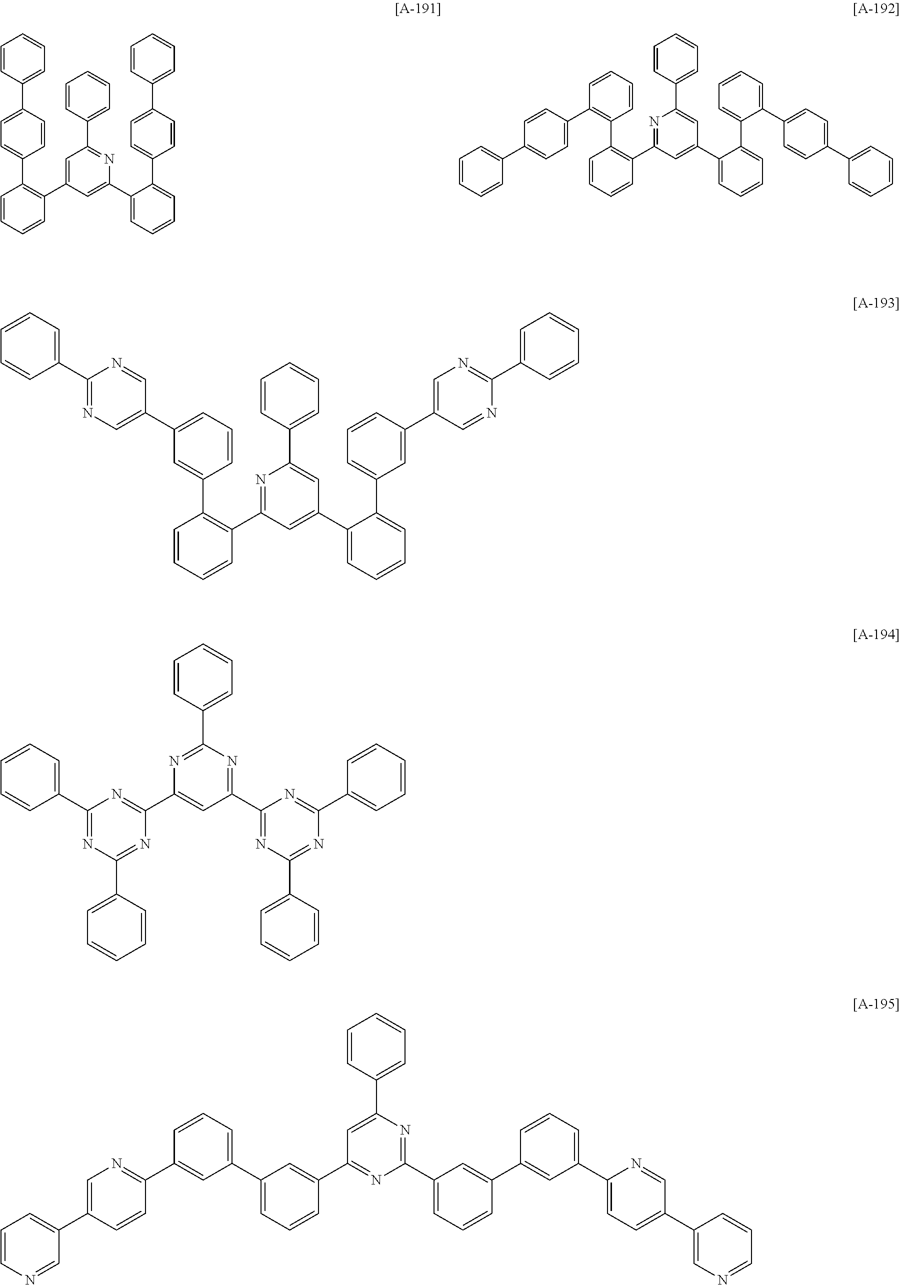 [SMILES: c1ccc(-c2ccc(-c3ccccc3-c3cc(-c4ccccc4)nc(-c4ccccc4-c4ccc(-c5ccccc5)cc4)c3)cc2)cc1 ; c1ccc(-c2ccc(-c3ccccc3-c3ccccc3-c3cc(-c4ccccc4)nc(-c4ccccc4-c4ccccc4-c4ccc(-c5ccccc5)cc4)c3)cc2)cc1 ; c1ccc(-c2cc(-c3ccccc3-c3cccc(-c4cnc(-c5ccccc5)nc4)c3)cc(-c3ccccc3-c3cccc(-c4cnc(-c5ccccc5)nc4)c3)n2)cc1 ; c1ccc(-c2nc(-c3nc(-c4ccccc4)nc(-c4ccccc4)n3)cc(-c3nc(-c4ccccc4)nc(-c4ccccc4)n3)n2)cc1 ; c1ccc(-c2cc(-c3cccc(-c4cccc(-c5ccc(-c6cccnc6)cn5)c4)c3)nc(-c3cccc(-c4cccc(-c5ccc(-c6cccnc6)cn5)c4)c3)n2)cc1]

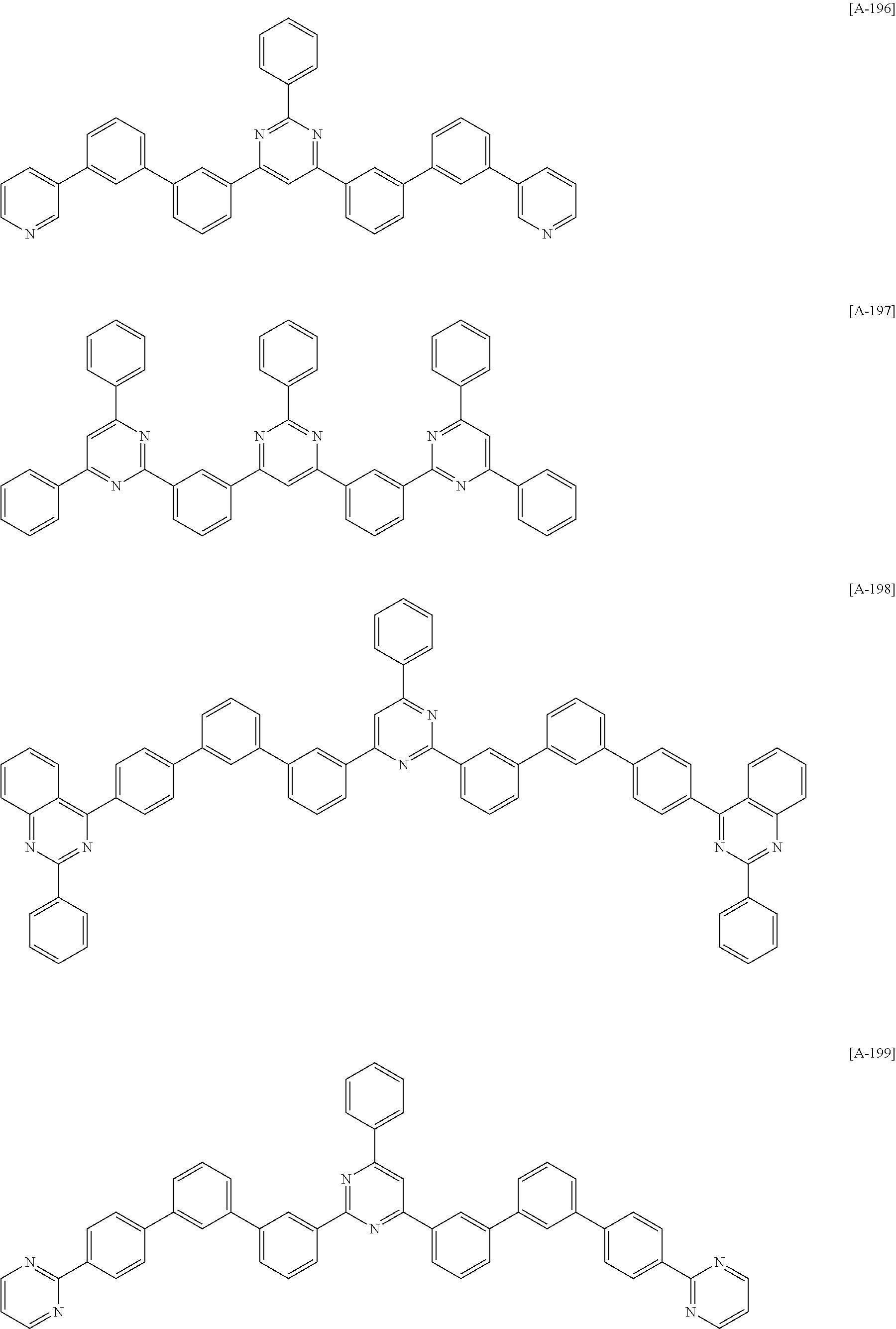 [SMILES: c1ccc(-c2nc(-c3cccc(-c4cccc(-c5cccnc5)c4)c3)cc(-c3cccc(-c4cccc(-c5cccnc5)c4)c3)n2)cc1 ; c1ccc(-c2cc(-c3ccccc3)nc(-c3cccc(-c4cc(-c5cccc(-c6nc(-c7ccccc7)cc(-c7ccccc7)n6)c5)nc(-c5ccccc5)n4)c3)n2)cc1 ; c1ccc(-c2cc(-c3cccc(-c4cccc(-c5ccc(-c6nc(-c7ccccc7)nc7ccccc67)cc5)c4)c3)nc(-c3cccc(-c4cccc(-c5ccc(-c6nc(-c7ccccc7)nc7ccccc67)cc5)c4)c3)n2)cc1 ; c1ccc(-c2cc(-c3cccc(-c4cccc(-c5ccc(-c6ncccn6)cc5)c4)c3)nc(-c3cccc(-c4cccc(-c5ccc(-c6ncccn6)cc5)c4)c3)n2)cc1]

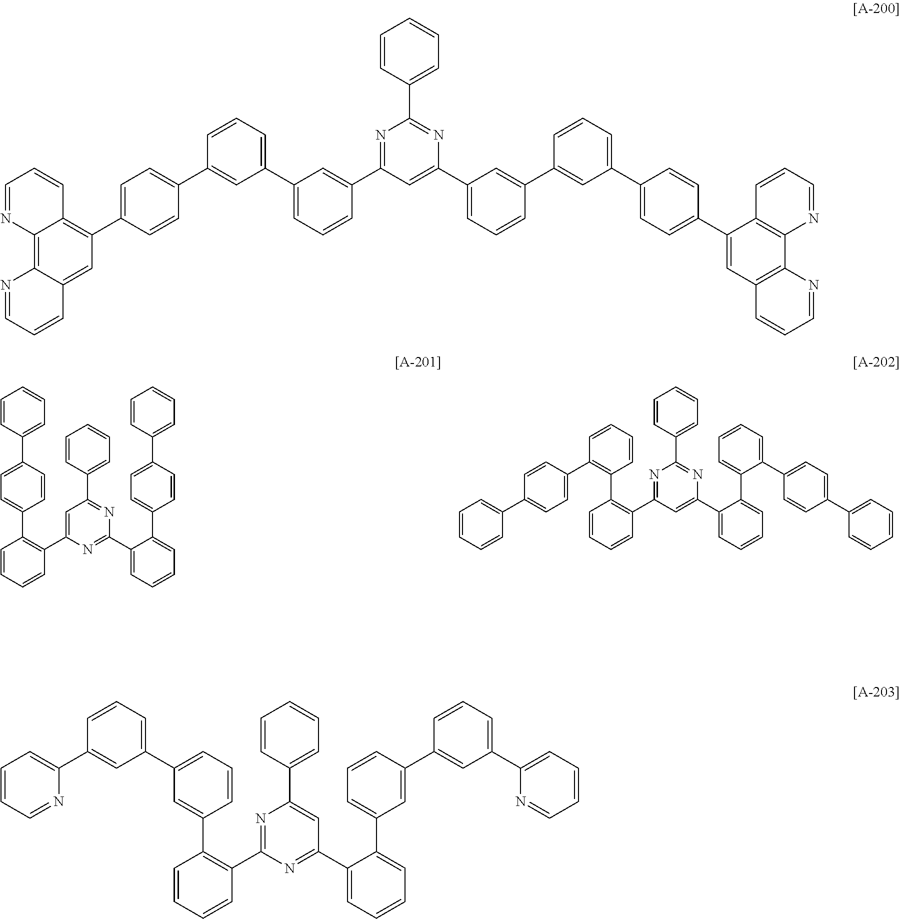 [SMILES: c1ccc(-c2nc(-c3cccc(-c4cccc(-c5ccc(-c6cc7cccnc7c7ncccc67)cc5)c4)c3)cc(-c3cccc(-c4cccc(-c5ccc(-c6cc7cccnc7c7ncccc67)cc5)c4)c3)n2)cc1 ; c1ccc(-c2ccc(-c3ccccc3-c3cc(-c4ccccc4)nc(-c4ccccc4-c4ccc(-c5ccccc5)cc4)n3)cc2)cc1 ; c1ccc(-c2ccc(-c3ccccc3-c3ccccc3-c3cc(-c4ccccc4-c4ccccc4-c4ccc(-c5ccccc5)cc4)nc(-c4ccccc4)n3)cc2)cc1 ; c1ccc(-c2cc(-c3ccccc3-c3cccc(-c4cccc(-c5ccccn5)c4)c3)nc(-c3ccccc3-c3cccc(-c4cccc(-c5ccccn5)c4)c3)n2)cc1]

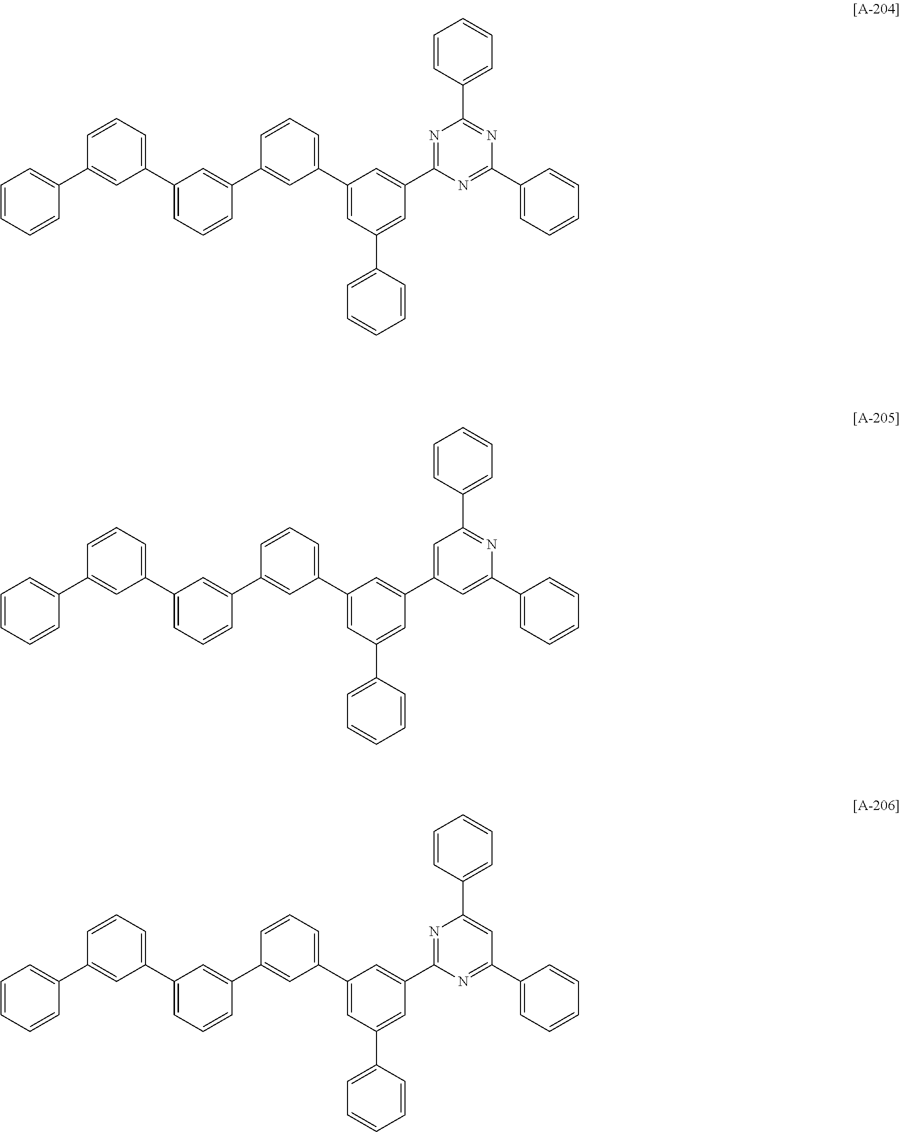 [SMILES: c1ccc(-c2cccc(-c3cccc(-c4cccc(-c5cc(-c6ccccc6)cc(-c6nc(-c7ccccc7)nc(-c7ccccc7)n6)c5)c4)c3)c2)cc1 ; c1ccc(-c2cccc(-c3cccc(-c4cccc(-c5cc(-c6ccccc6)cc(-c6cc(-c7ccccc7)nc(-c7ccccc7)c6)c5)c4)c3)c2)cc1 ; c1ccc(-c2cccc(-c3cccc(-c4cccc(-c5cc(-c6ccccc6)cc(-c6nc(-c7ccccc7)cc(-c7ccccc7)n6)c5)c4)c3)c2)cc1]

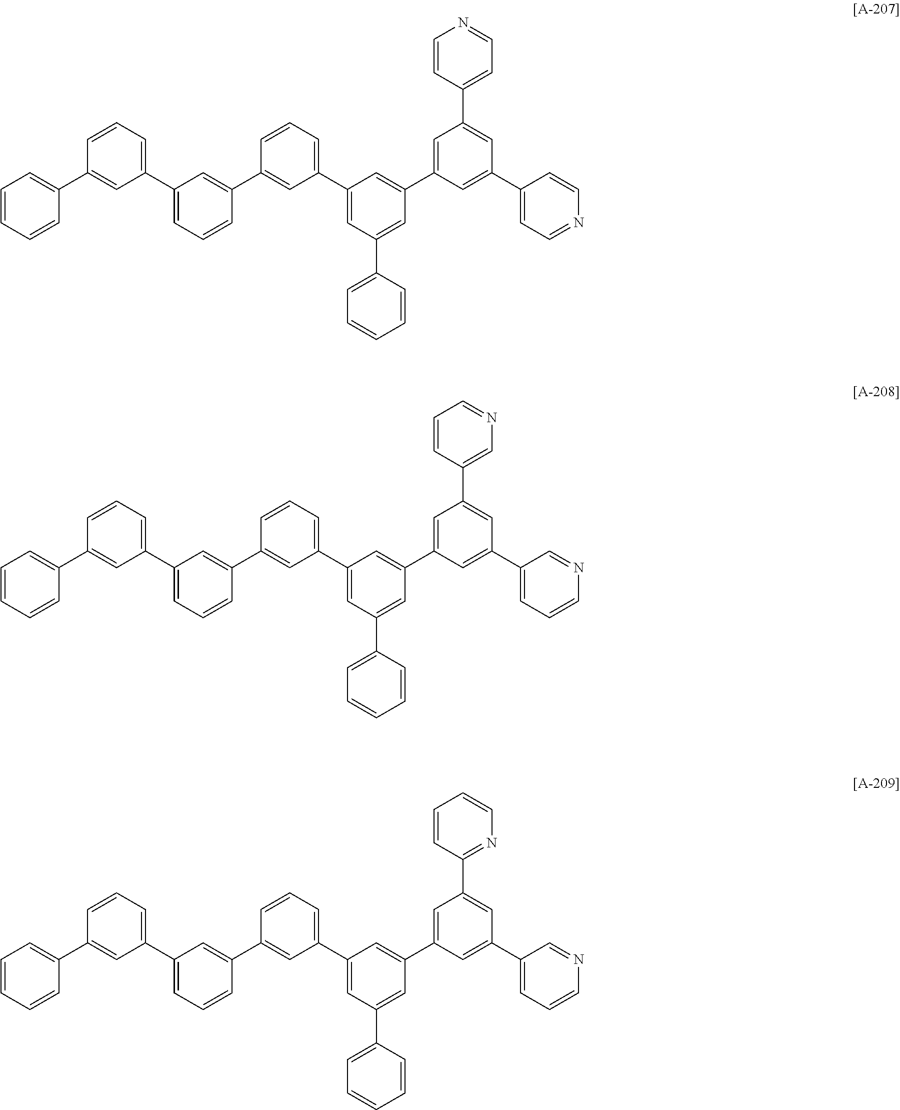 [SMILES: c1ccc(-c2cccc(-c3cccc(-c4cccc(-c5cc(-c6ccccc6)cc(-c6cc(-c7ccncc7)cc(-c7ccncc7)c6)c5)c4)c3)c2)cc1 ; c1ccc(-c2cccc(-c3cccc(-c4cccc(-c5cc(-c6ccccc6)cc(-c6cc(-c7cccnc7)cc(-c7cccnc7)c6)c5)c4)c3)c2)cc1 ; c1ccc(-c2cccc(-c3cccc(-c4cccc(-c5cc(-c6ccccc6)cc(-c6cc(-c7cccnc7)cc(-c7ccccn7)c6)c5)c4)c3)c2)cc1]

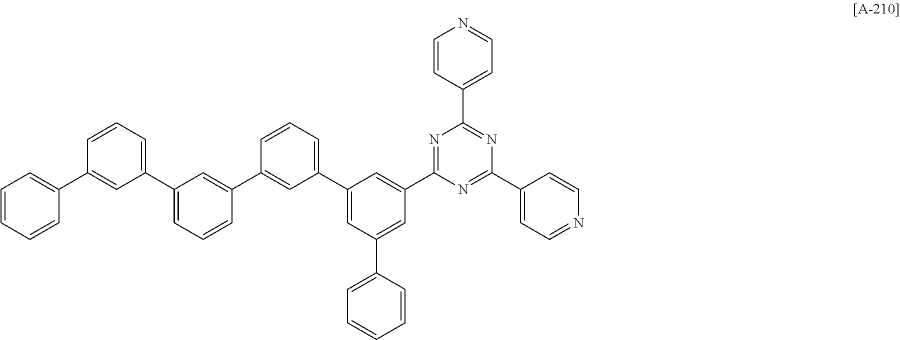 [SMILES: c1ccc(-c2cccc(-c3cccc(-c4cccc(-c5cc(-c6ccccc6)cc(-c6nc(-c7ccncc7)nc(-c7ccncc7)n6)c5)c4)c3)c2)cc1]

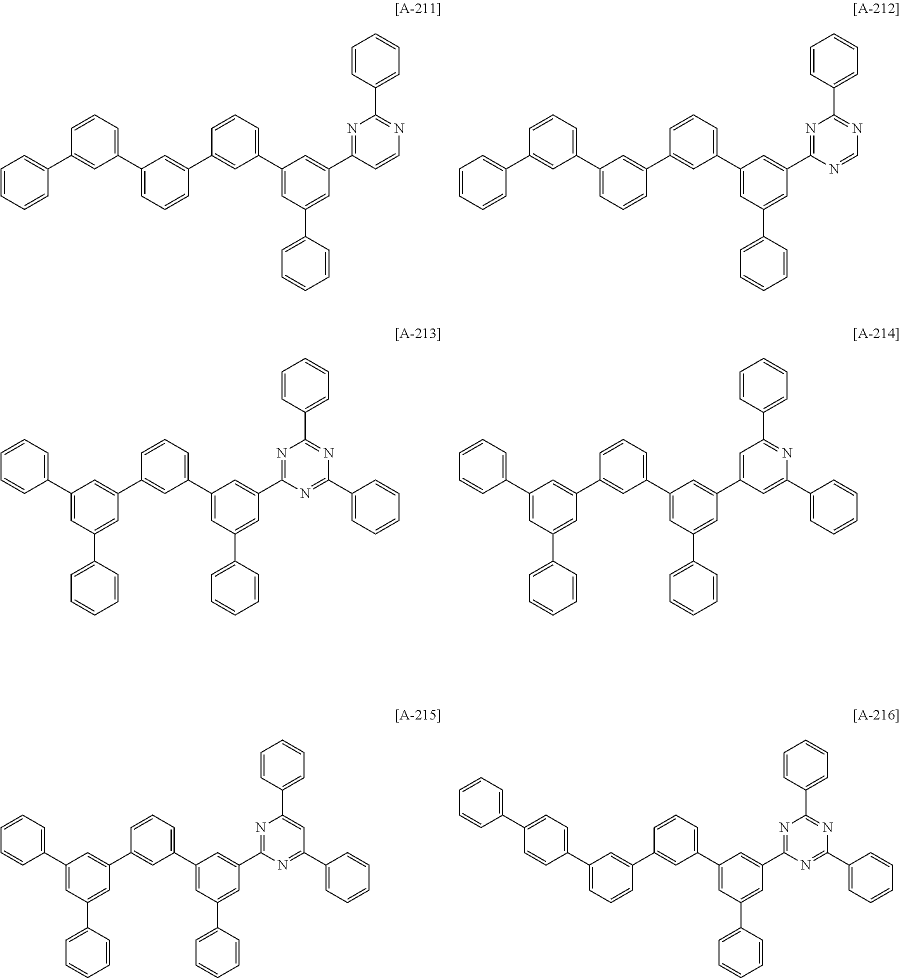

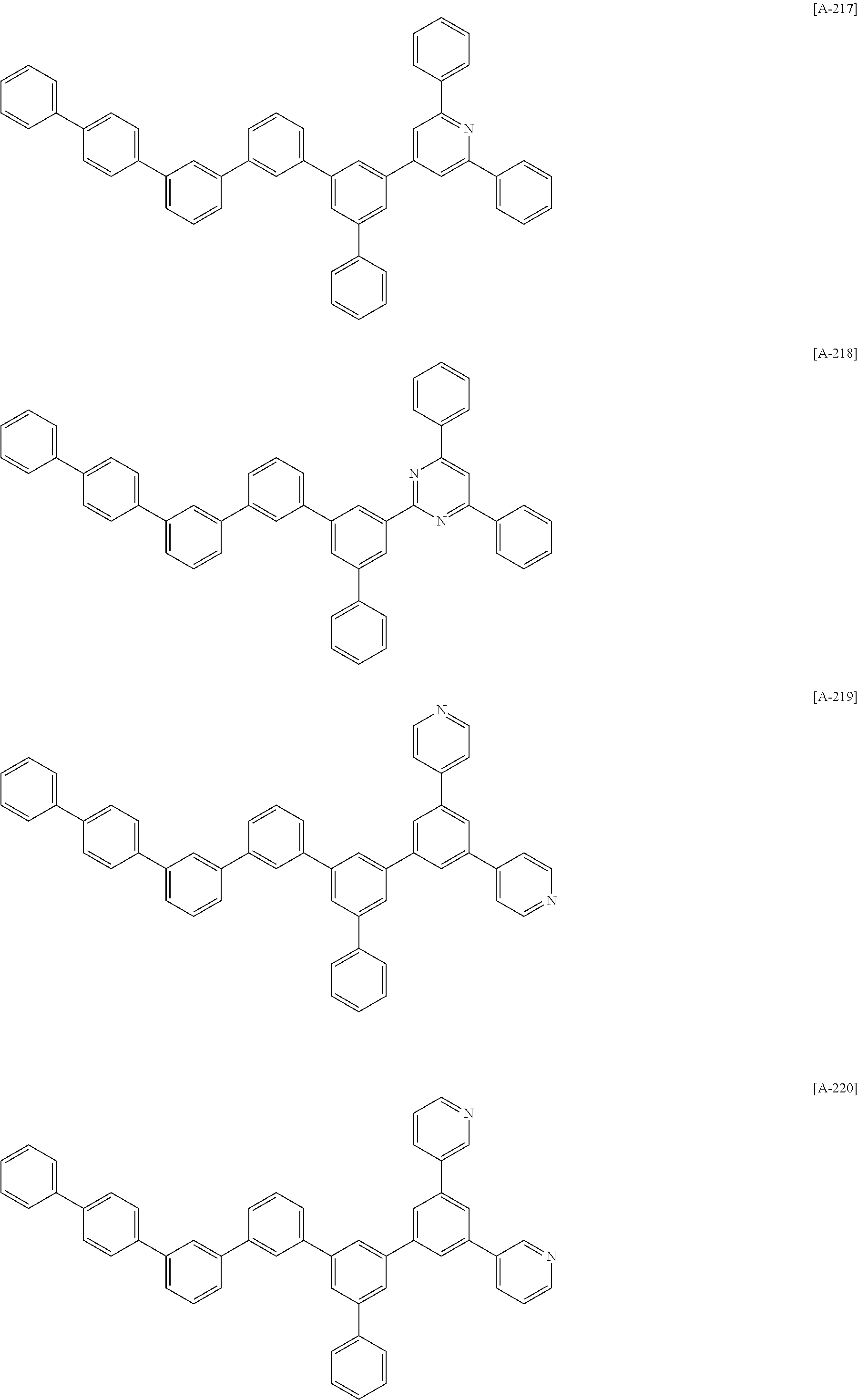 [SMILES: c1ccc(-c2ccc(-c3cccc(-c4cccc(-c5cc(-c6ccccc6)cc(-c6cc(-c7ccccc7)nc(-c7ccccc7)c6)c5)c4)c3)cc2)cc1 ; c1ccc(-c2ccc(-c3cccc(-c4cccc(-c5cc(-c6ccccc6)cc(-c6nc(-c7ccccc7)cc(-c7ccccc7)n6)c5)c4)c3)cc2)cc1 ; c1ccc(-c2ccc(-c3cccc(-c4cccc(-c5cc(-c6ccccc6)cc(-c6cc(-c7ccncc7)cc(-c7ccncc7)c6)c5)c4)c3)cc2)cc1 ; c1ccc(-c2ccc(-c3cccc(-c4cccc(-c5cc(-c6ccccc6)cc(-c6cc(-c7cccnc7)cc(-c7cccnc7)c6)c5)c4)c3)cc2)cc1]

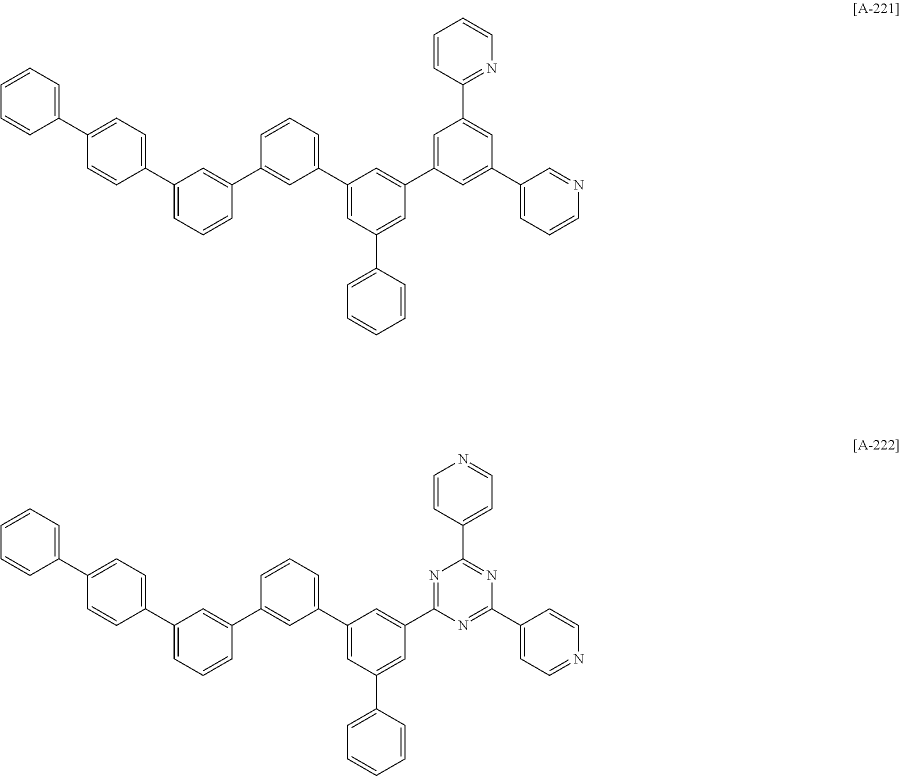 [SMILES: c1ccc(-c2ccc(-c3cccc(-c4cccc(-c5cc(-c6ccccc6)cc(-c6cc(-c7cccnc7)cc(-c7ccccn7)c6)c5)c4)c3)cc2)cc1 ; c1ccc(-c2ccc(-c3cccc(-c4cccc(-c5cc(-c6ccccc6)cc(-c6nc(-c7ccncc7)nc(-c7ccncc7)n6)c5)c4)c3)cc2)cc1]

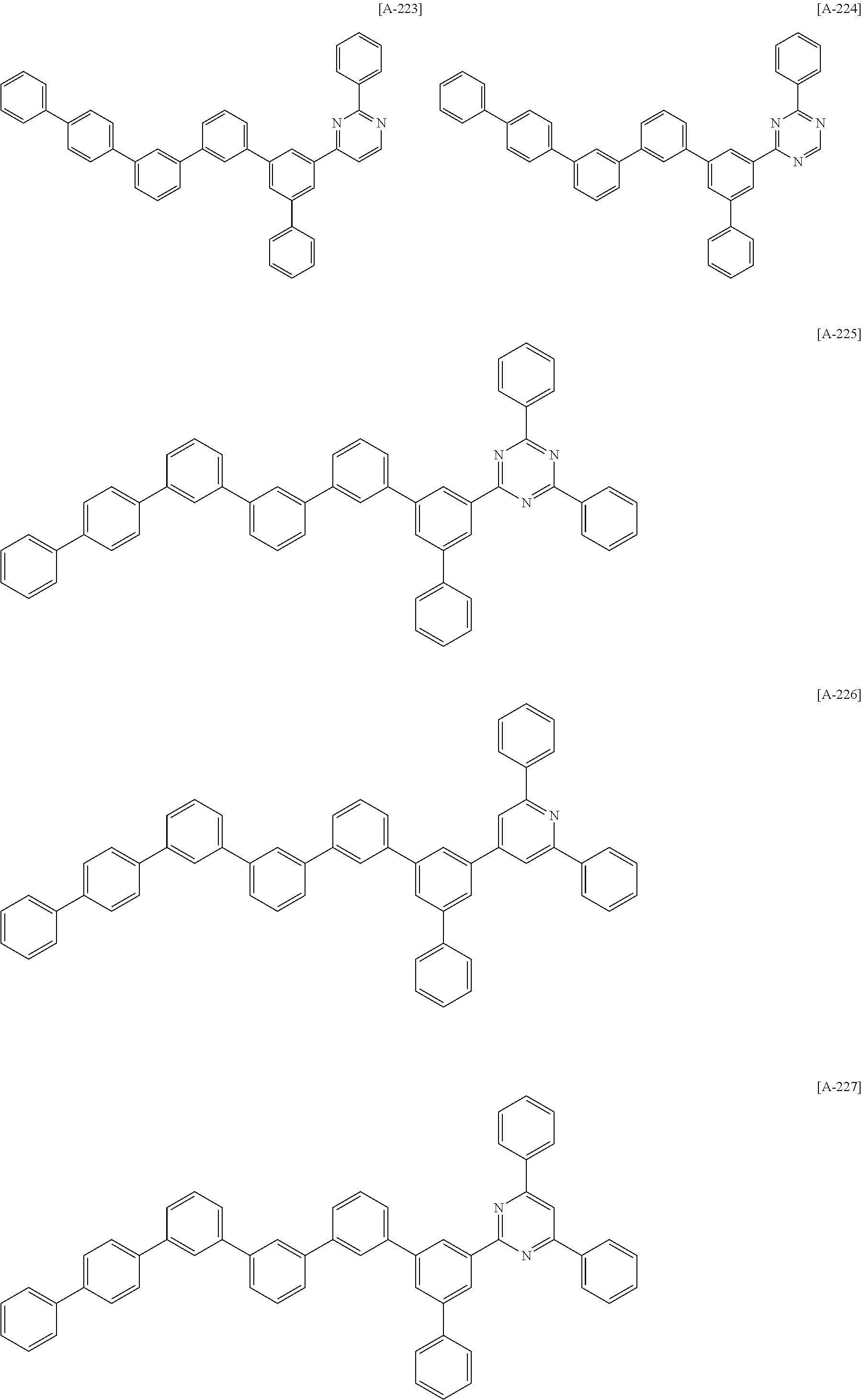

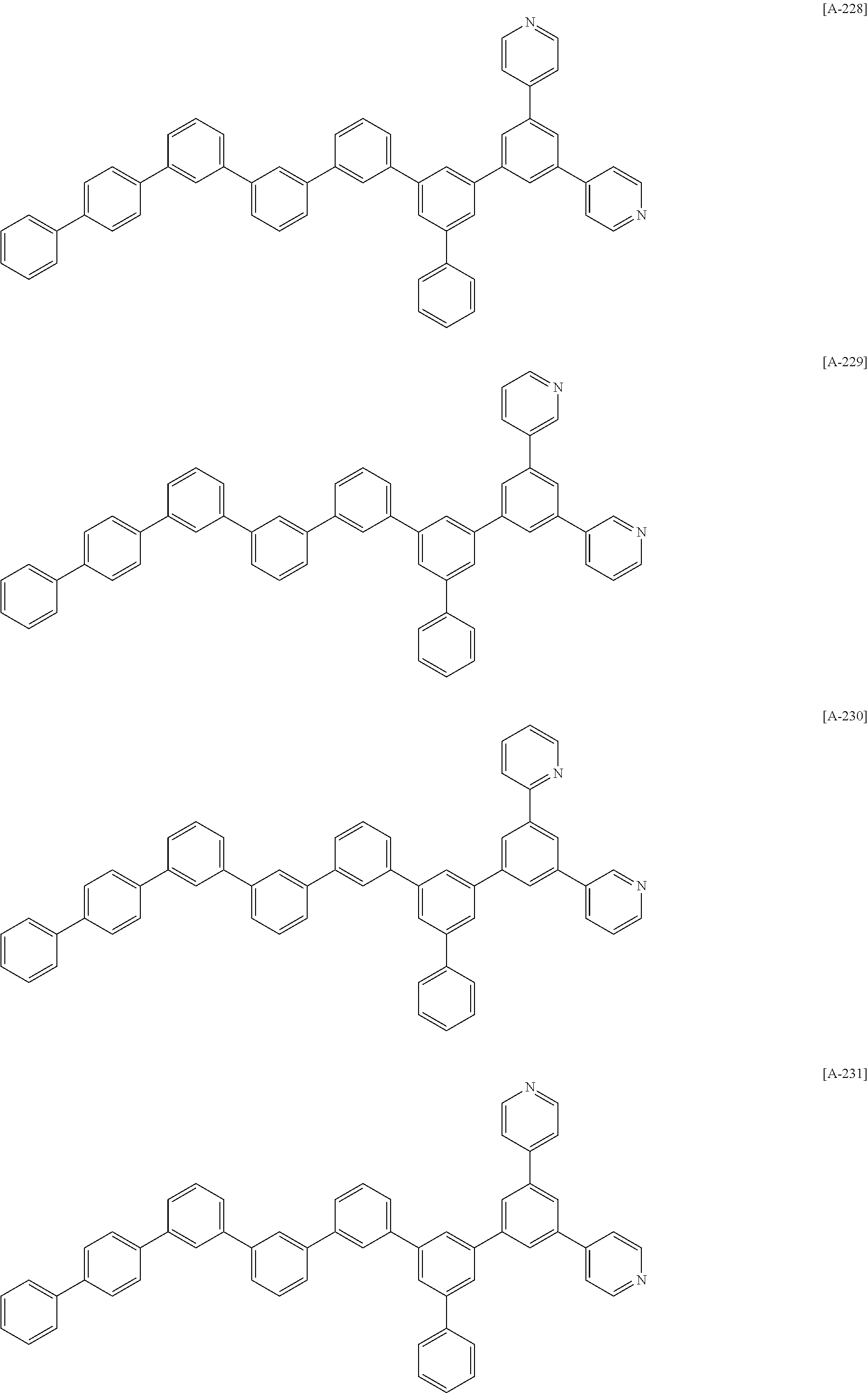 [SMILES: c1ccc(-c2ccc(-c3cccc(-c4cccc(-c5cccc(-c6cc(-c7ccccc7)cc(-c7cc(-c8ccncc8)cc(-c8ccncc8)c7)c6)c5)c4)c3)cc2)cc1 ; c1ccc(-c2ccc(-c3cccc(-c4cccc(-c5cccc(-c6cc(-c7ccccc7)cc(-c7cc(-c8cccnc8)cc(-c8cccnc8)c7)c6)c5)c4)c3)cc2)cc1 ; c1ccc(-c2ccc(-c3cccc(-c4cccc(-c5cccc(-c6cc(-c7ccccc7)cc(-c7cc(-c8cccnc8)cc(-c8ccccn8)c7)c6)c5)c4)c3)cc2)cc1 ; c1ccc(-c2ccc(-c3cccc(-c4cccc(-c5cccc(-c6cc(-c7ccccc7)cc(-c7cc(-c8ccncc8)cc(-c8ccncc8)c7)c6)c5)c4)c3)cc2)cc1]

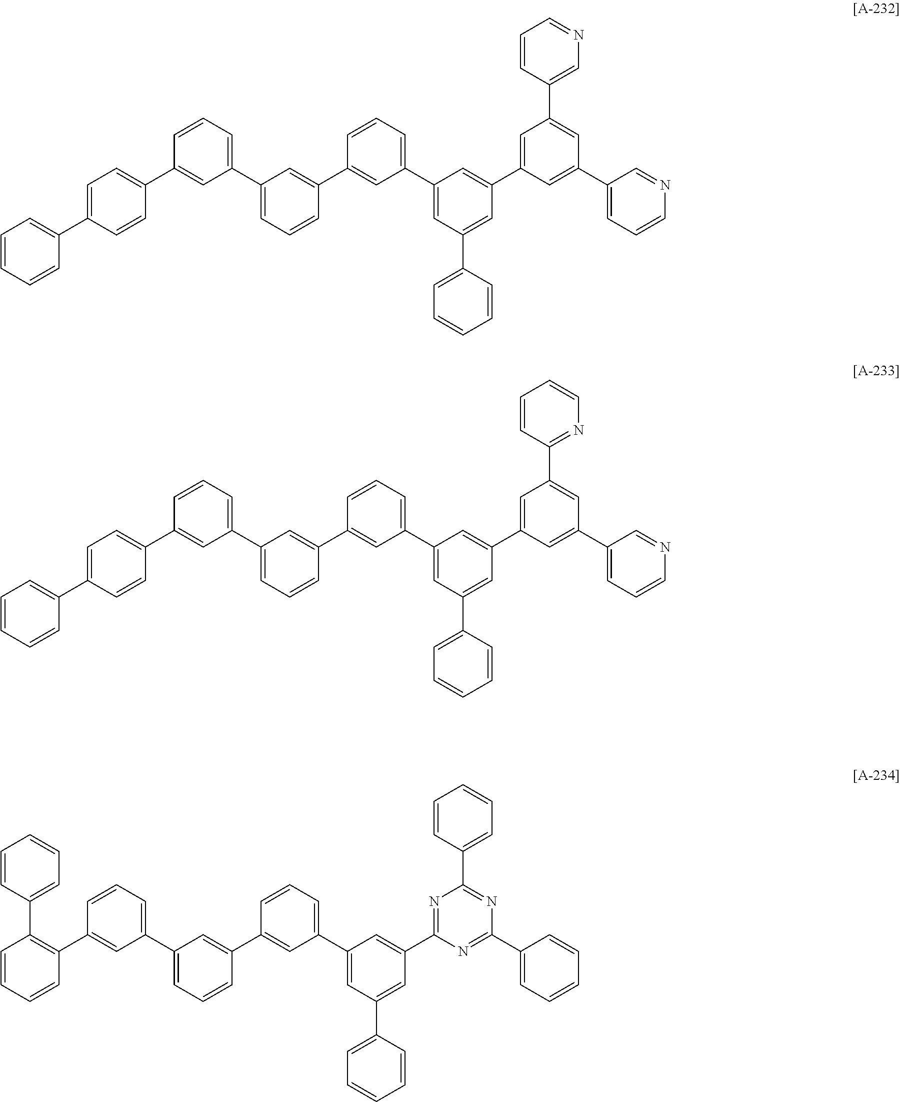 [SMILES: c1ccc(-c2ccc(-c3cccc(-c4cccc(-c5cccc(-c6cc(-c7ccccc7)cc(-c7cc(-c8cccnc8)cc(-c8cccnc8)c7)c6)c5)c4)c3)cc2)cc1 ; c1ccc(-c2ccc(-c3cccc(-c4cccc(-c5cccc(-c6cc(-c7ccccc7)cc(-c7cc(-c8cccnc8)cc(-c8ccccn8)c7)c6)c5)c4)c3)cc2)cc1 ; c1ccc(-c2cc(-c3cccc(-c4cccc(-c5cccc(-c6ccccc6-c6ccccc6)c5)c4)c3)cc(-c3nc(-c4ccccc4)nc(-c4ccccc4)n3)c2)cc1]

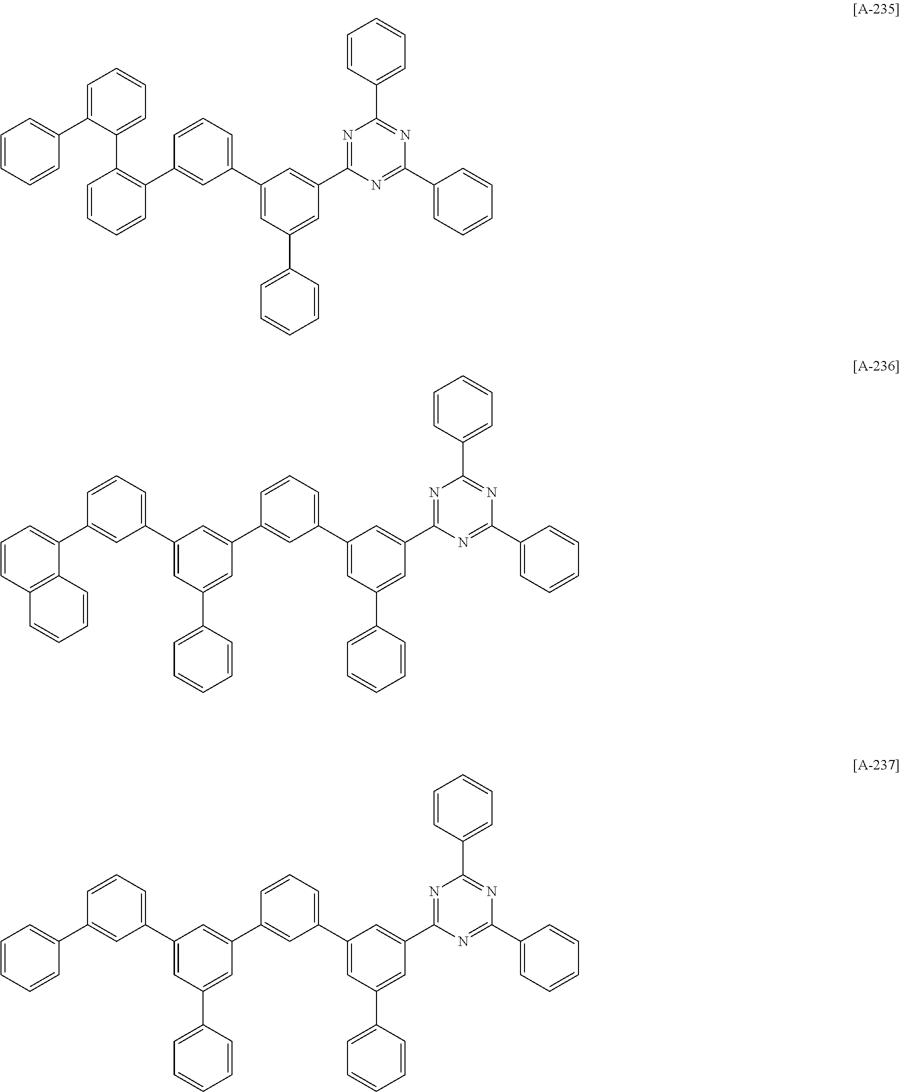 [SMILES: c1ccc(-c2cc(-c3cccc(-c4ccccc4-c4ccccc4-c4ccccc4)c3)cc(-c3nc(-c4ccccc4)nc(-c4ccccc4)n3)c2)cc1 ; c1ccc(-c2cc(-c3cccc(-c4cc(-c5ccccc5)cc(-c5nc(-c6ccccc6)nc(-c6ccccc6)n5)c4)c3)cc(-c3cccc(-c4cccc5ccccc45)c3)c2)cc1 ; c1ccc(-c2cccc(-c3cc(-c4ccccc4)cc(-c4cccc(-c5cc(-c6ccccc6)cc(-c6nc(-c7ccccc7)nc(-c7ccccc7)n6)c5)c4)c3)c2)cc1]

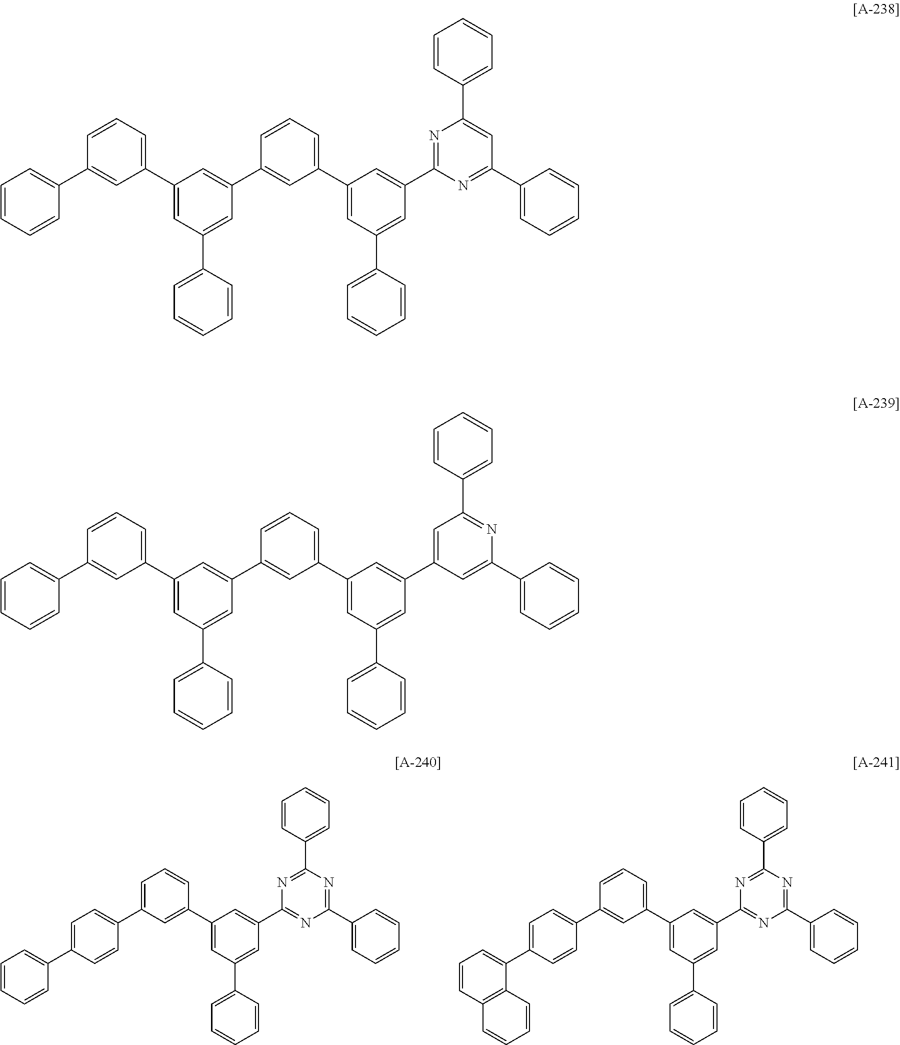 [SMILES: c1ccc(-c2cccc(-c3cc(-c4ccccc4)cc(-c4cccc(-c5cc(-c6ccccc6)cc(-c6nc(-c7ccccc7)cc(-c7ccccc7)n6)c5)c4)c3)c2)cc1 ; c1ccc(-c2cccc(-c3cc(-c4ccccc4)cc(-c4cccc(-c5cc(-c6ccccc6)cc(-c6cc(-c7ccccc7)nc(-c7ccccc7)c6)c5)c4)c3)c2)cc1 ; c1ccc(-c2ccc(-c3cccc(-c4cc(-c5ccccc5)cc(-c5nc(-c6ccccc6)nc(-c6ccccc6)n5)c4)c3)cc2)cc1 ; c1ccc(-c2cc(-c3cccc(-c4ccc(-c5cccc6ccccc56)cc4)c3)cc(-c3nc(-c4ccccc4)nc(-c4ccccc4)n3)c2)cc1]

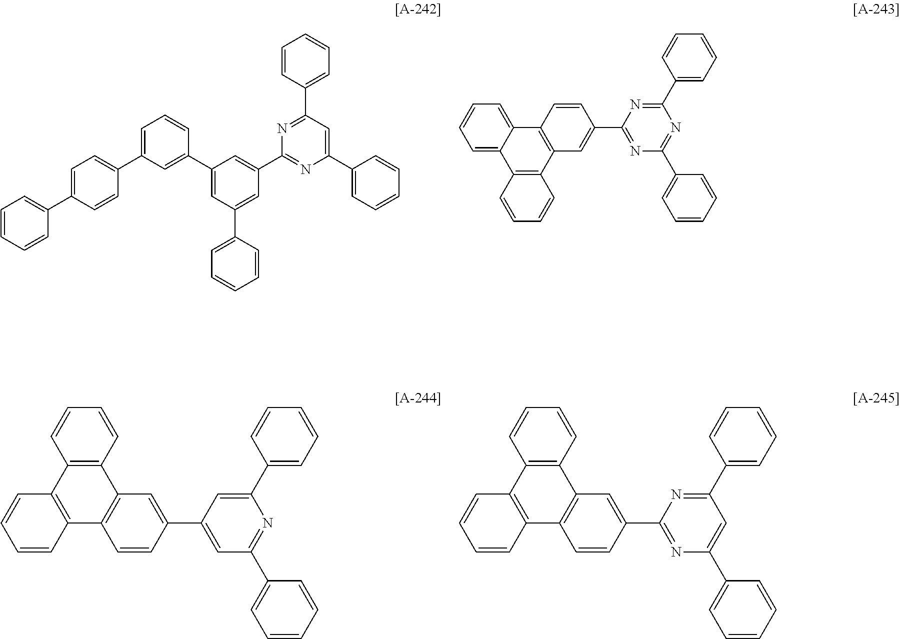 [SMILES: c1ccc(-c2ccc(-c3cccc(-c4cc(-c5ccccc5)cc(-c5nc(-c6ccccc6)cc(-c6ccccc6)n5)c4)c3)cc2)cc1 ; c1ccc(-c2nc(-c3ccccc3)nc(-c3ccc4c5ccccc5c5ccccc5c4c3)n2)cc1 ; c1ccc(-c2cc(-c3ccc4c5ccccc5c5ccccc5c4c3)cc(-c3ccccc3)n2)cc1 ; c1ccc(-c2cc(-c3ccccc3)nc(-c3ccc4c5ccccc5c5ccccc5c4c3)n2)cc1]

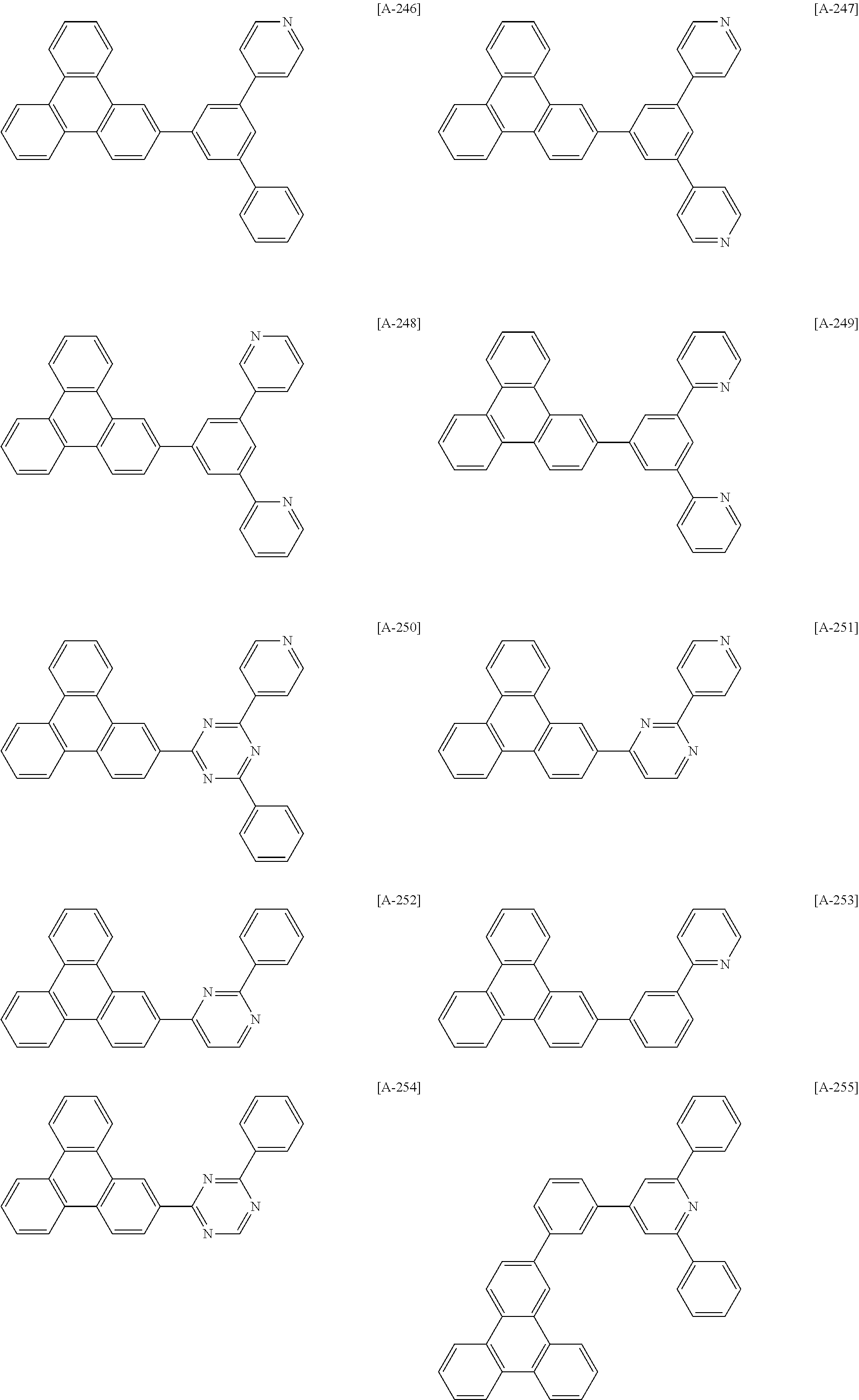

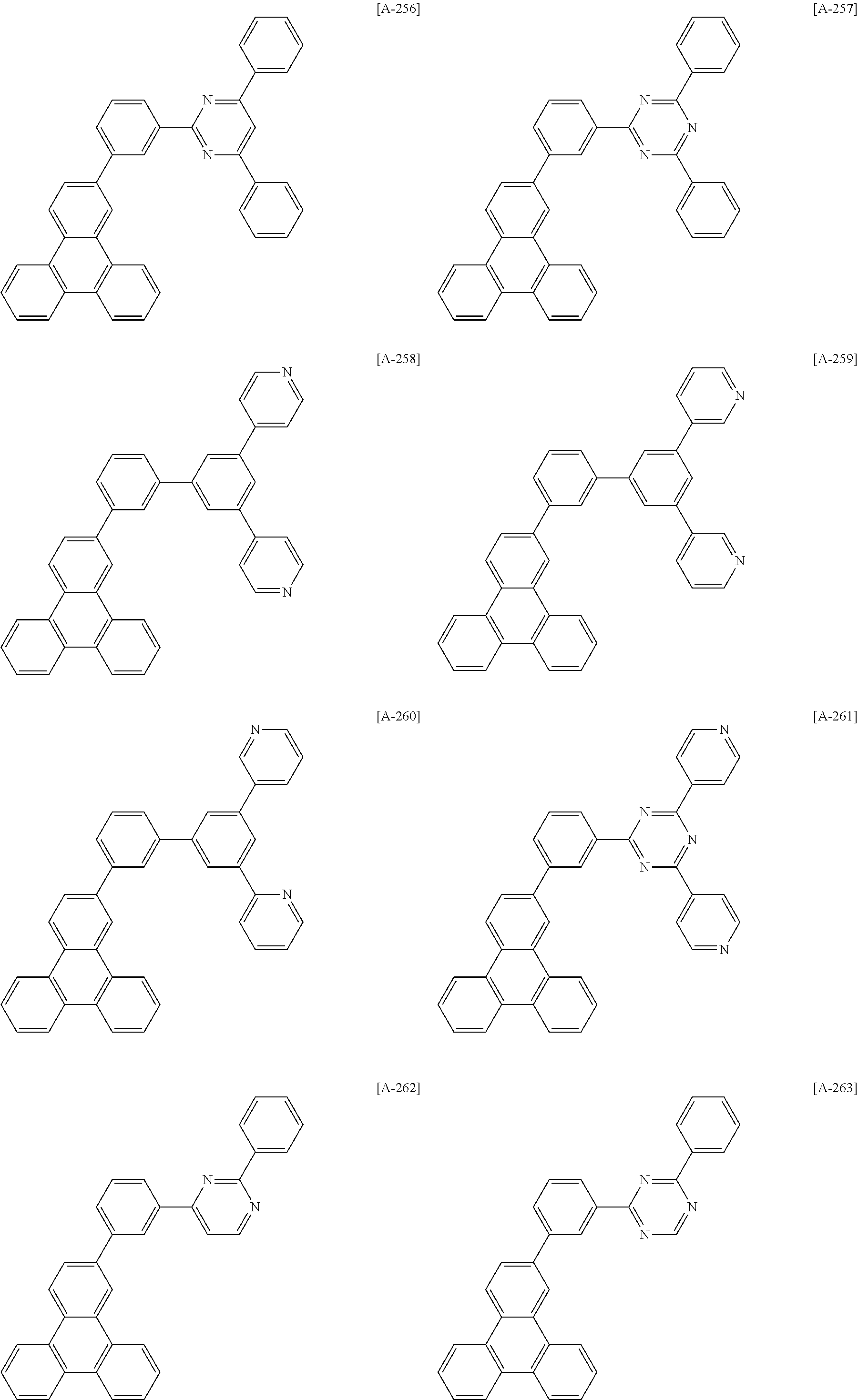

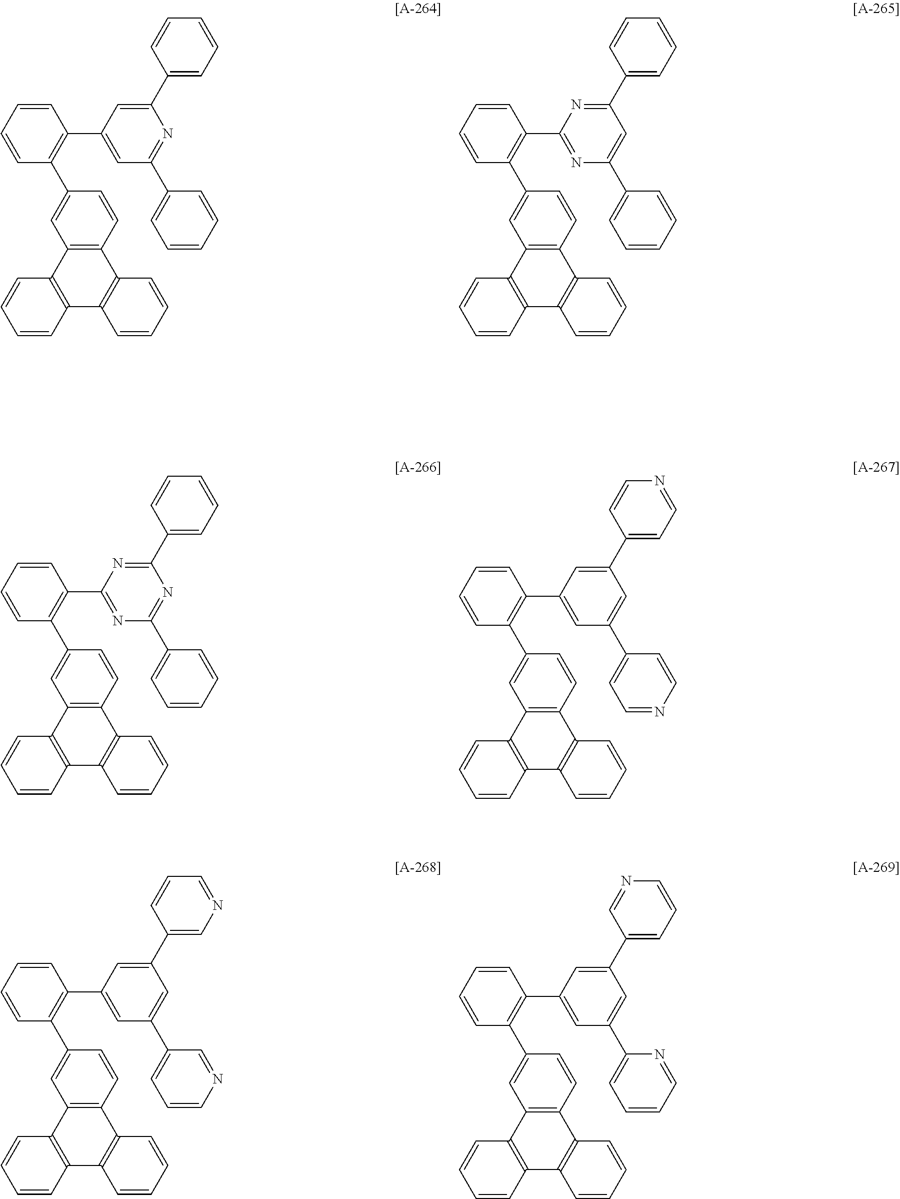 [SMILES: c1ccc(-c2cc(-c3ccccc3-c3ccc4c5ccccc5c5ccccc5c4c3)cc(-c3ccccc3)n2)cc1 ; c1ccc(-c2cc(-c3ccccc3)nc(-c3ccccc3-c3ccc4c5ccccc5c5ccccc5c4c3)n2)cc1 ; c1ccc(-c2nc(-c3ccccc3)nc(-c3ccccc3-c3ccc4c5ccccc5c5ccccc5c4c3)n2)cc1 ; c1ccc(-c2ccc3c4ccccc4c4ccccc4c3c2)c(-c2cc(-c3ccncc3)cc(-c3ccncc3)c2)c1 ; c1cncc(-c2cc(-c3cccnc3)cc(-c3ccccc3-c3ccc4c5ccccc5c5ccccc5c4c3)c2)c1 ; c1ccc(-c2cc(-c3cccnc3)cc(-c3ccccc3-c3ccc4c5ccccc5c5ccccc5c4c3)c2)nc1]

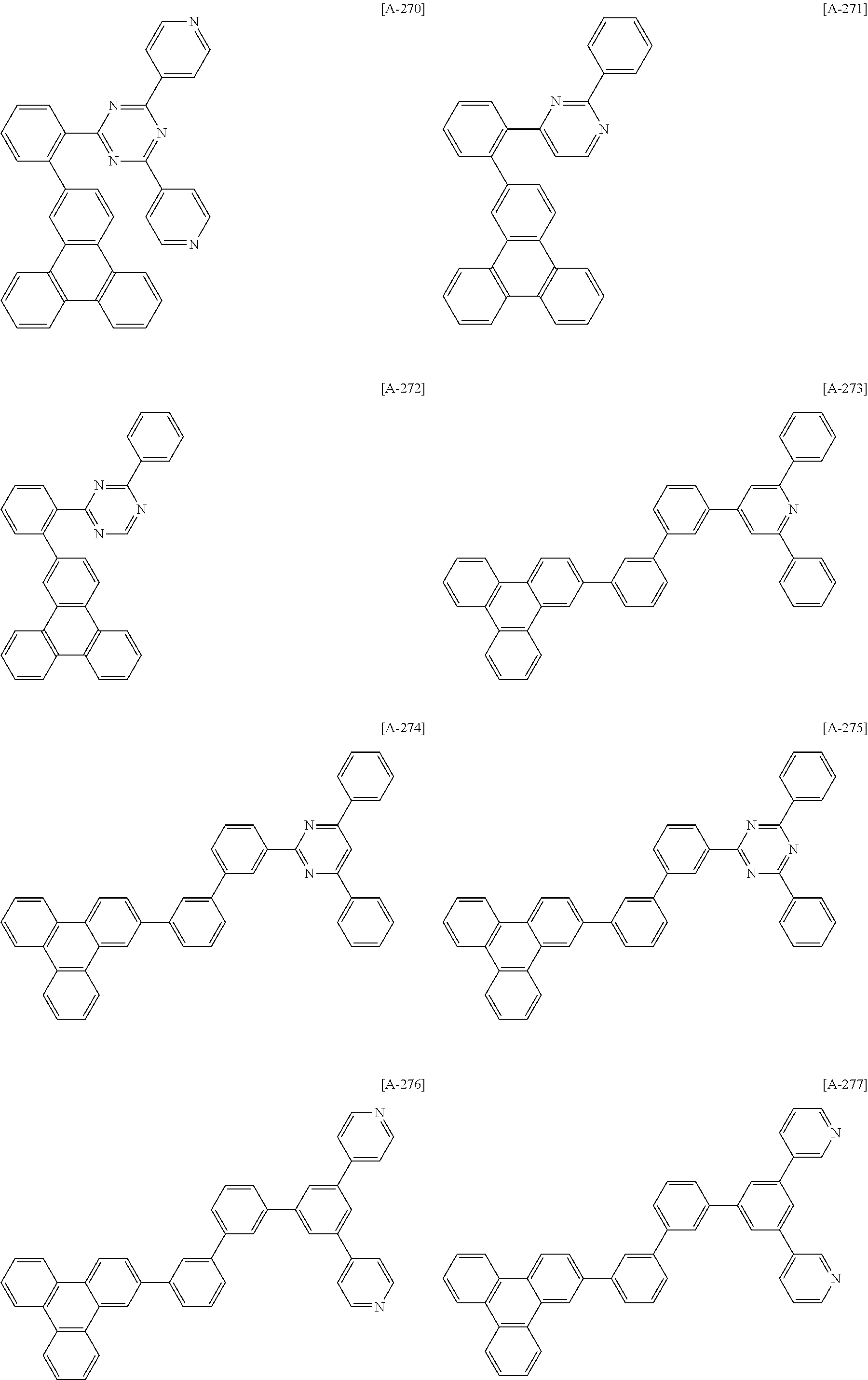

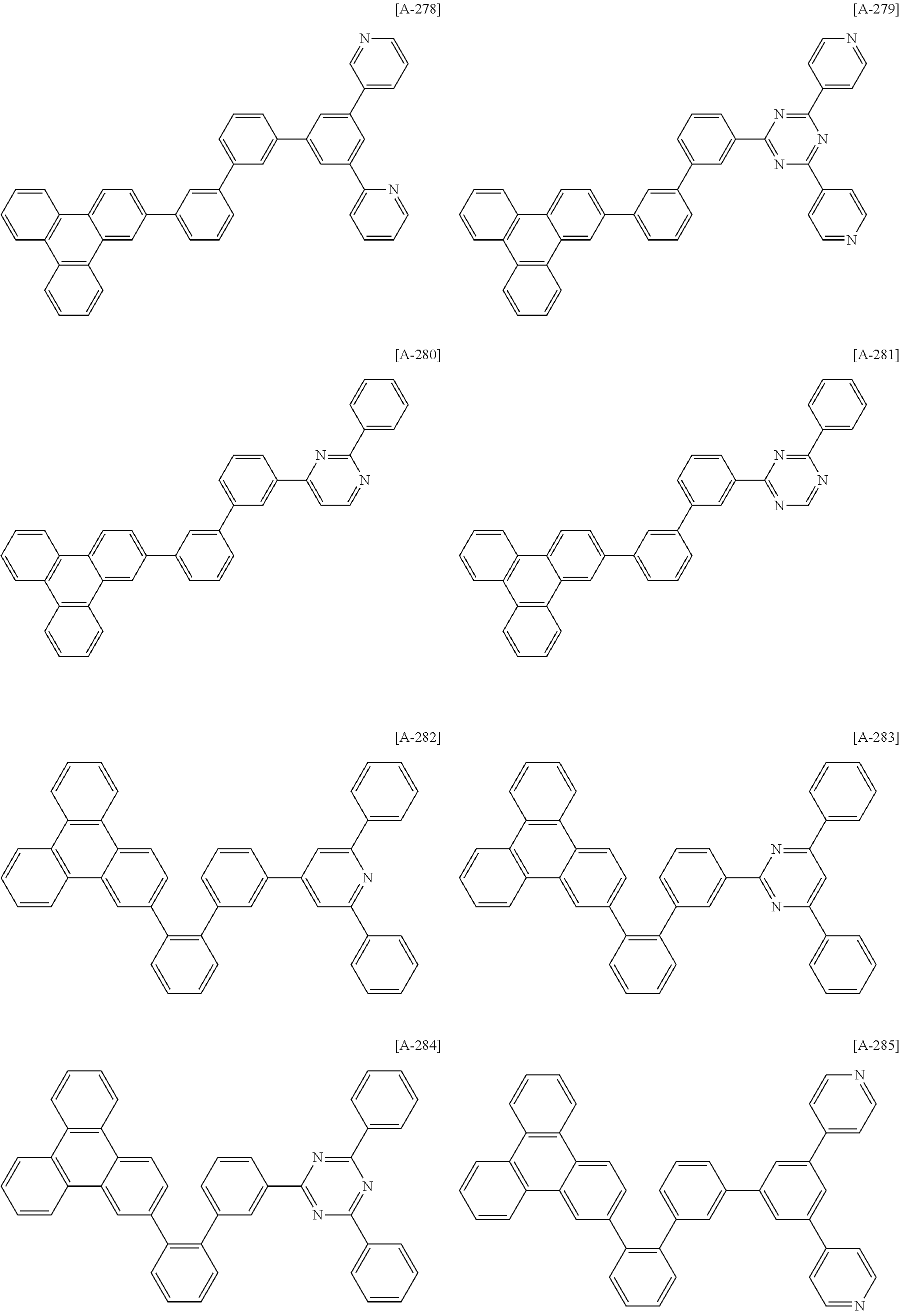

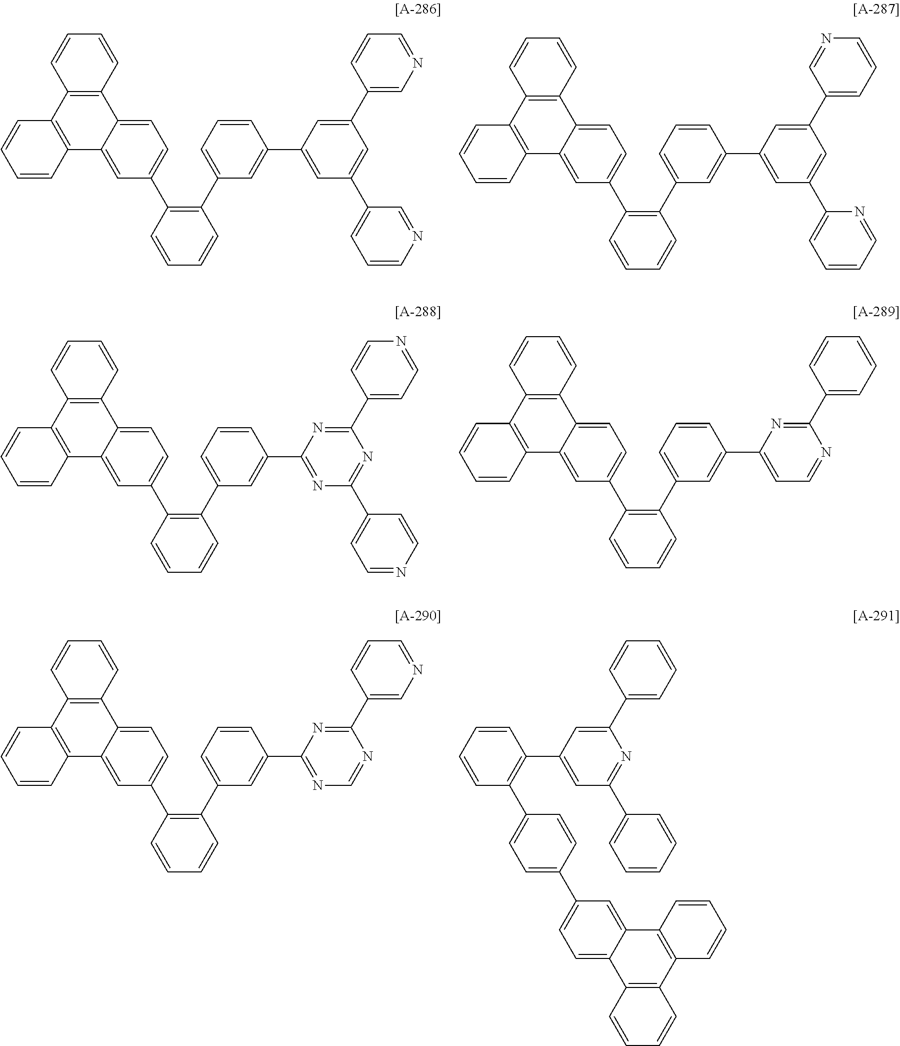 [SMILES: c1cncc(-c2cc(-c3cccnc3)cc(-c3cccc(-c4ccccc4-c4ccc5c6ccccc6c6ccccc6c5c4)c3)c2)c1 ; c1ccc(-c2cc(-c3cccnc3)cc(-c3cccc(-c4ccccc4-c4ccc5c6ccccc6c6ccccc6c5c4)c3)c2)nc1 ; c1cc(-c2nc(-c3ccncc3)nc(-c3ccncc3)n2)cc(-c2ccccc2-c2ccc3c4ccccc4c4ccccc4c3c2)c1 ; c1ccc(-c2nccc(-c3cccc(-c4ccccc4-c4ccc5c6ccccc6c6ccccc6c5c4)c3)n2)cc1 ; c1cncc(-c2ncnc(-c3cccc(-c4ccccc4-c4ccc5c6ccccc6c6ccccc6c5c4)c3)n2)c1 ; c1ccc(-c2cc(-c3ccccc3-c3ccc(-c4ccc5c6ccccc6c6ccccc6c5c4)cc3)cc(-c3ccccc3)n2)cc1]

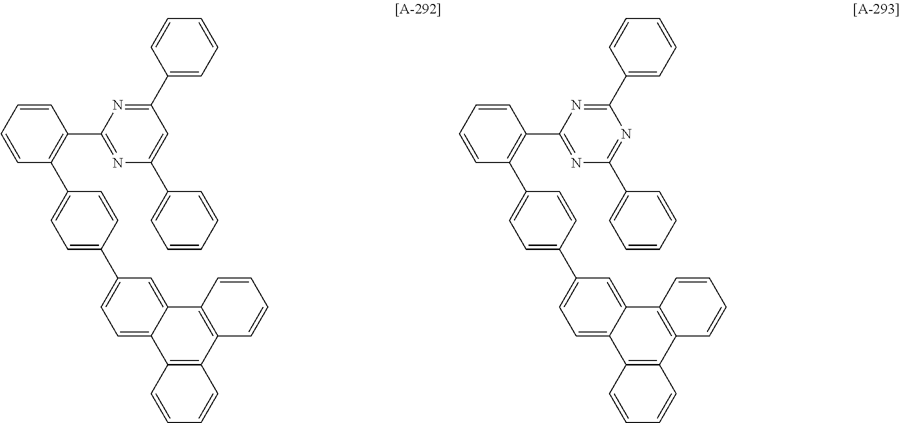 [SMILES: c1ccc(-c2cc(-c3ccccc3)nc(-c3ccccc3-c3ccc(-c4ccc5c6ccccc6c6ccccc6c5c4)cc3)n2)cc1 ; c1ccc(-c2nc(-c3ccccc3)nc(-c3ccccc3-c3ccc(-c4ccc5c6ccccc6c6ccccc6c5c4)cc3)n2)cc1]

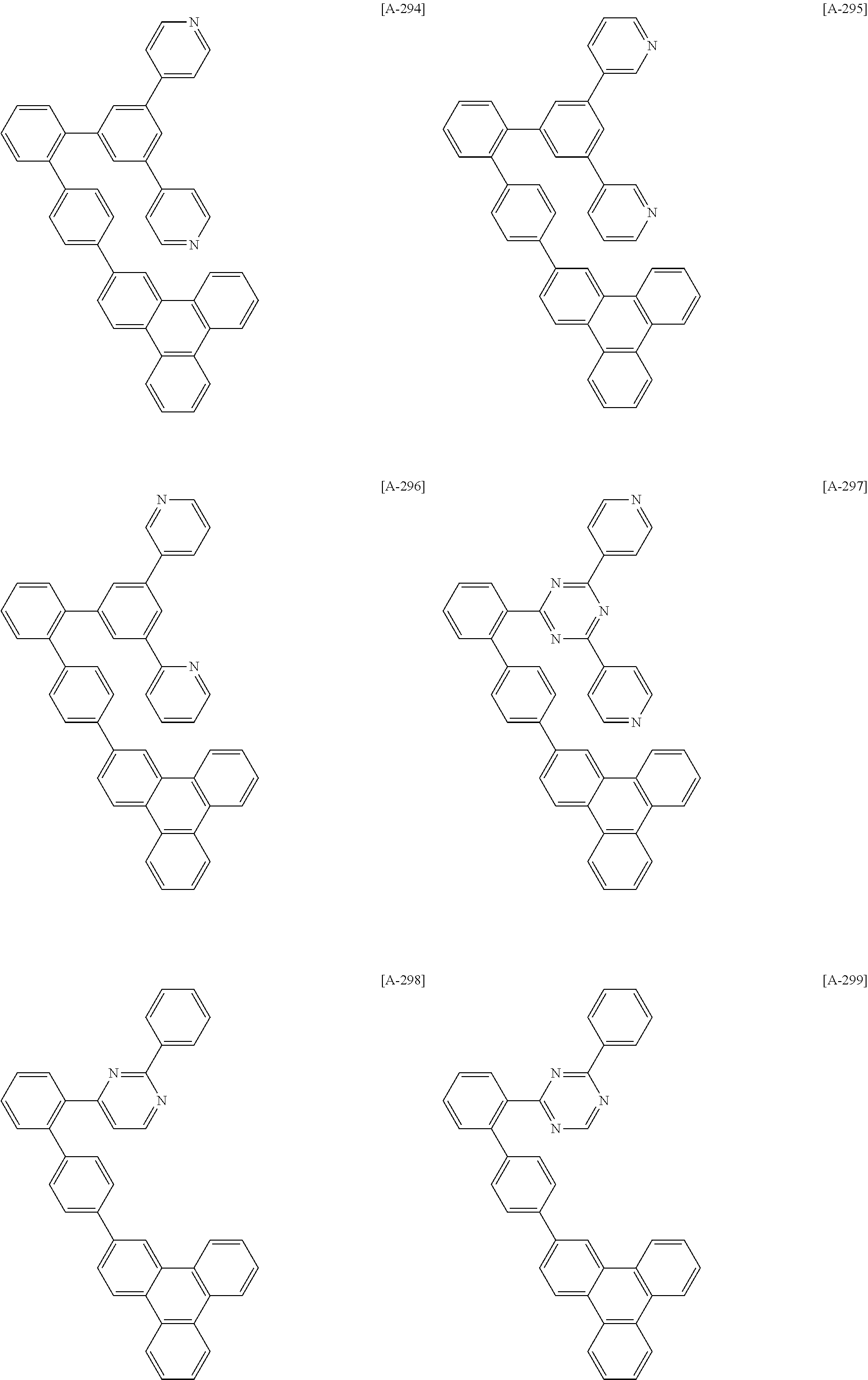 [SMILES: c1ccc(-c2cc(-c3ccncc3)cc(-c3ccncc3)c2)c(-c2ccc(-c3ccc4c5ccccc5c5ccccc5c4c3)cc2)c1 ; c1cncc(-c2cc(-c3cccnc3)cc(-c3ccccc3-c3ccc(-c4ccc5c6ccccc6c6ccccc6c5c4)cc3)c2)c1 ; c1ccc(-c2cc(-c3cccnc3)cc(-c3ccccc3-c3ccc(-c4ccc5c6ccccc6c6ccccc6c5c4)cc3)c2)nc1 ; c1ccc(-c2nc(-c3ccncc3)nc(-c3ccncc3)n2)c(-c2ccc(-c3ccc4c5ccccc5c5ccccc5c4c3)cc2)c1 ; c1ccc(-c2nccc(-c3ccccc3-c3ccc(-c4ccc5c6ccccc6c6ccccc6c5c4)cc3)n2)cc1 ; c1ccc(-c2ncnc(-c3ccccc3-c3ccc(-c4ccc5c6ccccc6c6ccccc6c5c4)cc3)n2)cc1]

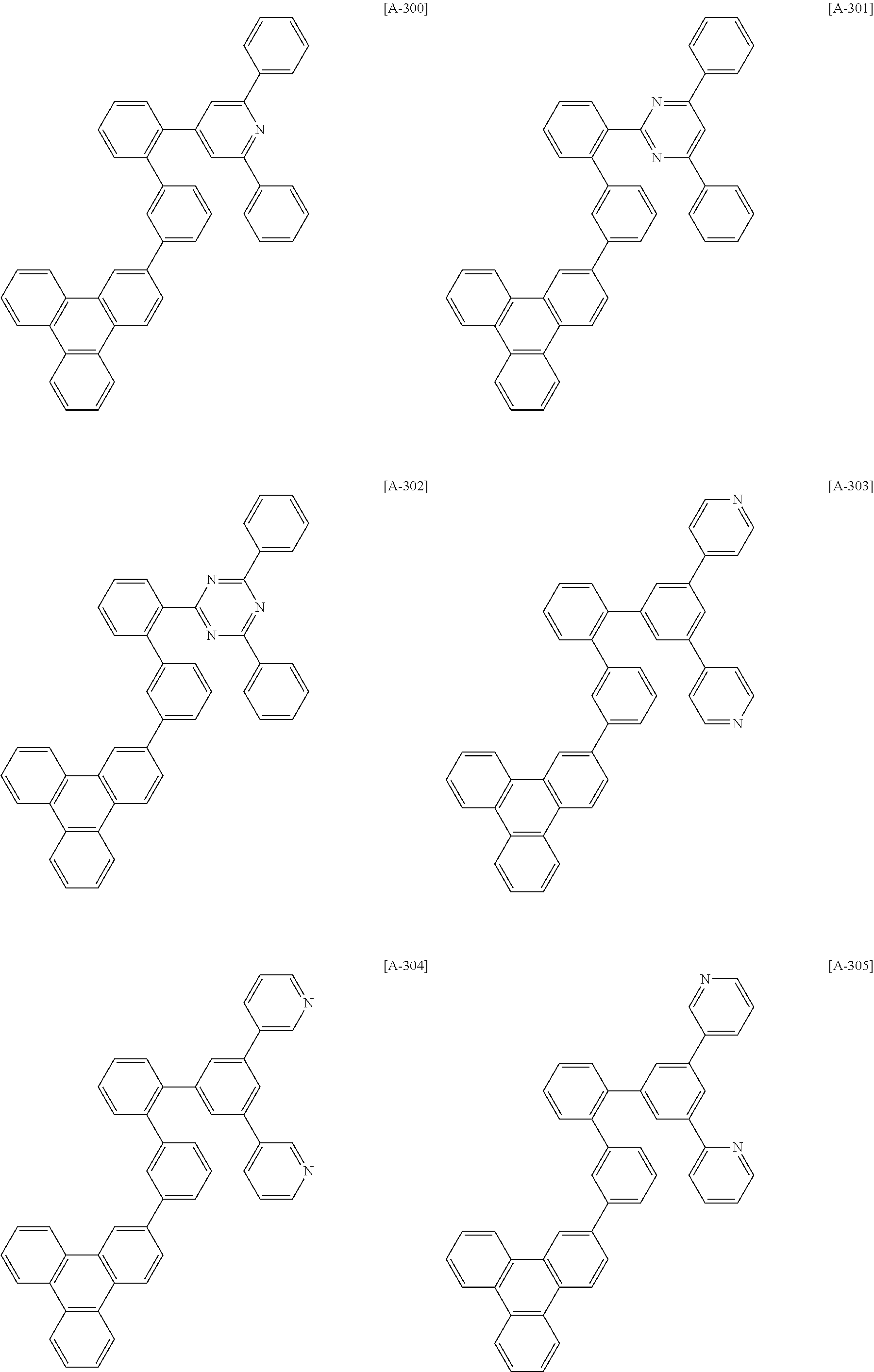 [SMILES: c1ccc(-c2cc(-c3ccccc3-c3cccc(-c4ccc5c6ccccc6c6ccccc6c5c4)c3)cc(-c3ccccc3)n2)cc1 ; c1ccc(-c2cc(-c3ccccc3)nc(-c3ccccc3-c3cccc(-c4ccc5c6ccccc6c6ccccc6c5c4)c3)n2)cc1 ; c1ccc(-c2nc(-c3ccccc3)nc(-c3ccccc3-c3cccc(-c4ccc5c6ccccc6c6ccccc6c5c4)c3)n2)cc1 ; c1cc(-c2ccc3c4ccccc4c4ccccc4c3c2)cc(-c2ccccc2-c2cc(-c3ccncc3)cc(-c3ccncc3)c2)c1 ; c1cncc(-c2cc(-c3cccnc3)cc(-c3ccccc3-c3cccc(-c4ccc5c6ccccc6c6ccccc6c5c4)c3)c2)c1 ; c1ccc(-c2cc(-c3cccnc3)cc(-c3ccccc3-c3cccc(-c4ccc5c6ccccc6c6ccccc6c5c4)c3)c2)nc1]

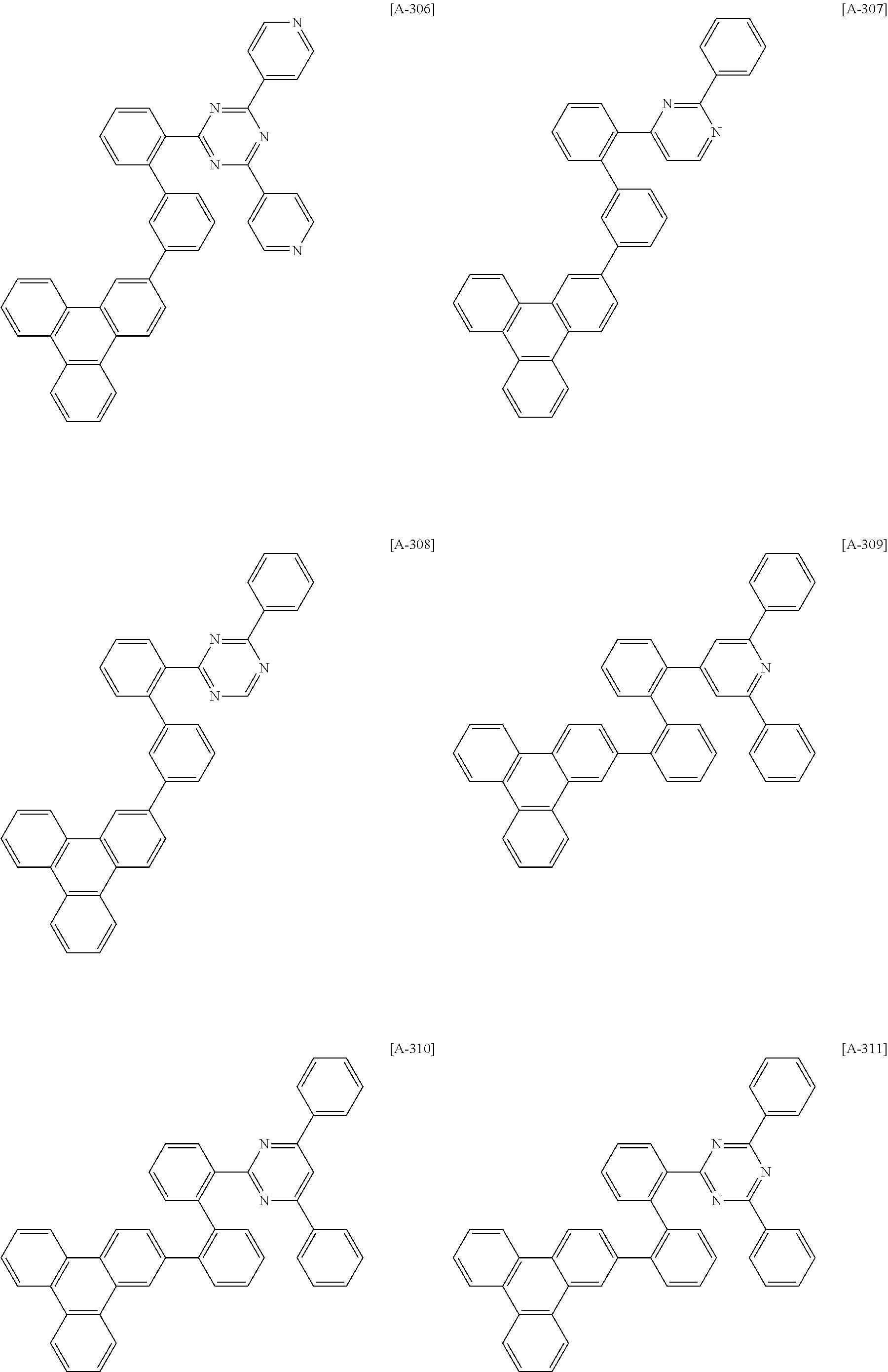 [SMILES: c1cc(-c2ccc3c4ccccc4c4ccccc4c3c2)cc(-c2ccccc2-c2nc(-c3ccncc3)nc(-c3ccncc3)n2)c1 ; c1ccc(-c2nccc(-c3ccccc3-c3cccc(-c4ccc5c6ccccc6c6ccccc6c5c4)c3)n2)cc1 ; c1ccc(-c2ncnc(-c3ccccc3-c3cccc(-c4ccc5c6ccccc6c6ccccc6c5c4)c3)n2)cc1 ; c1ccc(-c2cc(-c3ccccc3-c3ccccc3-c3ccc4c5ccccc5c5ccccc5c4c3)cc(-c3ccccc3)n2)cc1 ; c1ccc(-c2cc(-c3ccccc3)nc(-c3ccccc3-c3ccccc3-c3ccc4c5ccccc5c5ccccc5c4c3)n2)cc1 ; c1ccc(-c2nc(-c3ccccc3)nc(-c3ccccc3-c3ccccc3-c3ccc4c5ccccc5c5ccccc5c4c3)n2)cc1]

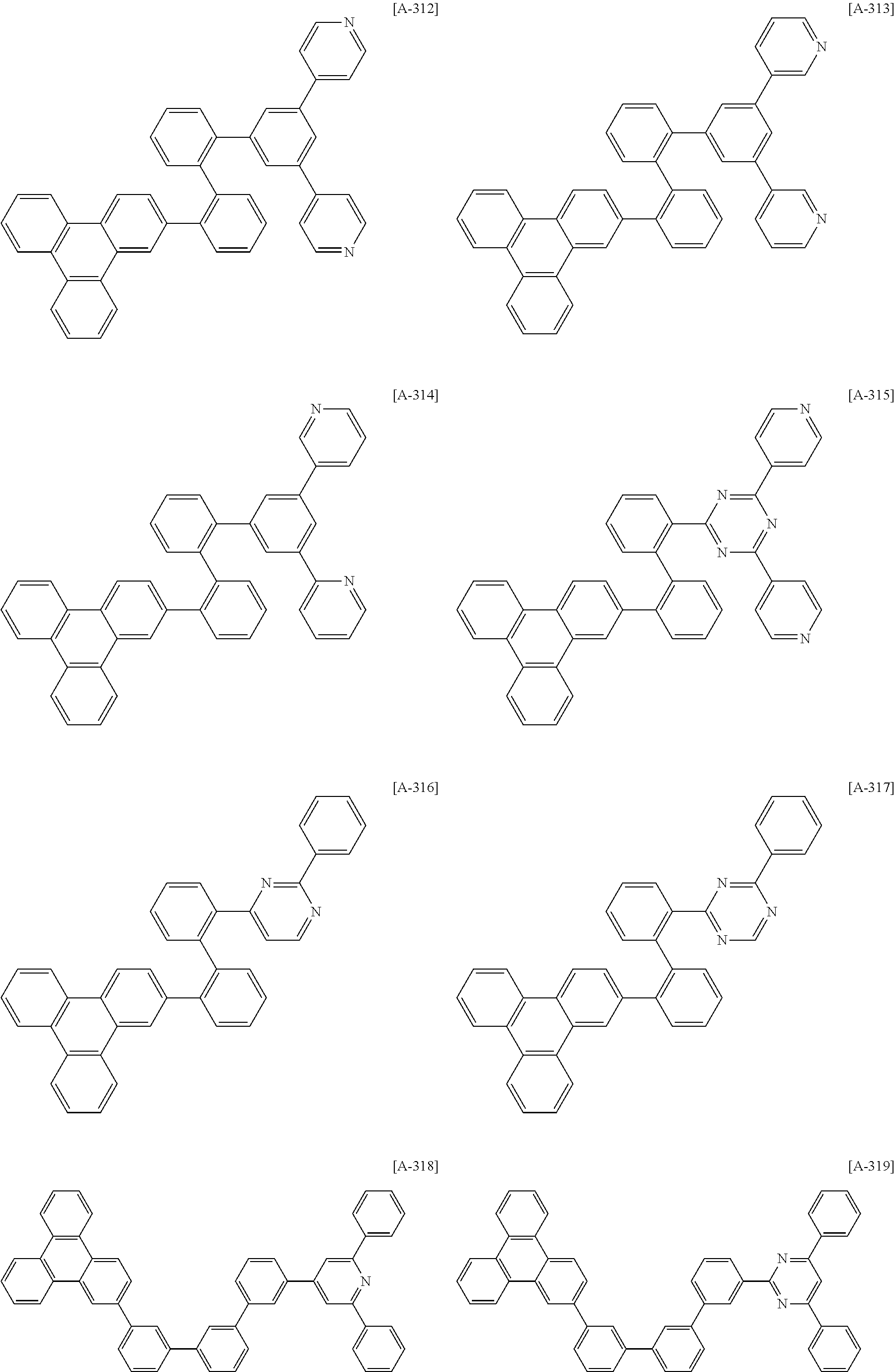

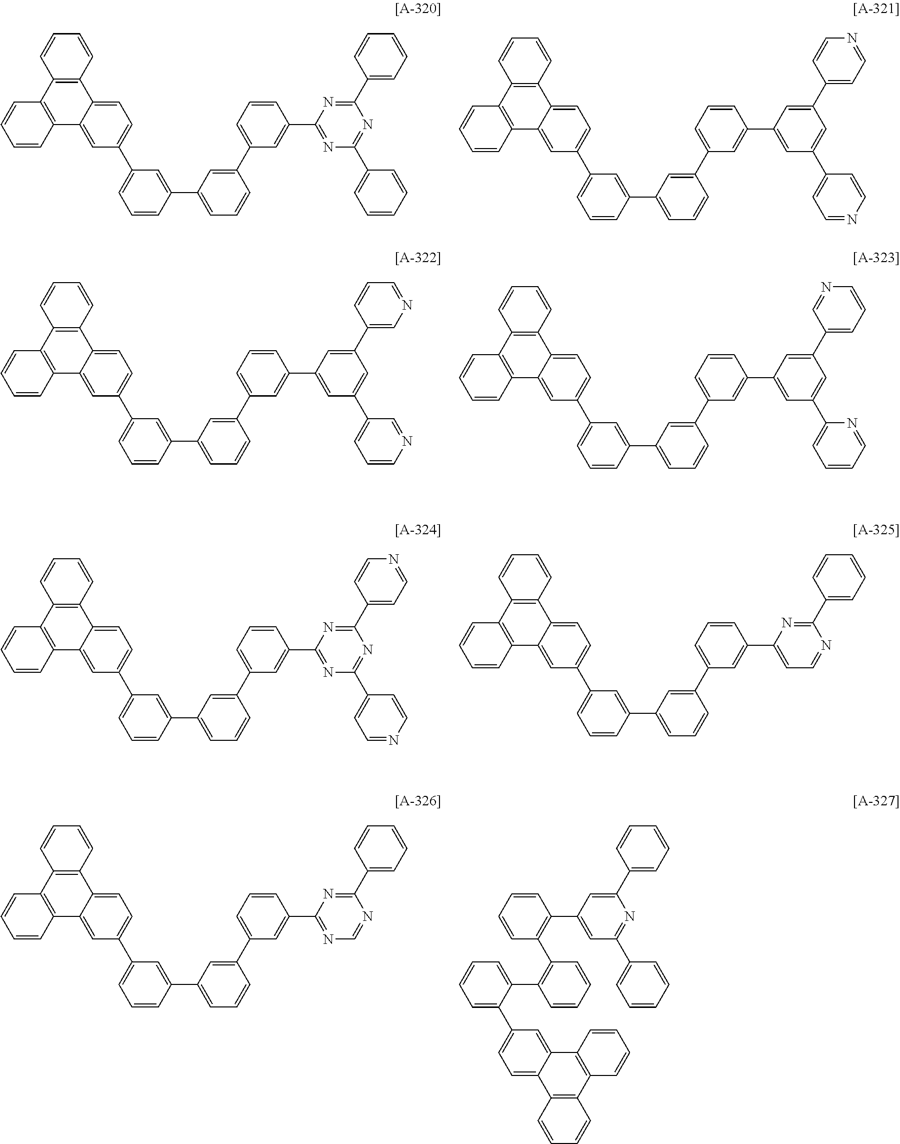

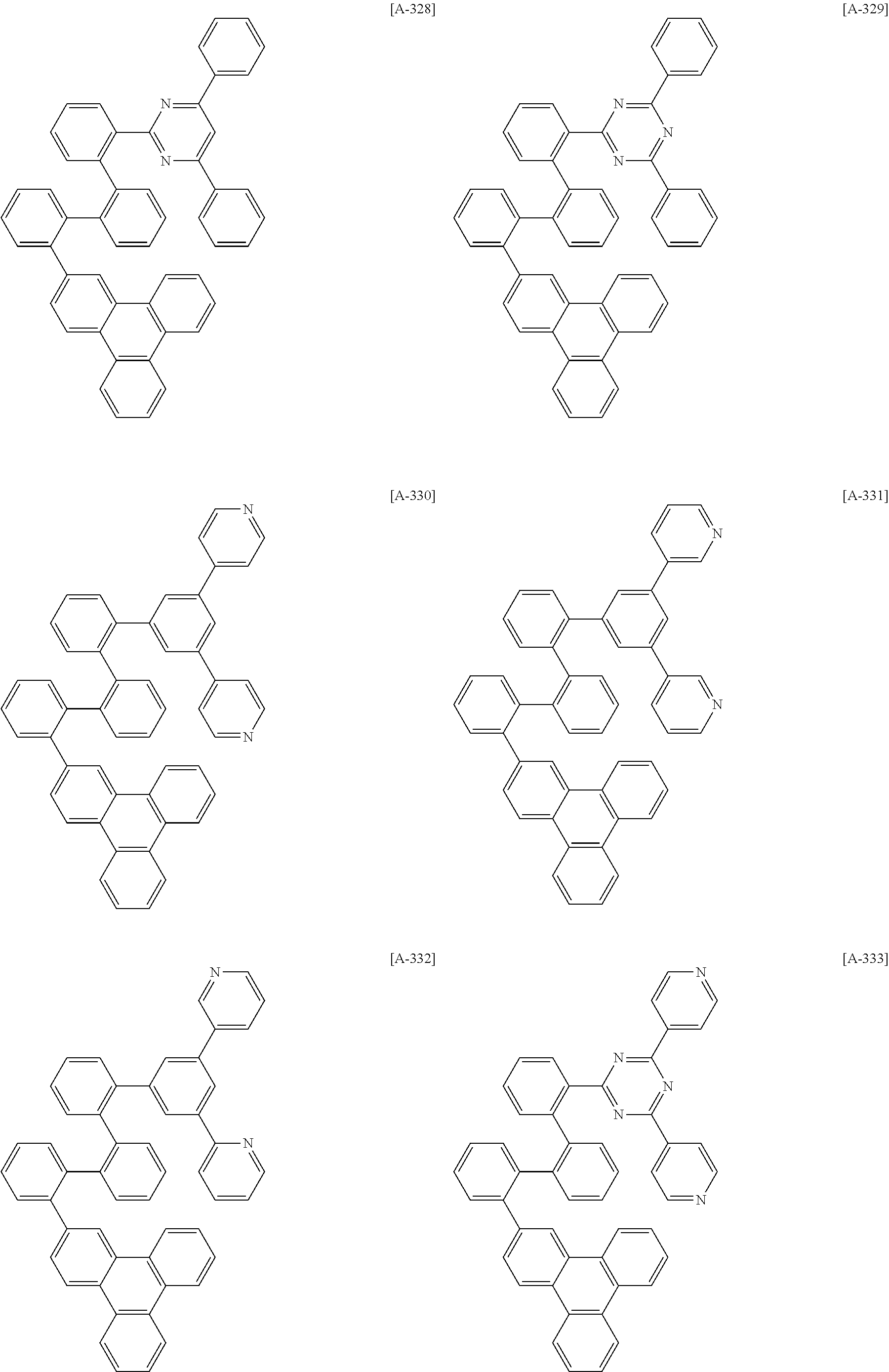

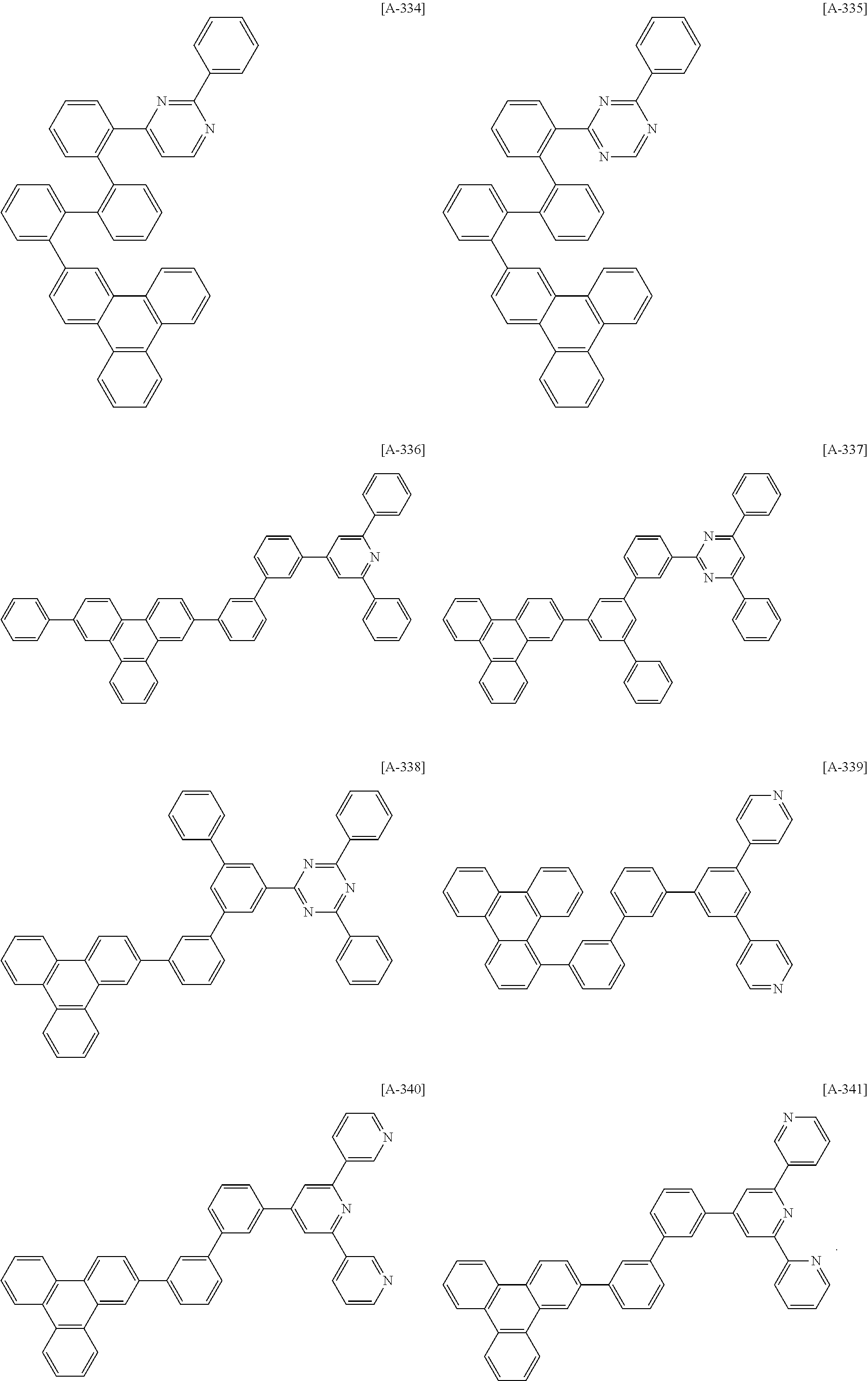

The first compound may be used with at least one second compound having a carbazole moiety or a carbazole derivative for an emission layer.

The carbazole derivative has a structure derived based on a carbazole moiety and indicates a fused carbazole moiety consisting of a combination of the moiety represented by Chemical Formula 3 and the moiety represented by Chemical Formula 4.

The second compound may be represented by Chemical Formula 2.

L³ to L⁶ of Chemical Formula 2 according to an embodiment may independently be a single bond, a substituted or unsubstituted C6 to C30 arylene group, or a substituted or unsubstituted C2 to C30 heteroarylene group, and specifically substituted or unsubstituted C6 to C30 arylene group. For example, they may be a single bond, a substituted or unsubstituted phenylene group, a substituted or unsubstituted biphenylene group, a substituted or unsubstituted terphenylene group, a substituted or unsubstituted quaterphenylene group, a substituted or unsubstituted naphthylene group, a substituted or unsubstituted anthracenylene group, a substituted or unsubstituted triphenylenyl group, or a substituted or unsubstituted phenanthrenylene group.

R⁷ to R¹⁰ of Chemical Formula 1 according to an embodiment may independently be hydrogen, deuterium, a substituted or unsubstituted C1 to C20 alkyl group, a substituted or unsubstituted C6 to C50 aryl group, a substituted or unsubstituted C2 to C50 heterocyclic group, or a combination thereof as described above, and

specifically is hydrogen, deuterium, a substituted or unsubstituted C6 to C30 aryl group, or a substituted or unsubstituted C2 to C50 heterocyclic group. For example, R⁷ to R¹⁰ may independently be selected from hydrogen, a substituted or unsubstituted phenyl group, or a substituted or unsubstituted biphenyl group, a substituted or unsubstituted triphenylene group, a substituted or unsubstituted fluorenyl group, a substituted or unsubstituted carbazolyl group, a substituted or unsubstituted dibenzofuranyl group, a substituted or unsubstituted dibenzothiophenyl group, or a combination thereof, but is not limited thereto.

Chemical Formula 2 may be, for example represented by at least one of Chemical Formulae 2-I to 2-III.

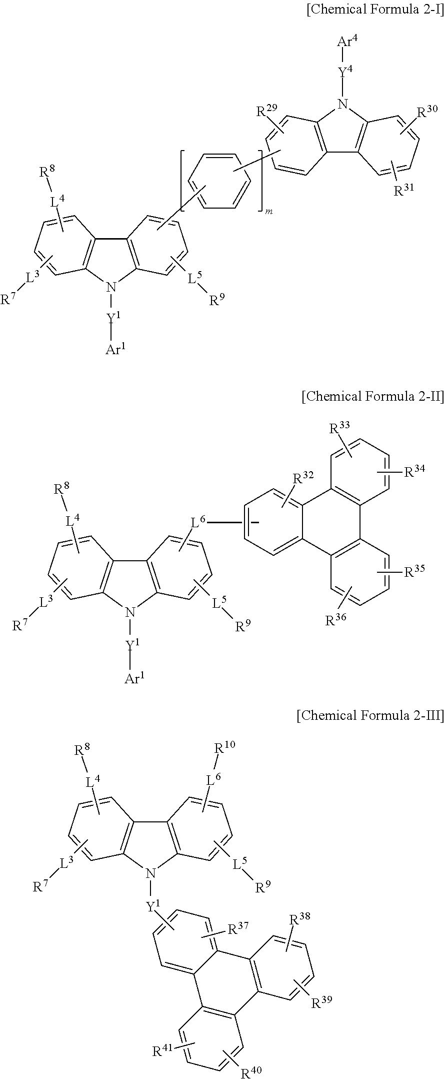

In Chemical Formulae 2-I to 2-III, L³ to L⁶, Y¹, and R⁷ to R¹⁰ are the same as above,

R²⁹ to R⁴¹ are independently hydrogen, deuterium, a substituted or unsubstituted C1 to C20 alkyl group, a substituted or unsubstituted C6 to C50 aryl group, a substituted or unsubstituted C2 to C50 heterocyclic group, or a combination thereof,

Y⁴ is a single bond, a substituted or unsubstituted C6 to C30 arylene group, a substituted or unsubstituted C2 to C30 heteroarylene group, or a combination thereof,

Ar¹ and Ar⁴ are independently a substituted or unsubstituted C6 to C30 aryl group, a substituted or unsubstituted benzofuranyl group, a substituted or unsubstituted benzothiophenyl group, a substituted or unsubstituted dibenzofuranyl group, a substituted or unsubstituted dibenzothiophenyl group, a substituted or unsubstituted carbazolyl group, or a combination thereof, and

m is an integer of 0 to 4,

wherein, “substituted” is the same as defined above.

Specifically, Ar¹ and Ar⁴ of Chemical Formula 2-I to 2-III may independently be a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted carbazolyl group, a substituted or unsubstituted benzofuranyl group, a substituted or unsubstituted benzothiophenyl group, a substituted or unsubstituted fluorenyl group, a substituted or unsubstituted dibenzofuranyl group, a substituted or unsubstituted dibenzothiophenyl group, a substituted or unsubstituted pyridinyl group, or a combination thereof.

Specifically, Chemical Formula 2-I may be one of structures of Group 3 and the *—Y¹— Ar¹ and *—Y⁴—Ar⁴ may be one of substituents of Group 4.

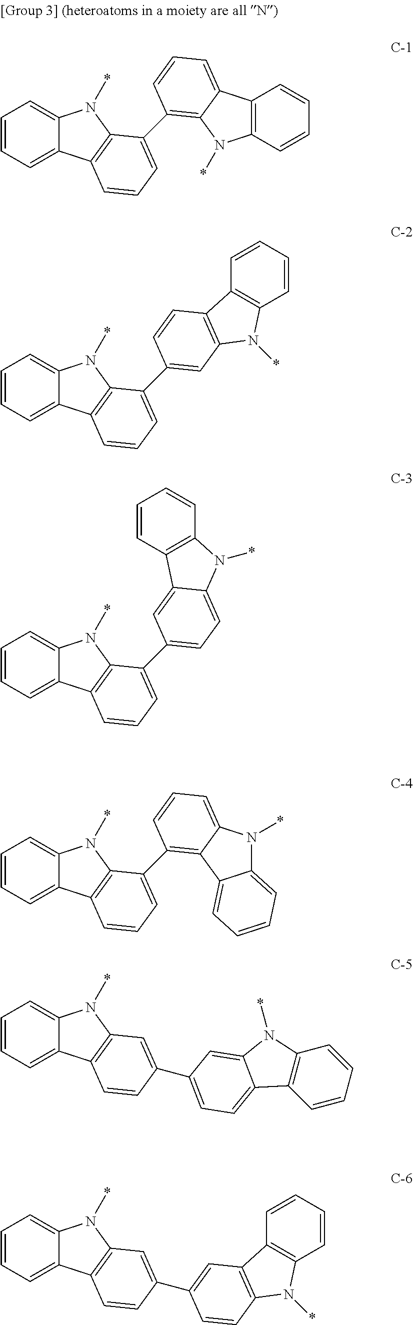 [SMILES: *n1c2ccccc2c2cccc(-c3cccc4c5ccccc5n(*)c34)c21 ; *n1c2ccccc2c2ccc(-c3cccc4c5ccccc5n(*)c34)cc21 ; *n1c2ccccc2c2cc(-c3cccc4c5ccccc5n(*)c34)ccc21 ; *n1c2ccccc2c2c(-c3cccc4c5ccccc5n(*)c34)cccc21 ; *n1c2ccccc2c2ccc(-c3ccc4c5ccccc5n(*)c4c3)cc21 ; *n1c2ccccc2c2cc(-c3ccc4c5ccccc5n(*)c4c3)ccc21]

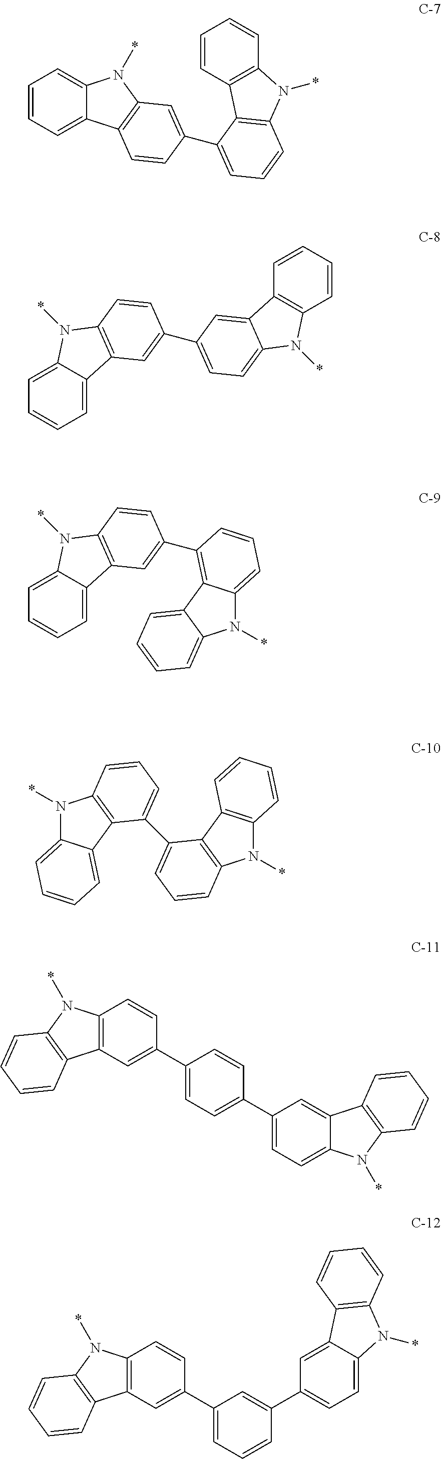 [SMILES: *n1c2ccccc2c2ccc(-c3cccc4c3c3ccccc3n4*)cc21 ; *n1c2ccccc2c2cc(-c3ccc4c(c3)c3ccccc3n4*)ccc21 ; *n1c2ccccc2c2cc(-c3cccc4c3c3ccccc3n4*)ccc21 ; *n1c2ccccc2c2c(-c3cccc4c3c3ccccc3n4*)cccc21 ; *n1c2ccccc2c2cc(-c3ccc(-c4ccc5c(c4)c4ccccc4n5*)cc3)ccc21 ; *n1c2ccccc2c2cc(-c3cccc(-c4ccc5c(c4)c4ccccc4n5*)c3)ccc21]

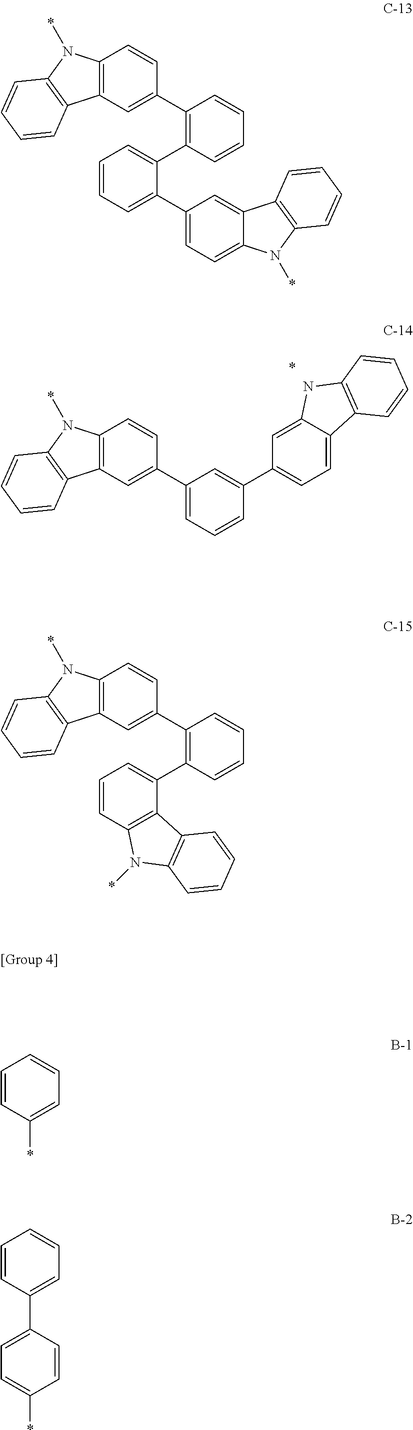 [SMILES: *n1c2ccccc2c2cc(-c3ccccc3-c3ccccc3-c3ccc4c(c3)c3ccccc3n4*)ccc21 ; *n1c2ccccc2c2cc(-c3cccc(-c4ccc5c(c4)[nH]c4ccccc45)c3)ccc21 ; *n1c2ccccc2c2cc(-c3ccccc3-c3cccc4c3c3ccccc3n4*)ccc21 ; *c1ccccc1 ; *c1ccc(-c2ccccc2)cc1]

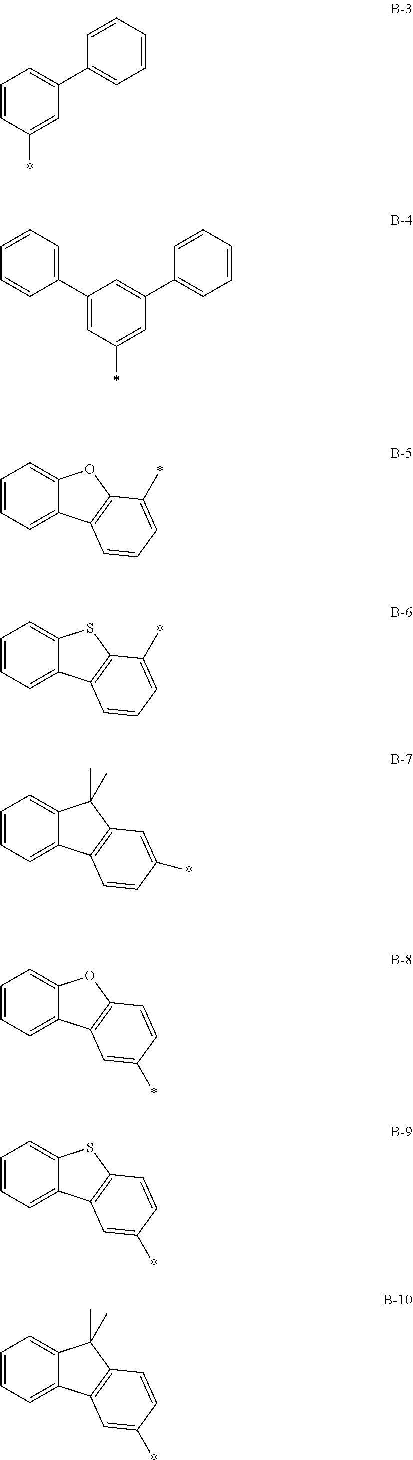

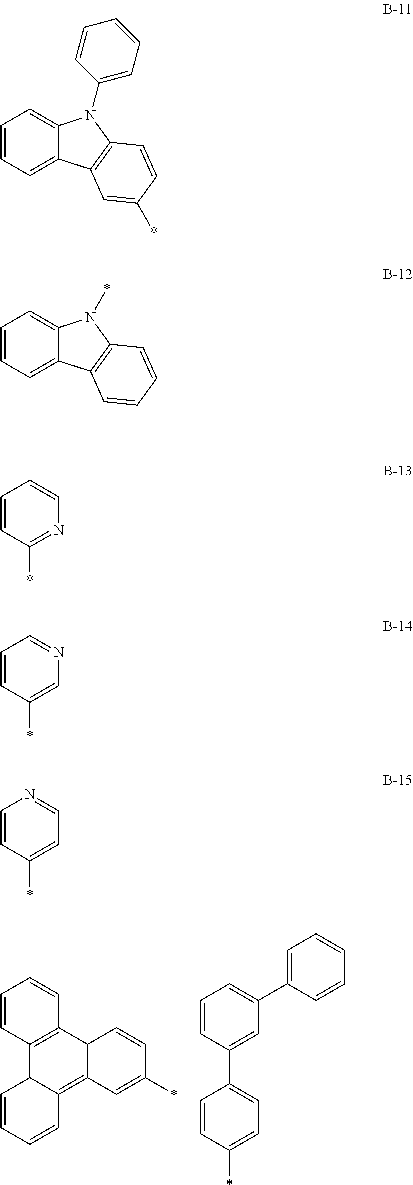

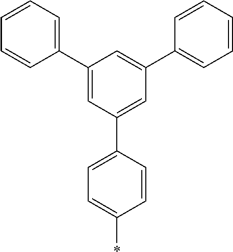 [SMILES: *c1ccc(-c2cc(-c3ccccc3)cc(-c3ccccc3)c2)cc1]

B-16 B-17 B-18

In Group 3 and Group 4, * is a linking point.

The second compound represented by Chemical Formula 2 may be, for example compounds of Group B to Group D, but is not limited thereto.

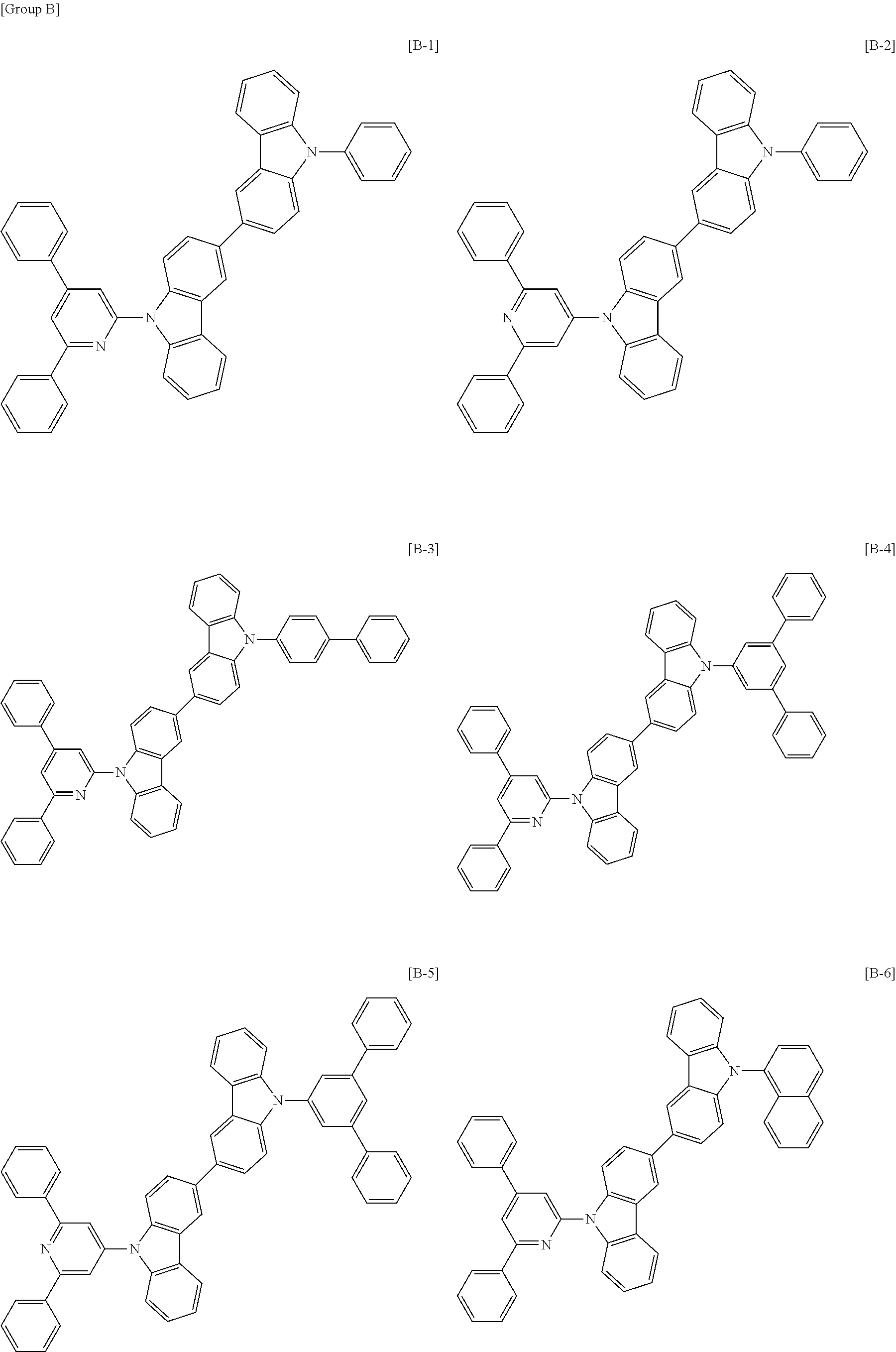

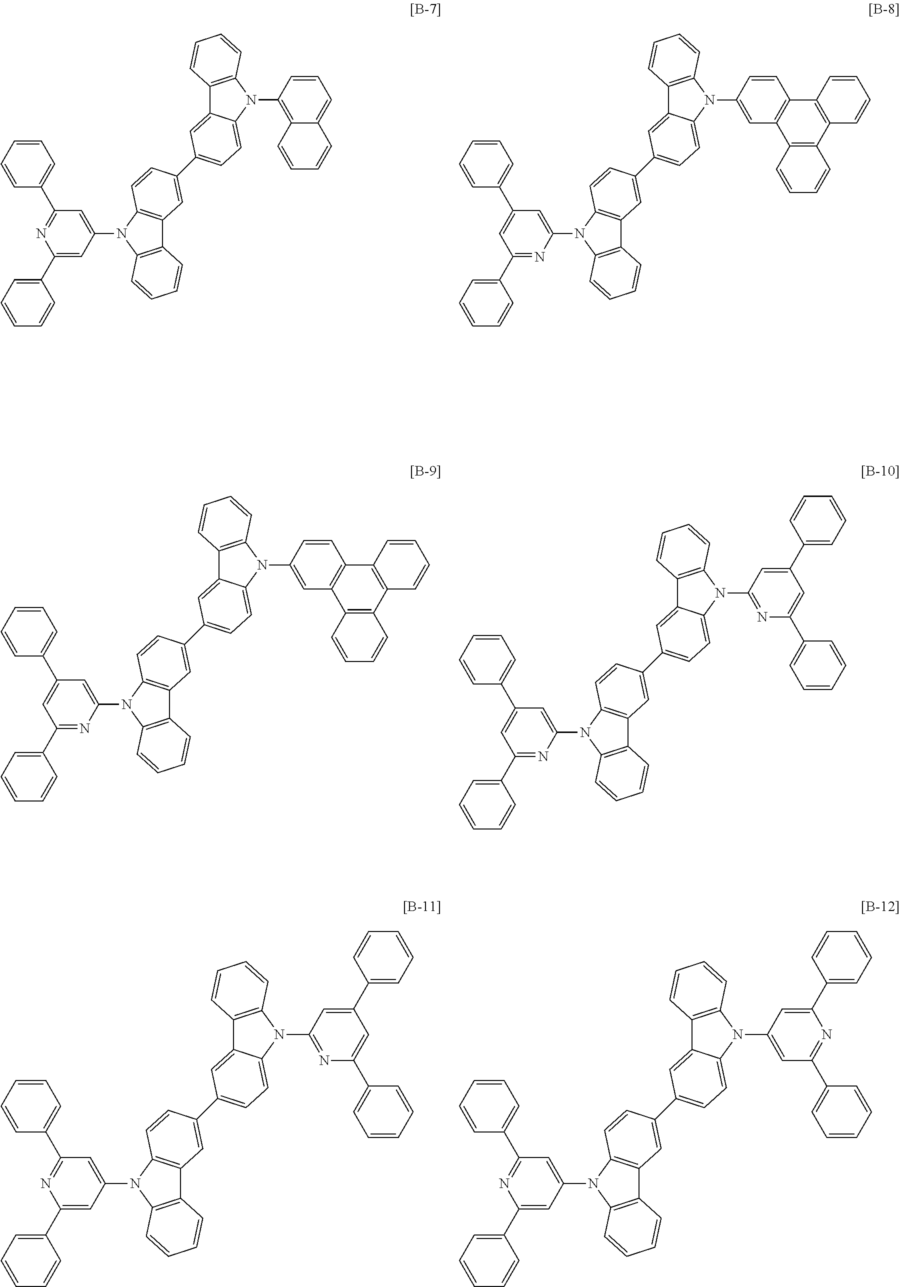

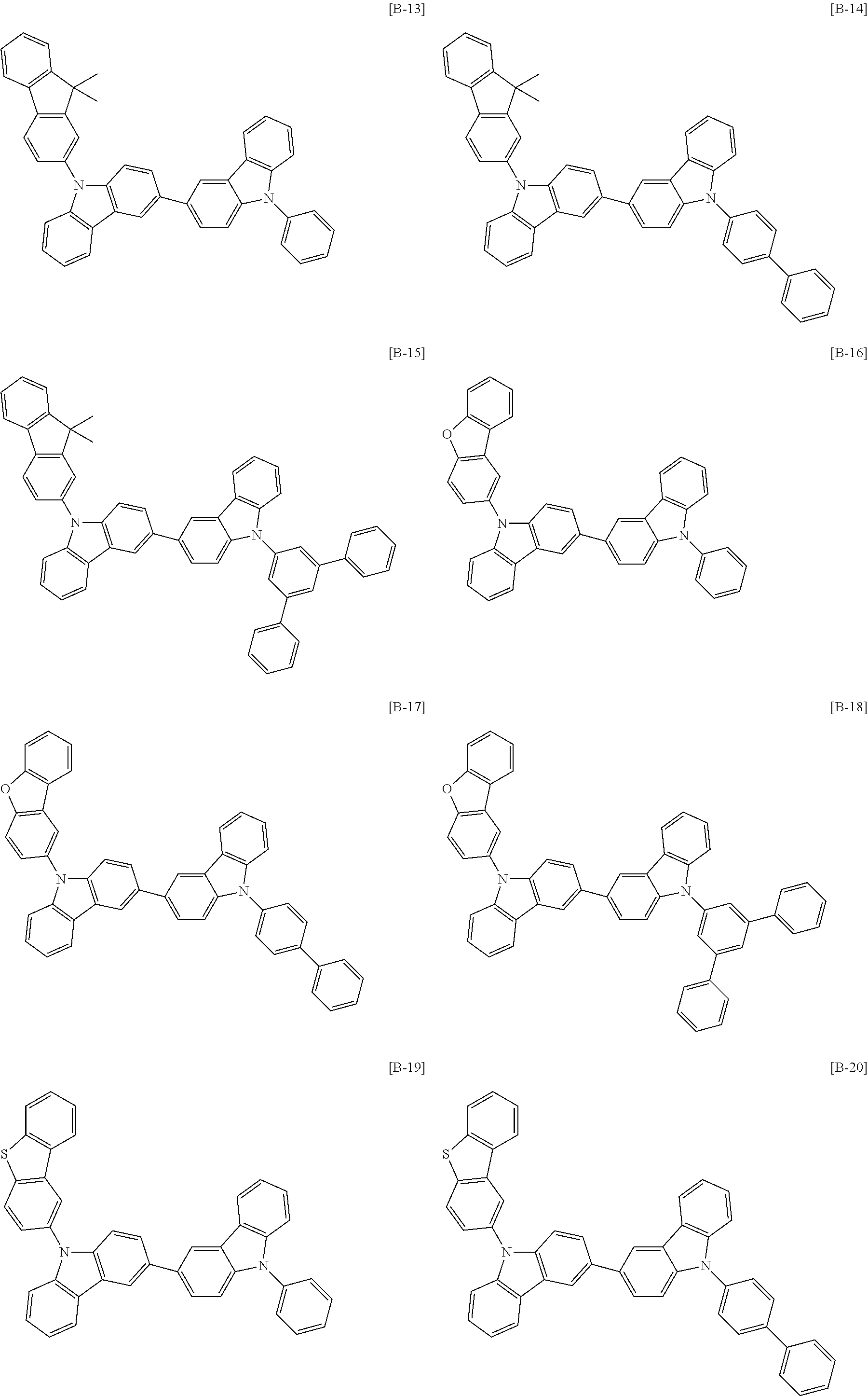

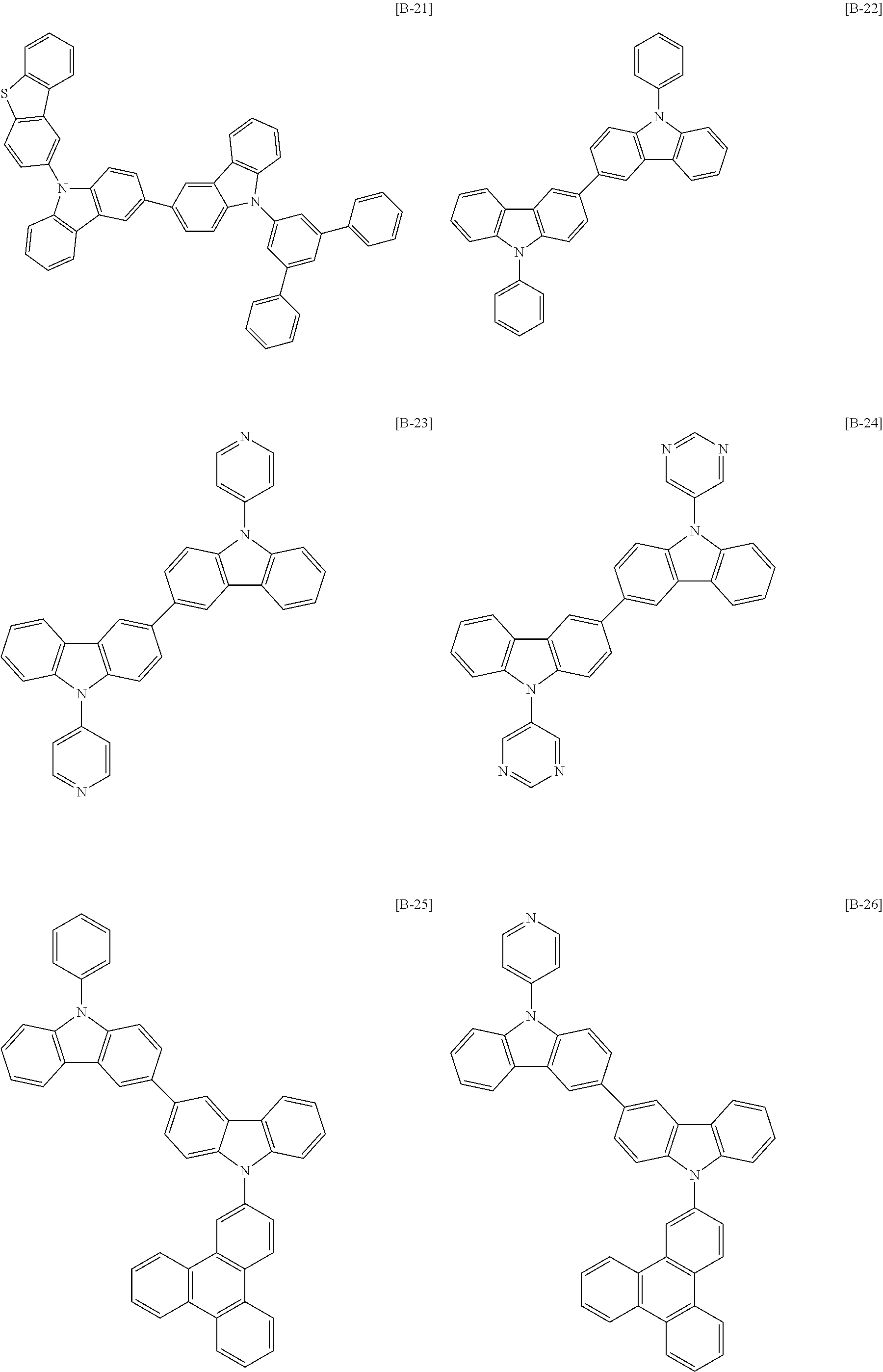 [SMILES: c1ccc(-c2cc(-c3ccccc3)cc(-n3c4ccccc4c4cc(-c5ccc6c(c5)c5ccccc5n6-c5ccc6sc7ccccc7c6c5)ccc43)c2)cc1 ; c1ccc(-n2c3ccccc3c3cc(-c4ccc5c(c4)c4ccccc4n5-c4ccccc4)ccc32)cc1 ; c1ccc2c(c1)c1cc(-c3ccc4c(c3)c3ccccc3n4-c3ccncc3)ccc1n2-c1ccncc1 ; c1ccc2c(c1)c1cc(-c3ccc4c(c3)c3ccccc3n4-c3cncnc3)ccc1n2-c1cncnc1 ; c1ccc(-n2c3ccccc3c3cc(-c4ccc5c(c4)c4ccccc4n5-c4ccc5c6ccccc6c6ccccc6c5c4)ccc32)cc1 ; c1ccc2c(c1)c1ccccc1c1cc(-n3c4ccccc4c4cc(-c5ccc6c(c5)c5ccccc5n6-c5ccncc5)ccc43)ccc21]

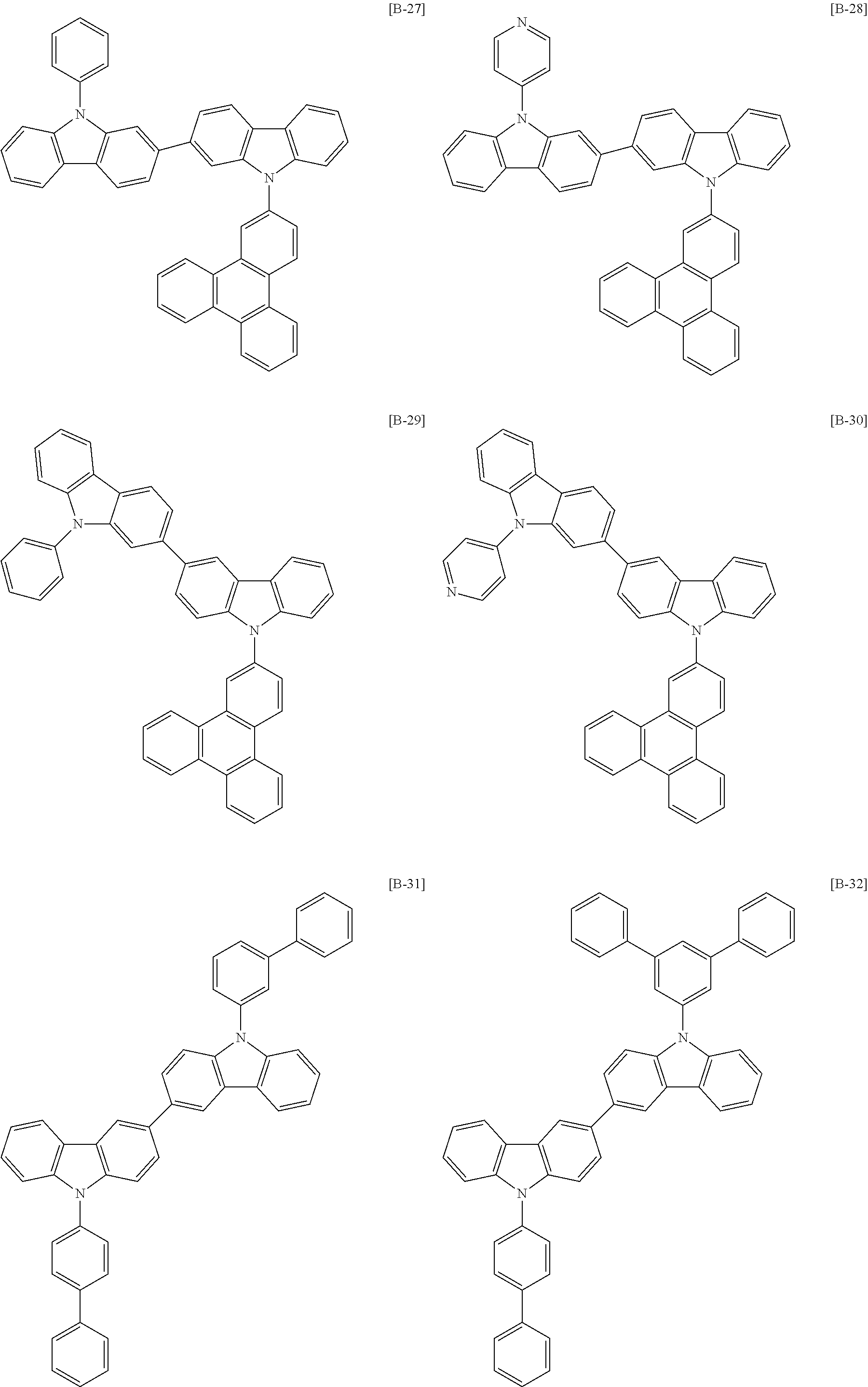

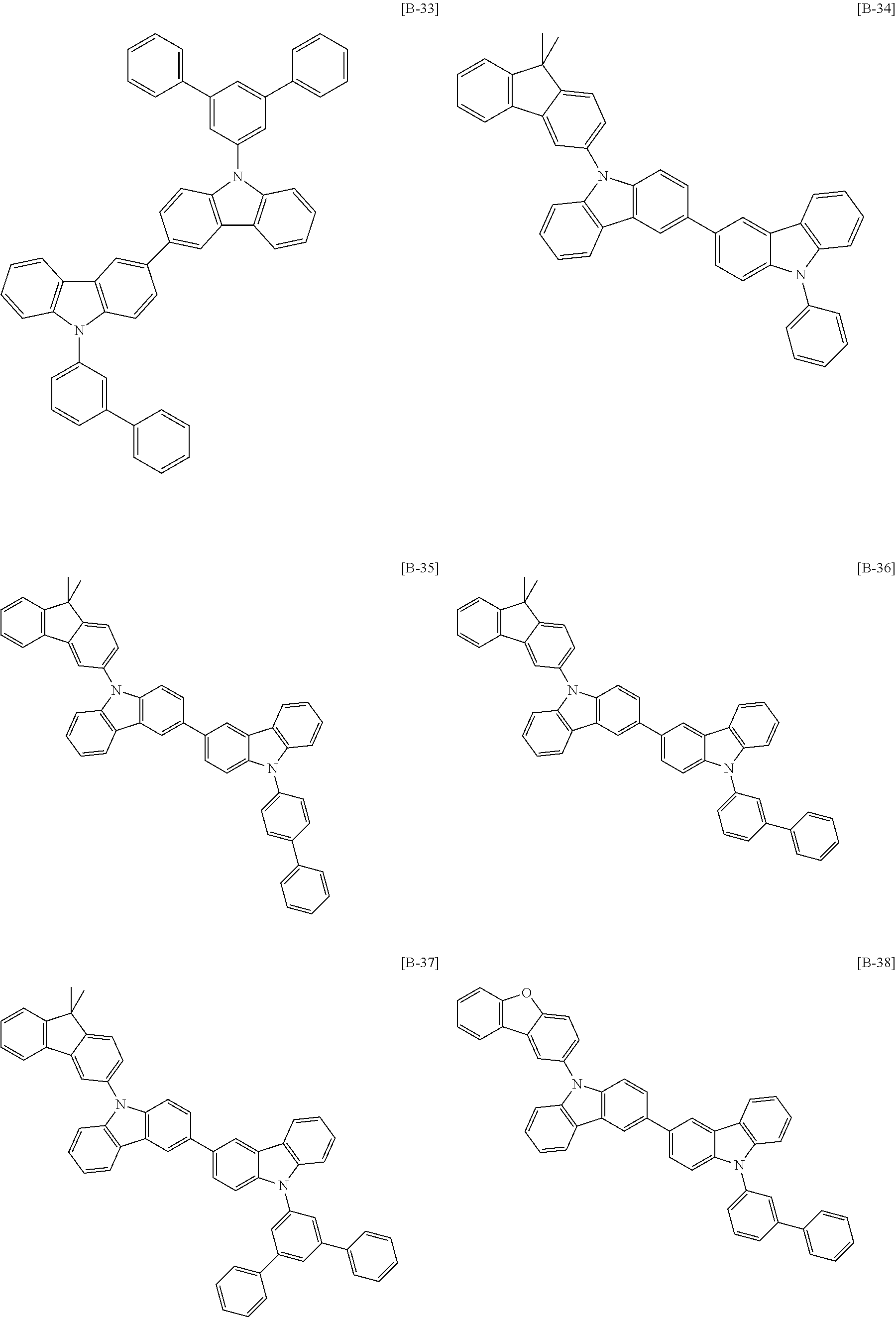

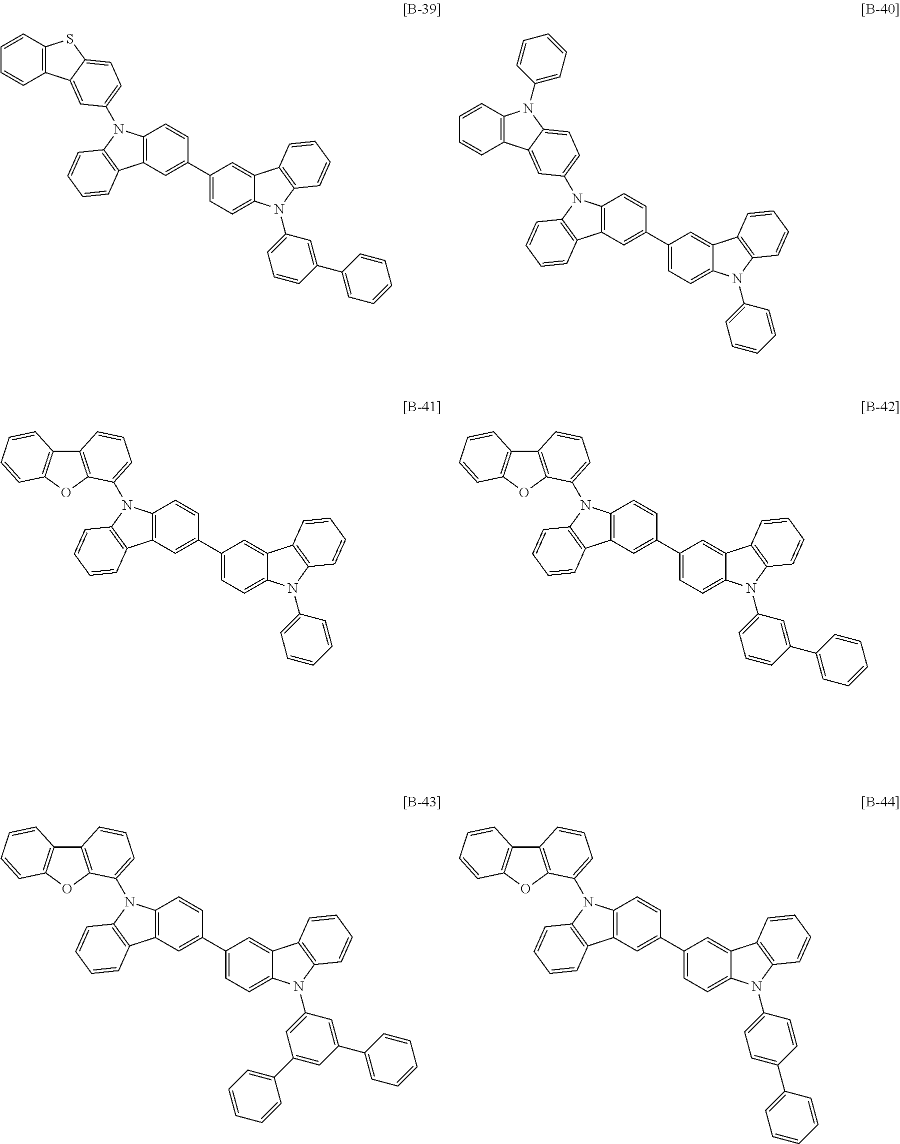

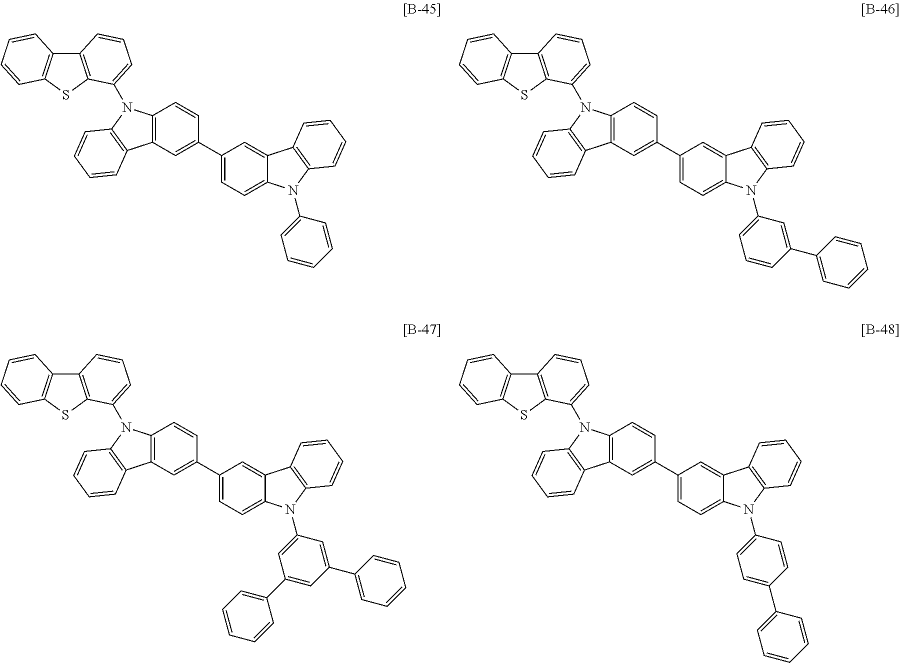 [SMILES: c1ccc(-n2c3ccccc3c3cc(-c4ccc5c(c4)c4ccccc4n5-c4cccc5c4sc4ccccc45)ccc32)cc1 ; c1ccc(-c2cccc(-n3c4ccccc4c4cc(-c5ccc6c(c5)c5ccccc5n6-c5cccc6c5sc5ccccc56)ccc43)c2)cc1 ; c1ccc(-c2cc(-c3ccccc3)cc(-n3c4ccccc4c4cc(-c5ccc6c(c5)c5ccccc5n6-c5cccc6c5sc5ccccc56)ccc43)c2)cc1 ; c1ccc(-c2ccc(-n3c4ccccc4c4cc(-c5ccc6c(c5)c5ccccc5n6-c5cccc6c5sc5ccccc56)ccc43)cc2)cc1]

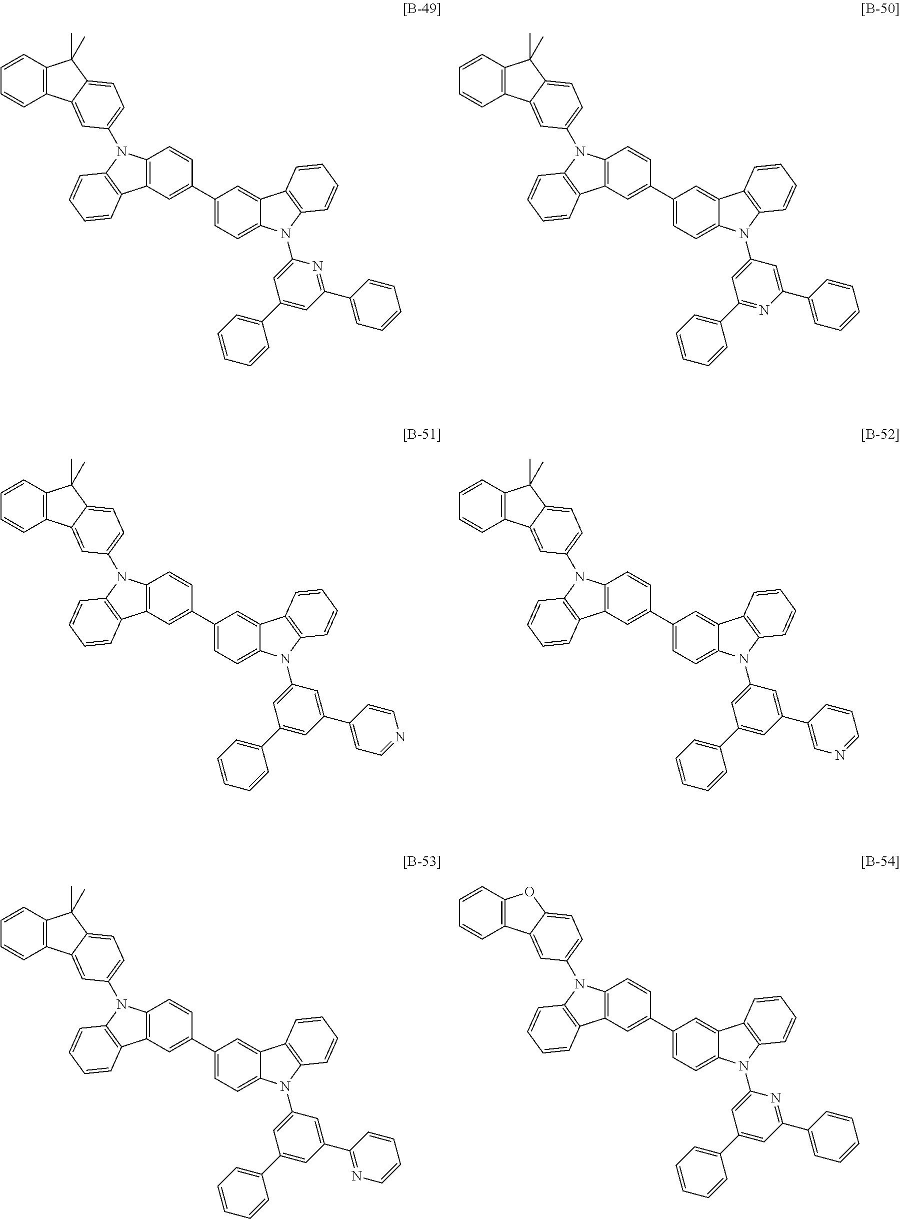

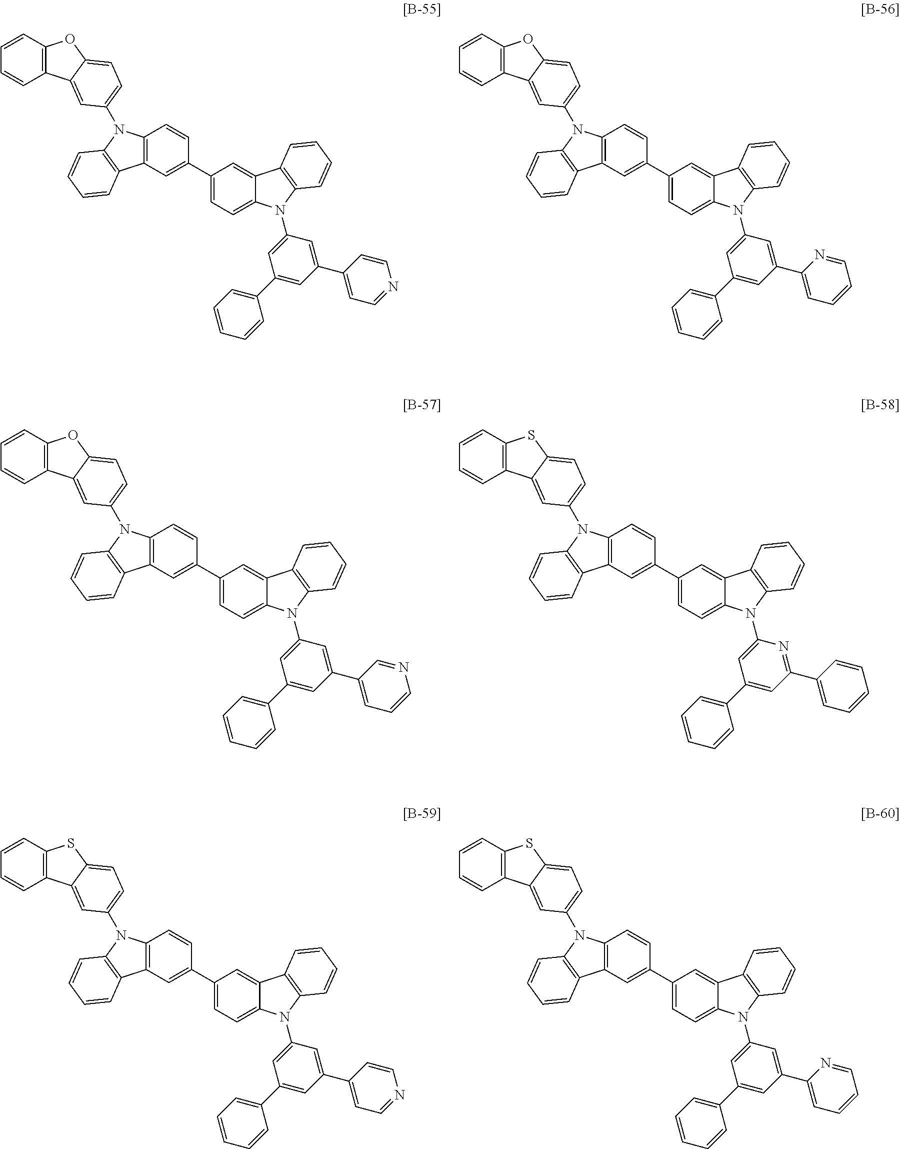

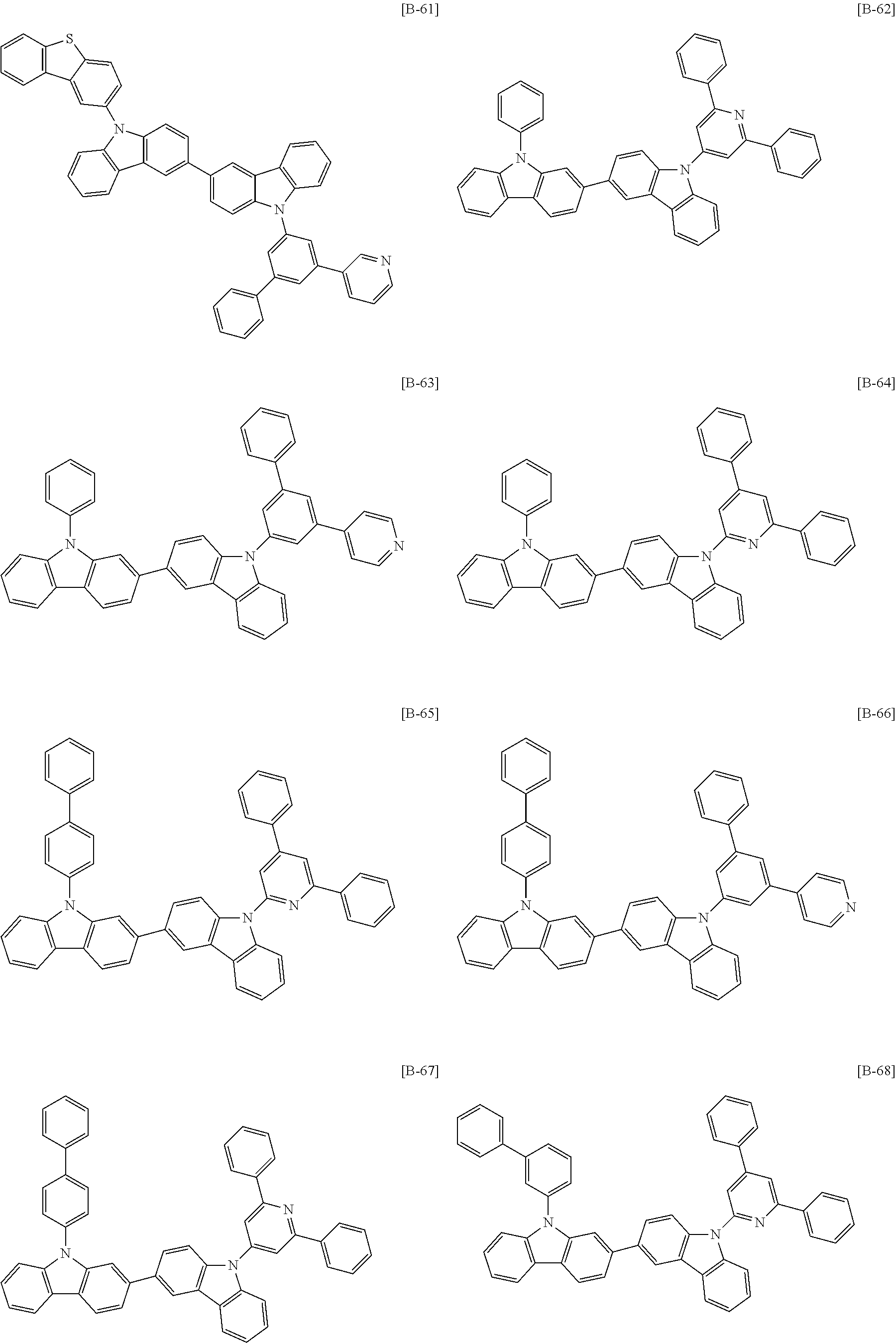

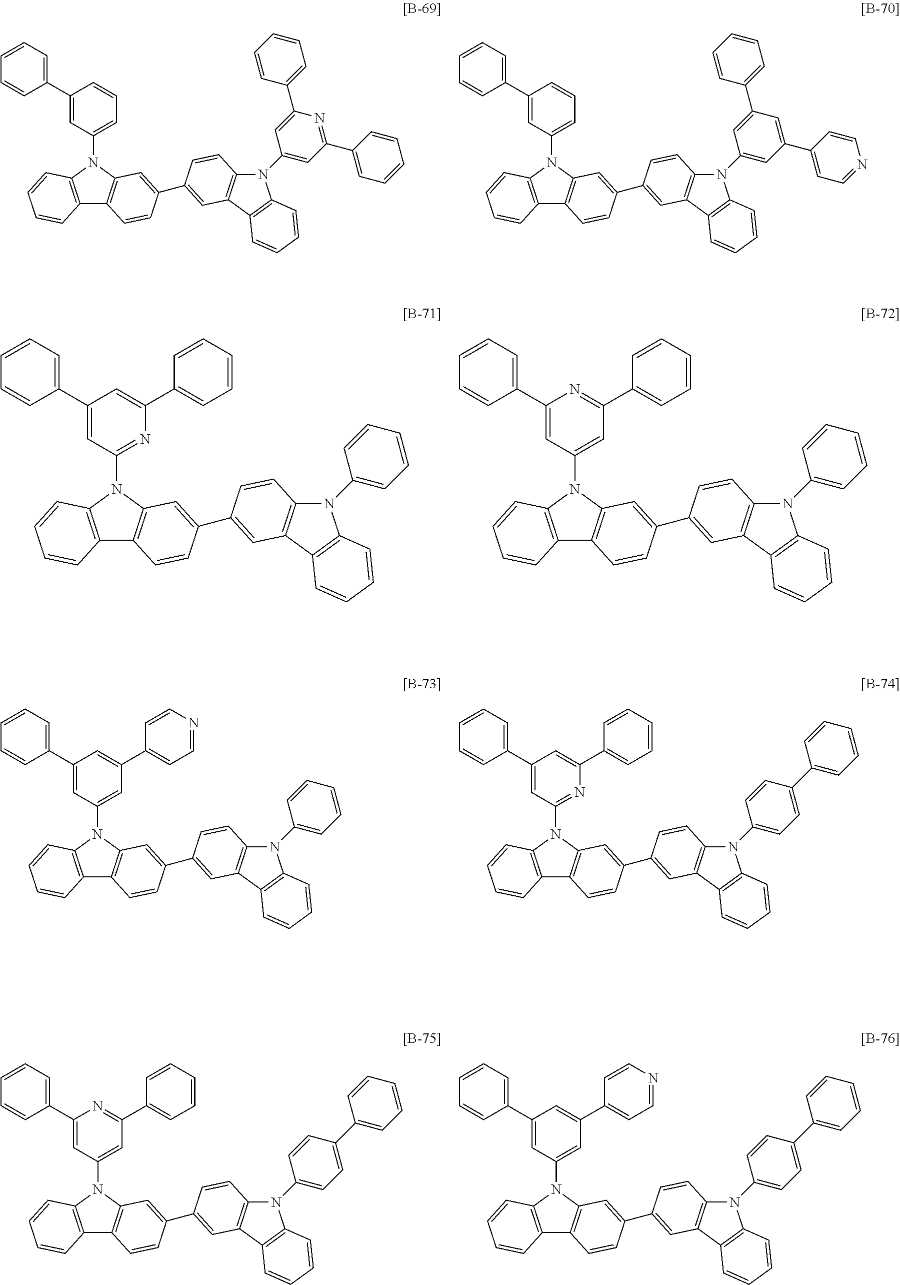

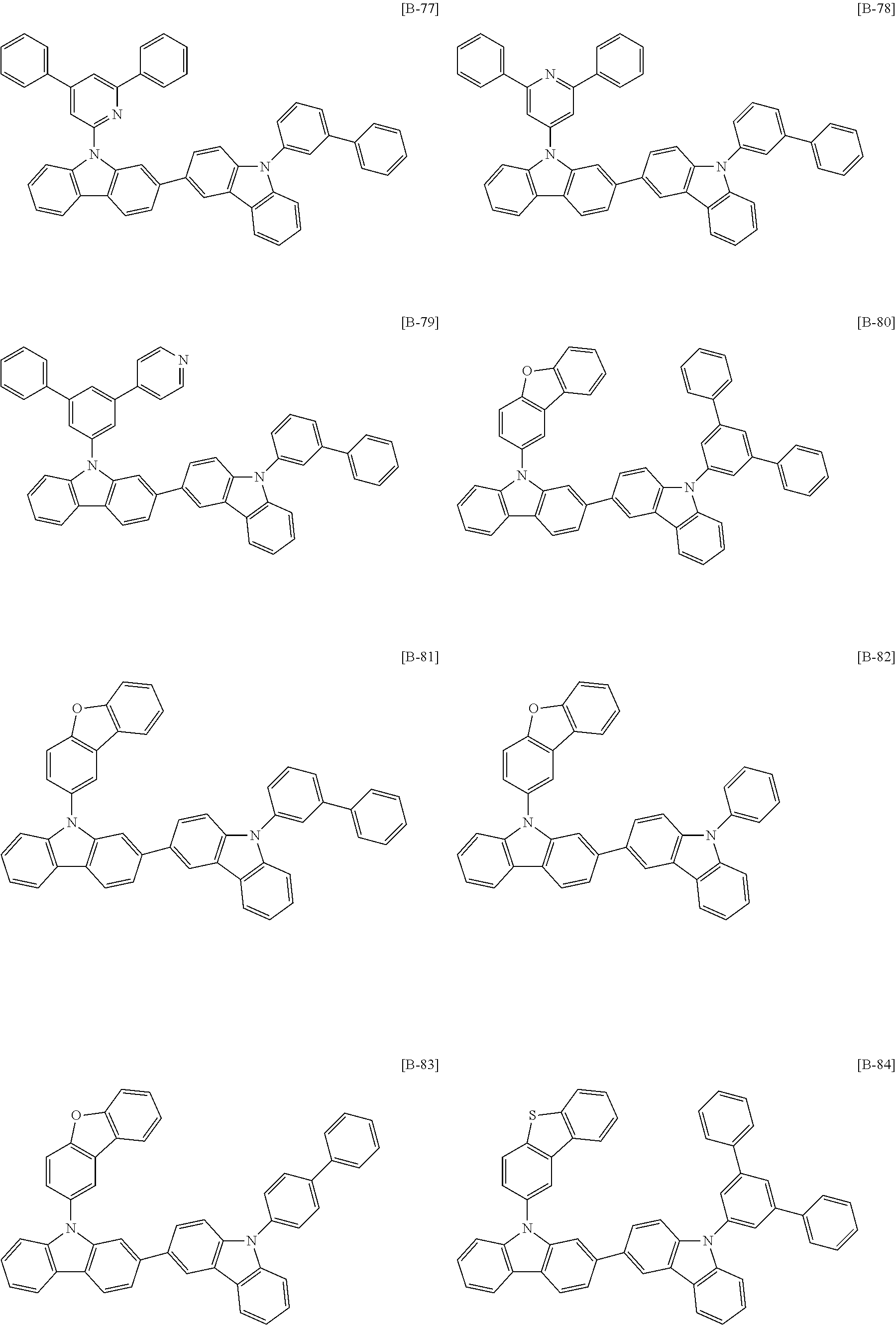

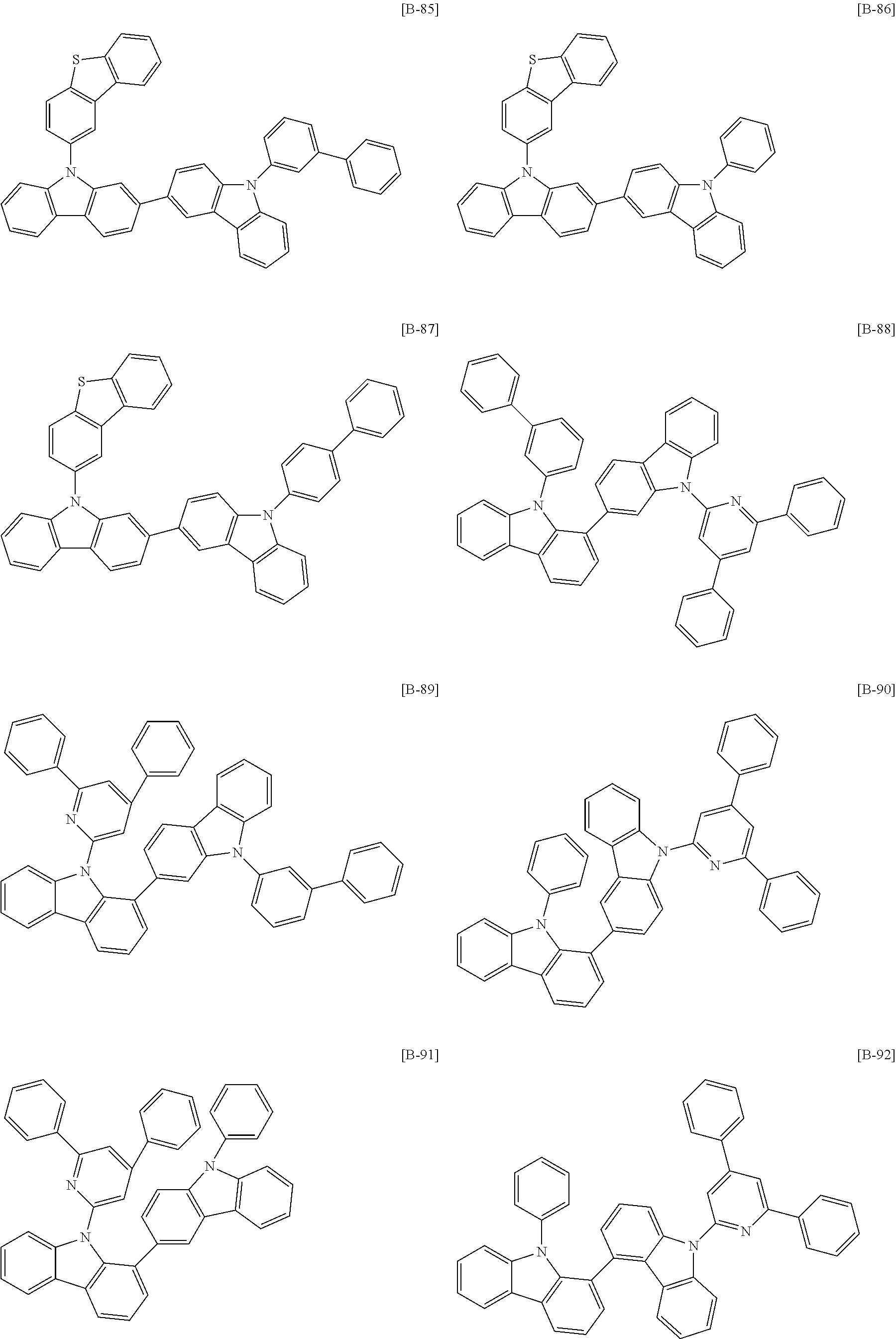

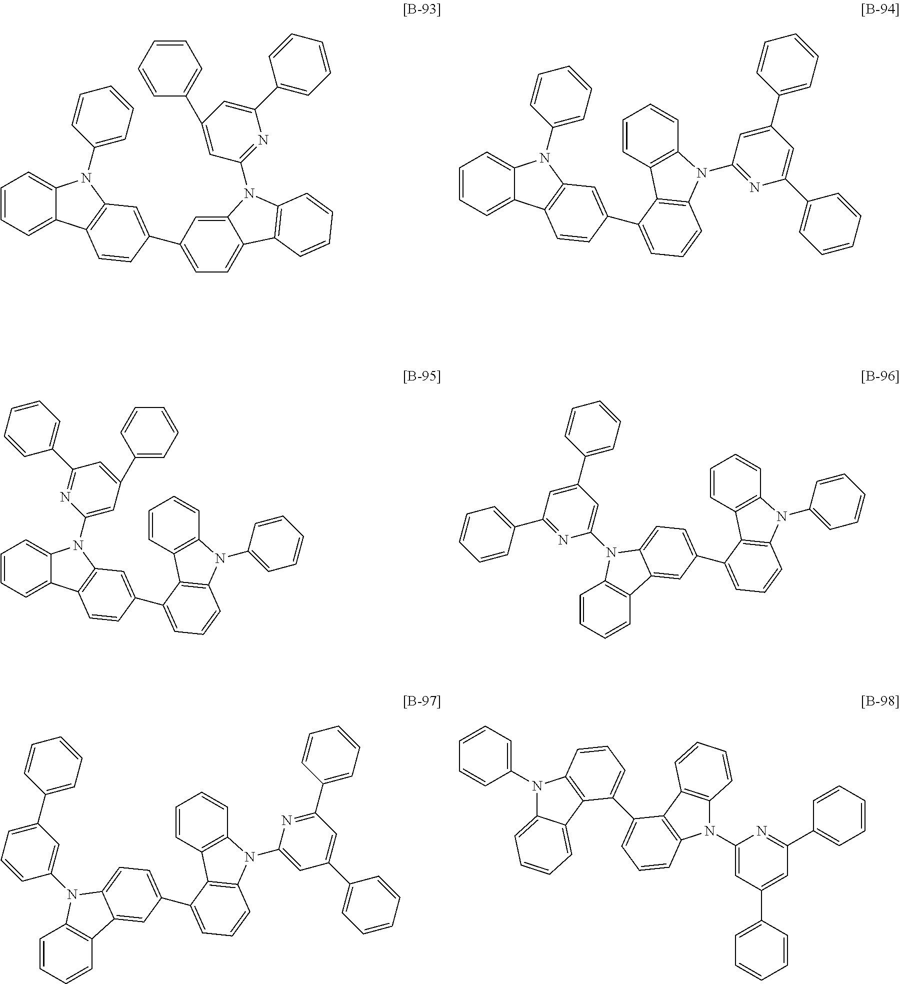

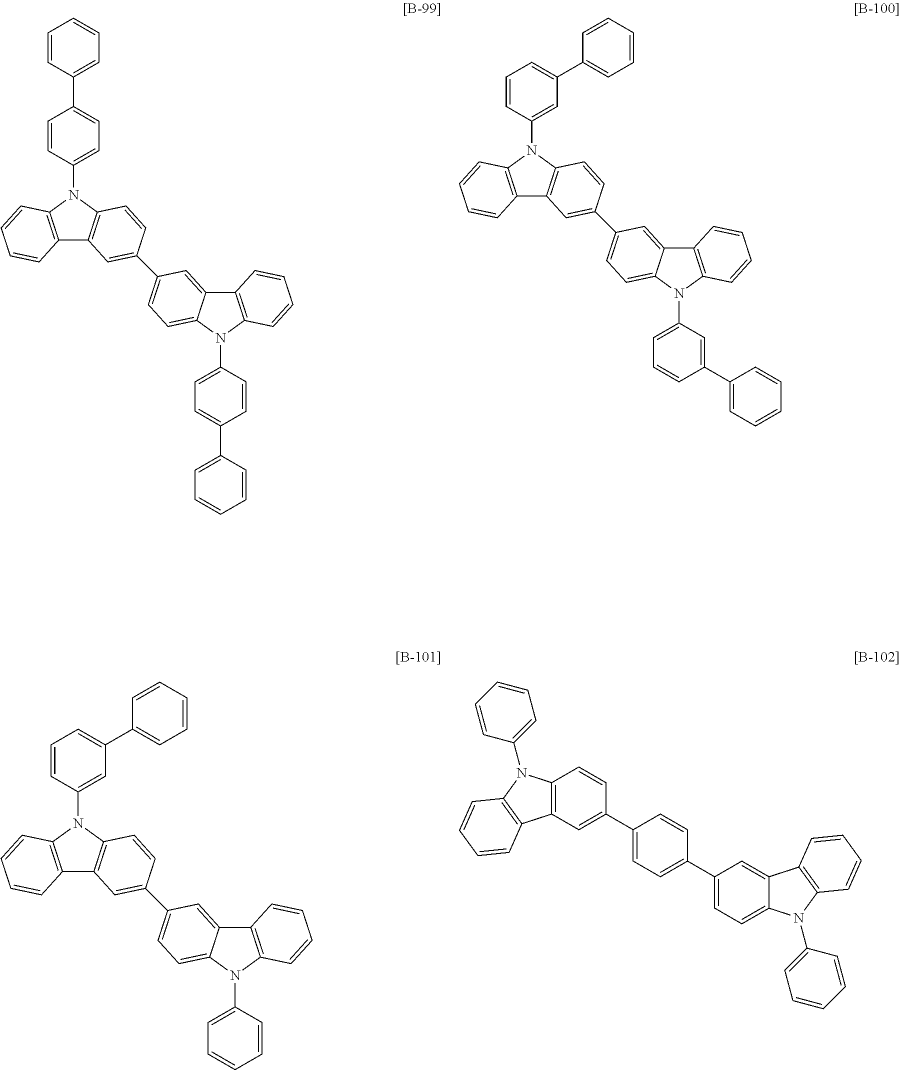 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2cccc(-n3c4ccccc4c4cc(-c5ccc6c(c5)c5ccccc5n6-c5ccccc5)ccc43)c2)cc1 ; c1ccc(-n2c3ccccc3c3cc(-c4ccc(-c5ccc6c(c5)c5ccccc5n6-c5ccccc5)cc4)ccc32)cc1]

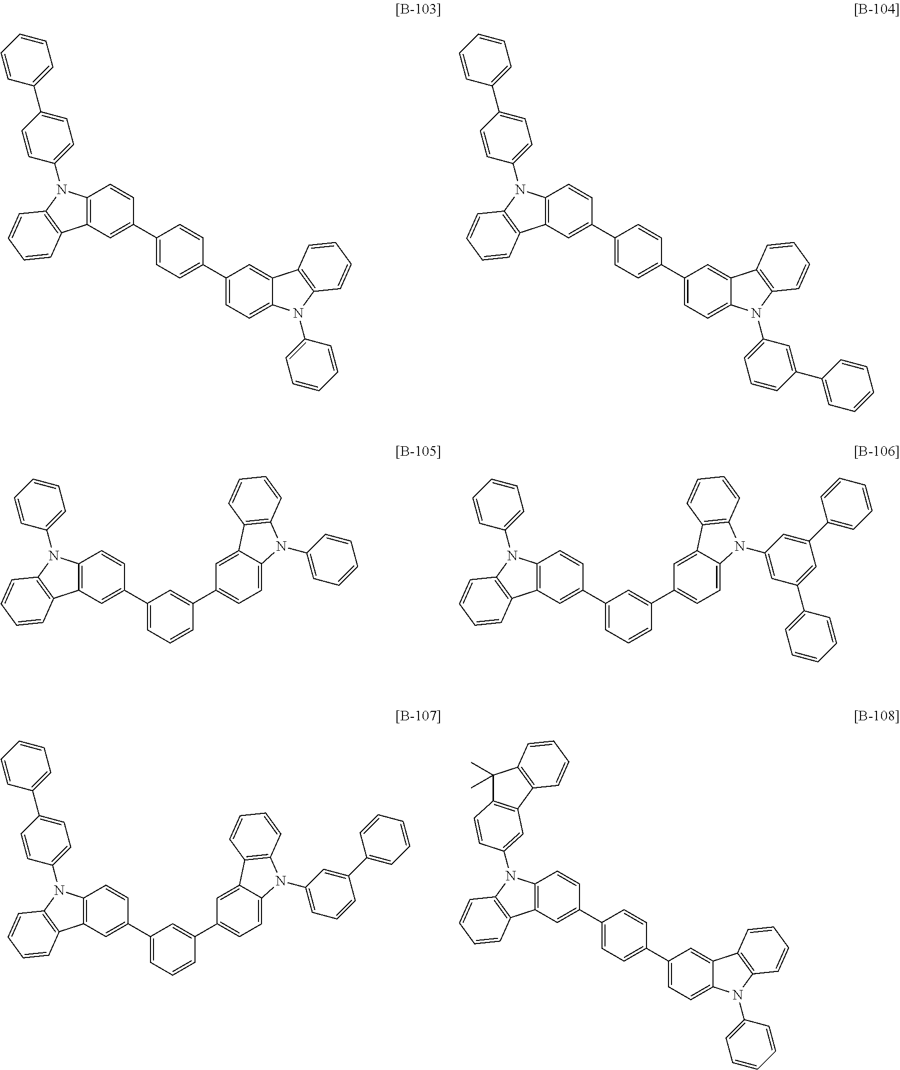

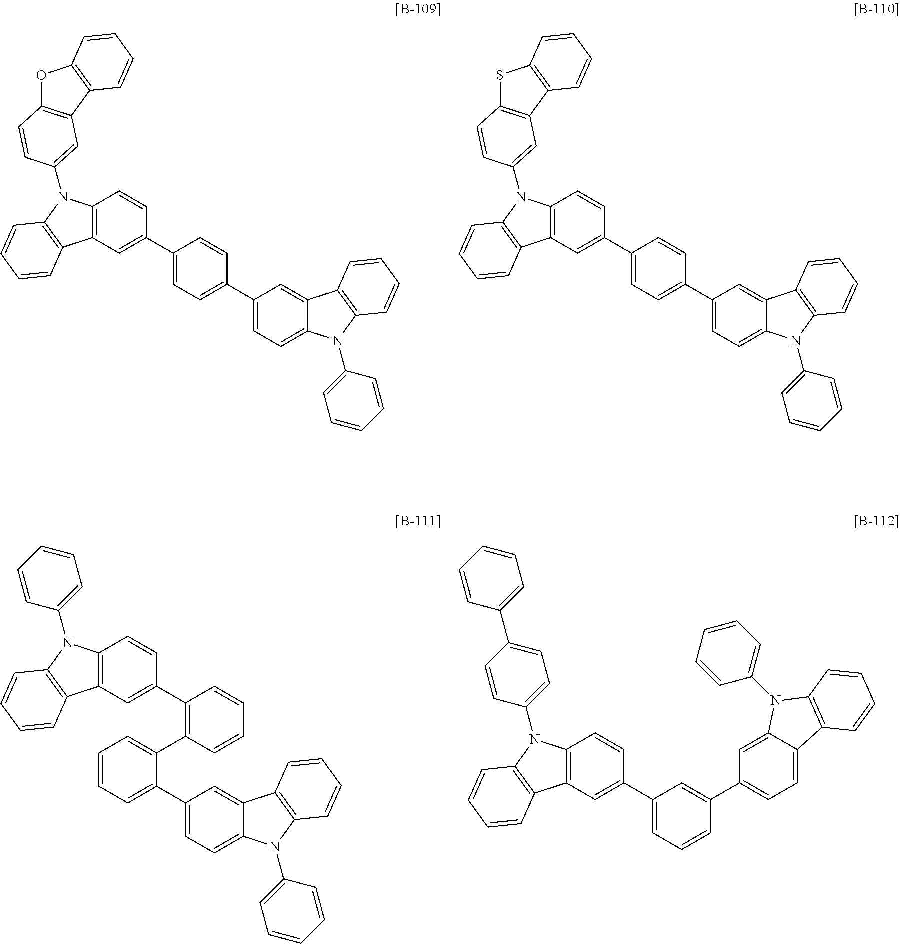 [SMILES: c1ccc(-n2c3ccccc3c3cc(-c4ccc(-c5ccc6c(c5)c5ccccc5n6-c5ccc6oc7ccccc7c6c5)cc4)ccc32)cc1 ; c1ccc(-n2c3ccccc3c3cc(-c4ccc(-c5ccc6c(c5)c5ccccc5n6-c5ccc6sc7ccccc7c6c5)cc4)ccc32)cc1 ; c1ccc(-n2c3ccccc3c3cc(-c4ccccc4-c4ccccc4-c4ccc5c(c4)c4ccccc4n5-c4ccccc4)ccc32)cc1 ; c1ccc(-c2ccc(-n3c4ccccc4c4cc(-c5cccc(-c6ccc7c8ccccc8n(-c8ccccc8)c7c6)c5)ccc43)cc2)cc1]

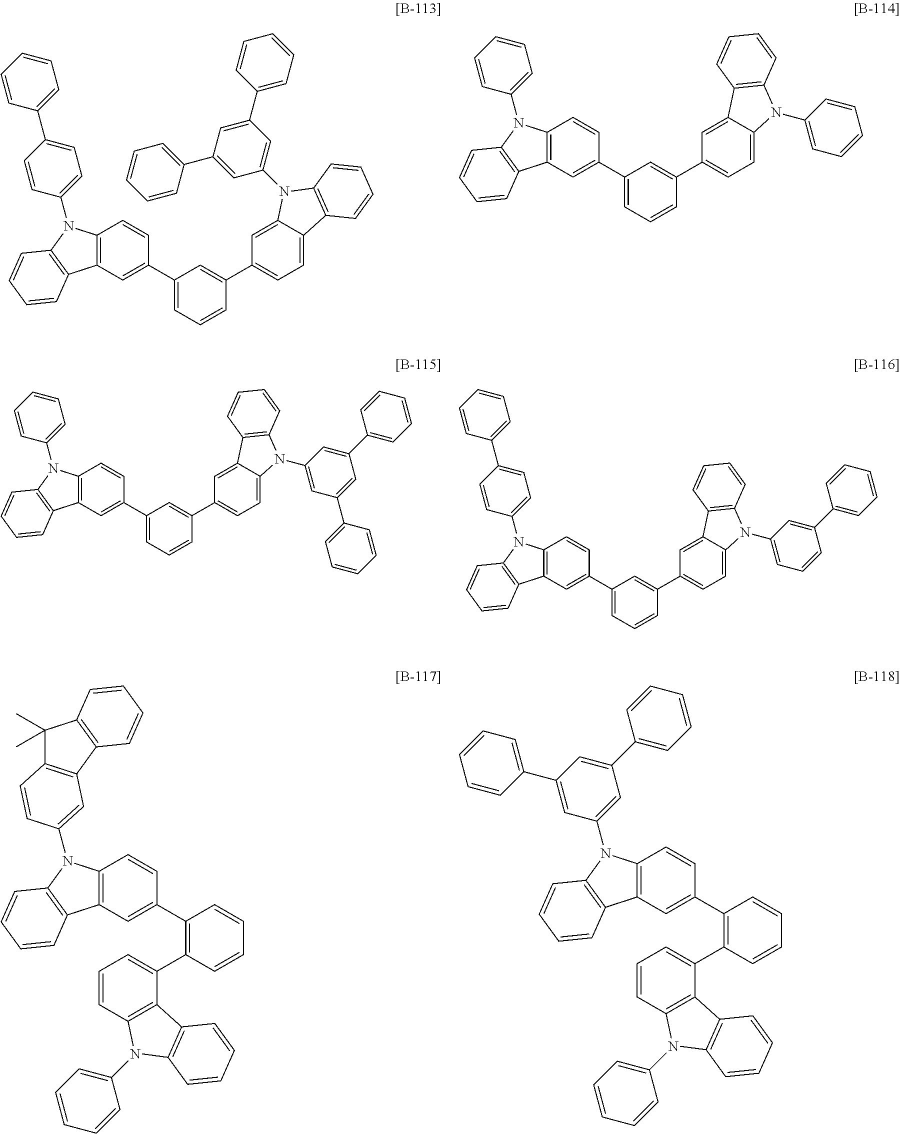

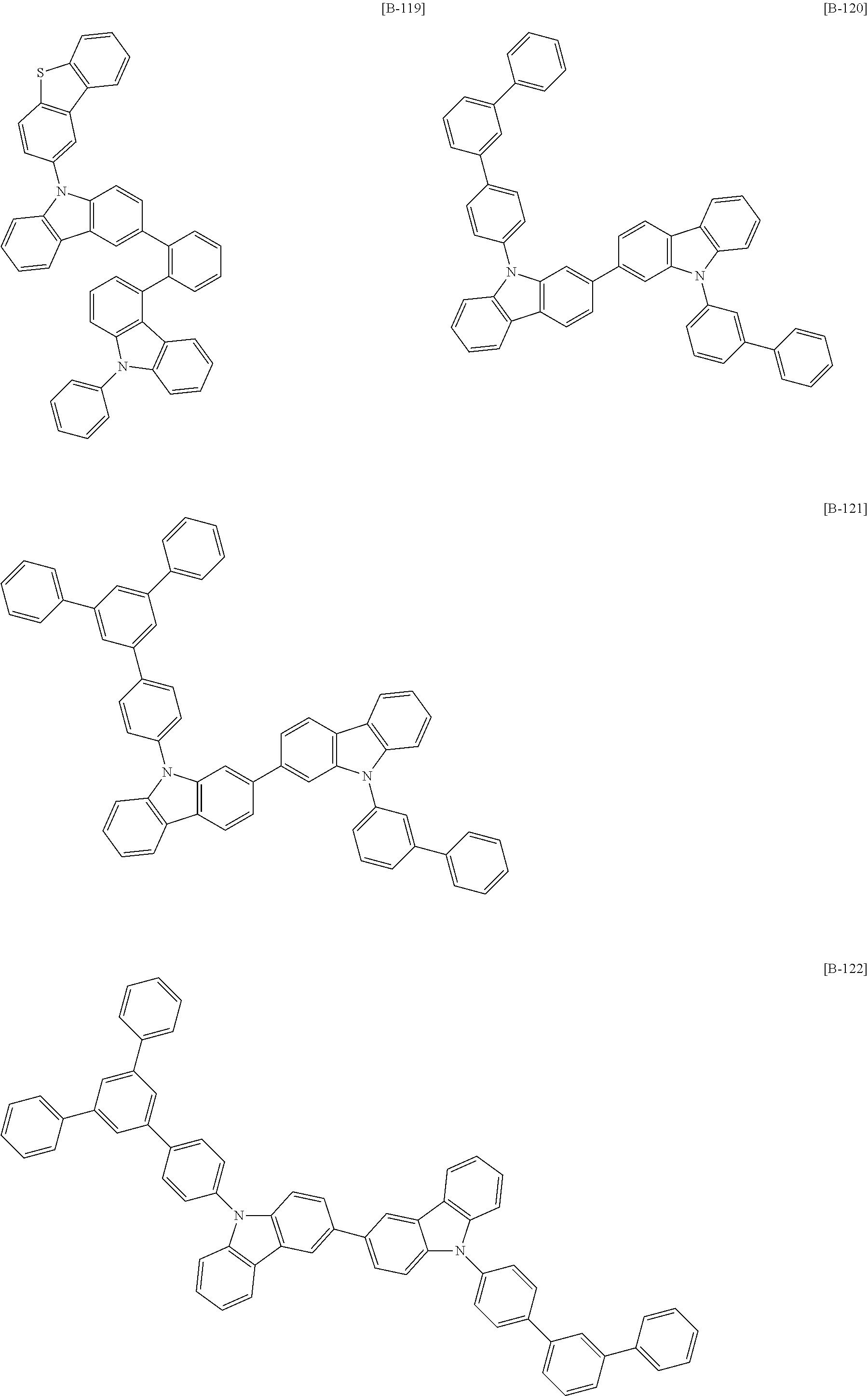 [SMILES: c1ccc(-n2c3ccccc3c3c(-c4ccccc4-c4ccc5c(c4)c4ccccc4n5-c4ccc5sc6ccccc6c5c4)cccc32)cc1 ; c1ccc(-c2cccc(-c3ccc(-n4c5ccccc5c5ccc(-c6ccc7c8ccccc8n(-c8cccc(-c9ccccc9)c8)c7c6)cc54)cc3)c2)cc1 ; c1ccc(-c2cc(-c3ccccc3)cc(-c3ccc(-n4c5ccccc5c5ccc(-c6ccc7c8ccccc8n(-c8cccc(-c9ccccc9)c8)c7c6)cc54)cc3)c2)cc1 ; c1ccc(-c2cccc(-c3ccc(-n4c5ccccc5c5cc(-c6ccc7c(c6)c6ccccc6n7-c6ccc(-c7cc(-c8ccccc8)cc(-c8ccccc8)c7)cc6)ccc54)cc3)c2)cc1]

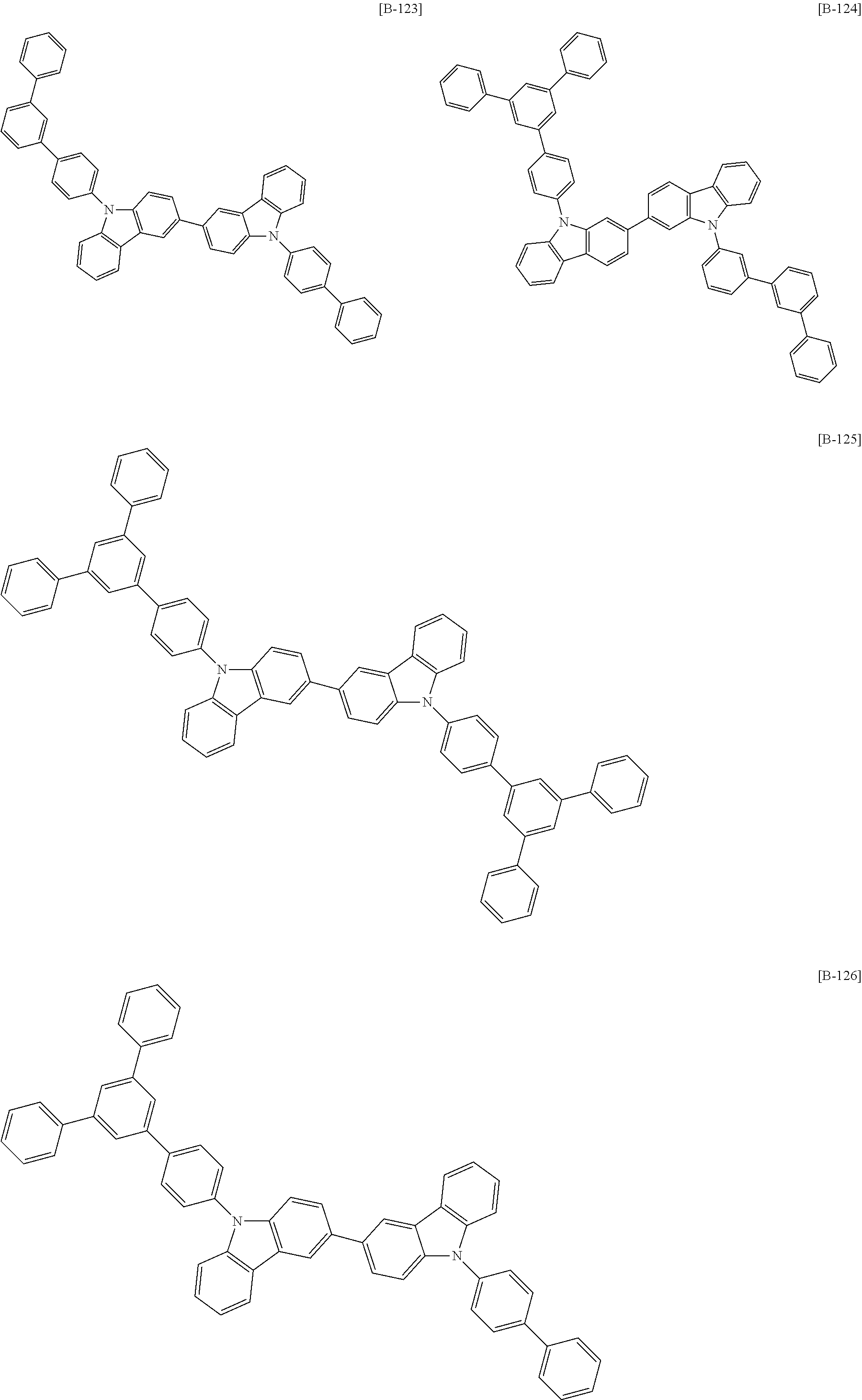 [SMILES: c1ccc(-c2ccc(-n3c4ccccc4c4cc(-c5ccc6c(c5)c5ccccc5n6-c5ccc(-c6cccc(-c7ccccc7)c6)cc5)ccc43)cc2)cc1 ; c1ccc(-c2cccc(-c3cccc(-n4c5ccccc5c5ccc(-c6ccc7c8ccccc8n(-c8ccc(-c9cc(-c%10ccccc%10)cc(-c%10ccccc%10)c9)cc8)c7c6)cc54)c3)c2)cc1 ; c1ccc(-c2cc(-c3ccccc3)cc(-c3ccc(-n4c5ccccc5c5cc(-c6ccc7c(c6)c6ccccc6n7-c6ccc(-c7cc(-c8ccccc8)cc(-c8ccccc8)c7)cc6)ccc54)cc3)c2)cc1 ; c1ccc(-c2ccc(-n3c4ccccc4c4cc(-c5ccc6c(c5)c5ccccc5n6-c5ccc(-c6cc(-c7ccccc7)cc(-c7ccccc7)c6)cc5)ccc43)cc2)cc1]

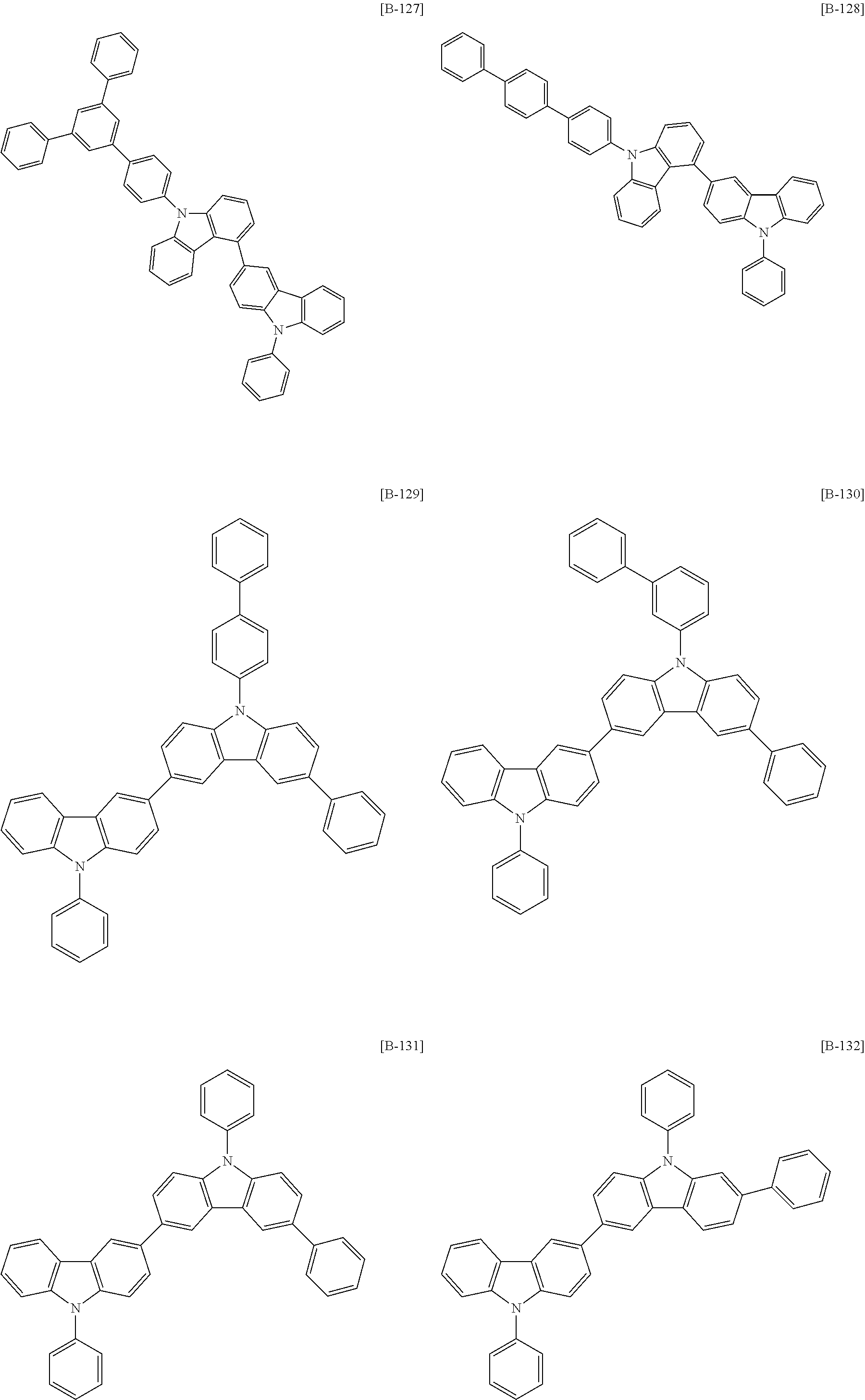

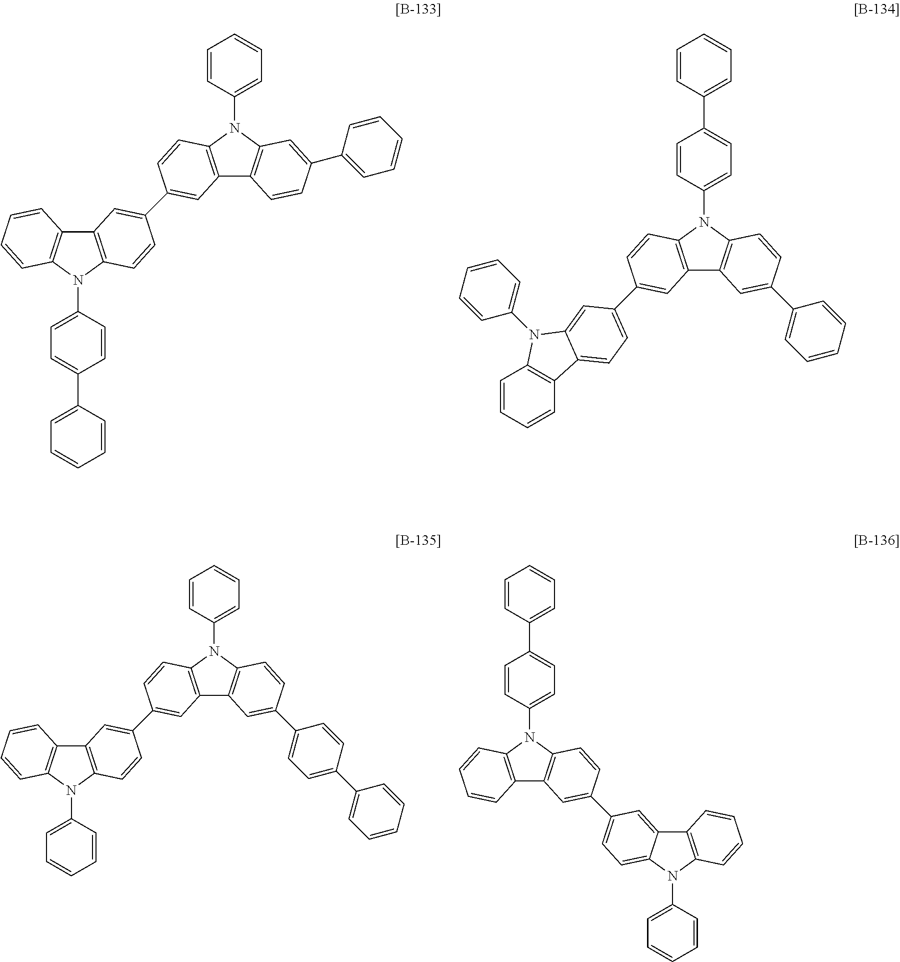 [SMILES: c1ccc(-c2ccc(-n3c4ccccc4c4cc(-c5ccc6c(c5)c5ccc(-c7ccccc7)cc5n6-c5ccccc5)ccc43)cc2)cc1 ; c1ccc(-c2ccc(-n3c4ccc(-c5ccccc5)cc4c4cc(-c5ccc6c7ccccc7n(-c7ccccc7)c6c5)ccc43)cc2)cc1 ; c1ccc(-c2ccc(-c3ccc4c(c3)c3cc(-c5ccc6c(c5)c5ccccc5n6-c5ccccc5)ccc3n4-c3ccccc3)cc2)cc1 ; c1ccc(-c2ccc(-n3c4ccccc4c4cc(-c5ccc6c(c5)c5ccccc5n6-c5ccccc5)ccc43)cc2)cc1]

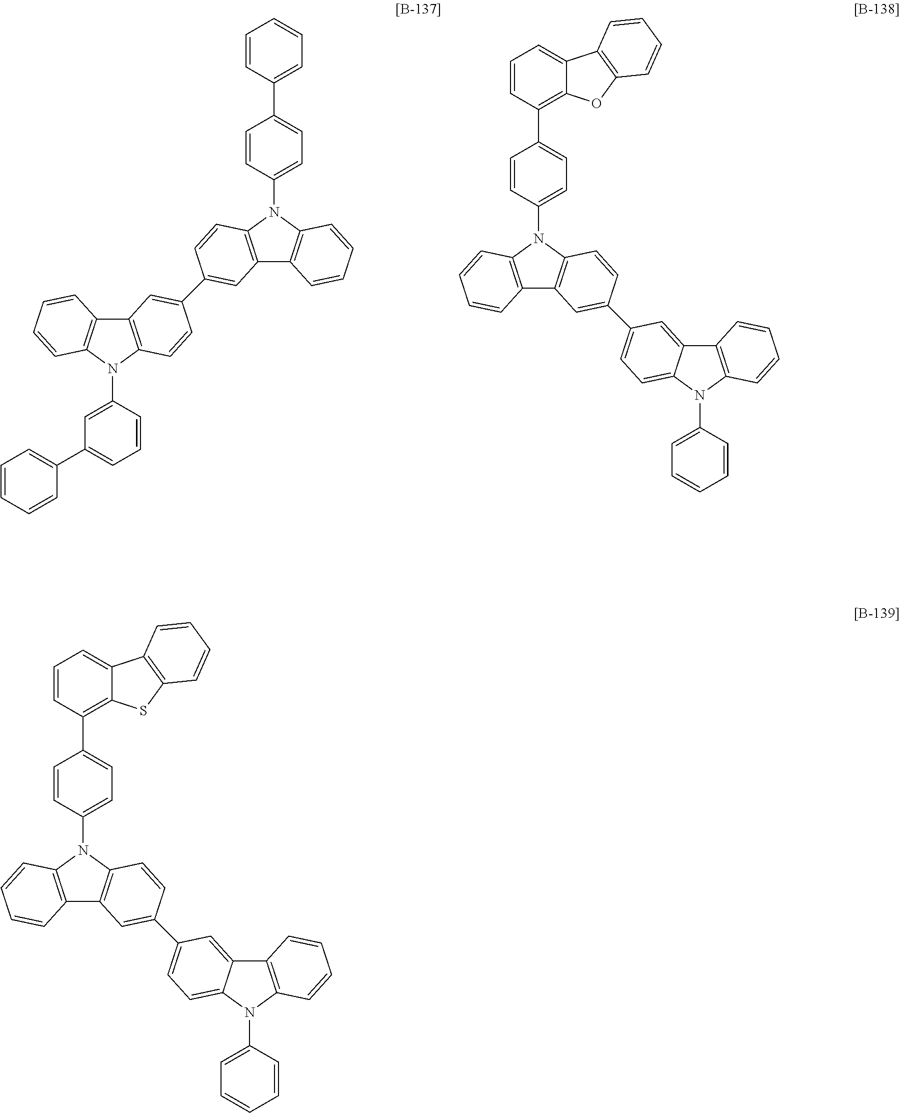 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-n2c3ccccc3c3cc(-c4ccc5c(c4)c4ccccc4n5-c4ccc(-c5cccc6c5oc5ccccc56)cc4)ccc32)cc1 ; c1ccc(-n2c3ccccc3c3cc(-c4ccc5c(c4)c4ccccc4n5-c4ccc(-c5cccc6c5sc5ccccc56)cc4)ccc32)cc1]

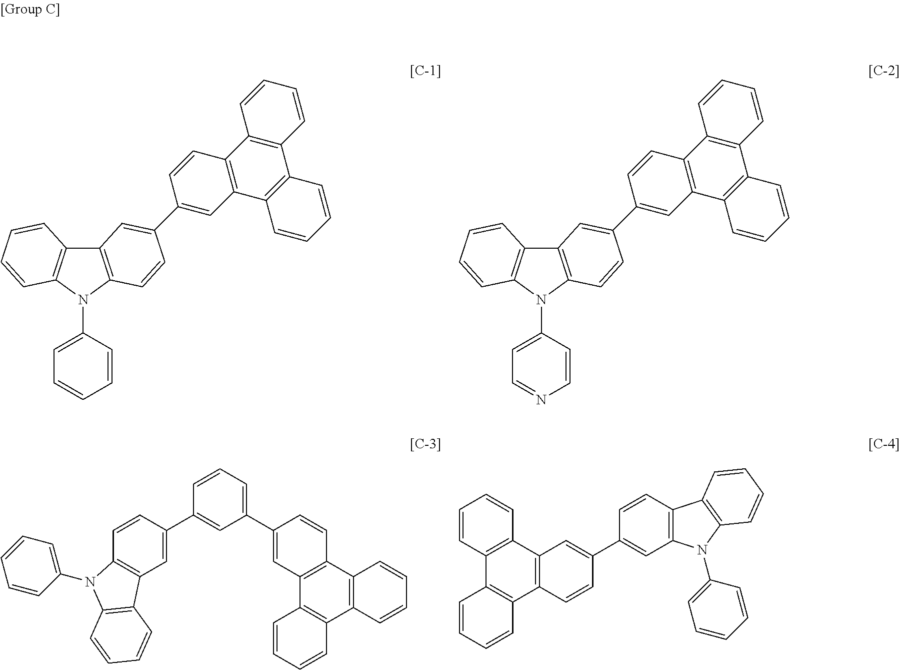 [SMILES: c1ccc(-n2c3ccccc3c3cc(-c4ccc5c6ccccc6c6ccccc6c5c4)ccc32)cc1 ; c1ccc2c(c1)c1ccccc1c1cc(-c3ccc4c(c3)c3ccccc3n4-c3ccncc3)ccc21 ; c1ccc(-n2c3ccccc3c3cc(-c4cccc(-c5ccc6c7ccccc7c7ccccc7c6c5)c4)ccc32)cc1 ; c1ccc(-n2c3ccccc3c3ccc(-c4ccc5c6ccccc6c6ccccc6c5c4)cc32)cc1]

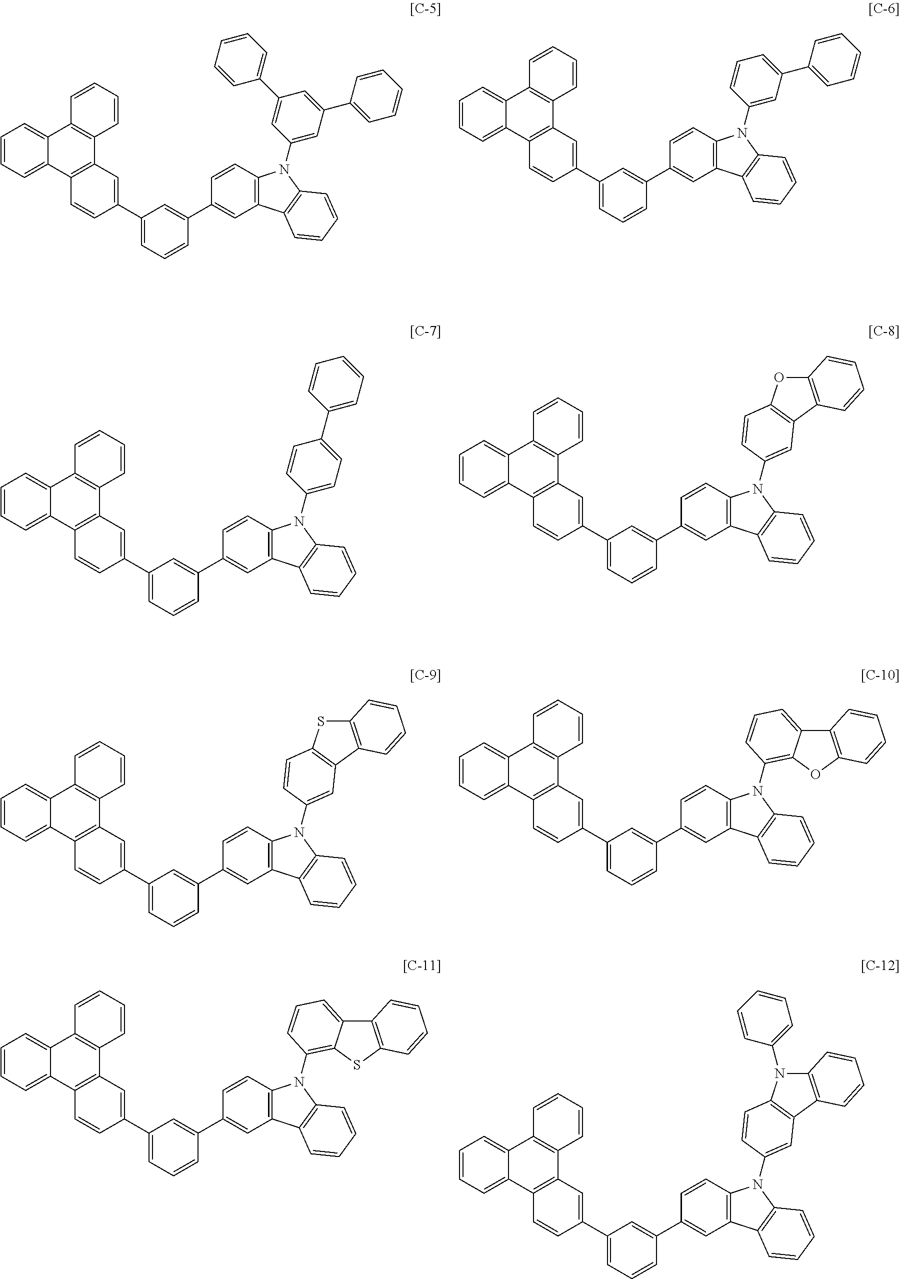

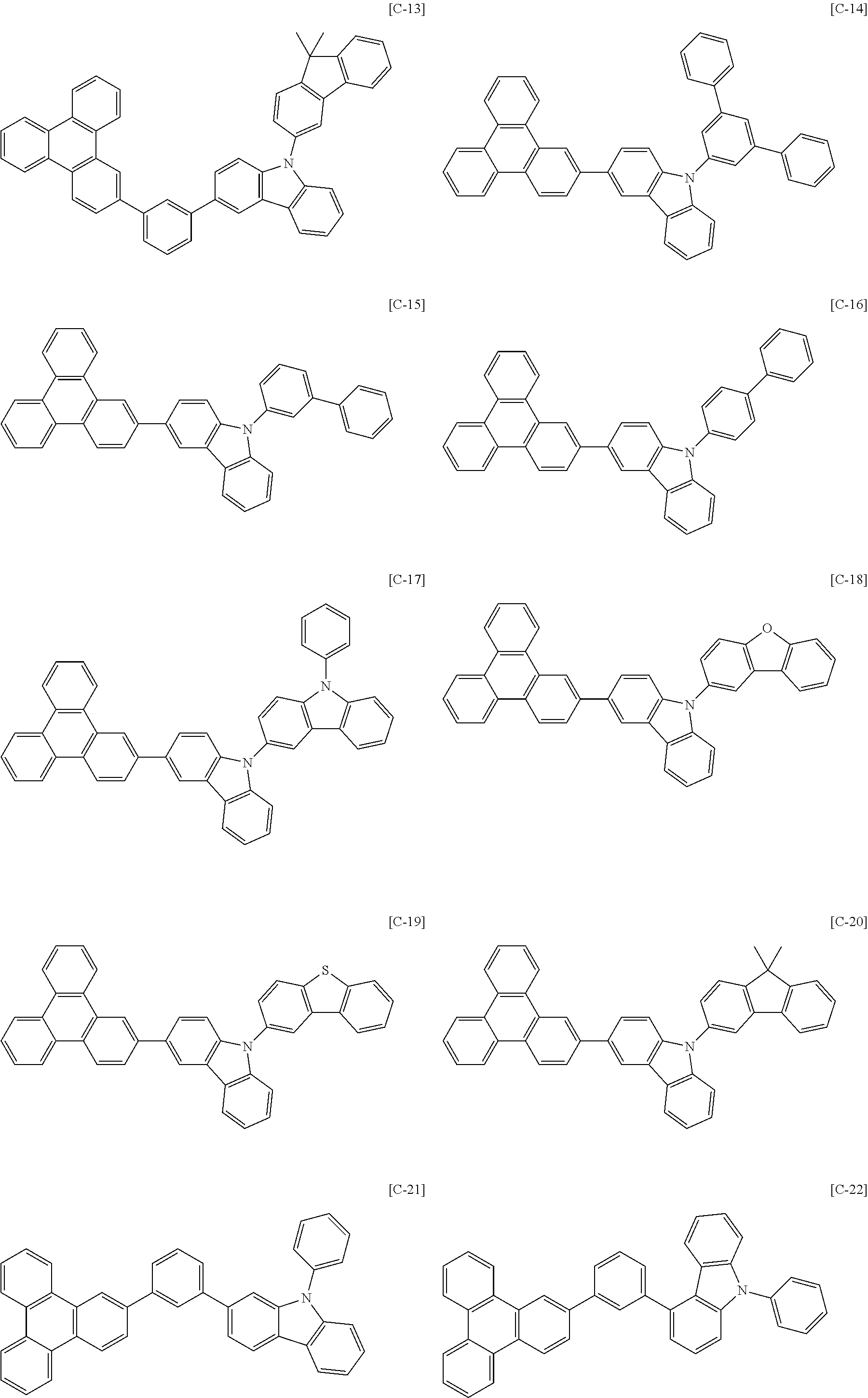

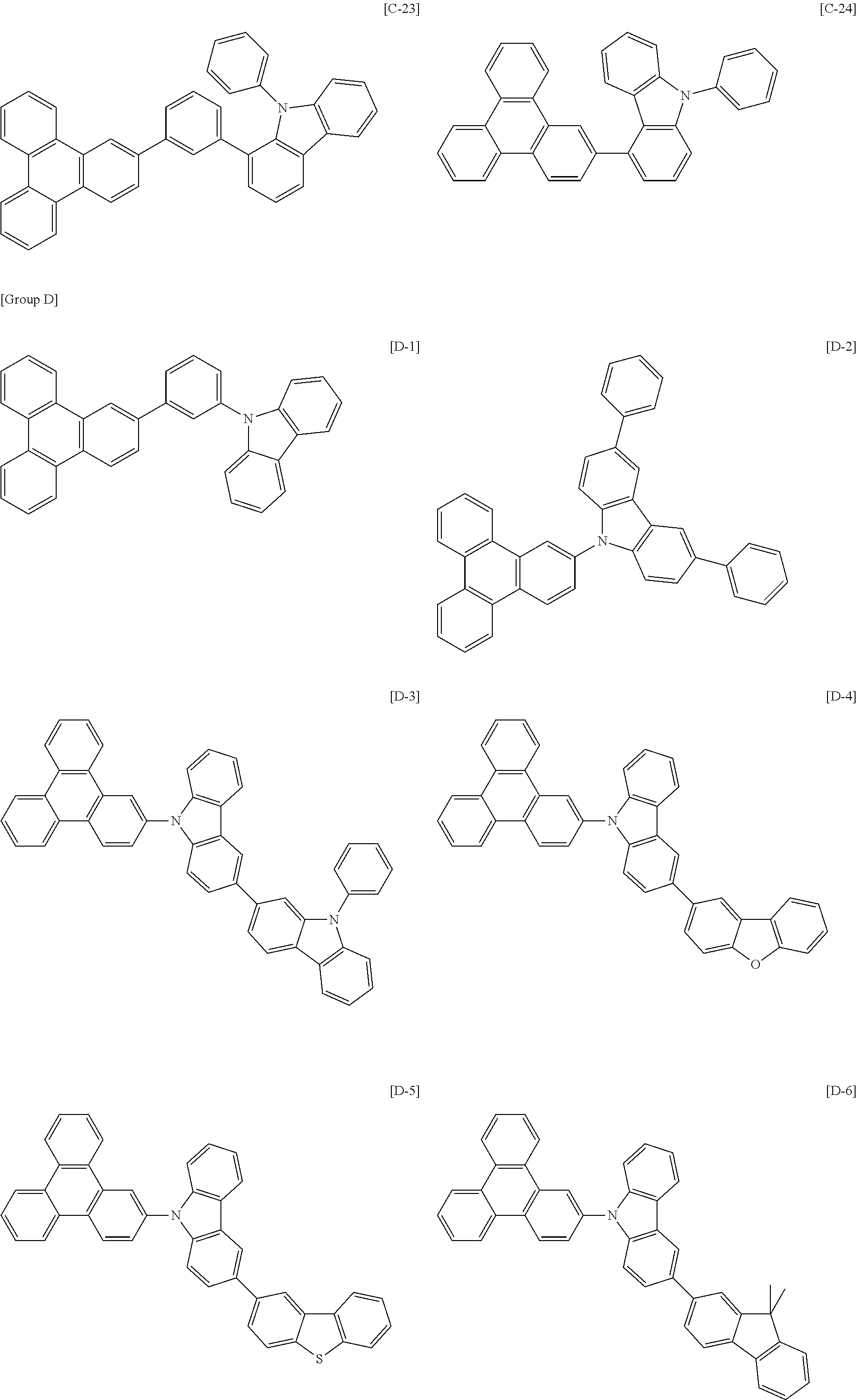

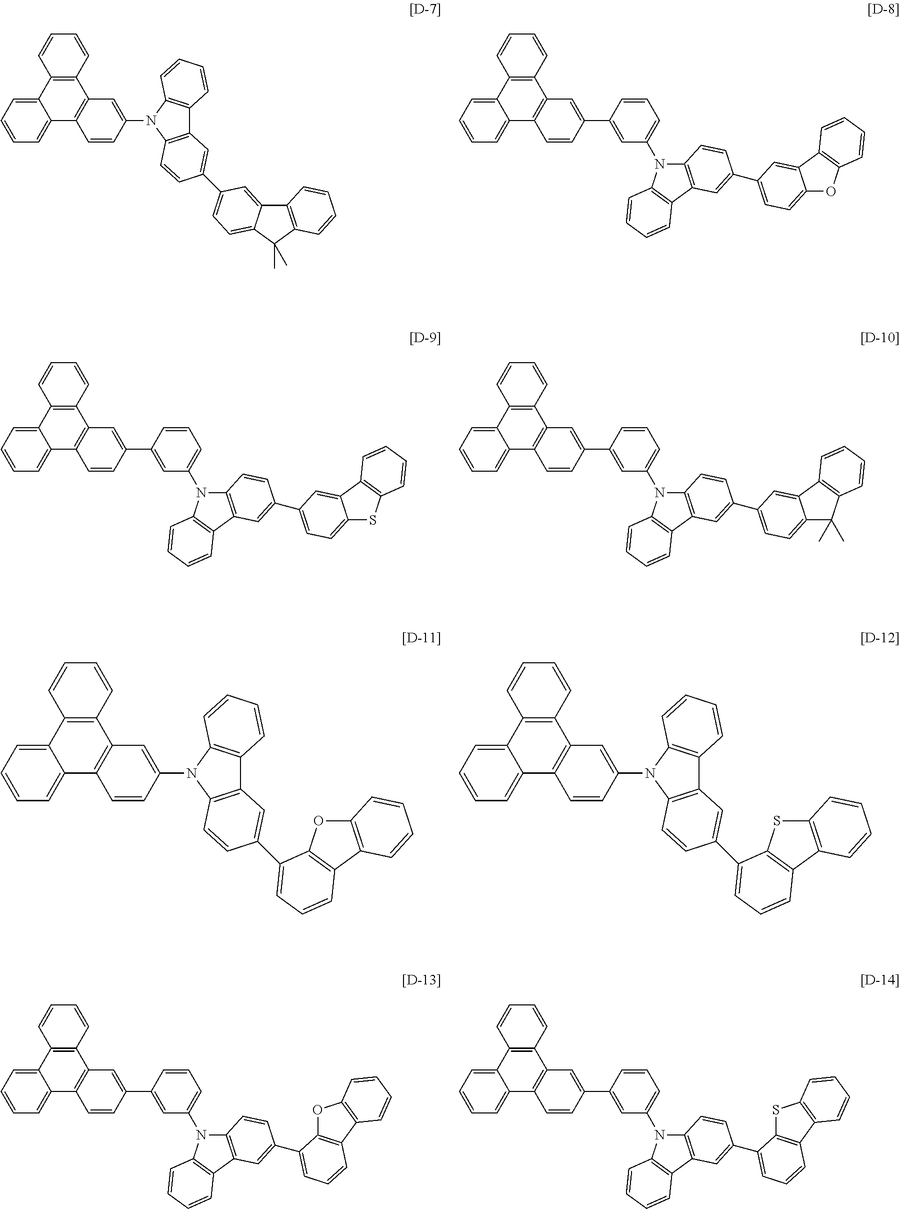

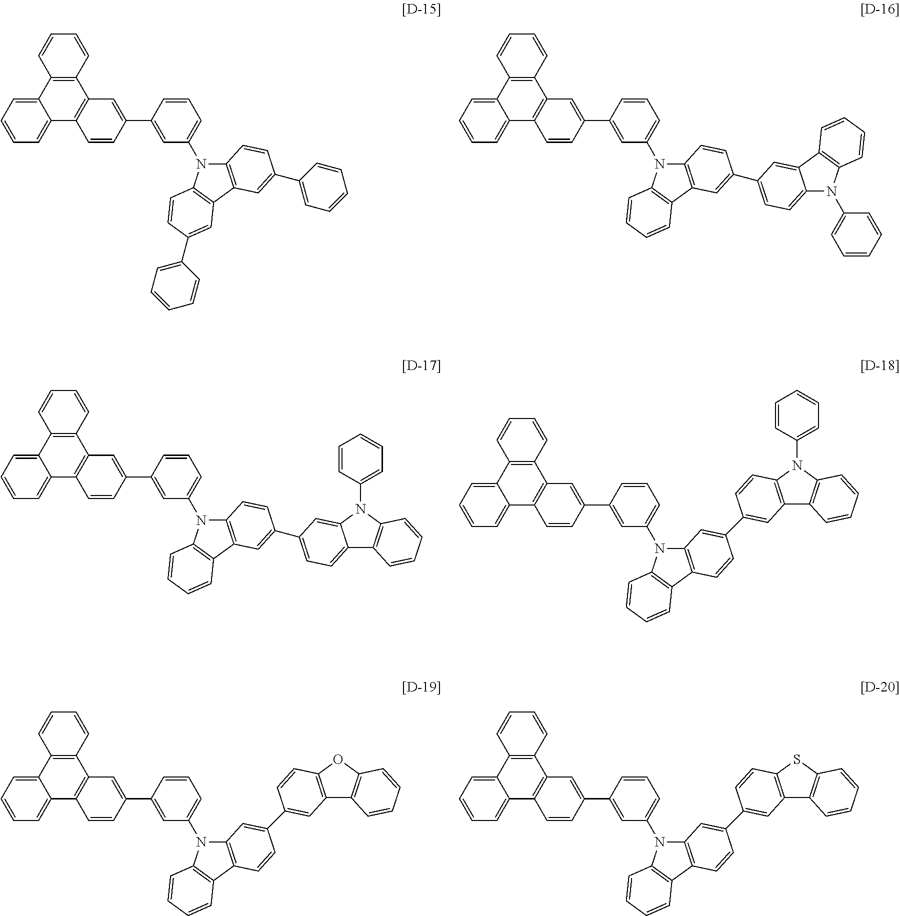

In addition, the second compound may be represented by a combination of the moiety represented by Chemical Formula 3 and the moiety represented by Chemical Formula 4.

The second compound consisting of a combination of the moiety represented by Chemical Formula 3 and the moiety represented by Chemical Formula 4 may be, for example, represented by at least one of Chemical Formulae 3-I to 3-V, but is not limited thereto.

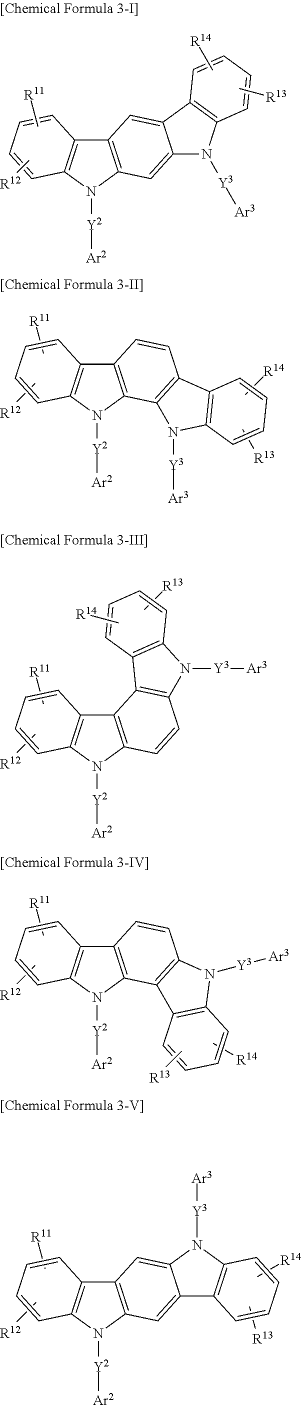

In Chemical Formulae 3-I to 3-V, Y², Y³, Ar², and R¹¹ to R¹⁴ are the same as described above. Ar³ is the same as Ar².

Specifically, Ar² and Ar³ of Chemical Formulae 3-I to 3-V may independently be a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted triphenylenyl group, a substituted or unsubstituted carbazolyl group, a substituted or unsubstituted benzofuranyl group, a substituted or unsubstituted benzothiophenyl group, a substituted or unsubstituted fluorenyl group, a substituted or unsubstituted dibenzofuranyl group, a substituted or unsubstituted dibenzothiophenyl group, a substituted or unsubstituted pyridinyl group, or a combination thereof.

The second compound consisting of a combination of the moiety represented by Chemical Formula 3 and the moiety represented by Chemical Formula 4 may be, for example compounds of Group E, but is not limited thereto.

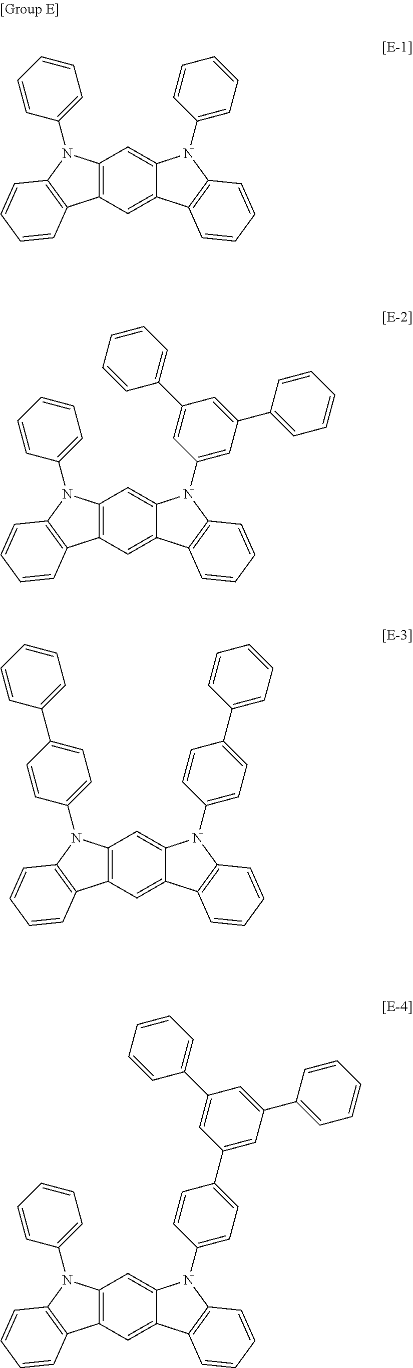 [SMILES: c1ccc(-n2c3ccccc3c3cc4c5ccccc5n(-c5ccccc5)c4cc32)cc1 ; c1ccc(-c2cc(-c3ccccc3)cc(-n3c4ccccc4c4cc5c6ccccc6n(-c6ccccc6)c5cc43)c2)cc1 ; c1ccc(-c2ccc(-n3c4ccccc4c4cc5c6ccccc6n(-c6ccc(-c7ccccc7)cc6)c5cc43)cc2)cc1 ; c1ccc(-c2cc(-c3ccccc3)cc(-c3ccc(-n4c5ccccc5c5cc6c7ccccc7n(-c7ccccc7)c6cc54)cc3)c2)cc1]

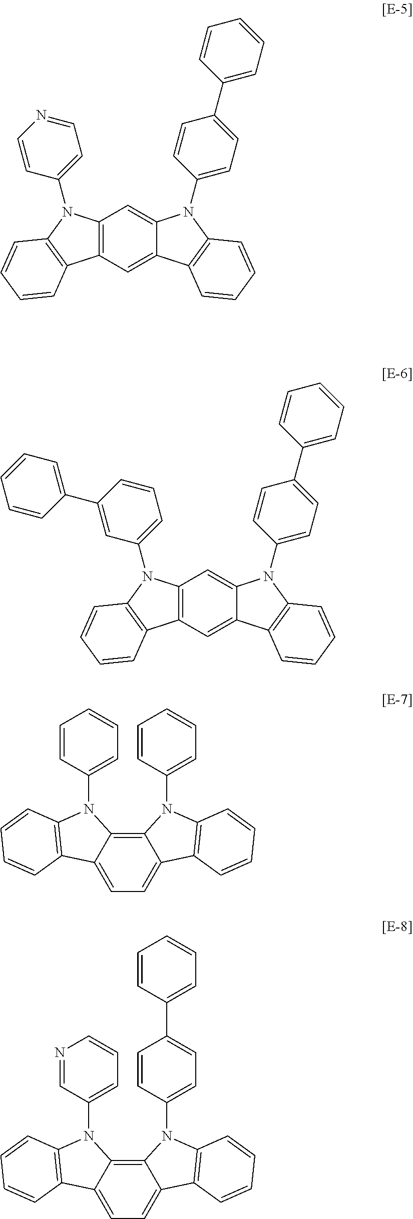

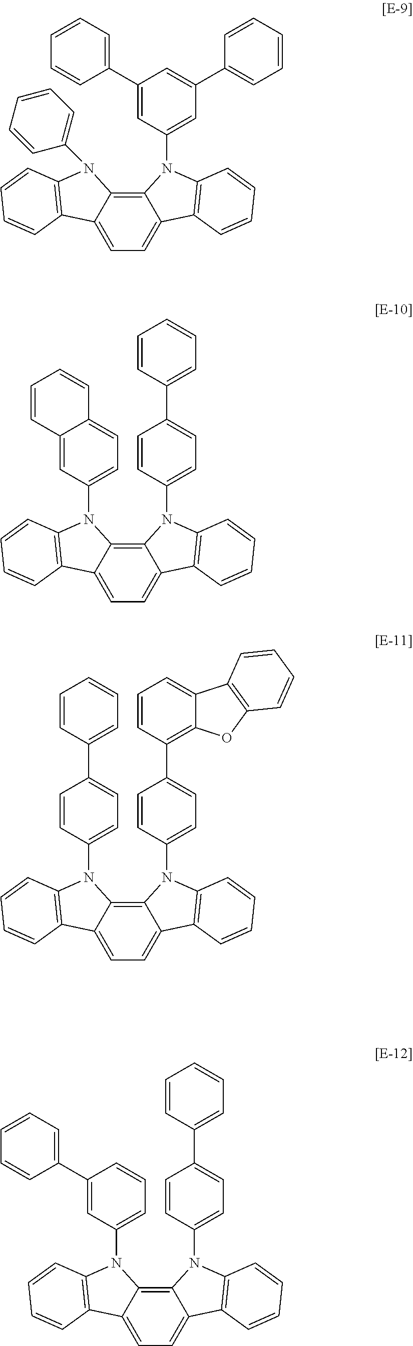 [SMILES: c1ccc(-c2cc(-c3ccccc3)cc(-n3c4ccccc4c4ccc5c6ccccc6n(-c6ccccc6)c5c43)c2)cc1 ; c1ccc(-c2ccc(-n3c4ccccc4c4ccc5c6ccccc6n(-c6ccc7ccccc7c6)c5c43)cc2)cc1 ; c1ccc(-c2ccc(-n3c4ccccc4c4ccc5c6ccccc6n(-c6ccc(-c7cccc8c7oc7ccccc78)cc6)c5c43)cc2)cc1 ; c1ccc(-c2ccc(-n3c4ccccc4c4ccc5c6ccccc6n(-c6cccc(-c7ccccc7)c6)c5c43)cc2)cc1]

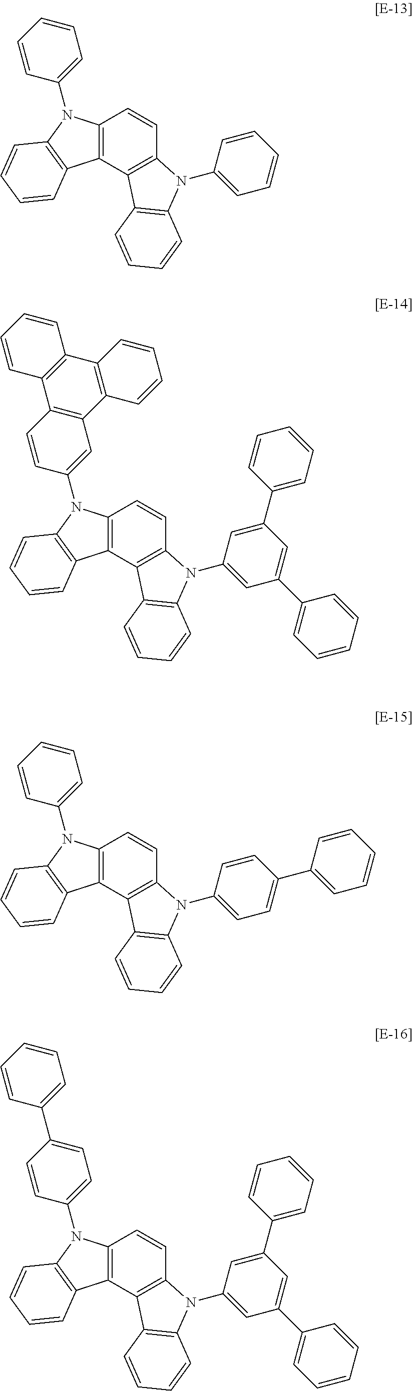 [SMILES: c1ccc(-n2c3ccccc3c3c4c5ccccc5n(-c5ccccc5)c4ccc32)cc1 ; c1ccc(-c2cc(-c3ccccc3)cc(-n3c4ccccc4c4c5c6ccccc6n(-c6ccc7c8ccccc8c8ccccc8c7c6)c5ccc43)c2)cc1 ; c1ccc(-c2ccc(-n3c4ccccc4c4c5c6ccccc6n(-c6ccccc6)c5ccc43)cc2)cc1 ; c1ccc(-c2ccc(-n3c4ccccc4c4c5c6ccccc6n(-c6cc(-c7ccccc7)cc(-c7ccccc7)c6)c5ccc43)cc2)cc1]

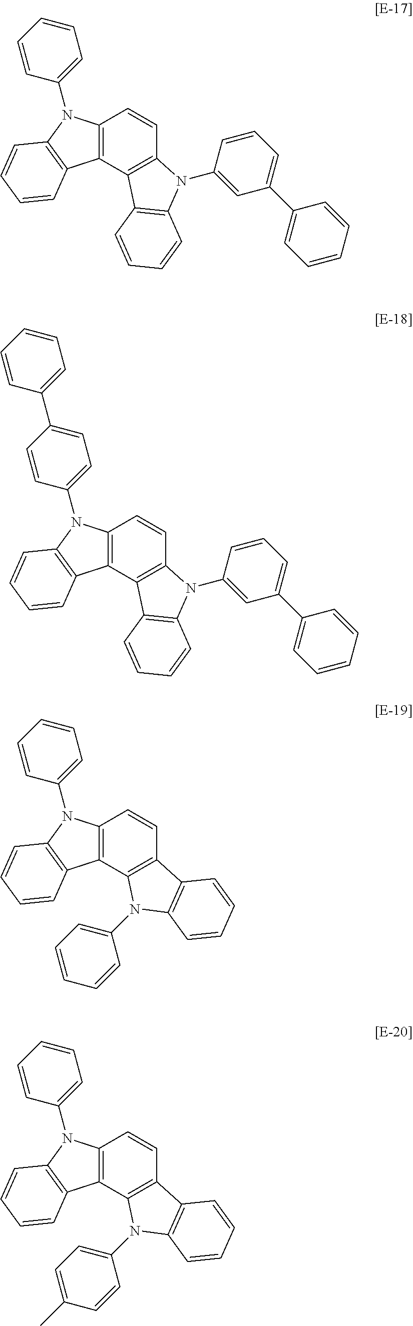 [SMILES: c1ccc(-c2cccc(-n3c4ccccc4c4c5c6ccccc6n(-c6ccccc6)c5ccc43)c2)cc1 ; c1ccc(-c2ccc(-n3c4ccccc4c4c5c6ccccc6n(-c6cccc(-c7ccccc7)c6)c5ccc43)cc2)cc1 ; c1ccc(-n2c3ccccc3c3c2ccc2c4ccccc4n(-c4ccccc4)c23)cc1 ; Cc1ccc(-n2c3ccccc3c3ccc4c(c5ccccc5n4-c4ccccc4)c32)cc1]

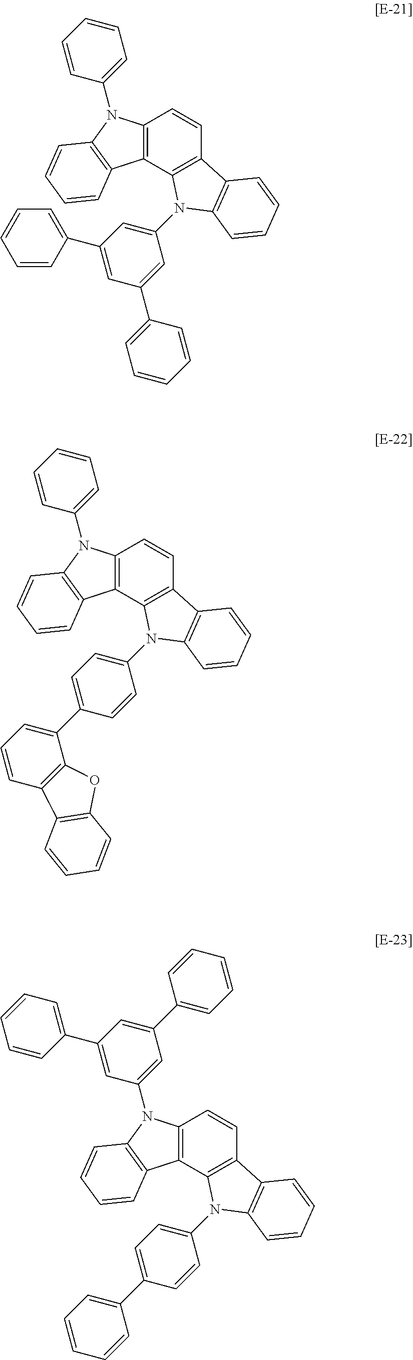 [SMILES: c1ccc(-c2cc(-c3ccccc3)cc(-n3c4ccccc4c4ccc5c(c6ccccc6n5-c5ccccc5)c43)c2)cc1 ; c1ccc(-n2c3ccccc3c3c2ccc2c4ccccc4n(-c4ccc(-c5cccc6c5oc5ccccc56)cc4)c23)cc1 ; c1ccc(-c2ccc(-n3c4ccccc4c4ccc5c(c6ccccc6n5-c5cc(-c6ccccc6)cc(-c6ccccc6)c5)c43)cc2)cc1]

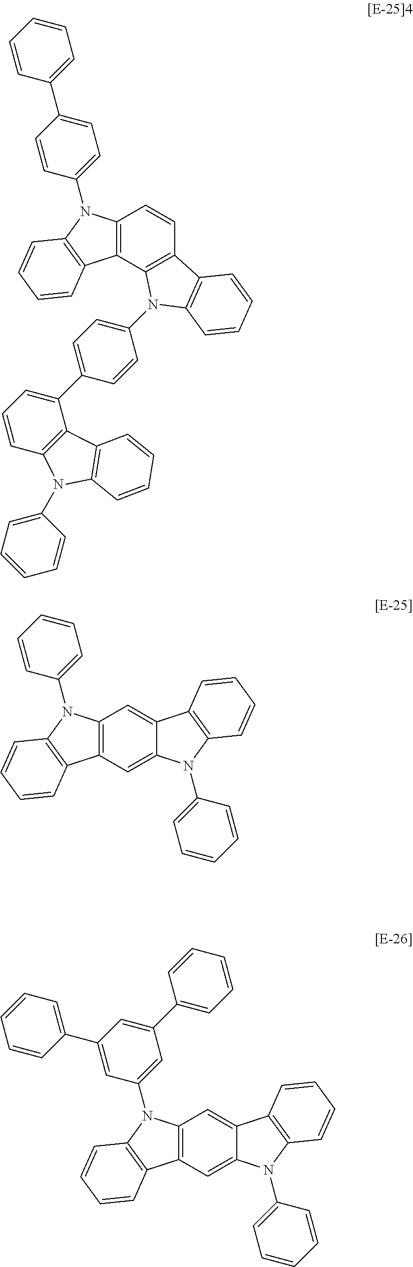

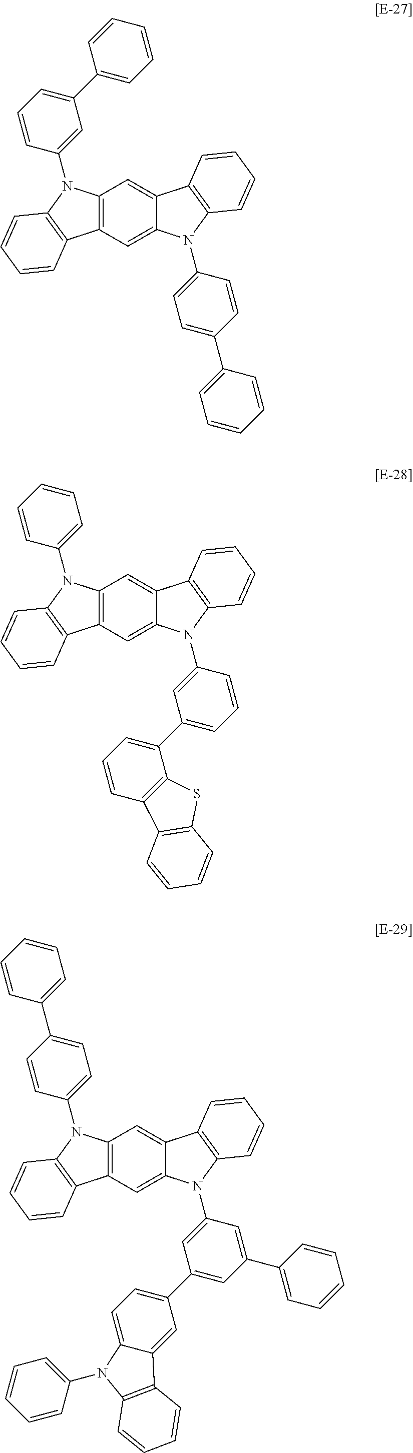 [SMILES: c1ccc(-c2ccc(-n3c4ccccc4c4cc5c(cc43)c3ccccc3n5-c3cccc(-c4ccccc4)c3)cc2)cc1 ; c1ccc(-n2c3ccccc3c3cc4c(cc32)c2ccccc2n4-c2cccc(-c3cccc4c3sc3ccccc34)c2)cc1 ; c1ccc(-c2ccc(-n3c4ccccc4c4cc5c(cc43)c3ccccc3n5-c3cc(-c4ccccc4)cc(-c4ccc5c(c4)c4ccccc4n5-c4ccccc4)c3)cc2)cc1]

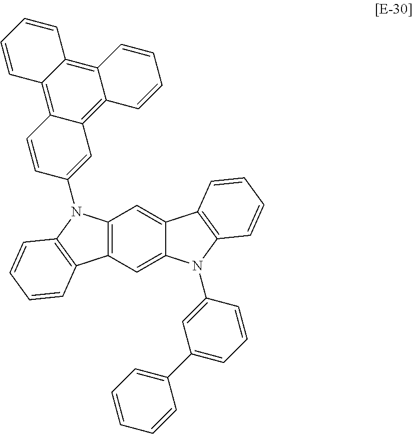 [SMILES: c1ccc(-c2cccc(-n3c4ccccc4c4cc5c(cc43)c3ccccc3n5-c3ccc4c5ccccc5c5ccccc5c4c3)c2)cc1]

The second compound is a compound having bipolar characteristics in which hole characteristics are relatively strong, and thus, luminous efficiency and life-span characteristics may be improved by increasing charge mobility and stability when used with the first compound in an emission layer. In addition, charge mobility may be controlled by adjusting a ratio of the second compound having hole characteristics and the first compound. Since the hole characteristic of the second compound is relatively determined with a relationship to the first compound, a substituent having weak electron characteristics such as a substituted or unsubstituted pyridinyl group may be included at any position of R⁷ to R¹⁰ and Ar¹ of Chemical Formula 2.

The first compound and the second compound may be, for example included in a weight ratio of about 1:9 to about 9:1, specifically about 2:8 to about 8:2, about 3:7 to about 7:3, about 4:6 to about 6:4, and about 5:5.

In an embodiment of the invention, the first compound and the second compound may be, for example included in a weight ratio of about 4:1 to about 1:1, specifically about 3:1 to about 1:1 or about 7:3 to about 1:1. Within the ranges, bipolar characteristics are realized and efficiency and life-span may be simultaneously improved.

The emission layer may further include a third compound in addition to the first compound and the second compound as a host.

The third compound may be represented by Chemical Formula 5.

Chemical Formula 5 may be, for example represented by Chemical Formulae 5-I or 5-II according to linking or non-linking of the R^{h}, Rⁱ, R^{l}, and R^{m}.

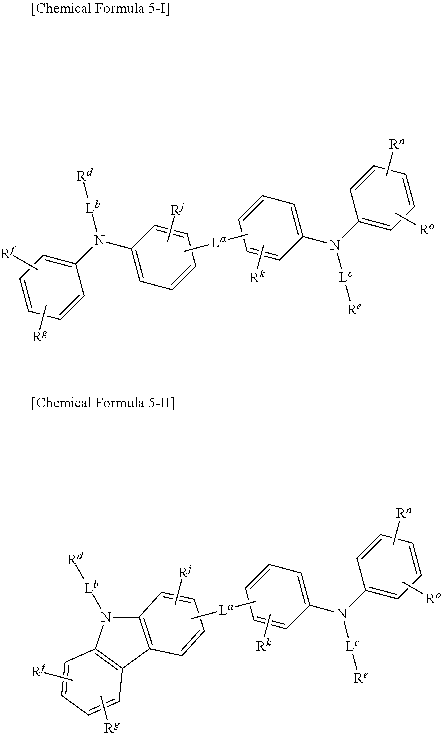

In Chemical Formulae 5-I to 5-II, R^{d} to R^{g}, R^{j}, R^{k}, Rⁿ, R^{o}, and L^{a} to L^{e} are the same as described above.

In Chemical Formula 5 according to an embodiment R^{d} to R^{o} may independently be hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted C6 to C30 arylamine group, or a combination thereof,

specifically, R^{f}, R^{g}, R^{j}, and R^{k} are independently hydrogen, and R^{d}, R^{e}, Rⁿ, and R^{o} are independently hydrogen, a substituted or unsubstituted C6 to C30 aryl group, a substituted or unsubstituted C2 to C30 heterocyclic group, or a combination thereof.

For example, the R^{d}, R^{e}, Rⁿ, and R^{o} are independently, a substituted or unsubstituted phenyl group, a substituted or unsubstituted biphenyl group, a substituted or unsubstituted terphenyl group, a substituted or unsubstituted quaterphenyl group, a substituted or unsubstituted naphthyl group, a substituted or unsubstituted anthracenyl group, a substituted or unsubstituted spirofluorenyl group, a substituted or unsubstituted triphenylene group, a substituted or unsubstituted fluorenyl group, a substituted or unsubstituted dibenzofuranyl group, a substituted or unsubstituted dibenzothiophenyl group, a substituted or unsubstituted carbazolyl group, or a combination thereof.

L^{a} to L^{c} of Chemical Formula 5 according to an embodiment may independently be a single bond, a substituted or unsubstituted C6 to C30 arylene group, or a combination thereof, and

specifically, a single bond, a substituted or unsubstituted phenylene group, or a substituted or unsubstituted biphenylene group, or a combination thereof.

For example, the substituted or unsubstituted phenylene group, or the substituted or unsubstituted biphenylene group may be selected from linking groups of Group 2.

The third compound represented by Chemical Formula 5 may be, for example, compounds of Groups F and G, but is not limited thereto.

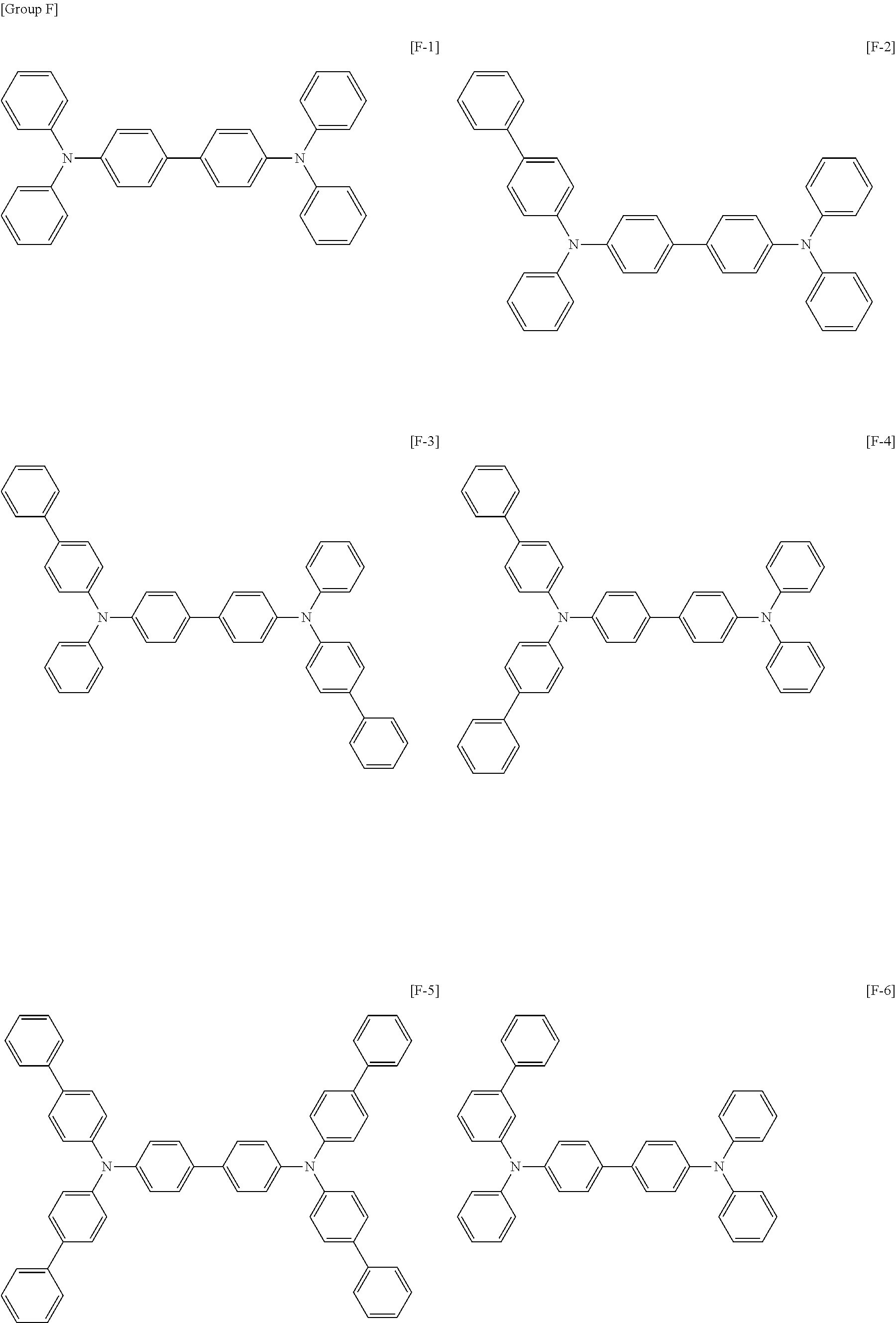 [SMILES: c1ccc(N(c2ccccc2)c2ccc(-c3ccc(N(c4ccccc4)c4ccccc4)cc3)cc2)cc1 ; c1ccc(-c2ccc(N(c3ccccc3)c3ccc(-c4ccc(N(c5ccccc5)c5ccccc5)cc4)cc3)cc2)cc1 ; c1ccc(-c2ccc(N(c3ccccc3)c3ccc(-c4ccc(N(c5ccccc5)c5ccc(-c6ccccc6)cc5)cc4)cc3)cc2)cc1 ; c1ccc(-c2ccc(N(c3ccc(-c4ccccc4)cc3)c3ccc(-c4ccc(N(c5ccccc5)c5ccccc5)cc4)cc3)cc2)cc1 ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(-c2cccc(N(c3ccccc3)c3ccc(-c4ccc(N(c5ccccc5)c5ccccc5)cc4)cc3)c2)cc1]

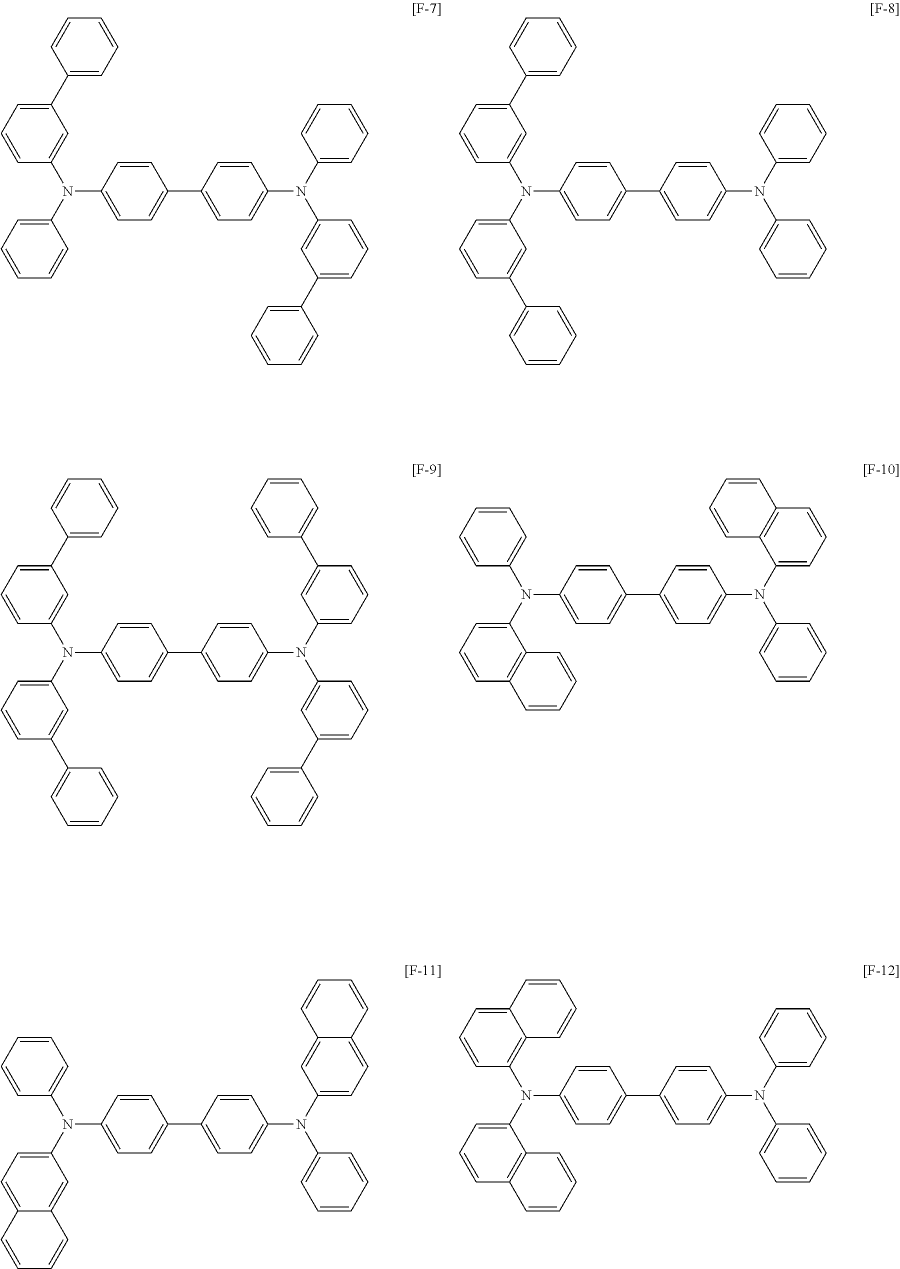

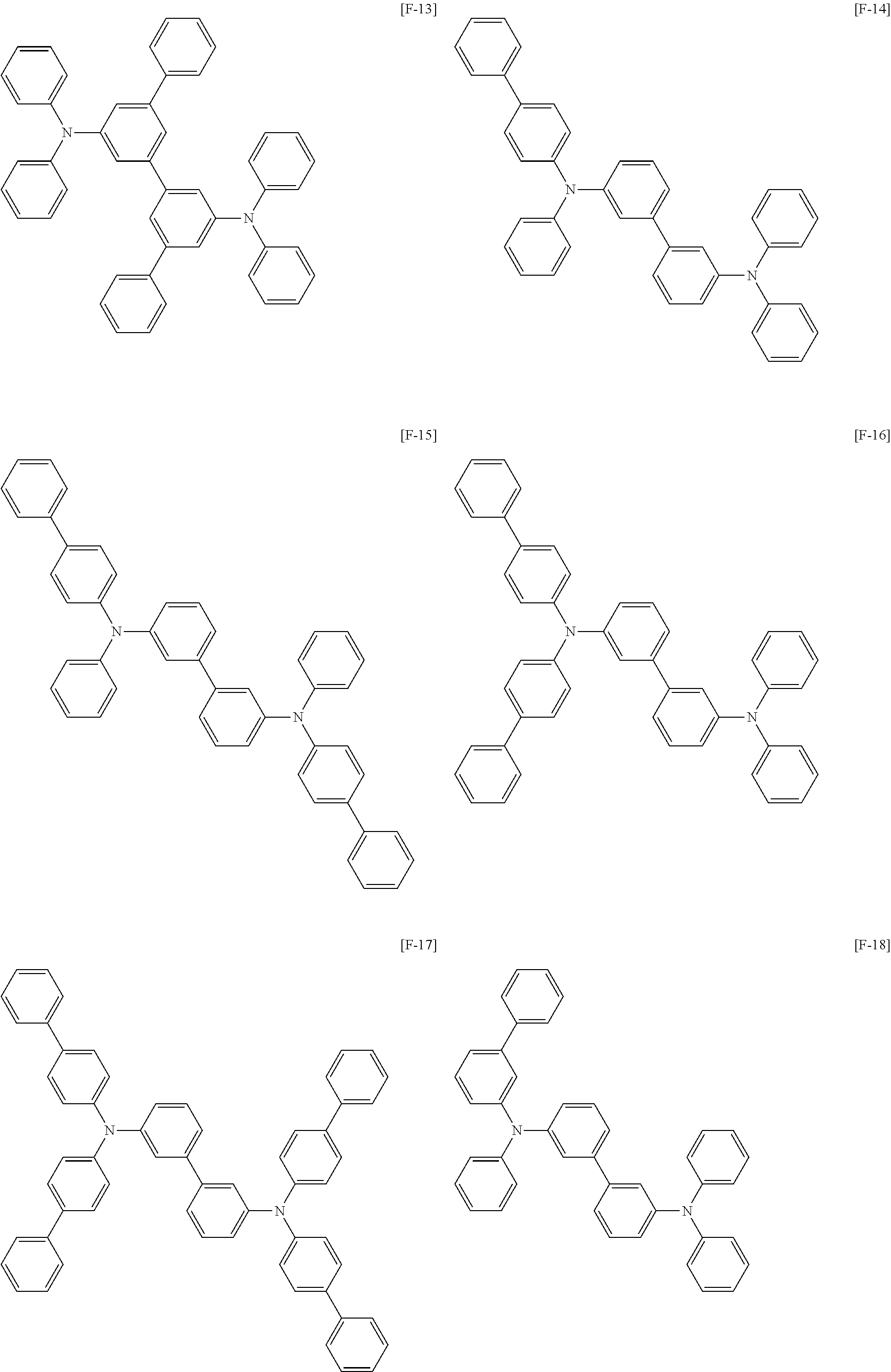

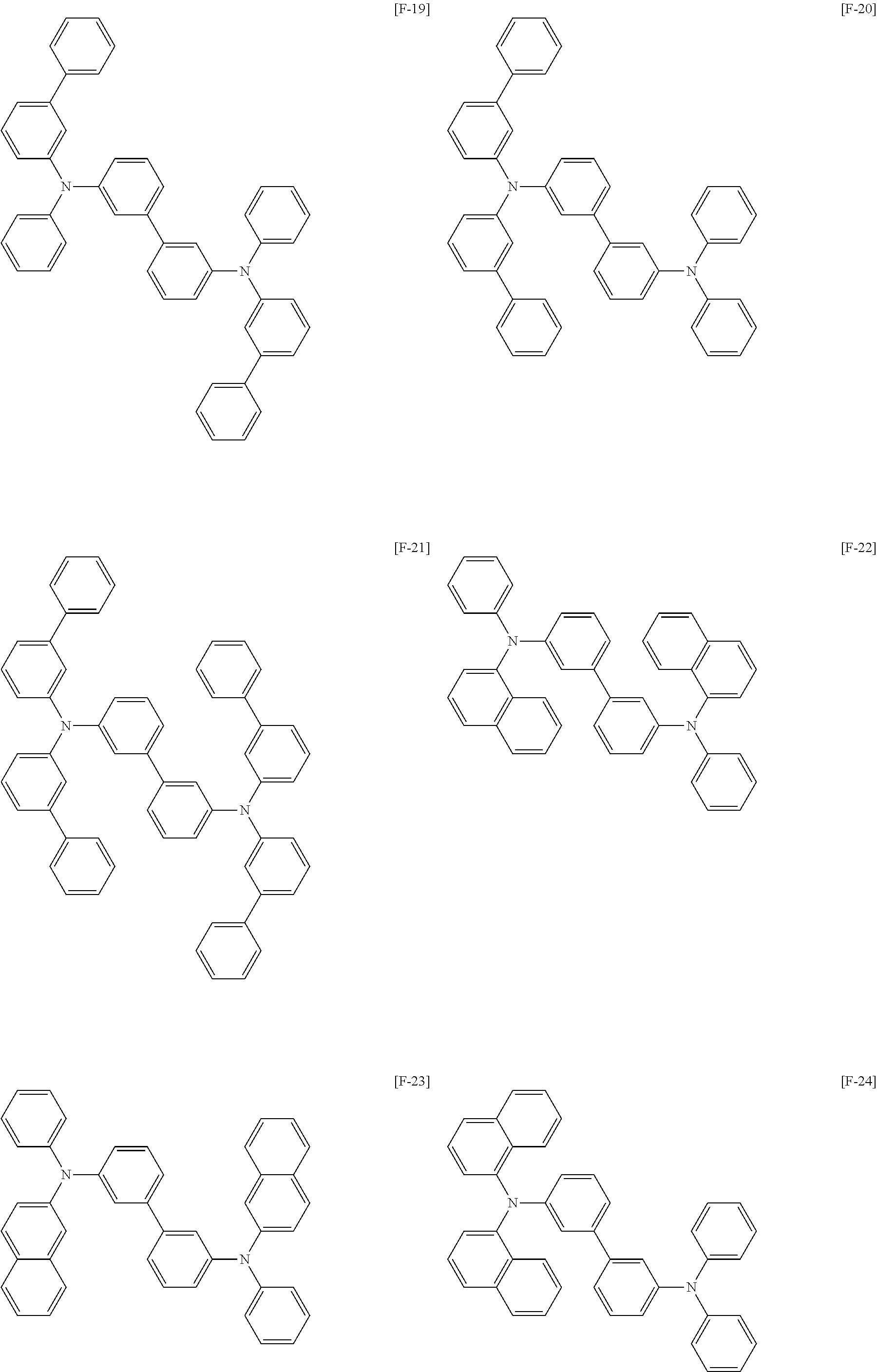

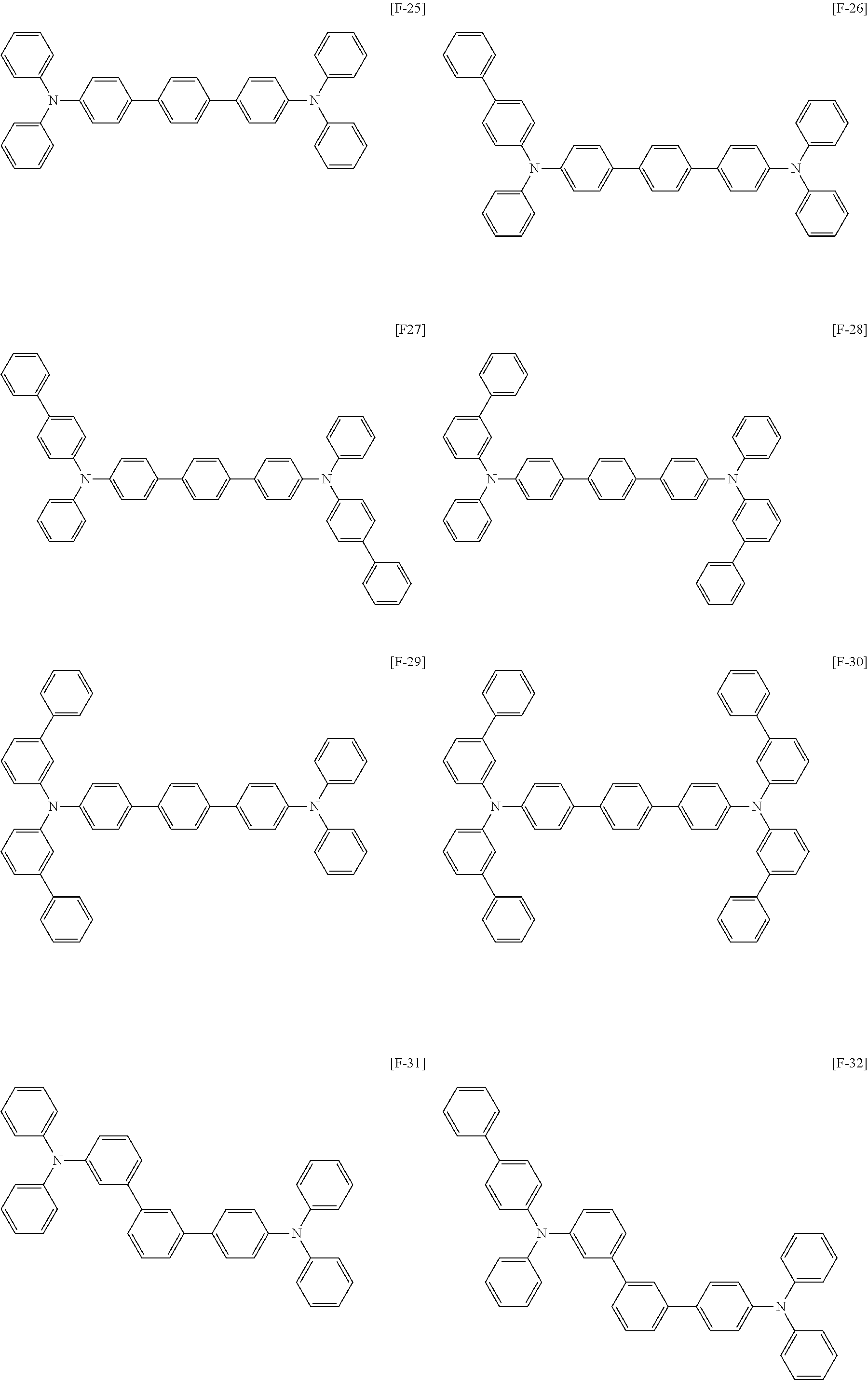

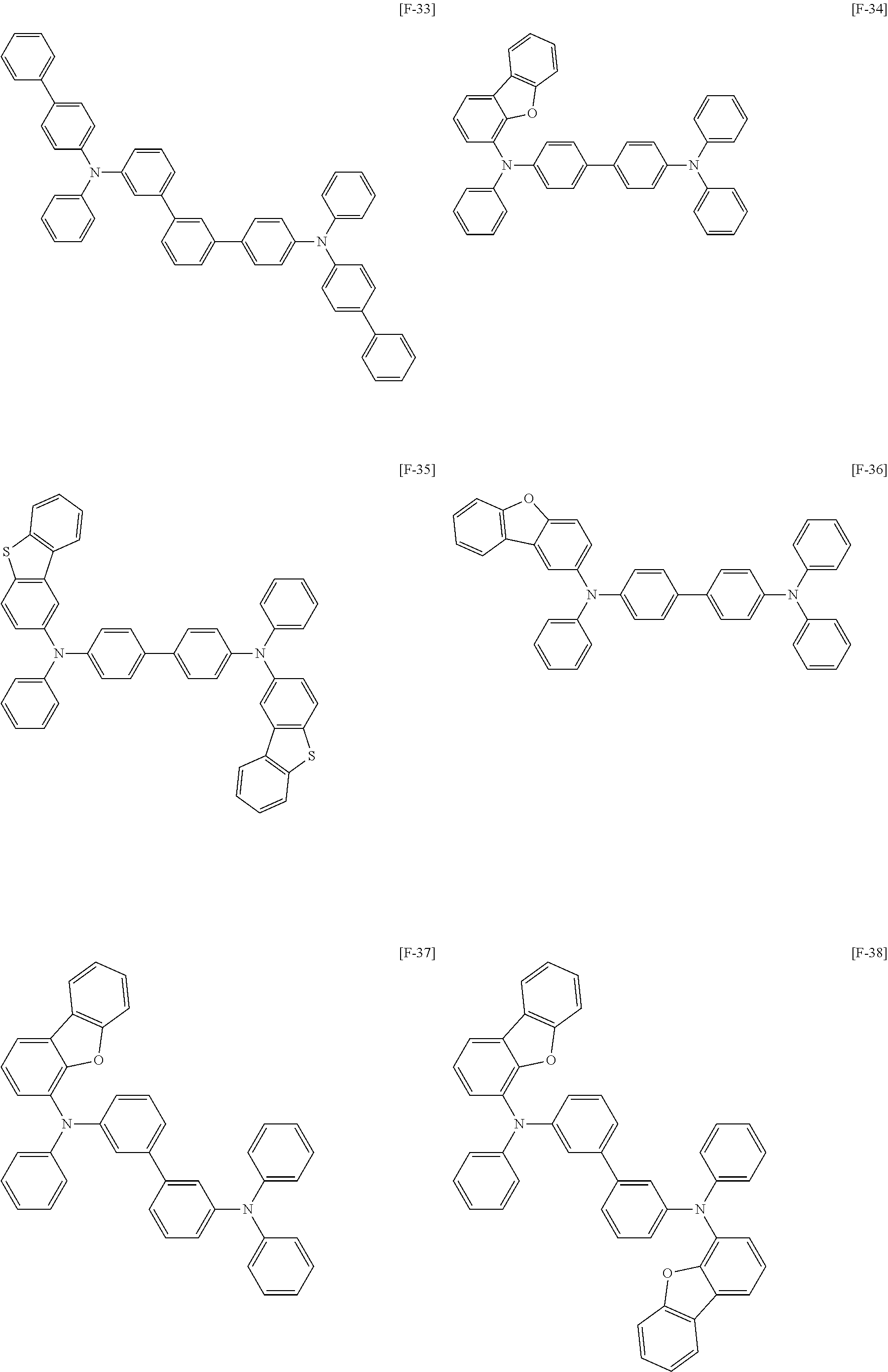 [SMILES: c1ccc(-c2ccc(N(c3ccccc3)c3ccc(-c4cccc(-c5cccc(N(c6ccccc6)c6ccc(-c7ccccc7)cc6)c5)c4)cc3)cc2)cc1 ; c1ccc(N(c2ccccc2)c2ccc(-c3ccc(N(c4ccccc4)c4cccc5c4oc4ccccc45)cc3)cc2)cc1 ; c1ccc(N(c2ccc(-c3ccc(N(c4ccccc4)c4ccc5sc6ccccc6c5c4)cc3)cc2)c2ccc3sc4ccccc4c3c2)cc1 ; c1ccc(N(c2ccccc2)c2ccc(-c3ccc(N(c4ccccc4)c4ccc5oc6ccccc6c5c4)cc3)cc2)cc1 ; c1ccc(N(c2ccccc2)c2cccc(-c3cccc(N(c4ccccc4)c4cccc5c4oc4ccccc45)c3)c2)cc1 ; c1ccc(N(c2cccc(-c3cccc(N(c4ccccc4)c4cccc5c4oc4ccccc45)c3)c2)c2cccc3c2oc2ccccc23)cc1]

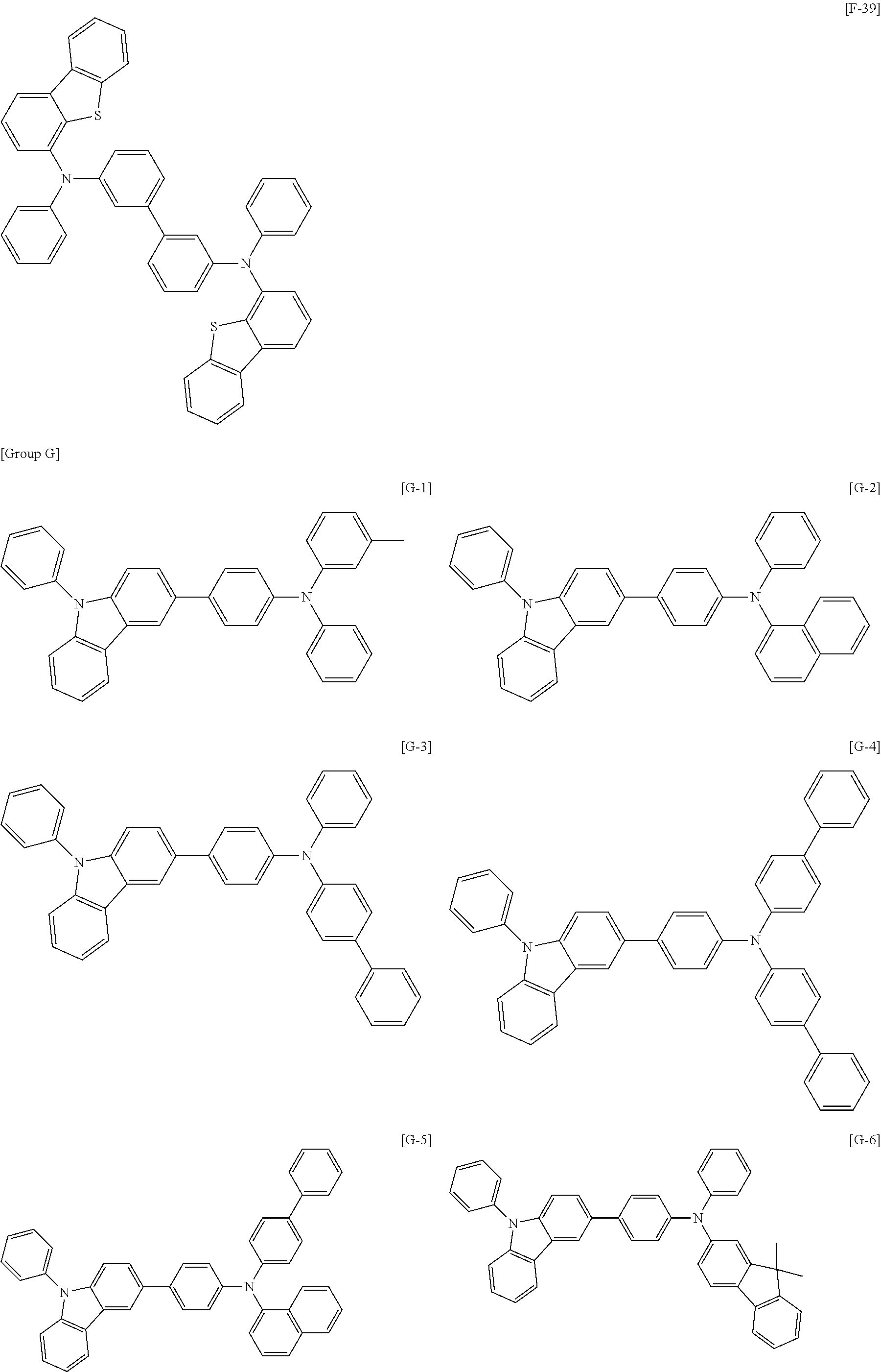

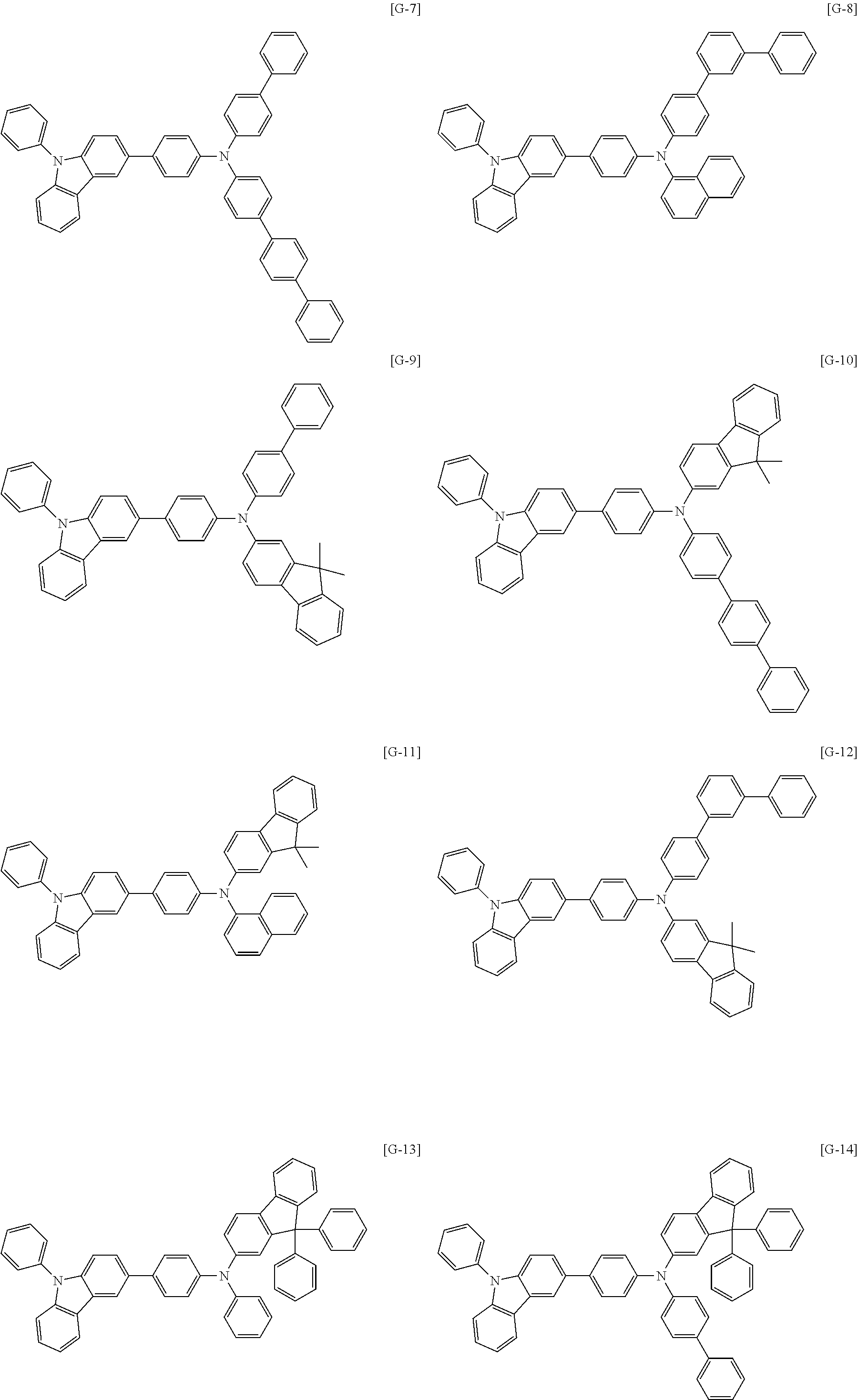

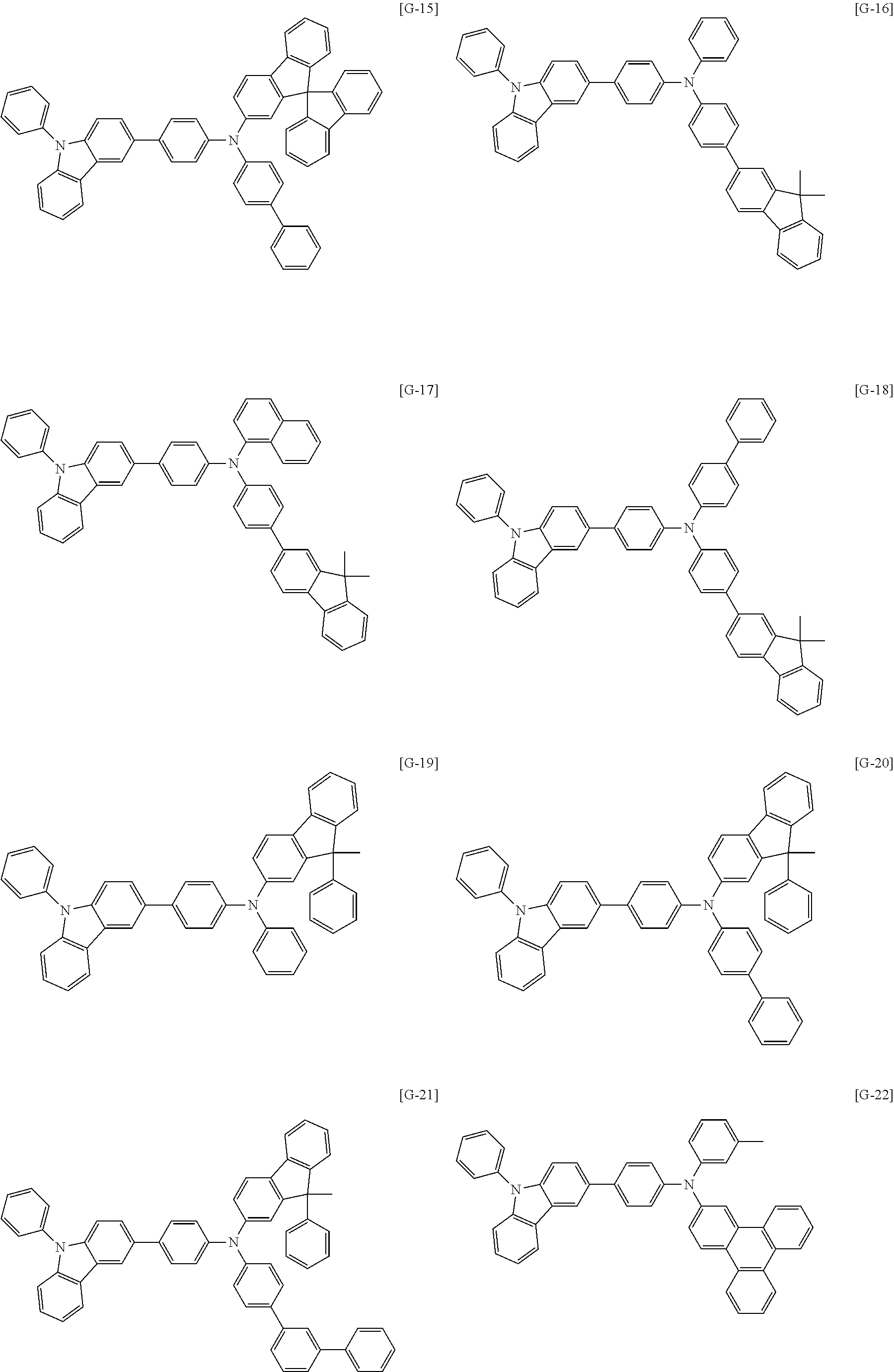

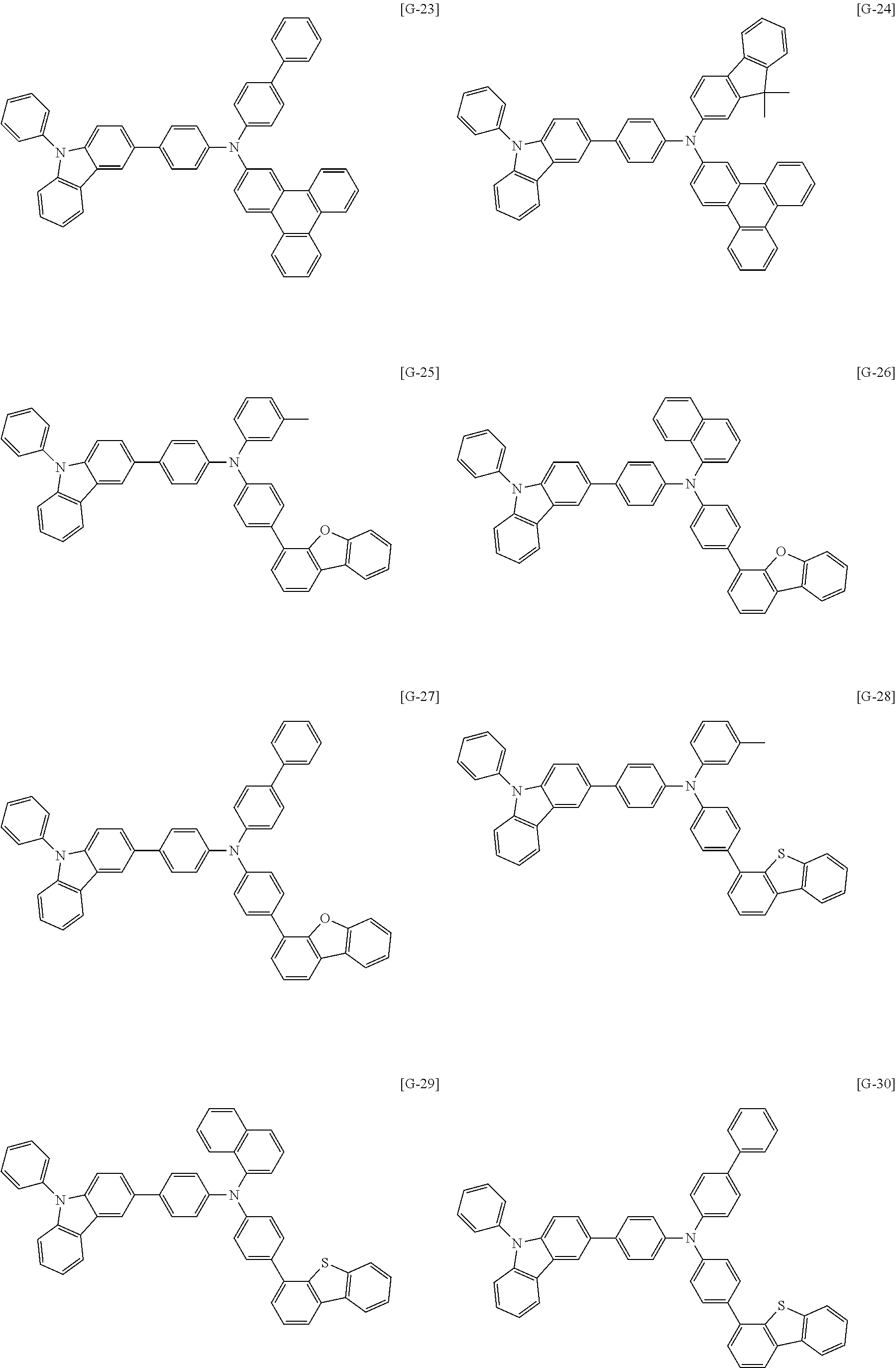

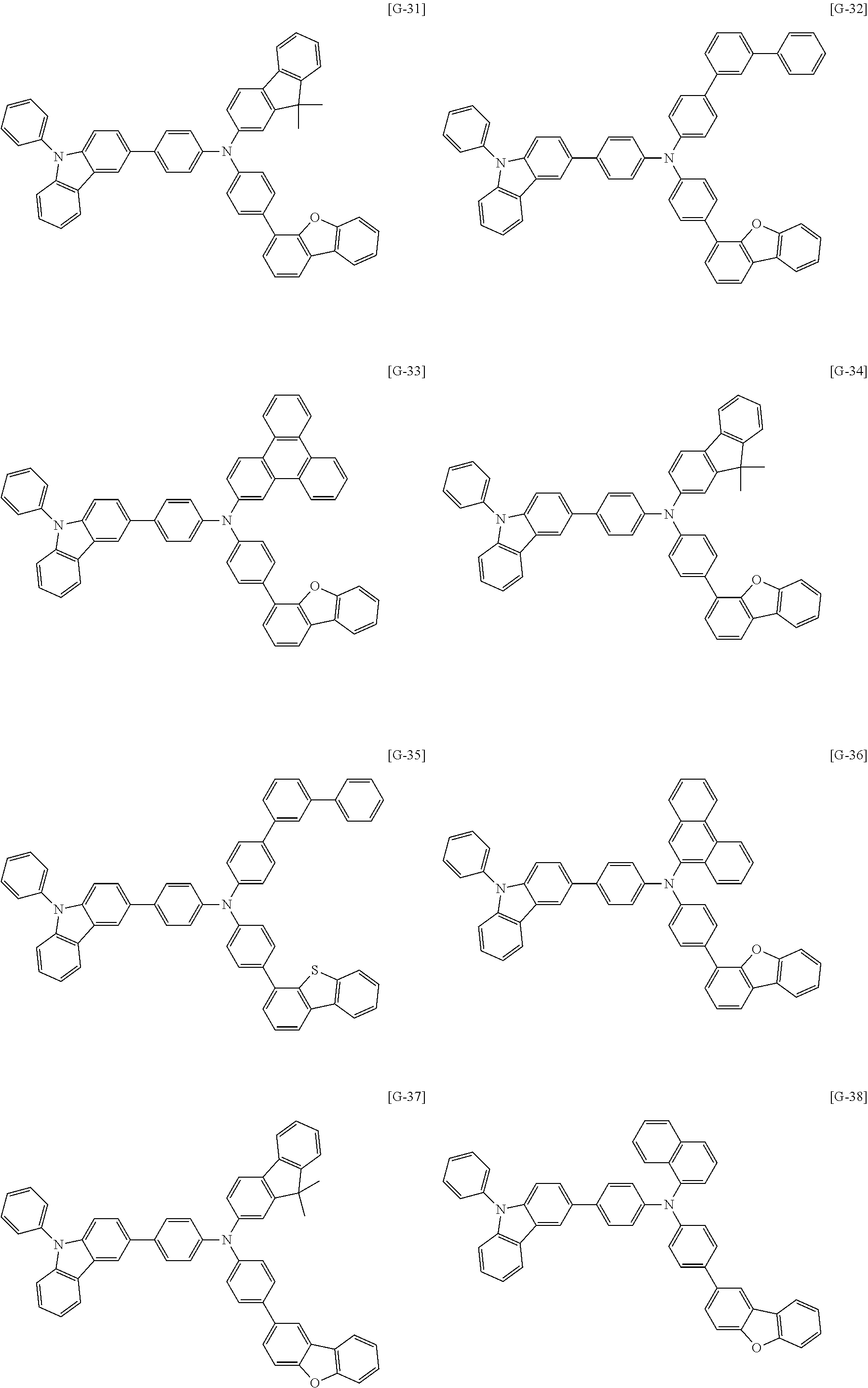

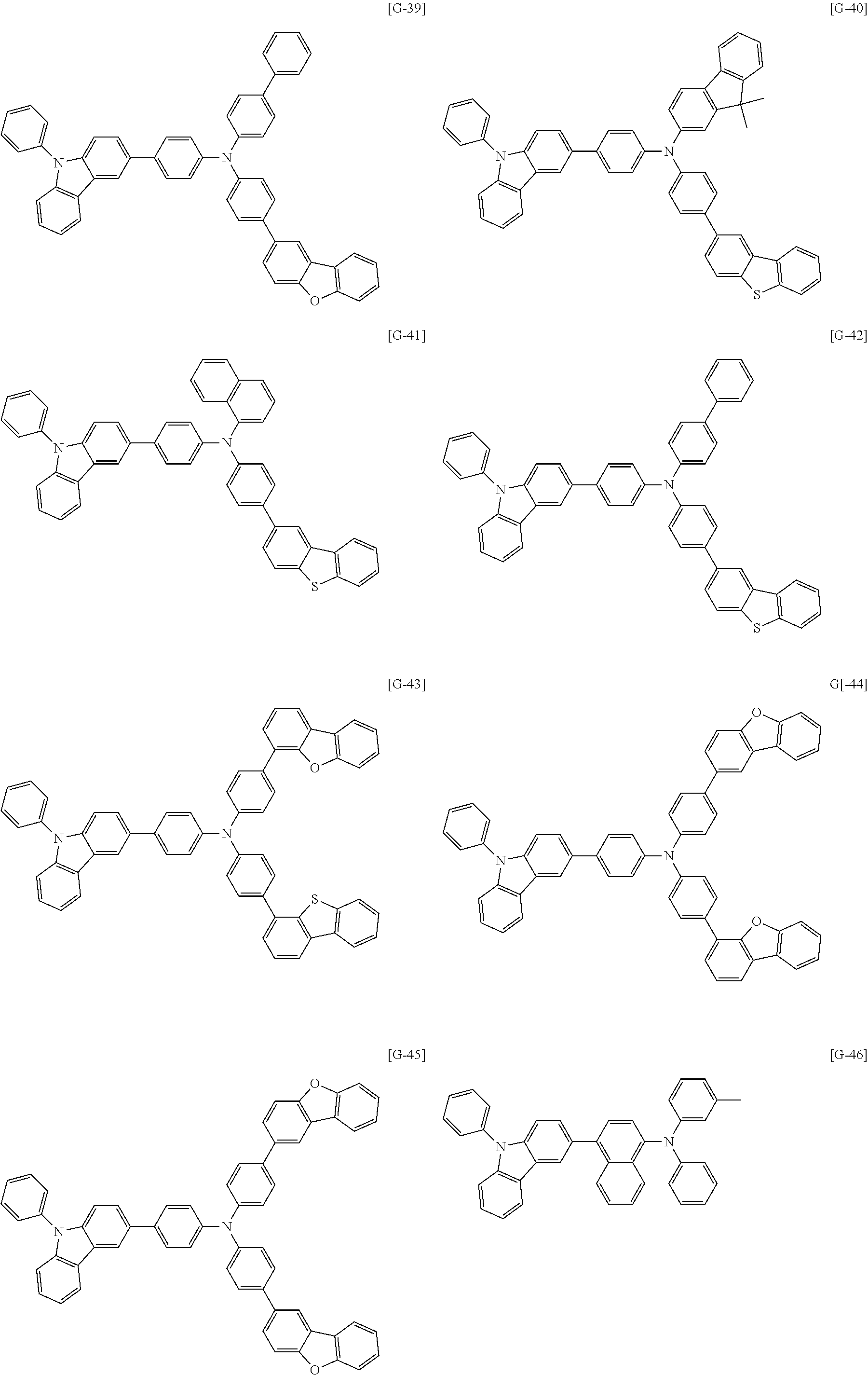

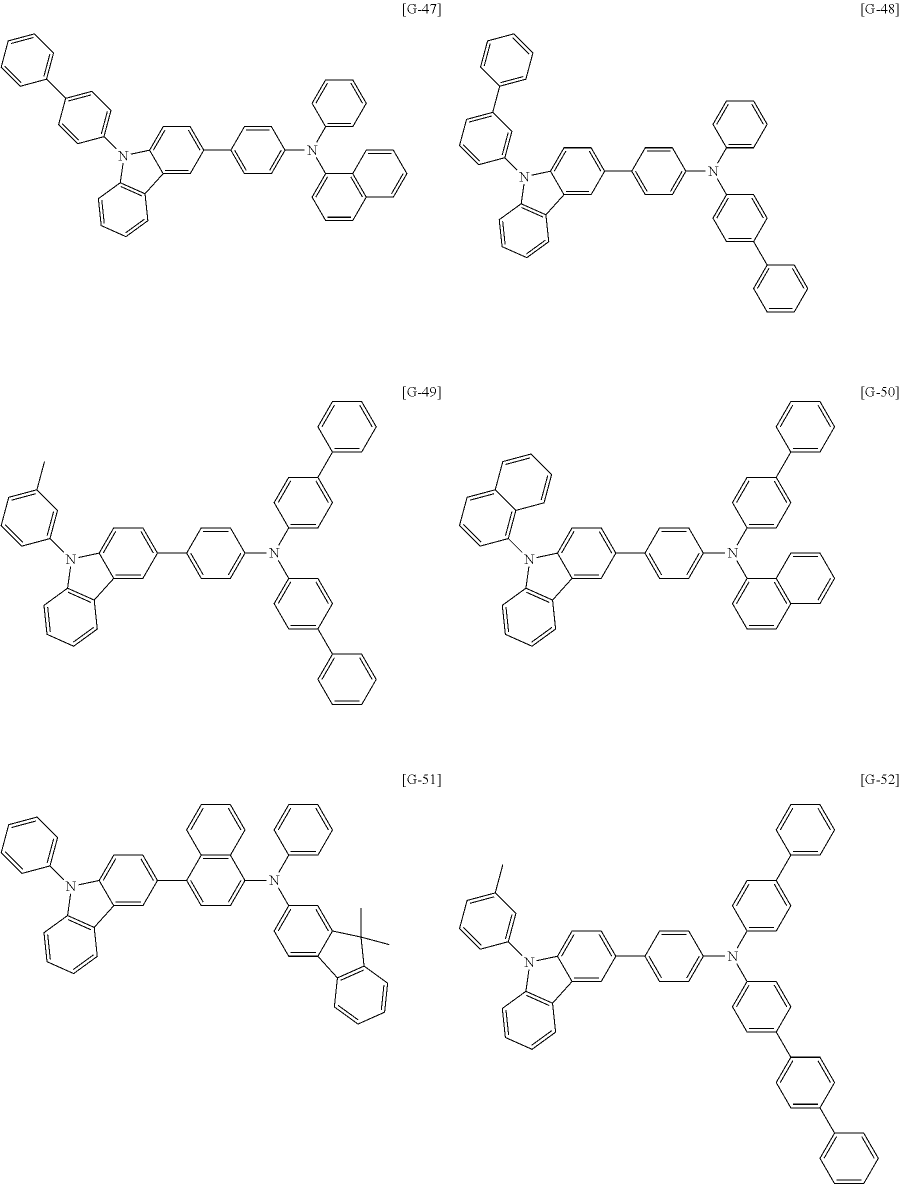

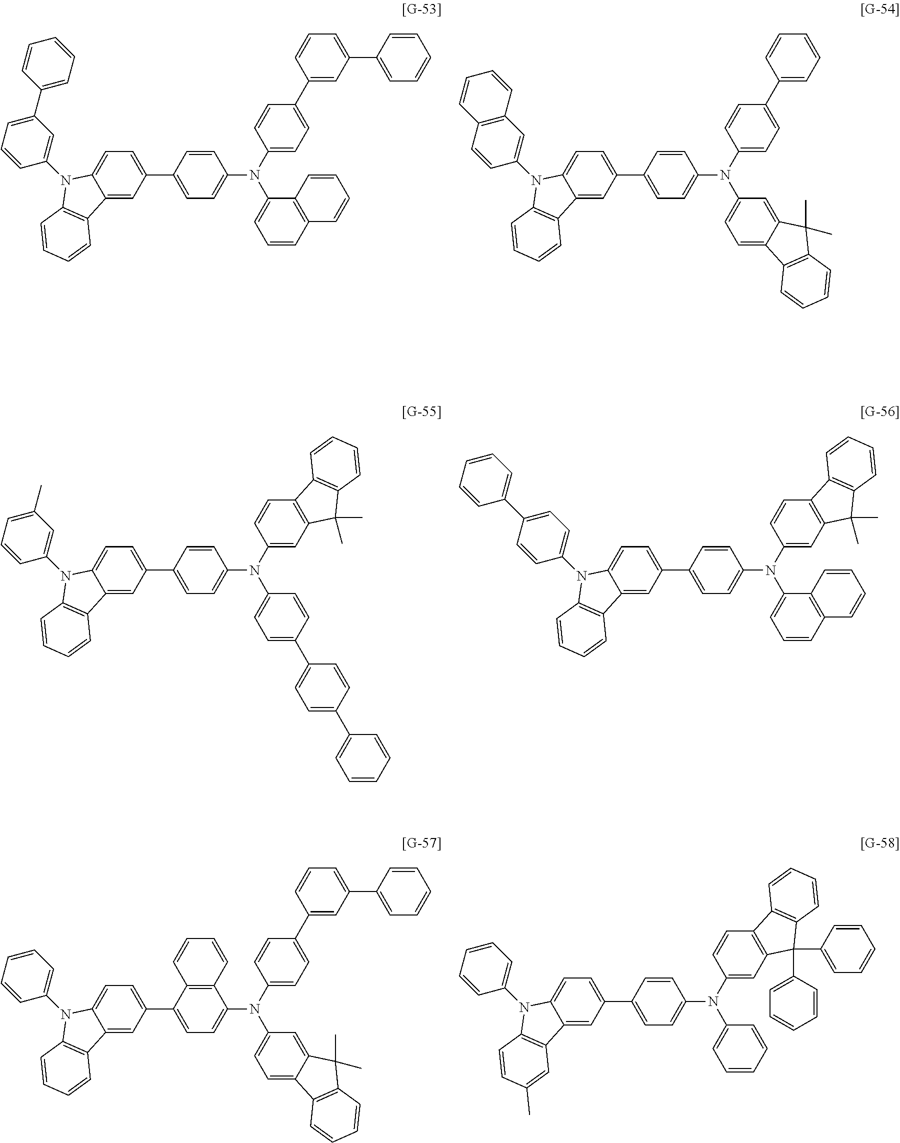

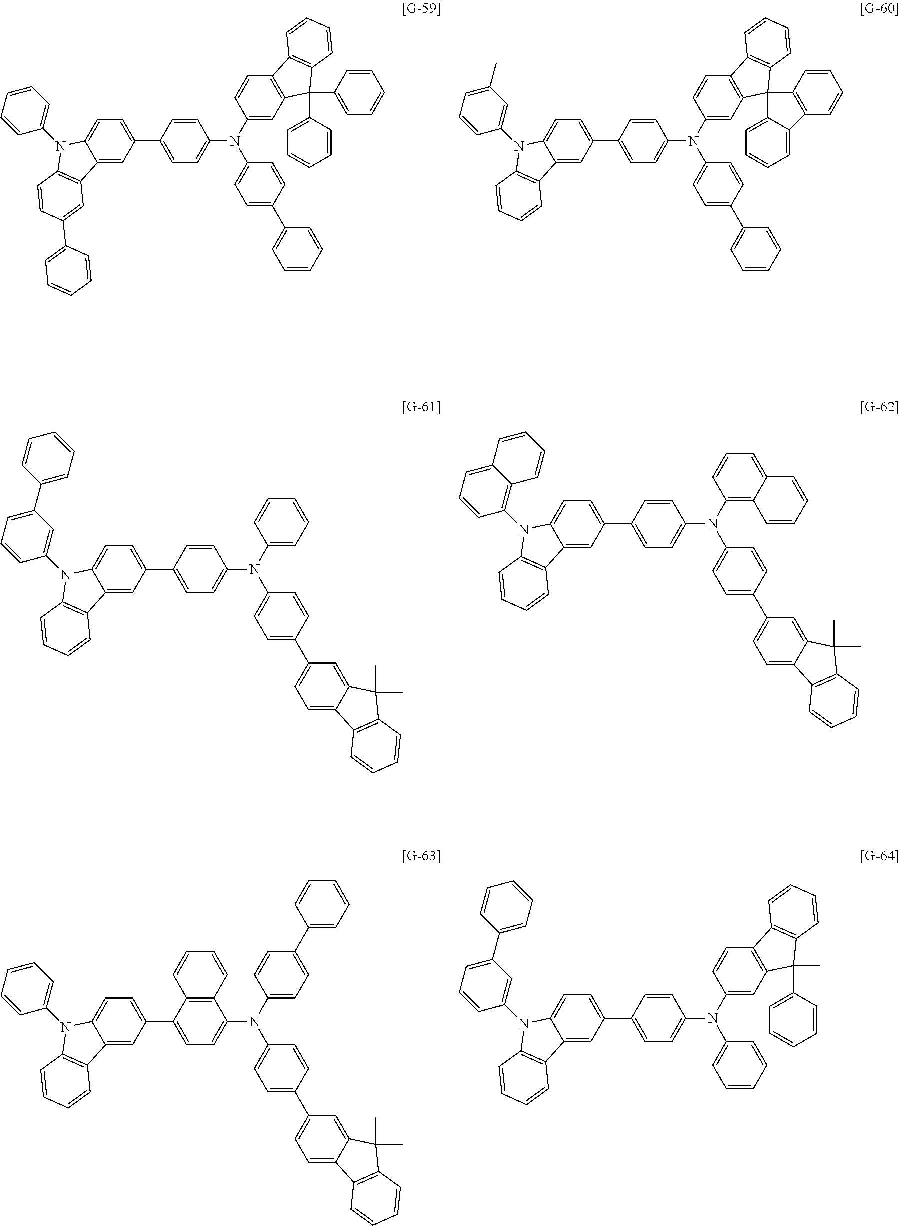

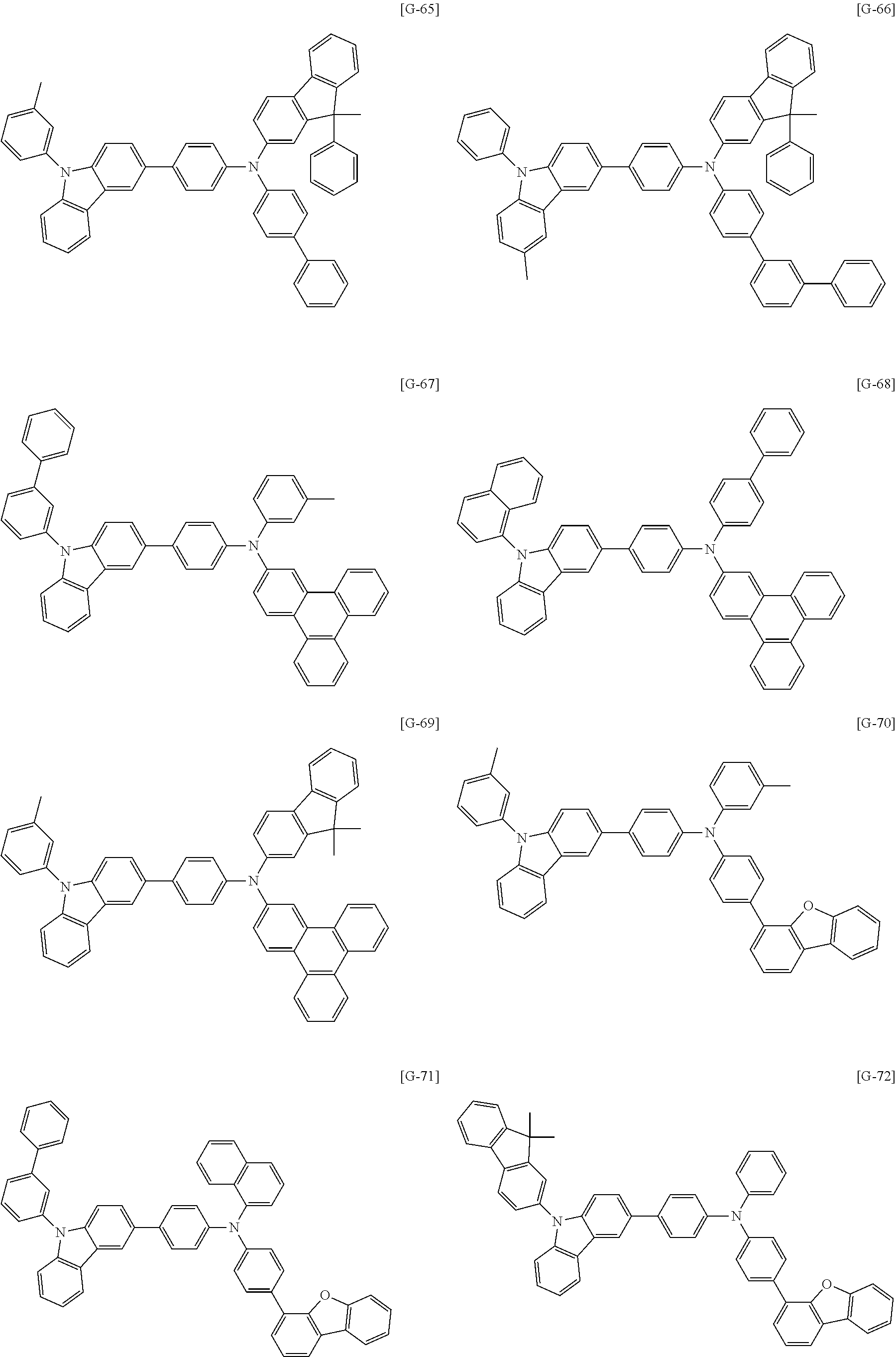

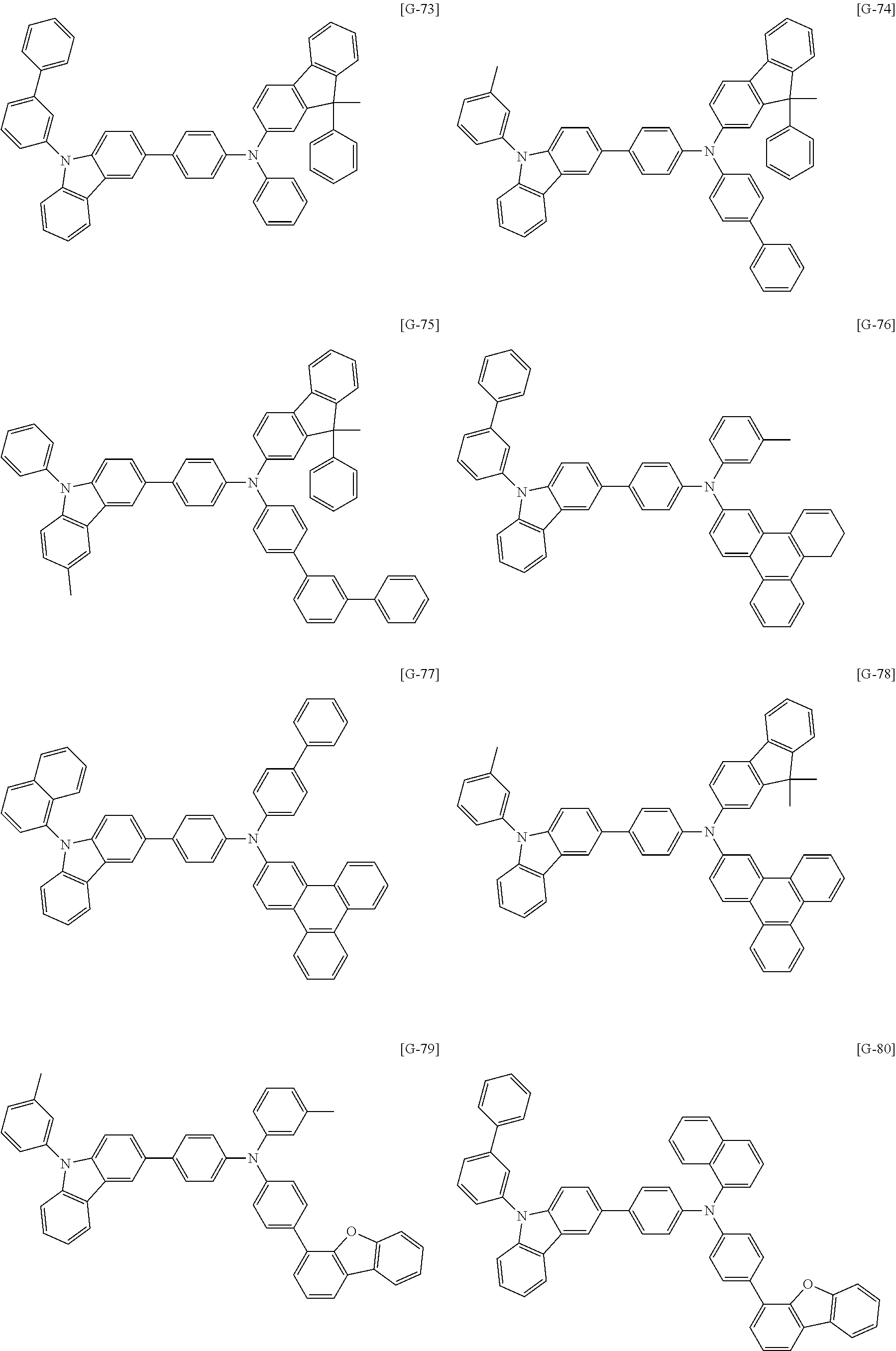

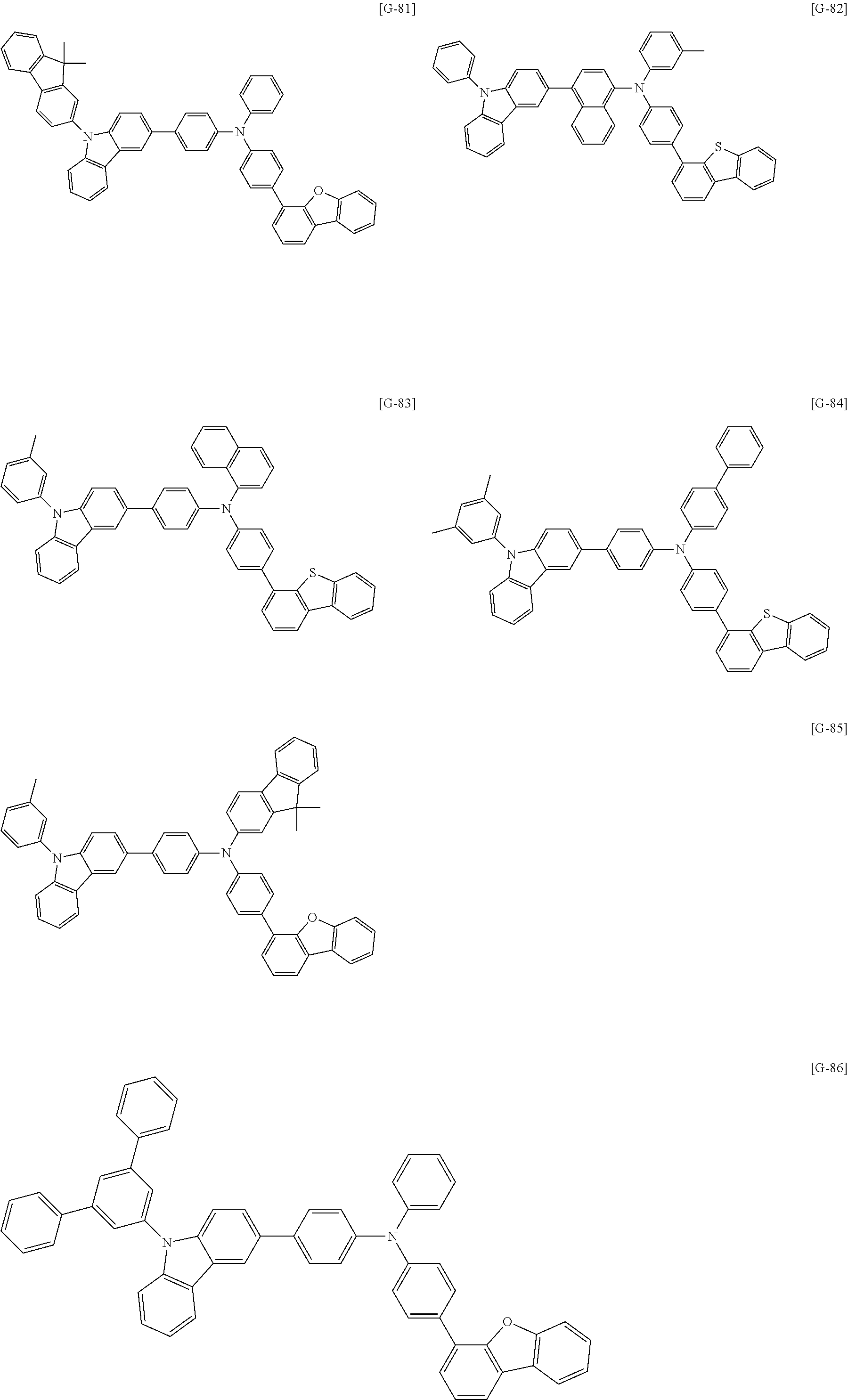

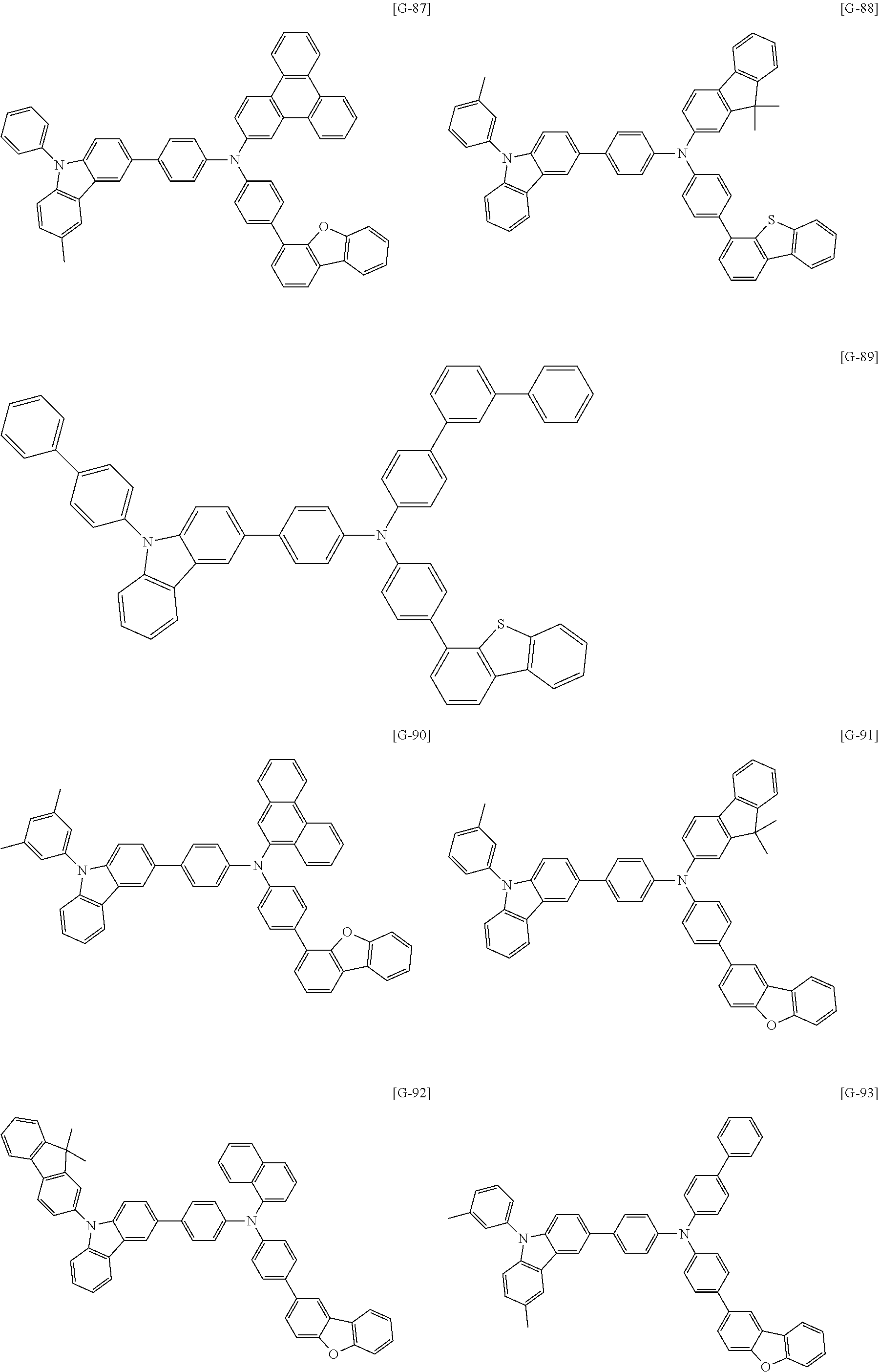

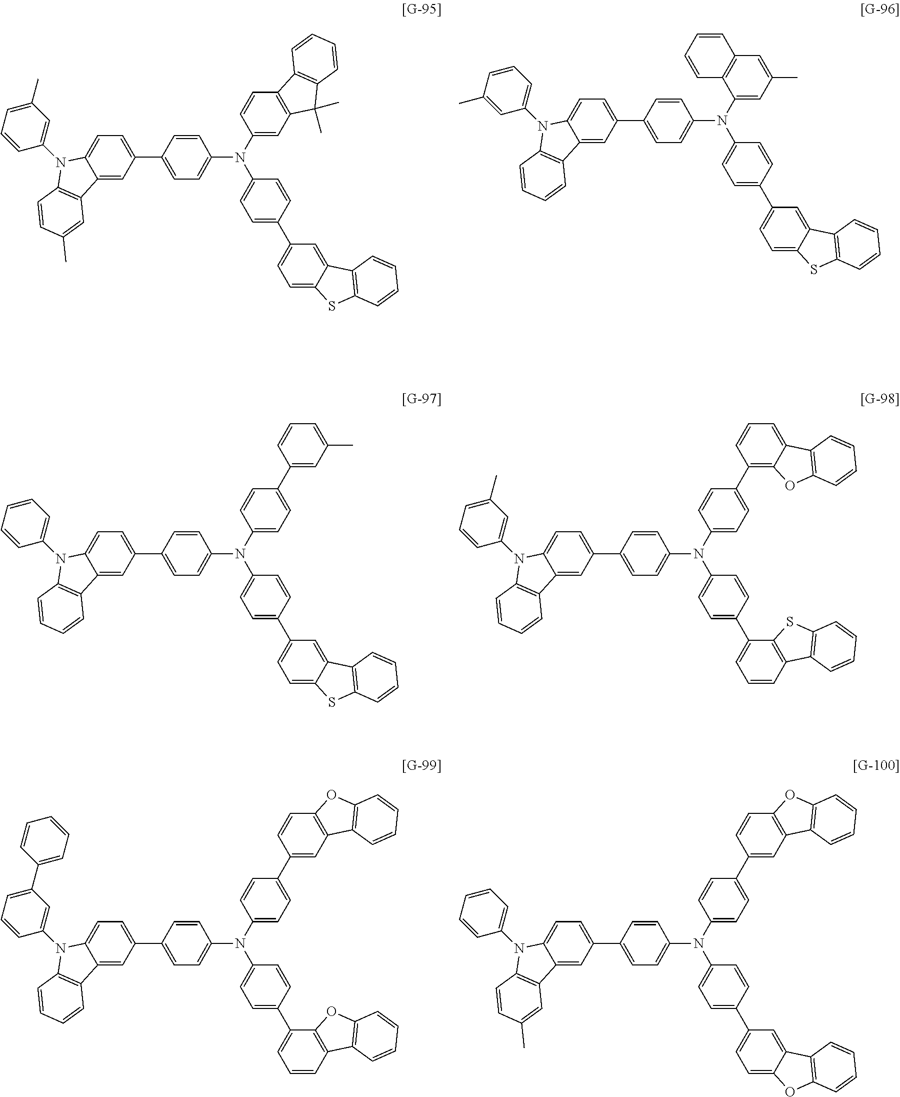

The composition for an organic optoelectronic device according to an embodiment including the first compound having bipolar characteristics in which electron characteristics are relatively strong, the second compound having bipolar characteristics in which hole characteristics are relatively strong, and a third compound having excellent hole injection and hole transport capability is included in an emission layer, and thereby the trap phenomenon between a dopant and a host is decreased or minimized and an injection wall of a hole between hole transport layer and the emission layer is lowered, and a driving voltage is remarkably reduced and luminous efficiency performance of a device is improved.

According to an embodiment, the emission layer includes the first compound, the second compound, and the third compound simultaneously as a host, and

the first compound may be specifically represented by Chemical Formula 1-I or Chemical Formula 1-III, the second compound may be represented by Chemical Formula 2-I, and the third compound may be represented by one of Chemical Formula 5-I or Chemical Formula 5-II.

More specifically, the first compound may be represented by Chemical Formula 1-IB or 1-IIIA, and Chemical Formula 1-IB may be, for more specific examples represented by Chemical Formula 1-I-3b.

The composition of the first compound and second compound and the third compound may be included in a weight ratio of about 90:10 to about 10:90, and specifically about 90:10 to about 10:90, about 85:15 to about 15:85, about 80:20 to about 20:80, about 70:30 to about 30:70, about 60:40 to about 40:60, or about 50:50. In an embodiment of the invention, a composition of the first compound and second compound and the third compound may be included in a weight ratio of about 95:5 to about 1:1, about 95:5 to about 6:4, about 9:1 to about 7:3.

Preferably, a composition of the first compound and second compound and the third compound may be included in a weight ratio of about 90:10, about 85:15, about 80:20, or about 70:30.

Within the ranges, bipolar characteristics are realized more effectively and efficiency and life-span may be simultaneously improved and a driving voltage may be reduced remarkably.

On the other hand, the first compound and second compound may be included in a weight ratio of about 1:10 to about 10:1, specifically about 2:8 to about 8:2, about 3:7 to about 7:3, about 4:6 to about 6:4 and about 5:5. Preferably, the first compound and the second compound may be included in a weight ratio of about 3:7, about 4:6, or about 5:5.

The emission layer may further include one or more compound in addition to the first compound and the second compound.

The emission layer may further include a dopant. The dopant is mixed with a host in a small amount to cause light emission, and may be generally a material such as a metal complex that emits light by multiple excitation into a triplet or more. The dopant may be, for example, an inorganic, organic, or organic/inorganic compound, and one or more kinds thereof may be used.

The dopant may be a red, green, or blue dopant, for example a phosphorescent dopant. The phosphorescent dopant may be an organic metal compound including Ir, Pt, Os, Ti, Zr, Hf, Eu, Tb, Tm, Fe, Co, Ni, Ru, Rh, Pd, or a combination thereof. The phosphorescent dopant may be, for example a compound represented by Chemical Formula Z, but is not limited thereto.

L₂MX [Chemical Formula Z]

In Chemical Formula Z, M is a metal, and L and X are the same or different, and are a ligand to form a complex compound with M.

The M may be, for example Ir, Pt, Os, Ti, Zr, Hf, Eu, Tb, Tm, Fe, Co, Ni, Ru, Rh, Pd, or a combination thereof, and the L and X may be, for example a bidendate ligand.

The composition may be applied to an organic layer of an organic optoelectronic device, for example the composition may be applied to an emission layer. For example, the composition may be applied to an emission layer as a host.

The composition may be formed using a dry film formation method or a solution process. The dry film formation method may be, for example a chemical vapor deposition (CVD) method, sputtering, plasma plating, and ion plating, and two or more compounds may be simultaneously formed into a film or compound having the same deposition temperature may be mixed and formed into a film. The solution process may be, for example inkjet printing, spin coating, slit coating, bar coating, and/or dip coating.

Hereinafter, an organic optoelectronic device including the composition is described.

The organic optoelectronic device may be any device to convert electrical energy into photoenergy and vice versa without particular limitation, and may be, for example selected from an organic light emitting diode, an organic photoelectric device, an organic solar cell, an organic transistor, an organic photo conductor drum, and an organic memory device.

The organic optoelectronic device includes an anode and a cathode facing each other, and at least one organic layer interposed between the anode and the cathode, wherein the organic layer includes the composition.

Hereinafter, an organic light emitting diode as one example of an organic optoelectronic device is described with reference to drawings.

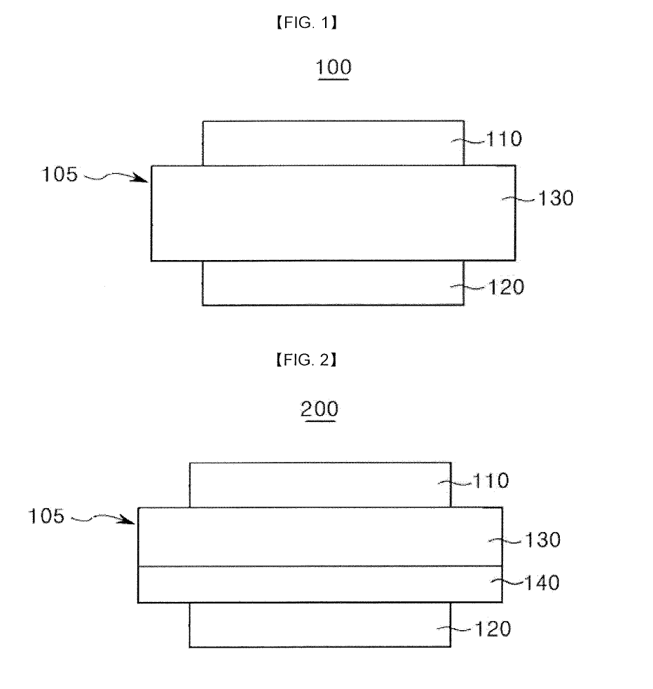

FIG. 1 is a cross-sectional view showing an organic light emitting diode according to an embodiment.

Referring to FIG. 1, an organic light emitting diode 100 according to an embodiment includes an anode 120 and a cathode 110 and facing each other and an organic layer 105 interposed between the anode 120 and the cathode 110.

The anode 120 may be made of a conductor having a large work function to help hole injection, and may be for example metal, metal oxide and/or a conductive polymer. The anode 120 may be, for example a metal nickel, platinum, vanadium, chromium, copper, zinc, gold, and the like or an alloy thereof; metal oxide such as zinc oxide, indium oxide, indium tin oxide (ITO), indium zinc oxide (IZO), and the like; a combination of metal and oxide such as ZnO and Al or SnO₂ and Sb; a conductive polymer such as poly(3-methylthiophene), poly(3,4-(ethylene-1,2-dioxy)thiophene) (PEDT), polypyrrole, and polyaniline, but is not limited thereto.

The cathode 110 may be made of a conductor having a small work function to help electron injection, and may be for example metal, metal oxide and/or a conductive polymer. The cathode 110 may be for example a metal or an alloy thereof such as magnesium, calcium, sodium, potassium, titanium, indium, yttrium, lithium, gadolinium, aluminum silver, tin, lead, cesium, barium, and the like; a multi-layer structure material such as LiF/Al, LiO₂/Al, LiF/Ca, LiF/Al, and BaF₂/Ca, but is not limited thereto.

The organic layer 105 includes an emission layer 130 including the composition.

FIG. 2 is a cross-sectional view showing an organic light emitting diode according to another embodiment.

Referring to FIG. 2, an organic light emitting diode 200 according to the present embodiment includes an anode 120 and a cathode 110 facing each other and an organic layer 105 interposed between the anode 120 and the cathode 110 like the above embodiment.

The organic layer 105 includes an emission layer 130 and an auxiliary layer 140 between the emission layer 130 and the anode 120. The auxiliary layer 140 helps charge injection and transfer between the anode 120 and the emission layer 130. The auxiliary layer 140 may be, for example an electron transport layer (ETL), an electron injection layer (EIL), and/or an electron transportauxiliary layer.

In FIGS. 1 and 2, at least one auxiliary layer as the organic layer 105 may be further included between the cathode 110 and the emission layer 130.

The organic light emitting diode may be applied to an organic light emitting diode (OLED) display.

Hereinafter, the embodiments are illustrated in more detail with reference to examples. These examples, however, are not in any sense to be interpreted as limiting the scope of the invention.

Hereinafter, starting materials and reactants used in Synthesis Examples and Examples of the present disclosure are purchased from Sigma-Aldrich Corp. or TCI unless particularly mentioned.

Synthesis of First Compound

(Synthesis of Intermediate)

Synthesis of Intermediate I-1

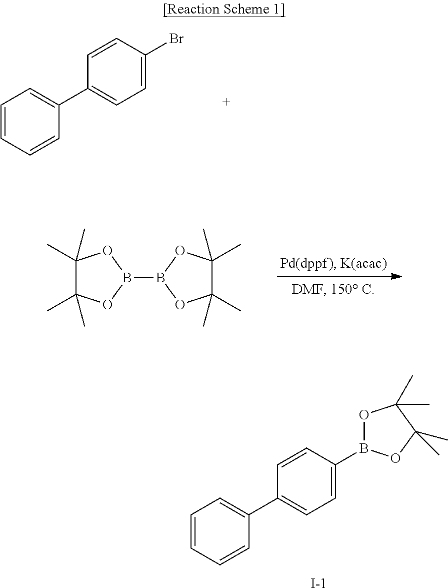

4-bromo-1,1′-biphenyl (20 g, 86 mmol) was dissolved in 1 L of dimethylforamide (DMF) in a nitrogen environment, bis(pinacolato)diboron (26 g, 103 mmol), (1,1′-bis(diphenylphosphine)ferrocene)dichloropalladium (II) (Pd(dppf)) (0.7 g, 0.86 mmol), and potassium acetate (K(acac)) (21 g, 215 mmol) were added thereto, and the mixture was heated and refluxed at 150° C. for 5 hours. When the reaction was complete, water was added to the reaction solution, and the mixture was filtered and then, dried in a vacuum oven. The obtained residue was separated and purified through flash column chromatography to obtain an intermediate I-1 (20 g and 85%).

HRMS (70 eV, EI+): m/z calcd for C18H21BO2: 280.1635, found: 280

Elemental Analysis: C, 77%; H, 8%

Synthesis of Intermediate I-2

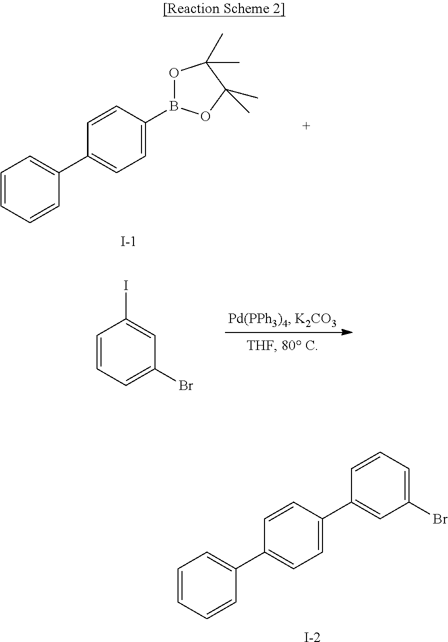

The intermediate I-1 (20 g, 71 mmol) was dissolved in THF (1 L) in a nitrogen environment, 1-bromo-3-iodobenzene (24 g, 85 mmol) and tetrakis(triphenylphosphine)palladium (Pd(PPh₃)₄) (0.8 mg, 0.7 mmol) were added thereto, and the mixture was stirred. Potassium carbonate (K₂CO₃) (24.5 g, 177 mmol) saturated in water was added thereto, and the resulting mixture was heated and refluxed at 80° C. for 12 hours. When the reaction was complete, water was added to the reaction solution, dichloromethane (DCM) was used for extraction, and an extract therefrom was treated with anhydrous MgSO₄ to remove moisture, filtered, and concentrated under a reduced pressure. The obtained residue was separated and purified through flash column chromatography to obtain an intermediate I-2 (30 g and 90%).

HRMS (70 eV, EI+): m/z calcd for C18H13Br: 309.1998, found 309 Elemental Analysis: C, 70%; H, 4%

Synthesis of Intermediate I-3

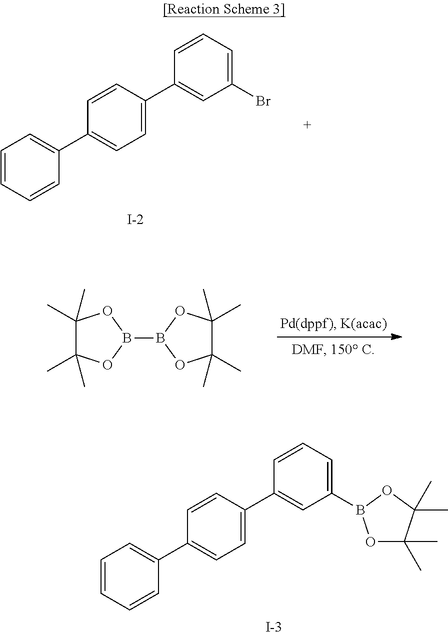

An intermediate I-3 (27 g and 93%) was obtained by reacting the intermediate I-2 (25 g, 81 mmol) in a nitrogen environment according to the same synthesis method as the intermediate I-1.

HRMS (70 eV, EI+): m/z calcd for C24H25BO2: 356.1948, found: 356

Elemental Analysis: C, 81%; H, 7%

Synthesis of Intermediate I-4

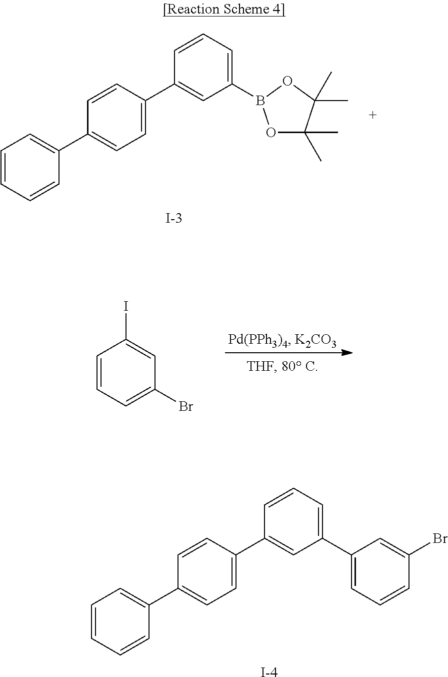

An intermediate I-4 (44 g and 89%) was obtained by reacting the intermediate I-3 (50 g, 140 mmol) in a nitrogen environment according to the same synthesis method as the intermediate I-2.

HRMS (70 eV, EI+): m/z calcd for C24H17Br: 384.0514, found 384 Elemental Analysis: C, 75%; H, 4%

Synthesis of Intermediate I-5

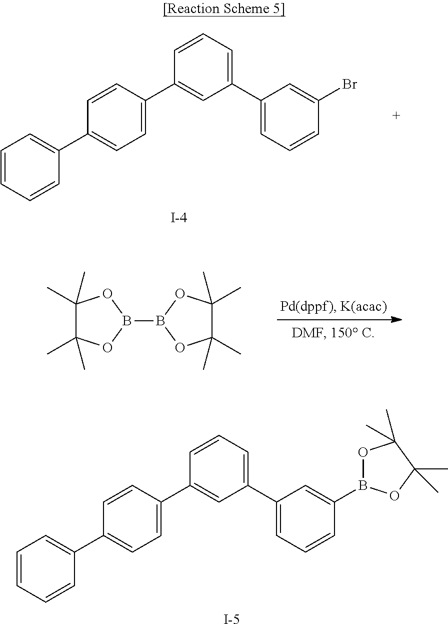

An intermediate I-5 (19 g and 85%) was obtained by reacting the intermediate I-4 (20 g, 52 mmol) in a nitrogen environment according to the same synthesis method as the intermediate I-1.

HRMS (70 eV, EI+): m/z calcd for C30H29BO2: 432.2261, found: 432

Elemental Analysis: C, 83%; H, 7%

Synthesis of Intermediate I-6

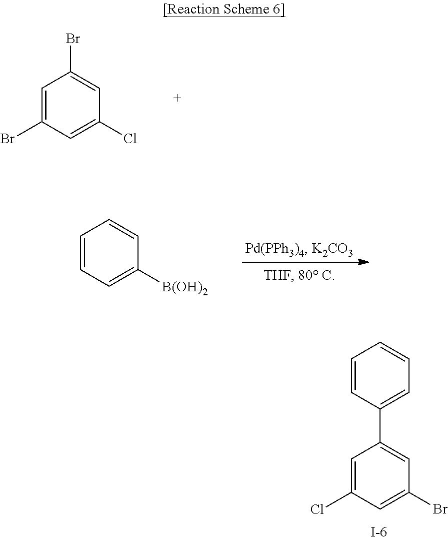

1,3-dibromo-5-chlorobenzene (100 g, 370 mmol) was dissolved in THF (2 L) in a nitrogen environment, phenylboronic acid (47.3 g, 388 mmol) and tetrakis(triphenylphosphine)palladium (Pd(PPh₃)₄) (1.5 g, 1.36 mmol) were added thereto, and the mixture was stirred. Potassium carbonate (K₂CO₃) (127 g, 925 mmol) saturated in water was added thereto, and the resulting mixture was heated and refluxed at 80° C. for 12 hours. When the reaction was complete, water was added to the reaction solution, the mixture was extracted with dichloromethane (DCM), and an extract therefrom was filtered and concentrated under a reduced pressure after removing moisture with anhydrous MgSO₄. The obtained residue was separated and purified through flash column chromatography to obtain an intermediate I-6 (49 g, 50%).

HRMS (70 eV, EI+): m/z calcd for C12H8BrCl: 265.9498, found 266 Elemental Analysis: C, 54%; H, 3%

Synthesis of Intermediate I-7

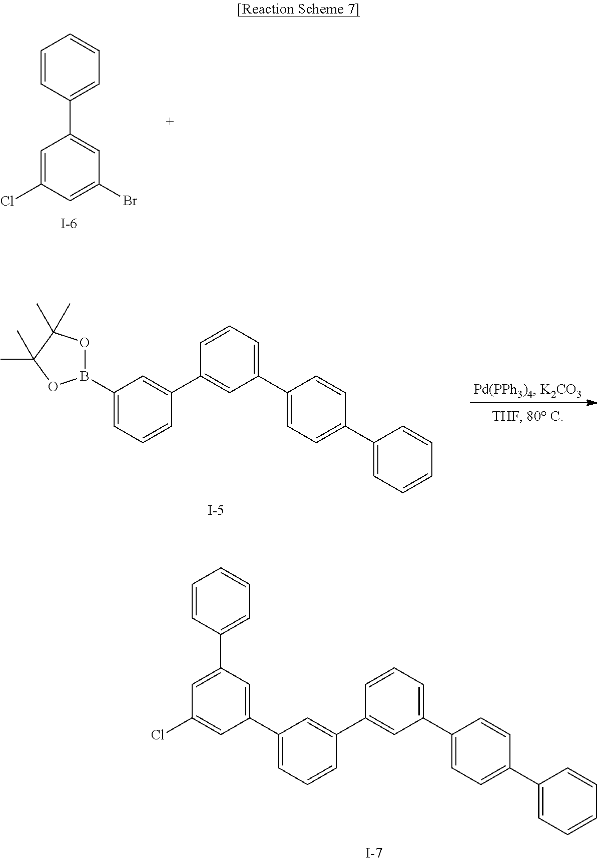

The intermediate I-6 (22.43 g, 83.83 mmol) was dissolved in THF (500 mL) in a nitrogen environment, the intermediate I-5 (50.7 g, 117.36 mmol) and tetrakis(triphenylphosphine)palladium (Pd(PPh₃)₄) (2.9 g, 2.5 mmol) were added thereto, and the mixture was stirred. Potassium carbonate (K₂CO₃) (46 g. 335.31 mmol) saturated in water was added thereto, and the resulting mixture was heated and refluxed at 80° C. for 12 hours. When the reaction was complete, water was added to the reaction solution, the mixture was extracted with dichloromethane (DCM), and an extract therefrom was filtered and concentrated under a reduced pressure after removing moisture with anhydrous MgSO₄. The obtained residue was separated and purified through flash column chromatography to obtain an intermediate I-7 (33 g and 81%).

HRMS (70 eV, EI+): m/z calcd for C36H25Cl: 492.1645, found: 492 Elemental Analysis: C, 88%; H, 5%

Synthesis of Intermediate I-8

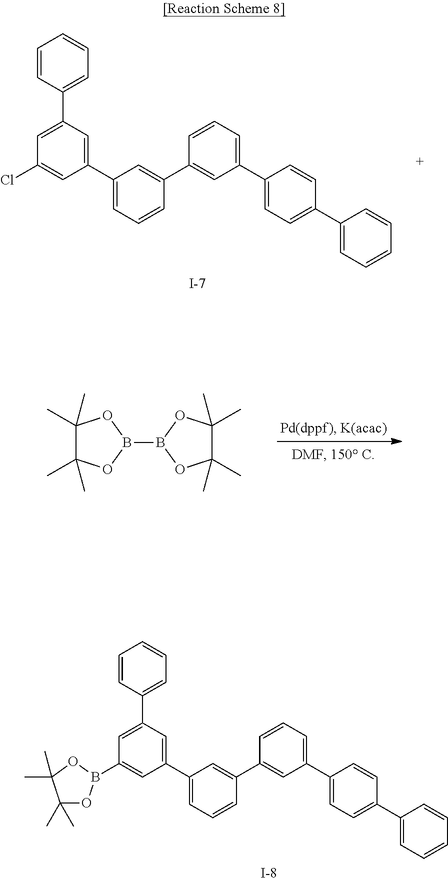

An intermediate I-8 (42 g and 85%) was obtained by reacting the intermediate I-7 (42 g, 85.8 mmol) in a nitrogen environment according to the same method as the intermediate I-1.

HRMS (70 eV, EI+): m/z calcd for C42H37BO2: 584.2887, found: 584.

Elemental Analysis: C, 86%; H, 6%

(Synthesis of Final Compound)

### SYNTHESIS EXAMPLE 1

### Synthesis of Compound A-275

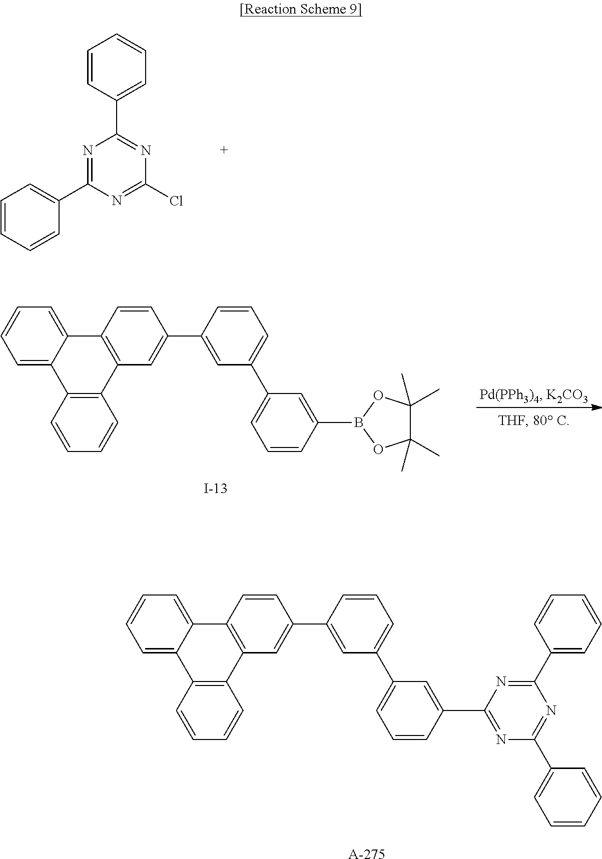

2-chloro-4,6-diphenyl-1,3,5-triazine (10.6 g, 39.5 mmol) was dissolved in THF (1 L) in a nitrogen environment, the intermediate I-13 (20 g, 39.5 mmol, manufactured with a reference to Synthesis Examples 1 to 7 of WO 2014/185598) and tetrakis(triphenylphosphine)palladium (Pd(PPh₃)₄) (0.46 g. 0.4 mmol) were added thereto, and the mixture was stirred. Potassium carbonate (K₂CO₃) (13.6 g, 98.8 mmol) saturated in water was added thereto, and the resulting mixture was heated and refluxed at 80° C. for 12 hours. When the reaction was complete, water was added to the reaction solution, dichloromethane (DCM) was used for extraction, and an extract therefrom was treated with anhydrous MgSO₄ to remove moisture, filtered, and concentrated under a reduced pressure. The obtained residue was separated and purified through flash column chromatography to obtain a compound A-275 (17.9 g, 74%).

HRMS (70 eV, EI+): m/z calcd for C45H29N3: 611.2361, found 611 Elemental Analysis: C, 88%; H, 5%

### SYNTHESIS EXAMPLE 2

### Synthesis of Compound A-216

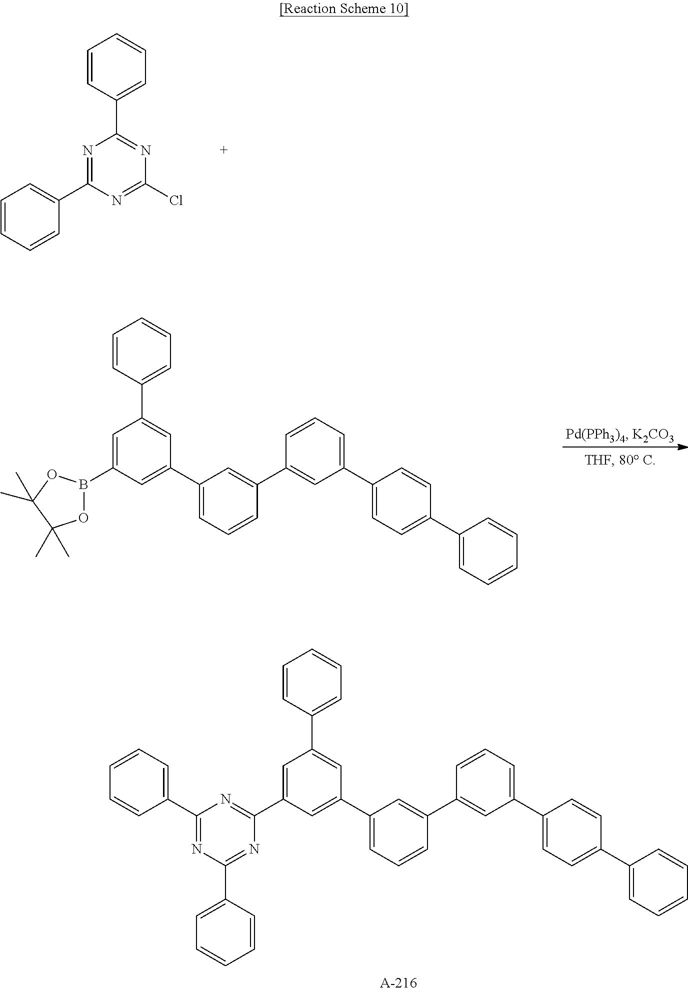 [SMILES: Clc1nc(-c2ccccc2)nc(-c2ccccc2)n1 ; CC1(C)OB(c2cc(-c3ccccc3)cc(-c3cccc(-c4cccc(-c5ccc(-c6ccccc6)cc5)c4)c3)c2)OC1(C)C ; c1ccc(-c2ccc(-c3cccc(-c4cccc(-c5cc(-c6ccccc6)cc(-c6nc(-c7ccccc7)nc(-c7ccccc7)n6)c5)c4)c3)cc2)cc1 ; "A-216"]

2-chloro-4,6-diphenyl-1,3,5-triazine (32 g, 76 mmol) was dissolved in THF (1 L) in a nitrogen environment, the intermediate I-8 (44 g, 76 mmol) and tetrakis(triphenylphosphine)palladium (Pd(PPh₃)₄) (0.88 g, 0.76 mmol) were added thereto, and the mixture was stirred. Potassium carbonate (K₂CO₃) (26 g, 190 mmol) saturated in water was added thereto, and the resulting mixture was heated and refluxed at 80° C. for 12 hours. When the reaction was complete, water was added to the reaction solution, dichloromethane (DCM) was used for extraction, and an extract was treated with anhydrous MgSO₄ to remove moisture, filtered, and concentrated under a reduced pressure. The obtained residue was separated and purified through flash column chromatography to obtain a compound A-216 (41 g and 80%).

HRMS (70 eV, EI+): m/z calcd for C51H35N3: 689.2831, found 689 Elemental Analysis: C. 89%; H, 5%

Synthesis of Second Compound

### SYNTHESIS EXAMPLE 3

### Synthesis of Compound B-31

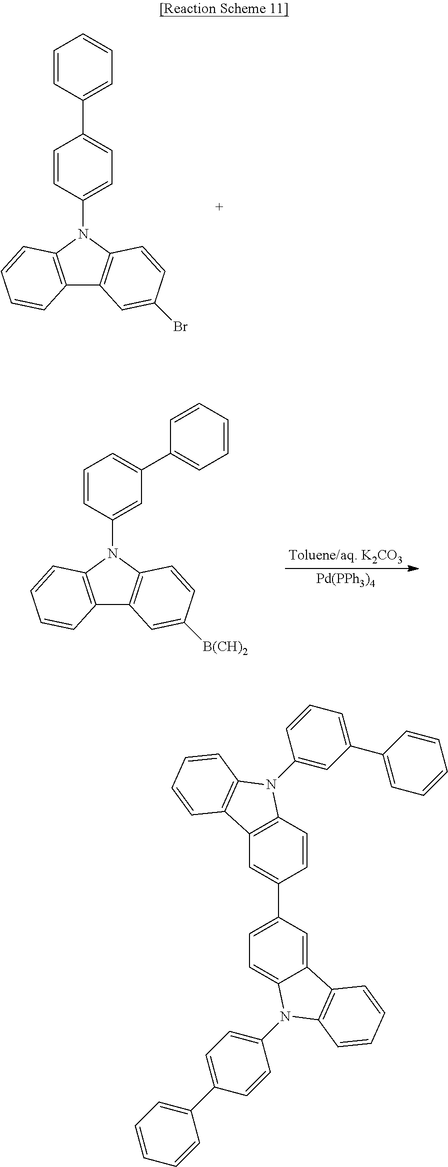

The compound, 9-[1,1′-biphenyl-4-yl]-3-bromo-9H-carbazole (12.33 g, 30.95 mmol) was dissolved in toluene (0.2 L) in a nitrogen environment, 9-([1,1′-biphenyl]-3-yl)-9H-carbazole-3-boronic acid (12.37 g, 34.05 mmol) and tetrakis(triphenylphosphine)palladium (1.07 g, 0.93 mmol) were added thereto, and the mixture was stirred. Potassium carbonate saturated in water (12.83 g, 92.86 mmol) was added thereto, and the mixture was heated and refluxed at 120° C. for 12 hours. When the reaction was complete, water was added to the reaction solution, dichloromethane (DCM) was used for extraction, and an extract therefrom was treated with anhydrous MgSO₄ to remove moisture, filtered, and concentrated under a reduced pressure. The obtained residue was separated and purified through flash column chromatography to obtain a compound B-31 (18.7 g, 92%).

HRMS (70 eV, EI+): m/z calcd for C48H32N2: 636.26, found: 636

Elemental Analysis: C, 91%; H, 5%

### SYNTHESIS EXAMPLE 4

### Synthesis of Compound B-130

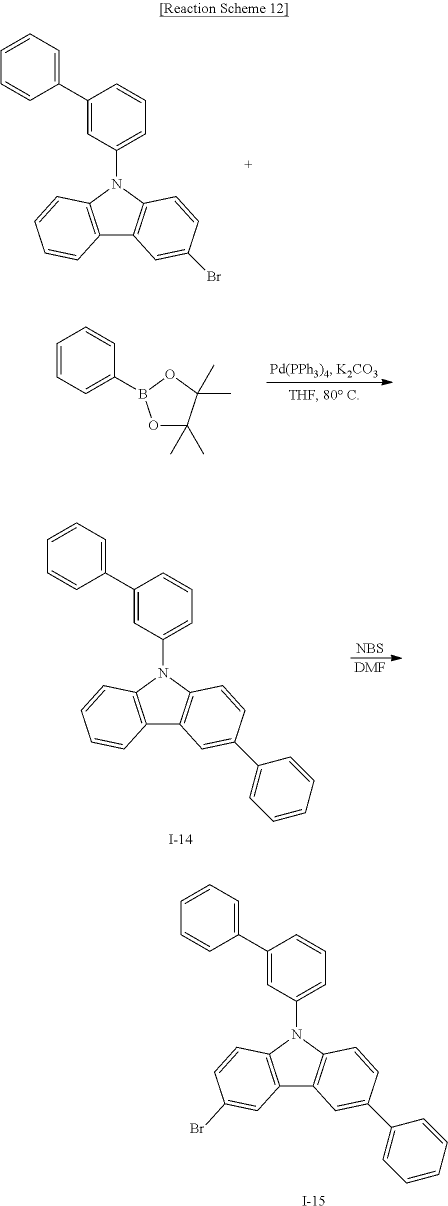

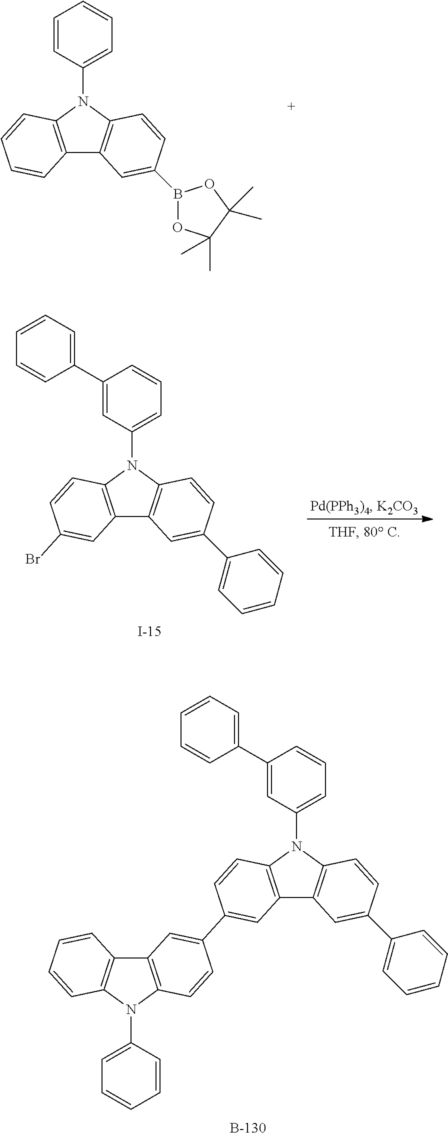

First Step: Synthesis of Compound 1-14

An intermediate I-14 (33 g, 77%) was obtained according to the same method as the method of synthesizing the compound B-31 by using 9-([1,1′-biphenyl]-3-yl)-3-bromo-9H-carbazole (43.2 g, 108.4 mmol) and 4,4,5,5,-tetramethyl-2-Phenyl-1,3,2-dioxaborolane (14.5 g, 119 mmol).

Second Step: Synthesis of Intermediate I-15

The intermediate I-14 (29.8 g, 75.28 mmol) was stirred with N-bromosuccinimide (14 g, 75.28 mmol) at room temperature. When the reaction was complete, water was added to the reaction solution, dichloromethane (DCM) was used for extraction, and an extract therefrom was treated with anhydrous MgSO₄ to remove moisture, filtered, and concentrated under a reduced pressure. The obtained residue was separated and purified through flash column chromatography to obtain an intermediate I-15 (29 g, 81%).

Third Step: Synthesis of Compound B-130

A compound B-130 (17 g, 79%) was synthesized according to the same method as the method of synthesizing the compound B-31 by using 9-phenyl-3-(4,4,5,5,-tetramethyl-1,3,2-dioxaborolan-2-yl)-9H-carbazole (9.7 g, 33.65 mmol) and the intermediate I-15 (16 g, 33.65 mmol).

HRMS (70 eV, EI+): m/z calcd for C48H32N2: 636.2565, found: 636

Elemental Analysis: C, 90%; 5%

Synthesis of Third Compound

### SYNTHESIS EXAMPLE 5

### Synthesis of Compound F-5

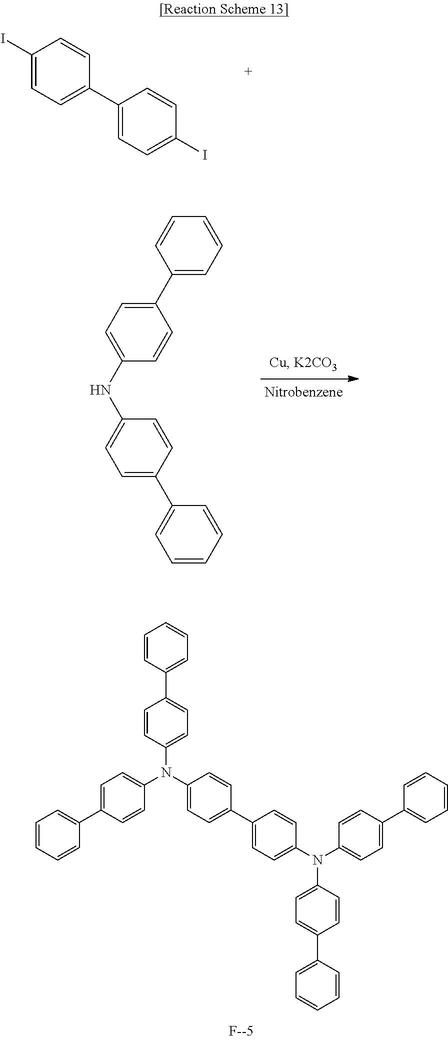 [SMILES: Ic1ccc(-c2ccc(I)cc2)cc1 ; c1ccc(-c2ccc(Nc3ccc(-c4ccccc4)cc3)cc2)cc1 ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1]

6.4g (yield 25%) of the Compound F-5 was synthesized according to Japanese Publication No. 1996-048656.

HRMS (70 eV, EI+): m/z calcd for C60H44N2: 792.0048, found: 792.

Elemental Analysis: C, 91%; H, 6%

### SYNTHESIS EXAMPLE 6

### Synthesis of Compound G-4

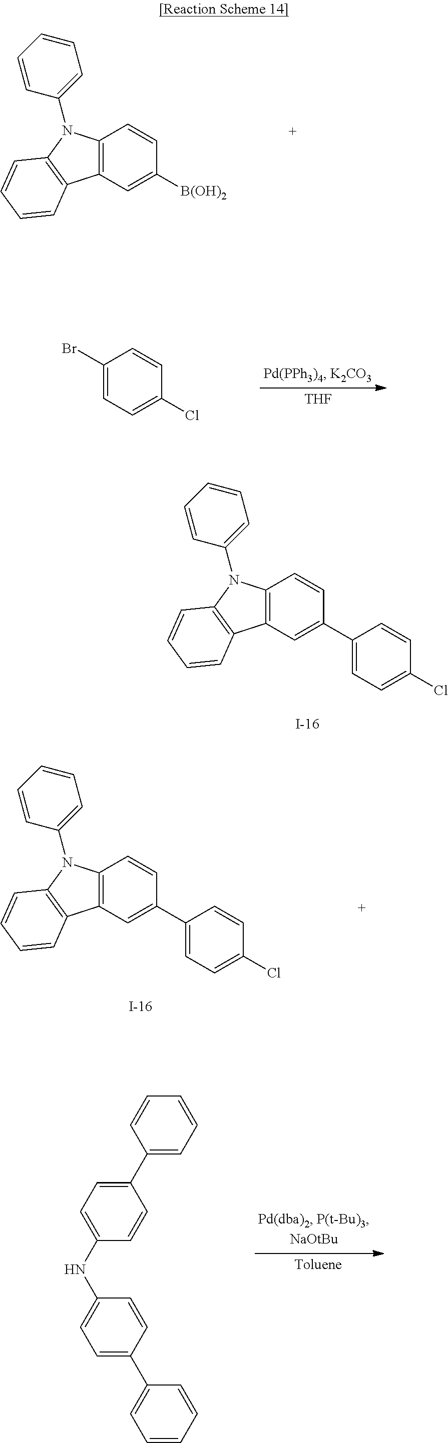

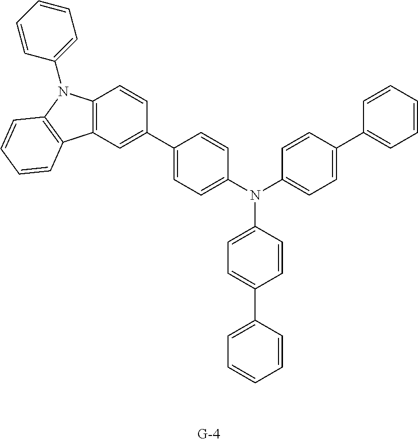 [SMILES: c1ccc(-c2ccc(N(c3ccc(-c4ccccc4)cc3)c3ccc(-c4ccc5c(c4)c4ccccc4n5-c4ccccc4)cc3)cc2)cc1]

First Step: Synthesis of Intermediate I-16

9-phenyl-9H-carbazol-3-yl boronic acid (100 g, 348 mmol) was dissolved in 0.9 L of tetrahydrofuran (THF) in a nitrogen environment, and 1-bromo-4-chlorobenzene (73.3 g, 383 mmol) and tetrakis(triphenylphosphine)palladium (4.02 g, 3.48 mmol) were added and stirred. Potassium carbonate (128 g, 870 mmol) saturated in water was added thereto, and the resulting mixture was heated and refluxed at 80° C. for 8 hours. When the reaction was complete, water was added to the reaction solution, dichloromethane (DCM) was used for extraction, and an extract therefrom was treated with anhydrous MgSO₄ to remove moisture, filtered, and concentrated under a reduced pressure. The obtained residue was separated and purified through flash column chromatography to obtain an intermediate I-16 (119 g, 97%).

HRMS (70 eV, EI+): m/z calcd for C24H16ClN: 353.0971, found: 353.

Elemental Analysis: C, 81%; H, 5%

Second Step: Synthesis of Compound G-4

The intermediate I-16 (20 g, 56.5 mmol) was dissolved in 0.2 L of toluene under a nitrogen environment, dibiphenyl-4-ylamine (18.2 g, 56.5 mmol) of Shenzhen gre-syn chemical technology (http://www.gre-syn.com/), bis(dibenzylideneacetone)palladium (0) (0.33 g, 0.57 mmol), tris-tert butylphosphine (0.58 g, 2.83 mmol), and sodium tert-butoxide (6.52 g, 67.8 mmol) were sequentially added, and the mixture was heated and refluxed at 100° C. for 15 hours. When the reaction was complete, water was added to the reaction solution, dichloromethane (DCM) was used for extraction, and an extract therefrom was treated with anhydrous MgSO₄ to remove moisture, filtered, and concentrated under a reduced pressure. The obtained residue was separated and purified through flash column chromatography to obtain a compound G-4 (32.5 g, 90%).

HRMS (70 eV, EI+): m/z calcd for C48H34N2: 638.2722, found: 638.

Elemental Analysis: C, 90%; H, 5%

### SYNTHESIS EXAMPLE 7

### Synthesis of Compound G-9

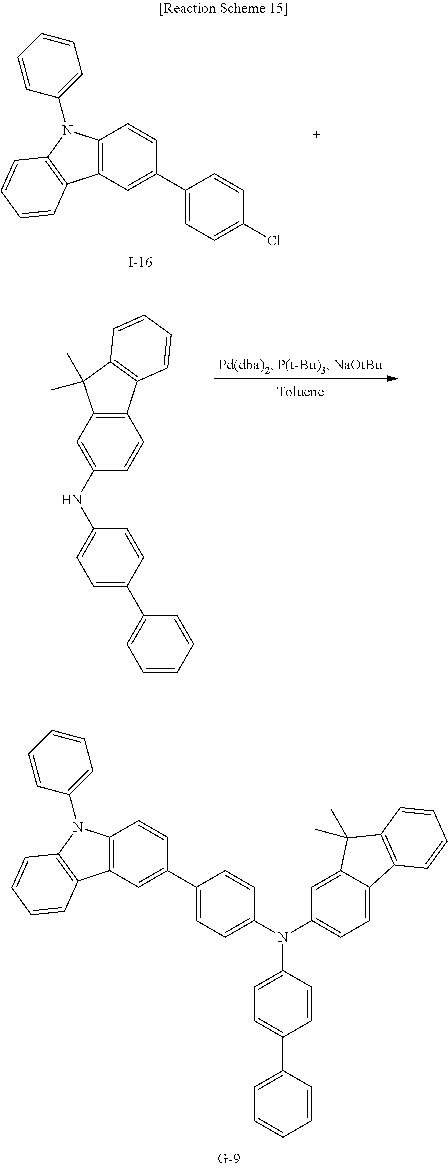

The intermediate I-16 (20 g, 56.5 mmol) was dissolved in 0.2 L of toluene under a nitrogen environment, N-(biphenyl-4-yl)-9,9-dimethyl-9H-fluoren-2-amine (20.4 g, 56.5 mmol) of Shenzhen gre-syn chemical technology (http://www.gre-syn.com/), bis(dibenzylideneacetone)palladium (0) (0.33 g, 0.57 mmol), tris-tert butylphosphine (0.58 g, 2.83 mmol), and sodium tert-butoxide (6.52 g, 67.8 mmol) were sequentially added, and the mixture was heated and refluxed at 100° C. for 13 hours. When the reaction was complete, water was added to the reaction solution, dichloromethane (DCM) was used for extraction, and an extract therefrom was treated with anhydrous MgSO₄ to remove moisture, filtered, and concentrated under a reduced pressure. The obtained residue was separated and purified through flash column chromatography to obtain a compound G-9 (33.8 g, 88%).

HRMS (70 eV, EI+): m/z calcd for C48H34N2: 678.3045. found: 678.

Elemental Analysis: C, 90%; H, 6%

### SYNTHESIS EXAMPLE 8

### Synthesis of Compound G-32

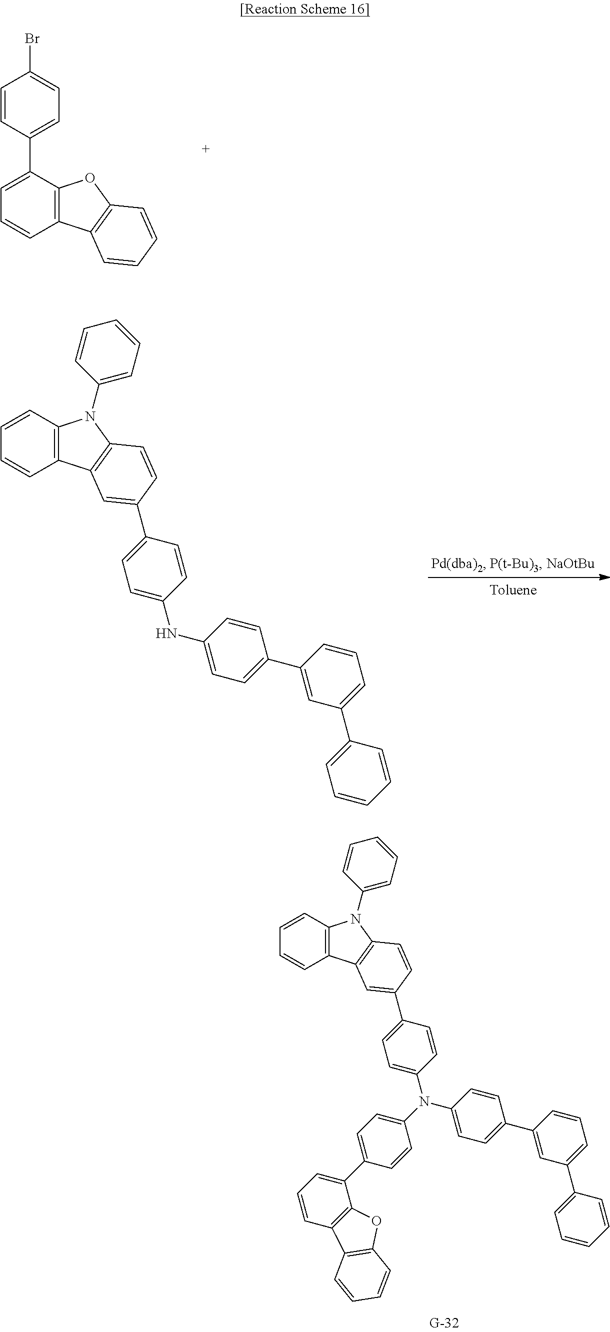

10.4g (yield 82%) of the Compound G-32 was synthesized according to KR 2011-0118542.

HRMS (70 eV, EI+): m/z calcd for C60H40N2: 804.3140, found: 804.

Elemental Analysis: C, 90%; H, 5%

Manufacture of Organic Light Emitting Diode

### EXAMPLE 1

ITO (indium tin oxide) was coated to be 1500 Å thick on a glass substrate, and the coated glass was ultrasonic wave-washed with a distilled water. After washed with distilled water, the glass substrate was ultrasonic wave-washed with a solvent such as isopropyl alcohol, acetone, methanol, and the like was moved to a plasma cleaner to clean the substrate by using oxygen plasma for 10 minutes and moved to a vacuum depositor. This ITO transparent electrode was used as an anode, a compound A was vacuum-deposited on the ITO substrate to form a 700 Å-thick hole injection layer, a compound B was deposited in a 50 Å thickness on the injection layer, a compound C was deposited in a 1020 Å thickness to form a hole transport layer. On the hole transport layer, a 400 Å-thick emission layer was formed through vacuum deposition by simultaneously using the first compound A-275 of Synthesis Example 1, the second compound B-31 of Synthesis Example 3, and the third compound G-9 of Synthesis Example 7 as a host and doping them with 10 wt % of tris(2-(3-biphenyl-yl)-pyridine)iridium (III). Herein, the compound A-275 and the compound B-31 were used in a weight ratio 5:5,

a composition of the compounds A-275 and B-31 and the compound G-9 were used in a weight ratio of 9:1.

Subsequently, on the emission layer, the compound D and Liq were simultaneously vacuum-deposited in a ratio of 1:1 to form a 300 Å-thick electron transport layer, and 15 Å Liq and 1200 Å A1 were sequentially vacuum-deposited on the electron transport layer to form a cathode to manufacture an organic light emitting diode.

The organic light emitting diode has a structure including five-layered organic thin layers, and specifically the following structure.

ITO/compound A (700 Å)/compound B (50 Å)/compound C (1020 Å)/EML[{(compound A-275: compound B-31=5:5 wt %):compound G-9}=9:1]:Ir(ppy)₃=X:X:10%]400 Å/compound D:Liq (300 Å)/Liq (15 Å)/A1 (1200 Å). (X=weight ratio)

Compound A: N4,N4′-diphenyl-N4,N4′-bis(9-phenyl-9H-carbazol-3-yl)biphenyl-4,4′-diamine

Compound B: 1,4,5,8,9,11-hexaazatriphenylene-hexacarbonitrile (HAT-CN),

Compound C: N-(biphenyl-4-yl)-9,9-dimethyl-N-(4-(9-phenyl-9H-carbazol-3-yl)phenyl)-9H-fluoren-2-amine

Compound D: 8-(4-(4,6-di(naphthalen-2-yl)-1,3,5-triazin-2-yl)phenyl)quinoline

### EXAMPLE 2

An organic light emitting diode was manufactured according to the same method as Example 1 except that a mixing ratio of a composition of the compound A-275 and the compound B-31 was changed into 7:3.

### EXAMPLE 3

An organic light emitting diode was manufactured according to the same method as Example 1 except that a mixing ratio of a composition of the compound A-275 and the compound B-31 and the compound G-9 was changed into 8:2.

### EXAMPLE 4

An organic light emitting diode was manufactured according to the same method as Example 1 except that the compound G-4 of Synthesis Example 6 was used instead of the third compound G-9 in an emission layer.

### EXAMPLE 5

An organic light emitting diode was manufactured according to the same method as Example 1 except that the compound G-32 of Synthesis Example 8 was used instead of the third compound G-9 in an emission layer.

### EXAMPLE 6

An organic light emitting diode was manufactured according to the same method as Example 1 except that the compound F-5 of Synthesis Example 5 was used instead of the third compound G-9 in an emission layer.

### REFERENCE EXAMPLE 1

An organic light emitting diode was manufactured according to the same method as Example 1 except that the second and third compounds were not used and the first compound was used as a single host.

### COMPARATIVE EXAMPLE 1

An organic light emitting diode was manufactured according to the same method as Example 1 except that the third compound was not used.

### COMPARATIVE EXAMPLE 2

An organic light emitting diode was manufactured according to the same method as Example 2 except that the third compound was not used.

Evaluation

Driving voltage and luminous efficiency characteristics of each organic light emitting diode according to Examples 1 to 6, Reference Example 1, and Comparative Examples 1 and 2 were evaluated.

The measurements were specifically performed in the following methods, and the results are shown in Table 1 and Table 2.

(1) Measurement of Current Density Change Depending on Voltage Change

Current values flowing in the unit device of the manufactured organic light emitting diodes were measured for, while increasing the voltage from 0V to 10V using a current-voltage meter (Keithley 2400), and the measured current values were divided by an area to provide the results.

(2) Measurement of Luminance Change Depending on Voltage Change

Luminance of the manufactured organic light emitting diodes was measured for luminance, while increasing the voltage from 0 V to 10 V using a luminance meter (Minolta Cs-1000 A).

(3) Measurement of Luminous Efficiency

Current efficiency (cd/A) at the same current density (10 mA/cm²) was calculated by using the luminance, current density, and voltages (V) from the items (1) and (2).

(4) Calculation of Luminous Efficiency Ratio

A luminous efficiency increase/decrease degree with a reference to luminous efficiency of Comparative Example 2 was calculated.

(5) Measurement of Driving Voltage

A driving voltage of each device was measured at 15 mA/cm² by using a current-voltage meter (Keithley 2400).

TABLE 1

| | | | | | Composition | | | Light |
|---|---|---|---|---|---|---|---|---|
| | | | First | | of First | | Light | emitting |
| | | | host:Second | | host and | Driving | emitting | efficiency |
| | First | Second | host | Third | second host:third | voltage | efficiency | ratio |
| Devices | host | host | (wt/wt) | host | host | (V) | (lm/W) | (%) |
| Reference | A-275 | — | 100:0 | — | — | 4.13 | 29.0 | 83% |
| Example 1 | | | | | | | | |
| Comparative | A-275 | B-31 | 50:50 | — | — | 4.41 | 34.9 | 100% |
| Example 1 | | | | | | | | |
| Example 1 | A-275 | B-31 | 50:50 | G-9 | 90:10 | 4.02 | 53.4 | 153% |
| Example 4 | A-275 | B-31 | 50:50 | G-4 | 90:10 | 3.84 | 41.5 | 119% |
| Example 5 | A-275 | B-31 | 50:50 | G-32 | 90:10 | 4.10 | 38.5 | 110% |
| Example 6 | A-275 | B-31 | 50:50 | F-5 | 90:10 | 3.90 | 38.0 | 109% |

TABLE 2

| | | | | | Composition | | | Light |
|---|---|---|---|---|---|---|---|---|
| | | | First | | of First | | Light | emitting |
| | | | host:Second | | host and | Driving | emitting | efficiency |
| | First | Second | host | Third | second host:third | voltage | efficiency | ratio |
| Devices | host | host | (wt/wt) | host | host | (V) | (lm/W) | (%) |
| Comparative | A-275 | B-31 | 70:30 | — | — | 4.01 | 33.7 | 100% |
| Example 2 | | | | | | | | |
| Example 2 | A-275 | B-31 | 70:30 | G-9 | 90:10 | 3.79 | 41.1 | 122% |
| Example 3 | A-275 | B-31 | 70:30 | G-9 | 80:20 | 3.71 | 41.7 | 124% |

Referring to Tables 1 and 2, the present invention when including a third host exhibits a lowered driving voltage and significantly increased luminous efficiency compared with Reference Example 1 using only first host and Comparative Examples 1 and 2 using only first and second hosts. This result is obtained by adding the third compound having excellent hole injection and hole transport capability according to the present invention as a host and thus minimizing a trap phenomenon due to an energy level difference between a dopant and a host and improving injection characteristics from the hole transport layer to the emission layer to provide an organic optoelectronic device having excellent luminous efficiency as well as remarkably lowering a driving voltage.

While this invention has been described in connection with what is presently considered to be practical example embodiments, it is to be understood that the invention is not limited to the disclosed embodiments, but, on the contrary, is intended to cover various modifications and equivalent arrangements included within the spirit and scope of the appended claims. Therefore, the aforementioned embodiments should be understood to be exemplary but not limiting the present invention in any way.

### DESCRIPTION OF SYMBOLS

100, 200: organic light emitting diode

105: organic layer

110: cathode

120: anode

130: emission layer

140: auxiliary layer

What is claimed is:

1. A composition for an organic optoelectronic device, comprising:
at least one first compound represented by Chemical Formula 1,
at least one second compound of a compound represented by Chemical Formula 2, and a compound consisting of a combination of a moiety represented by Chemical Formula 3 and a moiety represented by Chemical Formula 4, and
at least one third compound represented by Chemical Formula 5: 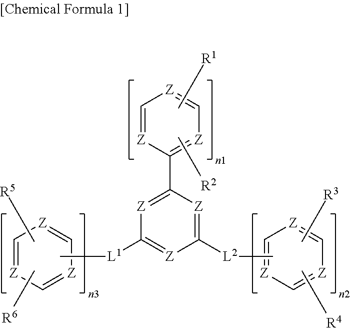
wherein, in Chemical Formula 1,
Z is independently N, C, or CR^{a},
at least one of Z is N,
R¹ to R⁶ and R^{a} are independently hydrogen, deuterium, a substituted or unsubstituted C1 to C10 alkyl group, a substituted or unsubstituted C6 to C30 aryl group, a substituted or unsubstituted C2 to C30 heteroaryl group, or a combination thereof,
R¹ to R⁶ and R^{a} are independently present or adjacent groups are linked to each other to provide a ring,
L¹ and L² are independently a single bond, a substituted or unsubstituted C6 to C30 arylene group, a substituted or unsubstituted C2 to C30 heteroarylene group, or a combination thereof,
n1 is 1,
n2 and n3 are independently an integer of 0 or 1, and
1≦n2+n3≦2; 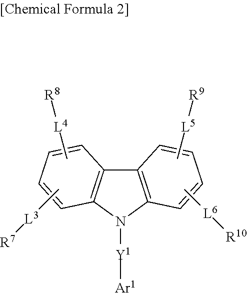
wherein, in Chemical Formula 2,
L³ to L⁶ and Y¹ are independently a single bond, a substituted or unsubstituted C6 to C30 arylene group, a substituted or unsubstituted C2 to C30 heteroarylene group, or a combination thereof,
Ar¹ is a substituted or unsubstituted C6 to C30 aryl group, a substituted or unsubstituted C2 to C30 heterocyclic group, or a combination thereof,
R⁷ to R¹⁰ are independently hydrogen, deuterium, a substituted or unsubstituted C1 to C20 alkyl group, a substituted or unsubstituted C6 to C50 aryl group, a substituted or unsubstituted C2 to C50 heterocyclic group, or a combination thereof, and
at least one of R⁷ to R¹⁰ and Ar¹ includes a substituted or unsubstituted triphenylene group or a substituted or unsubstituted carbazole group, 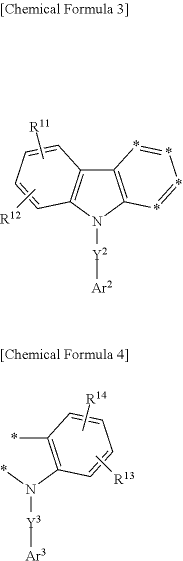
wherein, in Chemical Formulae 3 and 4,
Y² and Y³ are independently a single bond, a substituted or unsubstituted C6 to C30 arylene group, a substituted or unsubstituted C2 to C30 heteroarylene group, or a combination thereof,
Ar² and Ar³ are independently a substituted or unsubstituted C6 to C30 aryl group, a substituted or unsubstituted C2 to C30 heterocyclic group, or a combination thereof,
R¹¹ to R¹⁴ are independently hydrogen, deuterium, a substituted or unsubstituted C1 to C20 alkyl group, a substituted or unsubstituted C6 to C50 aryl group, a substituted or unsubstituted C2 to C50 heterocyclic group, or a combination thereof,
two adjacent *'s of Chemical Formula 3 are linked to two *'s of Chemical Formula 4 to provide a fused ring, and in Chemical Formula 3, *'s that do not provide a fused ring are independently CR^{c}, and
R^{c} is hydrogen, deuterium, a substituted or unsubstituted C1 to C10 alkyl group, a substituted or unsubstituted C6 to C12 aryl group, a substituted or unsubstituted C2 to C12 heterocyclic group, or a combination thereof; 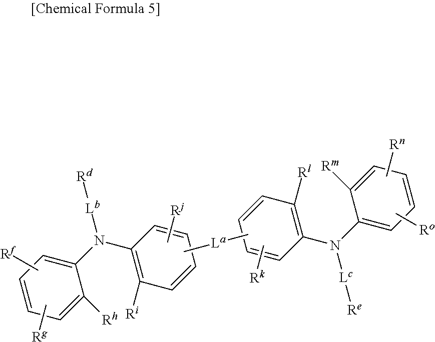
wherein, in Chemical Formula 5,
R^{d} and R^{e} are independently a substituted or unsubstituted C6 to C30 aryl group, a substituted or unsubstituted C2 to C30 heterocyclic group, or a combination thereof
R^{f} to R^{o} are independently hydrogen, deuterium, a substituted or unsubstituted C1 to C30 alkyl group, a substituted or unsubstituted C3 to C30 cycloalkyl group, a substituted or unsubstituted C6 to C30 aryl group, a substituted or unsubstituted C2 to C30 heterocyclic group, a substituted or unsubstituted C6 to C30 arylamine group, a substituted or unsubstituted C1 to C30 alkoxy group, a substituted or unsubstituted C3 to C40 silyl group, a substituted or unsubstituted C3 to C40 silyloxy group, a substituted or unsubstituted C1 to C30 alkylthiol group, a substituted or unsubstituted C6 to C30 arylthiol group, a halogen, a halogen-containing group, a cyano group, a hydroxyl group, an amino group, a nitro group, or a combination thereof,
R^{h}, Rⁱ, R^{l}, and R^{m} are independently present or are linked to each other to provide a ring, provided that when R^{h} and Rⁱ are linked to each other to provide a ring, R^{l} and R^{m} are not linked to each other, and when R^{l} and R^{m} are linked to each other to provide a ring, R^{h} and Rⁱ are not linked to each other, and
L^{a} to L^{c} are independently a single bond, a substituted or unsubstituted C6 to C30 arylene group, a substituted or unsubstituted C2 to C30 heteroarylene group, or a combination thereof,
wherein “substituted” of Chemical Formulae 1 to 5 refers to replacement of at least one hydrogen by deuterium, a halogen, a hydroxyl group, a C1 to C40 silyl group, a C1 to C30 alkyl group, a C3 to C30 cycloalkyl group, a C2 to C30 heterocycloalkyl group, a C6 to C30 aryl group, a C2 to C30 heterocyclic group, a C1 to C20 alkoxy group, a C1 to C10 trifluoroalkyl group, or a cyano group.

2. The composition of claim 1, wherein Chemical Formula 1 is represented by one of Chemical Formula 1-I to Chemical Formula 1-III: 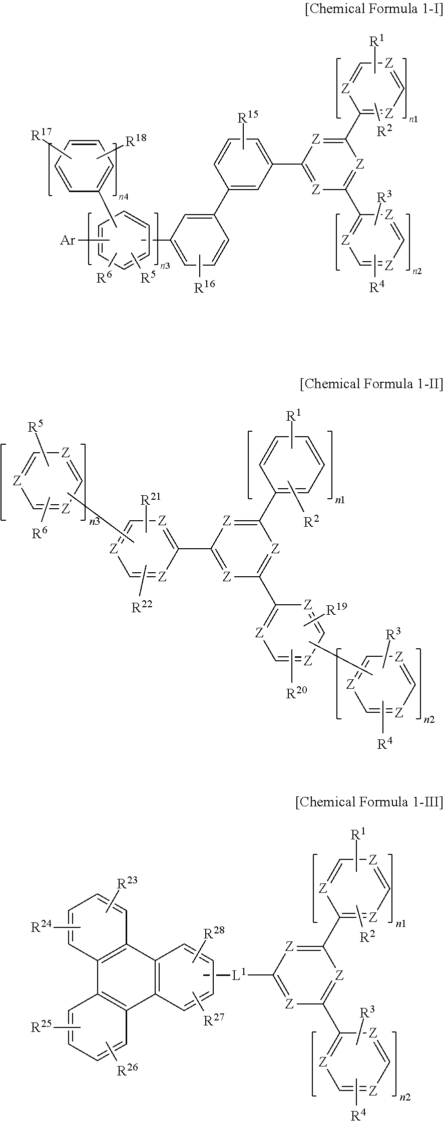
wherein, in Chemical Formulae 1-I to 1-III,
Z is independently N, C, or CR^{a},
at least one of Z is N,
R¹ to R⁶, R¹⁵ to R²⁸, and R^{a} are independently hydrogen, deuterium, a substituted or unsubstituted C1 to C10 alkyl group, a substituted or unsubstituted C6 to C12 aryl group, a substituted or unsubstituted C2 to C30 heteroaryl group, or a combination thereof
Ar is a substituted or unsubstituted C6 to C30 aryl group, a substituted or unsubstituted C2 to C30 heterocyclic group, or a combination thereof,
R¹ to R⁶, R^{a} and R¹⁷ and R¹⁸ are independently present or adjacent groups are linked to each other to provide a ring,
L¹ is a single bond, a substituted or unsubstituted C6 to C30 arylene group, a substituted or unsubstituted C2 to C30 heteroarylene group, or a combination thereof,
n1 is an integer of 1,
n2 is an integer of 0 or 1,
n3 and n4 are independently an integer of 0 to 2, and
1≦n2+n3≦2,
wherein “substituted” is the same as defined in claim 1.

3. The composition of claim 2, wherein Chemical Formula 1-I is represented by one of Chemical Formulae 1-IA to 1-IC: 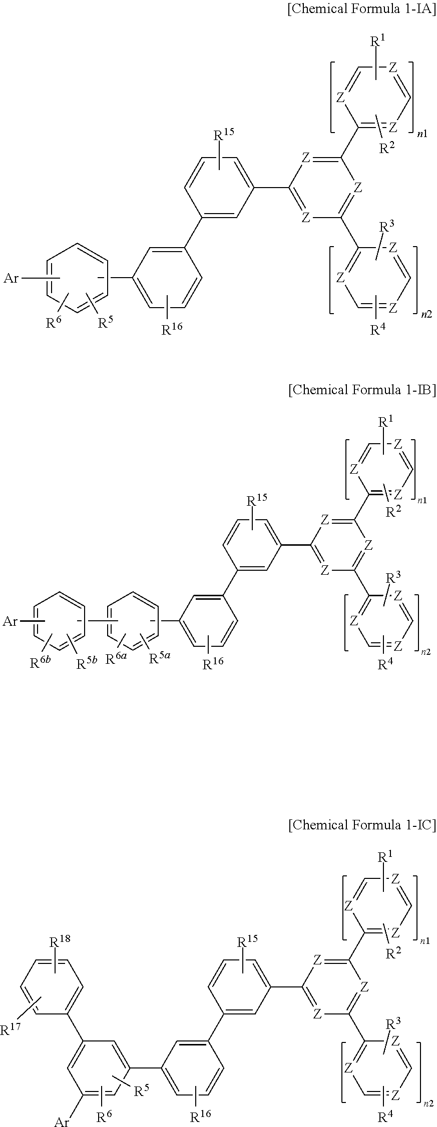
wherein, in Chemical Formulae 1-IA to 1-IC,
Z is independently N, C, or CR^{a},
at least one of Z is N,
R¹ to R⁶, R^{a}, R^{5a}, R^{6a}, R^{5b}, R^{6b}, and R¹⁵ to R¹⁸ are independently hydrogen, deuterium, a substituted or unsubstituted C1 to C10 alkyl group, a substituted or unsubstituted C6 to C12 aryl group, a substituted or unsubstituted C2 to C30 heteroaryl group, or a combination thereof,
R¹ to R⁶, R^{a}, R^{5a}, R^{6a}, R^{5b}, R^{6b}, R¹⁷ and R¹⁸ are independently present or adjacent groups are linked to each other to provide a ring,
Ar is a substituted or unsubstituted C6 to C30 aryl group, a substituted or unsubstituted C2 to C30 heterocyclic group, or a combination thereof
n1 is an integer of 1, and
n2 is an integer of 0 or 1,
wherein “substituted” refers to replacement of at least one hydrogen by deuterium, a halogen, a hydroxyl group, a C1 to C40 silyl group, a C1 to C30 alkyl group, a C3 to C30 cycloalkyl group, a C2 to C30 heterocycloalkyl group, a C6 to C30 aryl group, a C2 to C30 heterocyclic group, a C1 to C20 alkoxy group, a C1 to C10 trifluoroalkyl group, or a cyano group.

4. The composition of claim 2, wherein Chemical Formula 1-II is represented by Chemical Formulae 1-IIA or 1-IIB: 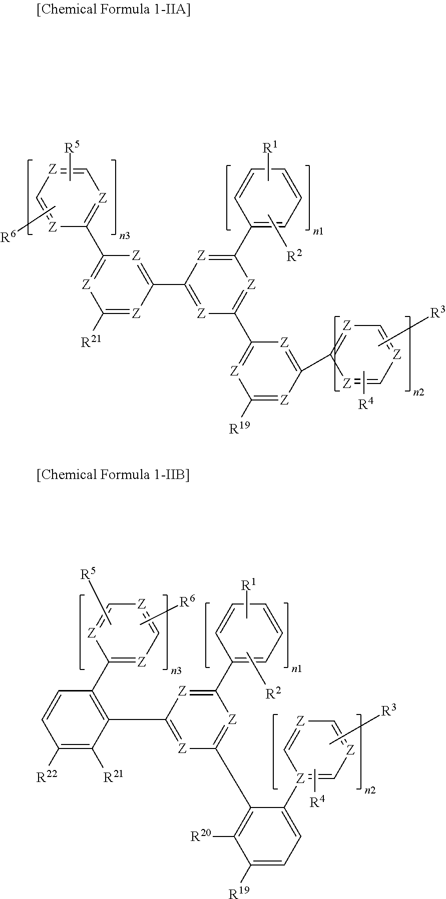
wherein, in Chemical Formulae 1-IIA and 1-IIB,
Z is independently N, C, or CR^{a},
at least one of Z is N,
R¹ to R⁶, R¹⁹ to R²² and R^{a} are independently hydrogen, deuterium, a substituted or unsubstituted C1 to C10 alkyl group, a substituted or unsubstituted C6 to C12 aryl group, a substituted or unsubstituted C2 to C30 heteroaryl group, or a combination thereof,
R¹ to R⁶, R¹⁹ to R²² and R^{a} are independently present or adjacent groups are linked to each other to provide a ring,
n1 is an integer of 1,
n2 and n3 are independently an integer of 0 or 1, and
1≦n2+n3≦2,
wherein “substituted” refers to replacement of at least one hydrogen by deuterium, a halogen, a hydroxyl group, a C1 to C40 silyl group, a C1 to C30 alkyl group, a C3 to C30 cycloalkyl group, a C2 to C30 heterocycloalkyl group, a C6 to C30 aryl group, a C2 to C30 heterocyclic group, a C1 to C20 alkoxy group, a C1 to C10 trifluoroalkyl group, or a cyano group.

5. The composition of claim 2, wherein Chemical Formula 1-III is represented by Chemical Formula 1-IIIA or 1-IIIB: 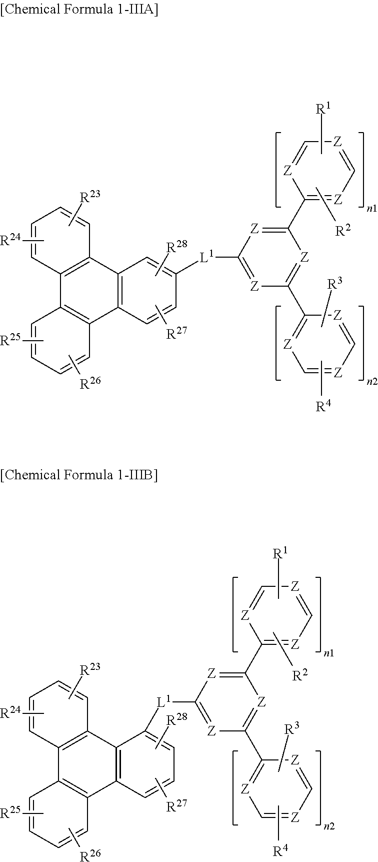
wherein, in Chemical Formulae 1-IIIA and 1-IIIB,
Z is independently N, C, or CR^{a},
at least one of Z is N,
R¹ to R⁴, R²³ to R²⁸, and R^{a} are independently hydrogen, deuterium, a substituted or unsubstituted C1 to C10 alkyl group, a substituted or unsubstituted C6 to C12 aryl group, a substituted or unsubstituted C2 to C30 heteroaryl group, or a combination thereof,
R¹ to R⁴, and R^{a} are independently present or adjacent groups are linked to each other to provide a ring,
L¹ is a single bond, a substituted or unsubstituted phenylene group, a substituted or unsubstituted biphenylene group or a substituted or unsubstituted terphenylene group,
n1 is an integer of 1, and
n2 is an integer of 0 or 1,
wherein “substituted” refers to replacement of at least one hydrogen by deuterium, a halogen, a hydroxyl group, a C1 to C40 silyl group, a C1 to C30 alkyl group, a C3 to C30 cycloalkyl group, a C2 to C30 heterocycloalkyl group, a C6 to C30 aryl group, a C2 to C30 heterocyclic group, a C1 to C20 alkoxy group, a C1 to C10 trifluoroalkyl group, or a cyano group.

6. The composition of claim 1, wherein Chemical Formula 2 is represented by one of Chemical Formulae 2-I to 2-III: 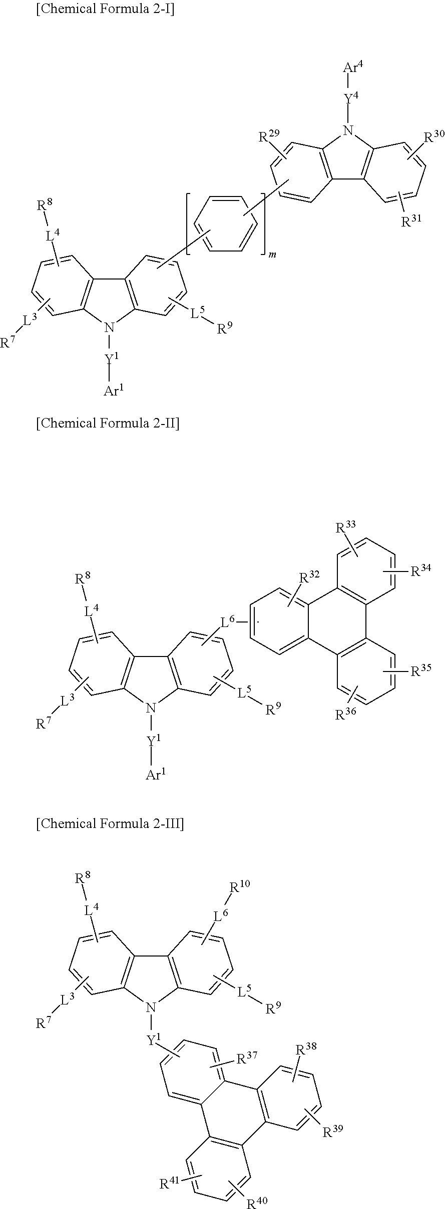
wherein, in Chemical Formulae 2-I to 2-III,
L³ to L⁶, Y¹ and Y⁴ are independently a single bond, a substituted or unsubstituted C6 to C30 arylene group, a substituted or unsubstituted C2 to C30 heteroarylene group, or a combination thereof,
Ar¹ and Ar⁴ are independently a substituted or unsubstituted C6 to C30 aryl group, a substituted or unsubstituted C2 to C30 heterocyclic group, or a combination thereof,
R⁷ to R¹⁰ and R²⁹ to R⁴¹ are independently hydrogen, deuterium, a substituted or unsubstituted C1 to C20 alkyl group, a substituted or unsubstituted C6 to C50 aryl group, a substituted or unsubstituted C2 to C50 heterocyclic group, or a combination thereof,
m is an integer of 0 to 4,
wherein “substituted” is the same as defined in claim 1.

7. The composition of claim 6, wherein Ar¹ and Ar⁴ of Chemical Formula 2-I to 2-III are independently a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted carbazolyl group, a substituted or unsubstituted benzofuranyl group, a substituted or unsubstituted benzothiophenyl group, a substituted or unsubstituted fluorenyl group, a substituted or unsubstituted dibenzofuranyl group, a substituted or unsubstituted dibenzothiophenyl group, a substituted or unsubstituted pyridinyl group, or a combination thereof.

8. The composition of claim 6, wherein Chemical Formula 2-I is one of structures of Group 3, the *—Y¹—Ar¹ and *—Y⁴—Ar⁴ are one of substituents of Group 4: 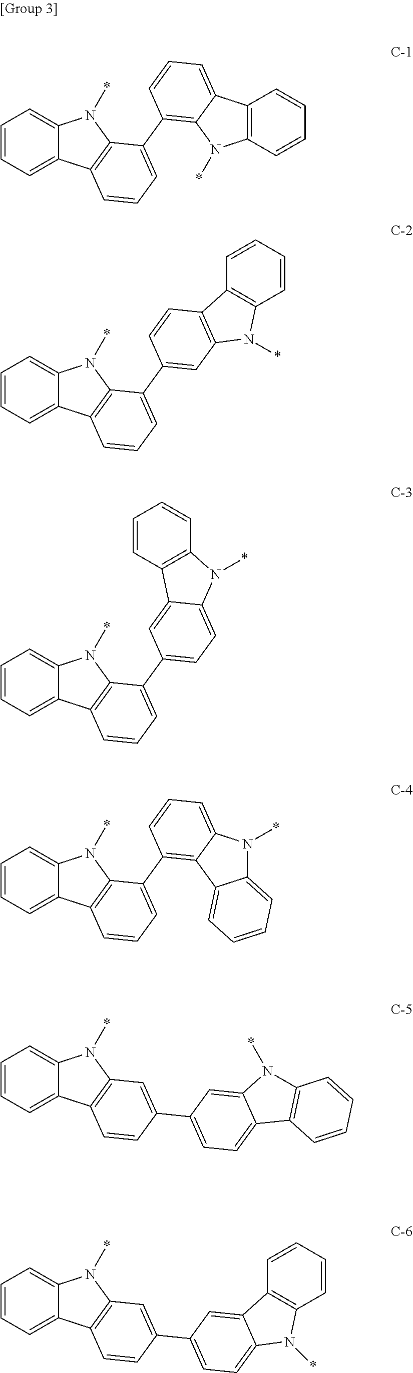 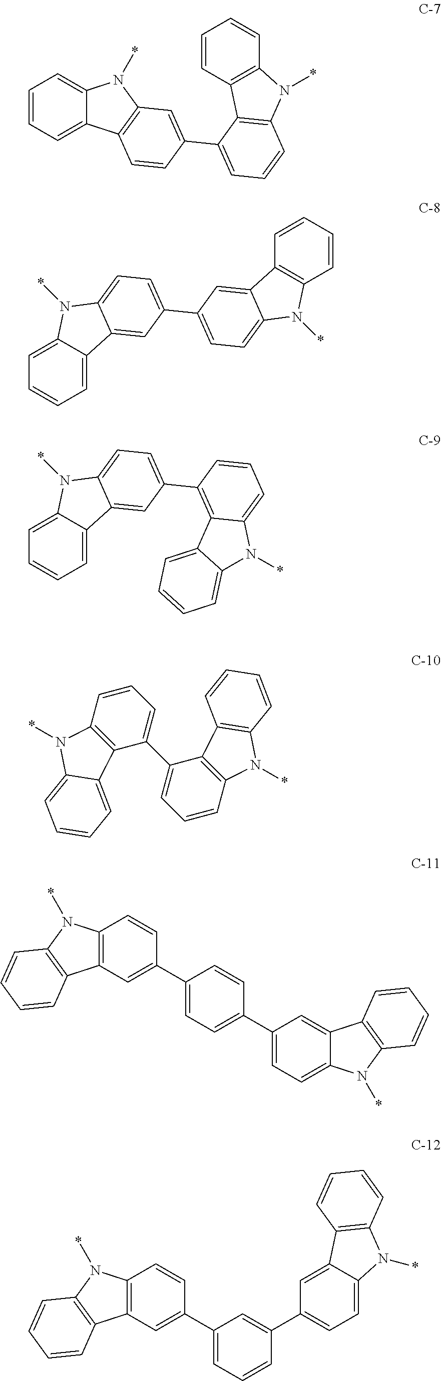 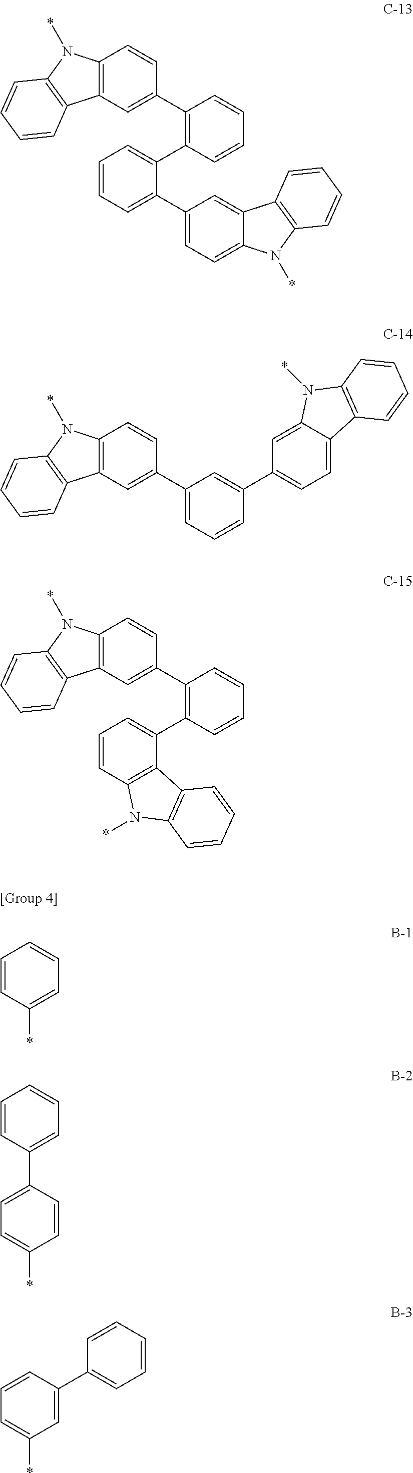 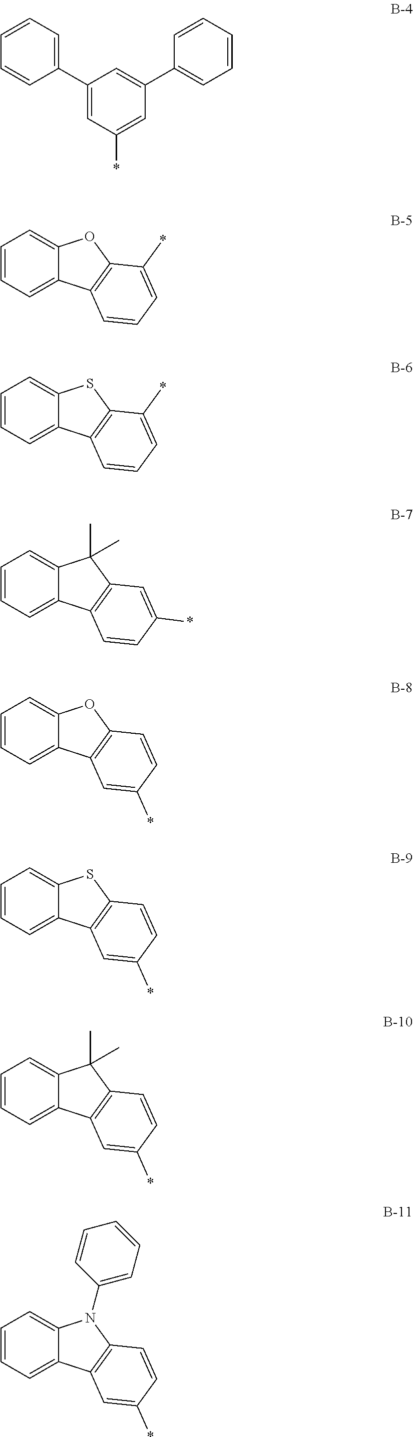 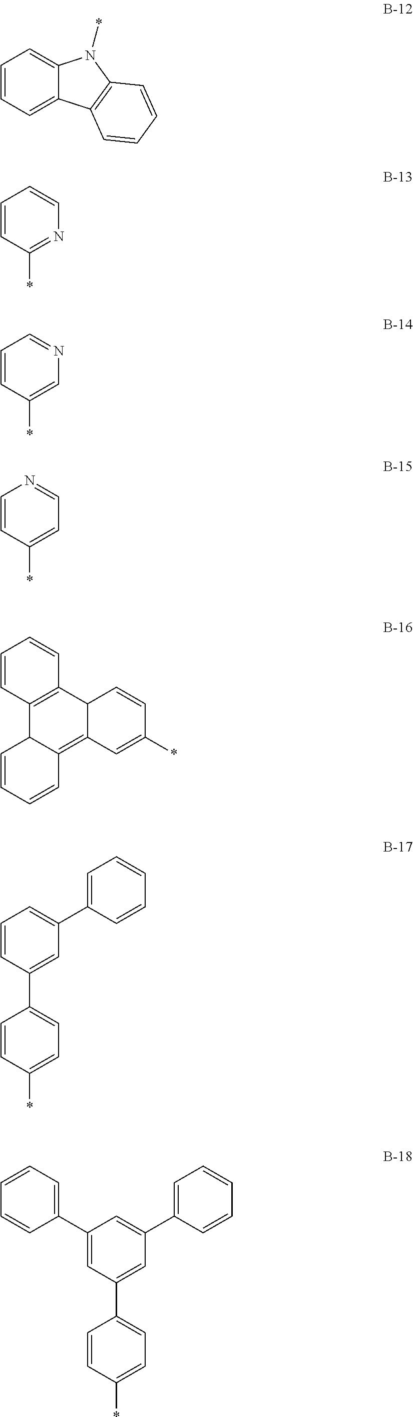
wherein, in Group 3 and Group 4, * is a linking point.

9. The composition of claim 1, wherein the second compound consisting of a combination of the moiety represented by Chemical Formula 3 and the moiety represented by Chemical Formula 4 is represented by one of Chemical Formulae 3-I to 3-V: 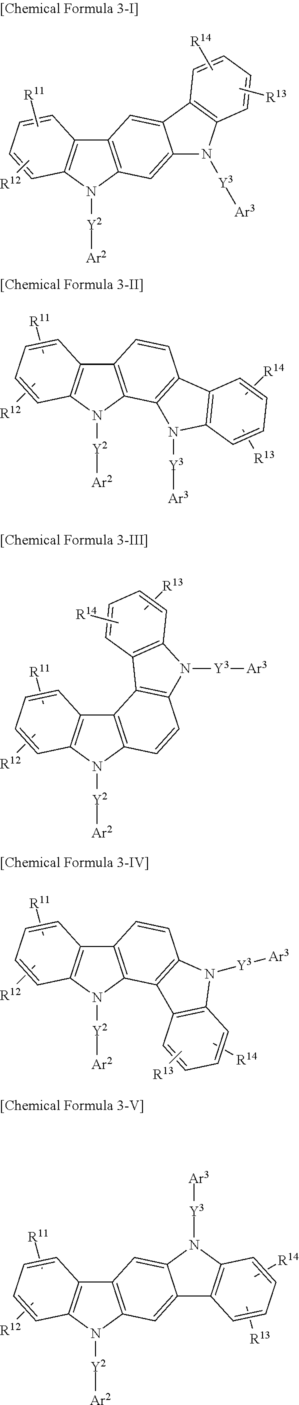
wherein, in Chemical Formulae 3-I to 3-V,
Y² and Y³ are independently a single bond, a substituted or unsubstituted C1 to C20 alkylene group, a substituted or unsubstituted C2 to C20 alkenylene group, a substituted or unsubstituted C6 to C30 arylene group, a substituted or unsubstituted C2 to C30 heteroarylene group, or a combination thereof,
Ar² and Ar³ are independently a substituted or unsubstituted C6 to C30 aryl group, a substituted or unsubstituted C2 to C30 heterocyclic group, or a combination thereof, and
R¹¹ to R¹⁴ are independently hydrogen, deuterium, a substituted or unsubstituted C1 to C20 alkyl group, a substituted or unsubstituted C6 to C50 aryl group, a substituted or unsubstituted C2 to C50 heterocyclic group, or a combination thereof,
wherein, “substituted” is the same as defined in claim 1.

10. The composition of claim 1, wherein Chemical Formula 5 is represented by Chemical Formulae 5-I to 5-II: 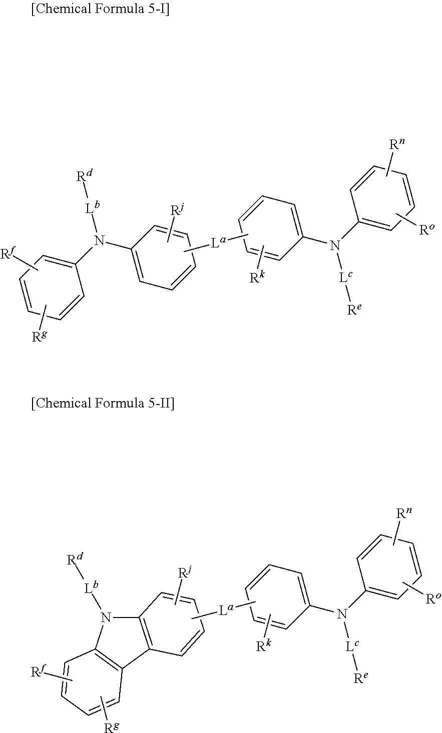
wherein, in Chemical Formulae 5-I to 5-II,
R^{d} and R^{e} are independently, a substituted or unsubstituted C6 to C30 aryl group, a substituted or unsubstituted C2 to C30 heterocyclic group, or a combination thereof,
R^{f}, R^{g}, R^{j}, R^{k}, Rⁿ, and R^{o} are independently hydrogen, deuterium, a substituted or unsubstituted C1 to C30 alkyl group, a substituted or unsubstituted C3 to C30 cycloalkyl group, a substituted or unsubstituted C6 to C30 aryl group, a substituted or unsubstituted C2 to C30 heterocyclic group, a substituted or unsubstituted C6 to C30 arylamine group, a substituted or unsubstituted C1 to C30 alkoxy group, a substituted or unsubstituted C3 to C40 silyl group, a substituted or unsubstituted C3 to C40 silyloxy group, a substituted or unsubstituted C1 to C30 alkylthiol group, a substituted or unsubstituted C6 to C30 arylthiol group, a halogen, a halogen-containing group, a cyano group, a hydroxyl group, an amino group, a nitro group, or a combination thereof, and
L^{a} to L^{c} are independently a single bond, a substituted or unsubstituted C6 to C30 arylene group, a substituted or unsubstituted C2 to C30 heteroarylene group, or a combination thereof,
wherein, “substituted” is the same as defined in claim 1.

11. The composition of claim 1, wherein R^{d} to R^{o} of Chemical Formula 5 are independently hydrogen, deuterium, a substituted or unsubstituted phenyl group, a substituted or unsubstituted biphenyl group, a substituted or unsubstituted terphenyl group, a substituted or unsubstituted quaterphenyl group, a substituted or unsubstituted naphthyl group, a substituted or unsubstituted anthracenyl group, a substituted or unsubstituted spirofluorenyl group, a substituted or unsubstituted triphenylene group, a substituted or unsubstituted fluorenyl group, a substituted or unsubstituted dibenzofuranyl group, a substituted or unsubstituted dibenzothiophenyl group, a substituted or unsubstituted carbazolyl group, or a combination thereof.

12. The composition of claim 1, the first compound is represented by Chemical Formula 1-I or Chemical Formula 1-III,
the second compound is represented by Chemical Formula 2-I, and
the third compound is represented by Chemical Formula 5-I or Chemical Formula 5-II: 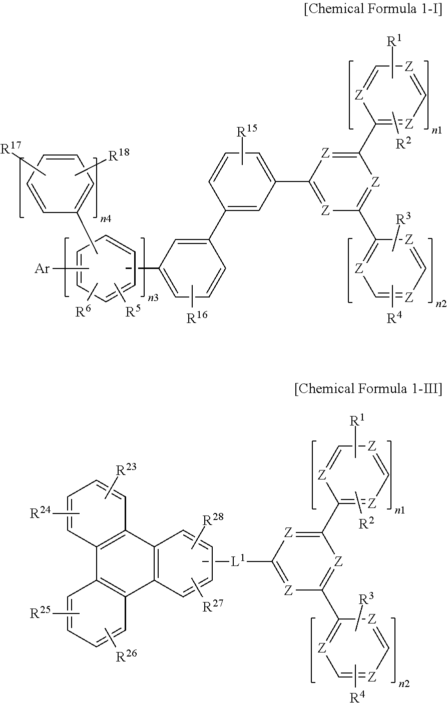
wherein, in Chemical Formulae 1-I and 1-III,
Z is independently N, C, or CR^{a},
at least one of Z is N,
R¹ to R⁶, R¹⁵ to R¹⁸, R²³ to R²⁸, and R^{a} are independently hydrogen, deuterium, a substituted or unsubstituted C1 to C10 alkyl group, or a substituted or unsubstituted C6 to C12 aryl group,
Ar is independently a substituted or unsubstituted C6 to C30 aryl group, a substituted or unsubstituted C2 to C30 heterocyclic group, or a combination thereof,
R¹ to R⁶, R¹⁵ to R¹⁸, R²³ to R²⁸, and R^{a} are independently present or adjacent groups are linked to each other to provide a ring,
L¹ is a single bond, a substituted or unsubstituted phenylene group, a substituted or unsubstituted biphenylene group, a substituted or unsubstituted terphenylene group, a substituted or unsubstituted quaterphenylene group, a substituted or unsubstituted naphthylene group, a substituted or unsubstituted anthracenylene group, a substituted or unsubstituted triphenylenyl group, or a substituted or unsubstituted phenanthrenylene group,
n1 is an integer of 1,
n2 and n3 are independently an integer of 0 or 1, and
n4 is an integer ranging from 0 to 2; 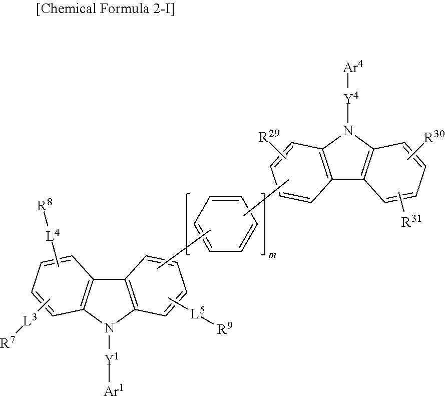
wherein, in Chemical Formula 2-I,
L³ to L⁵, Y¹ and Y⁴ are independently a single bond, or a substituted or unsubstituted C6 to C30 arylene group,
Ar¹ and Ar⁴ are independently a substituted or unsubstituted C6 to C30 aryl group, a substituted or unsubstituted C2 to C30 heterocyclic group, or a combination thereof
R⁷ to R⁹ and R²⁹ to R³¹ are independently hydrogen, deuterium, a substituted or unsubstituted C1 to C20 alkyl group, or a substituted or unsubstituted C6 to C30 aryl group, and
m is an integer of 0 or 1; 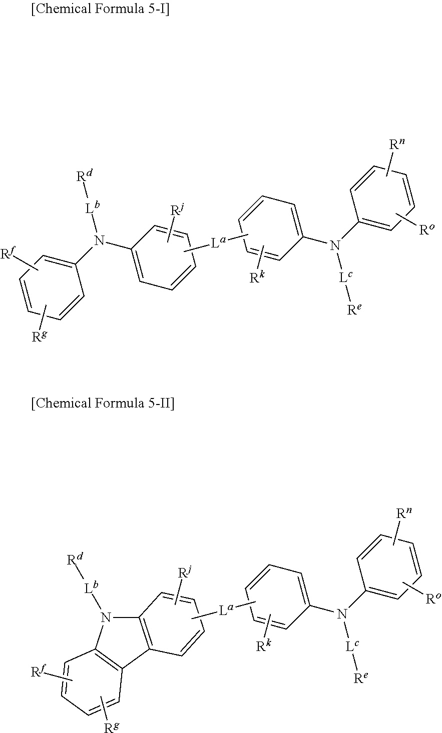
wherein, in Chemical Formulae 5-I and 5-II,
R^{d}, R^{e}, Rⁿ, and R^{o} are independently hydrogen, a substituted or unsubstituted phenyl group, a substituted or unsubstituted biphenyl group, a substituted or unsubstituted terphenyl group, a substituted or unsubstituted quaterphenyl group, a substituted or unsubstituted naphthyl group, a substituted or unsubstituted anthracenyl group, a substituted or unsubstituted spirofluorenyl group, a substituted or unsubstituted triphenylene group, a substituted or unsubstituted fluorenyl group, a substituted or unsubstituted dibenzofuranyl group, a substituted or unsubstituted dibenzothiophenyl group, a substituted or unsubstituted carbazolyl group, or a combination thereof,
R^{f}, R^{g}, R^{j}, and R^{k} are independently hydrogen, and
L^{a} to L^{c} are independently a single bond, a substituted or unsubstituted phenylene group, or a substituted or unsubstituted biphenylene group, or a combination thereof,
wherein, “substituted” is the same as defined in claim 1.

13. The composition of claim 12, wherein the first compound is represented by Chemical Formula 1-IB or 1-IIIA: 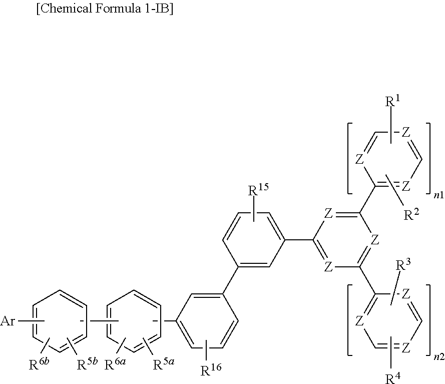
wherein, in Chemical Formula 1-IB,
Z is independently N, C, or CR^{a},
at least one of Z is N,
R¹ to R⁴, R¹⁵, R¹⁶, R^{5a}, R^{6a}, R^{5b}, and R^{6b} are independently hydrogen, deuterium, a substituted or unsubstituted C1 to C10 alkyl group, or a substituted or unsubstituted C6 to C12 aryl group,
Ar is independently a substituted or unsubstituted C6 to C30 aryl group, a substituted or unsubstituted C2 to C30 heterocyclic group, or a combination thereof,
R^{5a} and R^{6a} and R^{5b}, and R^{6b} are independently present or adjacent groups are linked to each other to provide a ring,
n1 is an integer of 1, and
n2 is an integer of 0 or 1; 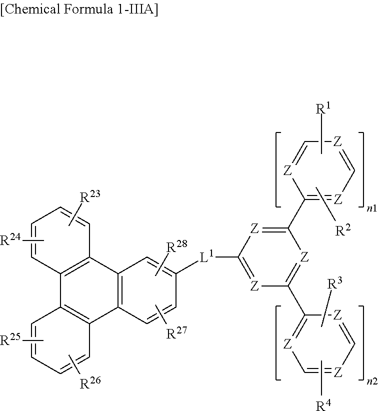
wherein, in Chemical Formula 1-IIIA,
Z is independently N, C, or CR^{a},
at least one of Z is N,
R¹ to R⁴, R²³ to R²⁸ and R^{a} are independently hydrogen, deuterium, a substituted or unsubstituted C1 to C10 alkyl group, a substituted or unsubstituted C6 to C12 aryl group, a substituted or unsubstituted C2 to C30 heteroaryl group, or a combination thereof
R¹ to R⁴ and R^{a} are independently present or adjacent groups are linked to each other to provide a ring,
L¹ is a single bond, a substituted or unsubstituted phenylene group, a substituted or unsubstituted biphenylene group, a substituted or unsubstituted terphenylene group, a substituted or unsubstituted quaterphenylene group, a substituted or unsubstituted naphthylene group, a substituted or unsubstituted anthracenylene group, a substituted or unsubstituted triphenylenyl group, or a substituted or unsubstituted phenanthrenylene group,
n1 is an integer of 1, and
n2 is an integer of 0 or 1;
wherein “substituted” refers to replacement of at least one hydrogen by deuterium, a halogen, a hydroxyl group, a C1 to C40 silyl group, a C1 to C30 alkyl group, a C3 to C30 cycloalkyl group, a C2 to C30 heterocycloalkyl group, a C6 to C30 aryl group, a C2 to C30 heterocyclic group, a C1 to C20 alkoxy group, a C1 to C10 trifluoroalkyl group, or a cyano group.

14. The composition of claim 1, wherein the composition further includes a phosphorescent dopant.

15. An organic optoelectronic device comprising
an anode and a cathode facing each other and
at least one organic layer between the anode and the cathode
wherein the organic layer includes the composition for an organic optoelectronic device of claim 1.

16. A display device comprising the organic optoelectronic device of claim 15.
